(19) United States
(12) Patent Application Publication
(10) Pub. No.: US 2025/0386728 A1
(43) Pub. Date: Dec. 18, 2025

(54) Organic Compound and Light-Emitting Device

(71) Applicant: Semiconductor Energy Laboratory Co., Ltd., Kanagawa-ken (JP)

(72) Inventors: Kazuki KAJIYAMA, Hadano (JP); Hiromitsu KIDO, Atsugi (JP); Sachiko KAWAKAMI, Atsugi (JP); Tomohiro KUBOTA, Atsugi (JP)

(73) Assignee: Semiconductor Energy Laboratory Co., Ltd., Kanagawa-ken (JP)

(21) Appl. No.: 19/233,242

(22) Filed: Jun. 10, 2025

(30) Foreign Application Priority Data
Jun. 14, 2024 (JP) 2024-096666

(51) Int. Cl. H10K 85/60; C07B 59/00; C07D 209/86; C09K 11/02; H10K 50/11

(52) U.S. Cl. CPC H10K 85/6572; C07B 59/002; C07D 209/86; C09K 11/02; H10K 85/622; H10K 85/626; H10K 50/11

(57) ABSTRACT

An organic compound with low synthesis cost and a light-emitting device with a long lifetime are provided. An organic compound represented by General Formula (G4) is provided. Note that Ar¹ and Ar² are each independently a group represented by any one of General Formulae (Ar-1) to (Ar-4) and include the same fused ring, and R¹ to R¹⁴ each independently represent hydrogen (including deuterium). In General Formulae (Ar-1) to (Ar-4), R¹⁵ to R⁵⁰ each independently represent hydrogen (including deuterium) or an alkyl group having 1 to 6 carbon atoms. Note that the organic compound represented by General Formula (G4) contains two or more deuteriums.

### BACKGROUND OF THE INVENTION

### 1. Field of the Invention

One embodiment of the present invention relates to an organic compound, a light-emitting device, a light-receiving device, a light-emitting and light-receiving device, a light-emitting apparatus, a light-emitting and light-receiving apparatus, a display device, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display device, a liquid crystal display device, a light-emitting apparatus, a lighting device, a power storage device, a memory device, an image capturing device, a driving method thereof, and a manufacturing method thereof.

### 2. Description of the Related Art

Light-emitting devices (organic EL devices) that include organic compounds and utilize electroluminescence (EL) have been put to practical use. In the basic structure of such light-emitting devices, an organic compound layer (EL layer) including a light-emitting material is sandwiched between a pair of electrodes. Carriers are injected by application of voltage to the device, and recombination energy of the carriers is used, whereby light emission can be obtained from the light-emitting material.

Such light-emitting devices are of self-luminous type and thus have advantages over liquid crystal devices such as high visibility and no need for backlight when used in pixels of a display device, and are suitable as devices used in flat panel displays. A display device including such a light-emitting device is also highly advantageous in that it can be thin and lightweight. Another feature of such a light-emitting device is that it has an extremely high response speed.

Display devices including light-emitting devices are suitable for a variety of electronic appliances and research and development of light-emitting devices have progressed for more favorable characteristics. For example, Patent Document 1 discloses a light-emitting device including an iridium complex containing deuterium, which achieves a long lifetime.

### REFERENCES

### Patent Document

[Patent Document 1] Japanese Published Patent Application No. 2015-227374

### Non-Patent Documents

[Non-Patent Document 1] Dolomanov, O. V., Bourhis, L. J., Gildea, R. J, Howard, J. A. K. & Puschmann, H. (2009), J. Appl. Cryst., 42, 339-341.
[Non-Patent Document 2] Sheldrick, G. M., (2015), Acta Cryst., A71, 3-8.
[Non-Patent Document 3] Sheldrick, G. M., (2015), Acta Cryst., C71, 3-8.

### SUMMARY OF THE INVENTION

Both an increase in the lifetime of a light-emitting device and an inhibition of an increase in the manufacturing cost of the light-emitting device are achieved. A device including an organic compound containing deuterium can have a longer lifetime than a device including an organic compound not containing deuterium. Meanwhile, unfortunately, the purity of a compound containing deuterium is difficult to increase and the synthesis cost is high. Thus, the manufacturing cost of the light-emitting device including the organic compound containing deuterium is likely to increase. In particular, a host material, which is a material used for a light-emitting layer of the light-emitting device, is one of the materials that need to be used in the largest amount among the materials included in the light-emitting device; thus, particularly in the case where the organic compound containing deuterium is used as the host material, the manufacturing cost of the light-emitting device might be increased.

In view of the above, an object of one embodiment of the present invention is to provide an organic compound containing deuterium with low synthesis cost. Another object of one embodiment of the present invention is to provide an organic compound with high purity. Another object of one embodiment of the present invention is to provide a synthesis method suitable for providing a high-purity organic compound. Another object of one embodiment of the present invention is to provide an organic compound having a high hole-transport property. Another object of one embodiment of the present invention to provide a light-emitting device with a long lifetime. Another object of one embodiment of the present invention is to provide a light-emitting device with high reliability.

Another object of one embodiment of the present invention is to provide a novel organic compound. Another object of one embodiment of the present invention is to provide a novel hole-transport material. Another object of one embodiment of the present invention is to provide a novel host material. Another object of one embodiment of the present invention is to provide a novel light-emitting device. Another object of one embodiment of the present invention is to provide a novel display device or a novel electronic appliance.

Note that the description of these objects does not preclude the existence of other objects. One embodiment of the present invention does not need to achieve all these objects. Other objects will be apparent from and can be derived from the description of the specification, the drawings, the claims, and the like.

One embodiment of the present invention is an organic compound represented by General Formula (G4).

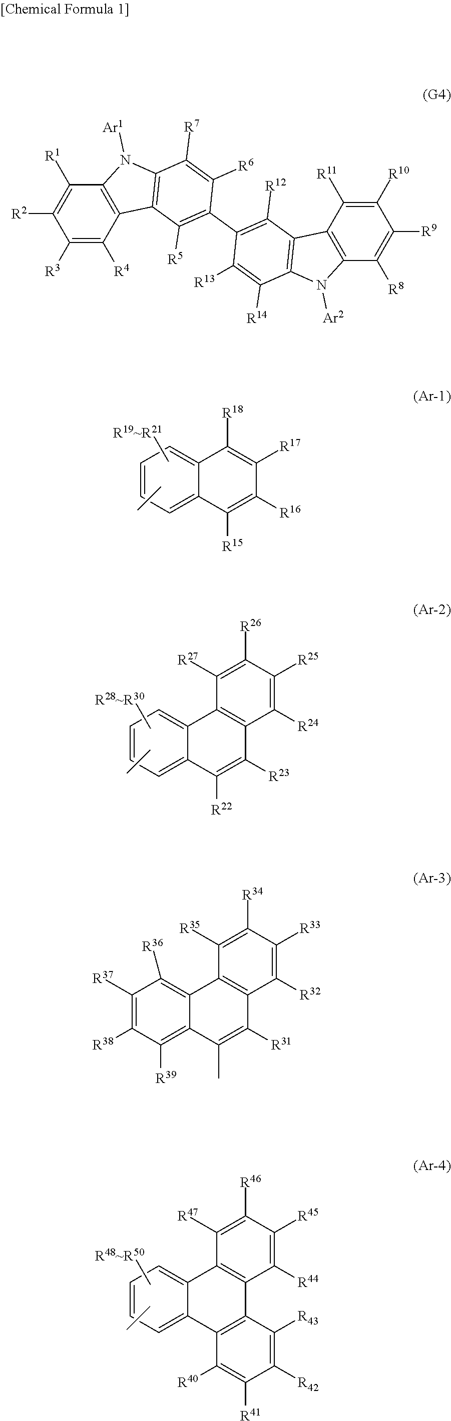

In General Formula (G4), Ar¹ and Ar² are each independently a group represented by any one of General Formulae (Ar-1) to (Ar-4) and include the same fused ring, and R¹ to R¹⁴ each independently represent hydrogen (including deuterium). In General Formulae (Ar-1) to (Ar-4), R¹⁵ to R⁵⁰ each independently represent hydrogen (including deuterium) or an alkyl group having 1 to 6 carbon atoms. Note that the organic compound represented by General Formula (G4) contains two or more deuteriums.

Another embodiment of the present invention is an organic compound represented by General Formula (G5).

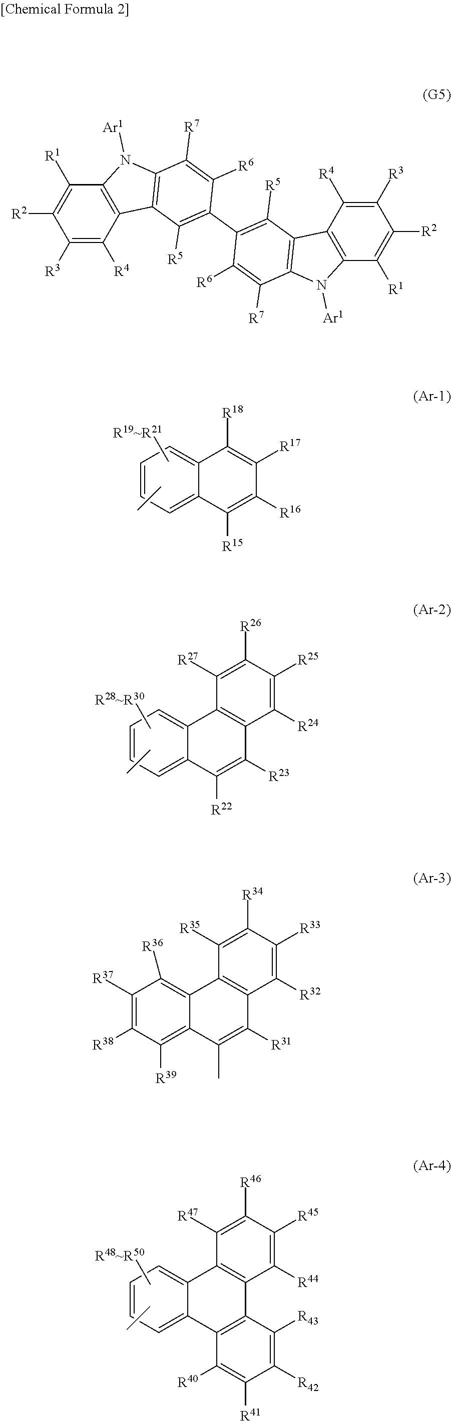

In General Formula (G5), Ar¹ is a group represented by any one of General Formulae (Ar-1) to (Ar-4), and R¹ to R⁷ each independently represent hydrogen (including deuterium). In General Formulae (Ar-1) to (Ar-4), R¹⁵ to R⁵⁰ each independently represent hydrogen (including deuterium) or an alkyl group having 1 to 6 carbon atoms. Note that the organic compound represented by General Formula (G5) contains two or more deuteriums.

Another embodiment of the present invention is an organic compound represented by General Formula (G6).

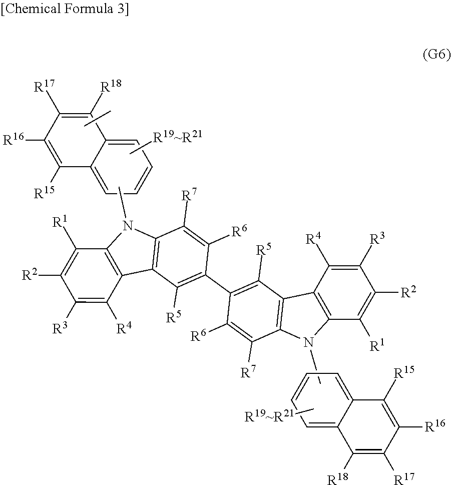

In General Formula (G6), R¹ to R⁷ each independently represent hydrogen (including deuterium), and R¹⁵ to R²¹ each independently represent hydrogen (including deuterium) or an alkyl group having 1 to 6 carbon atoms. Note that the organic compound represented by General Formula (G6) contains two or more deuteriums.

Another embodiment of the present invention is an organic compound represented by General Formula (G7).

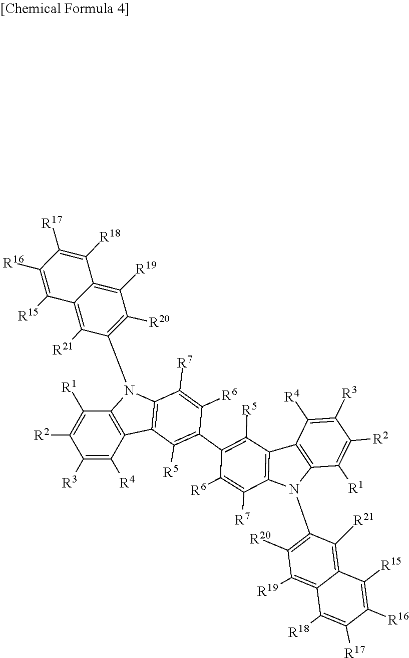

In General Formula (G7), R¹ to R⁷ each independently represent hydrogen (including deuterium), and R¹⁵ to R²¹ each independently represent hydrogen (including deuterium) or an alkyl group having 1 to 6 carbon atoms. Note that the organic compound represented by General Formula (G7) contains two or more deuteriums.

Another embodiment of the present invention is an organic compound represented by Structural Formula (200), (217), (260), (270), (278), or (287).

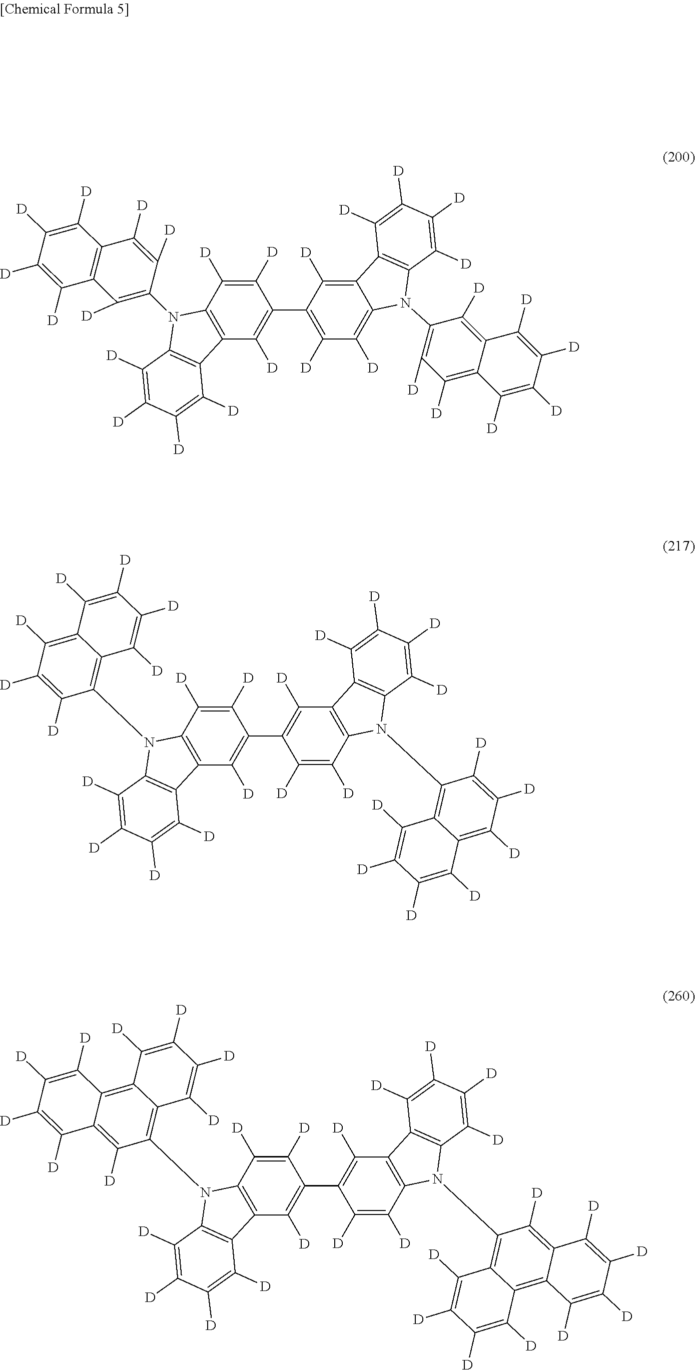

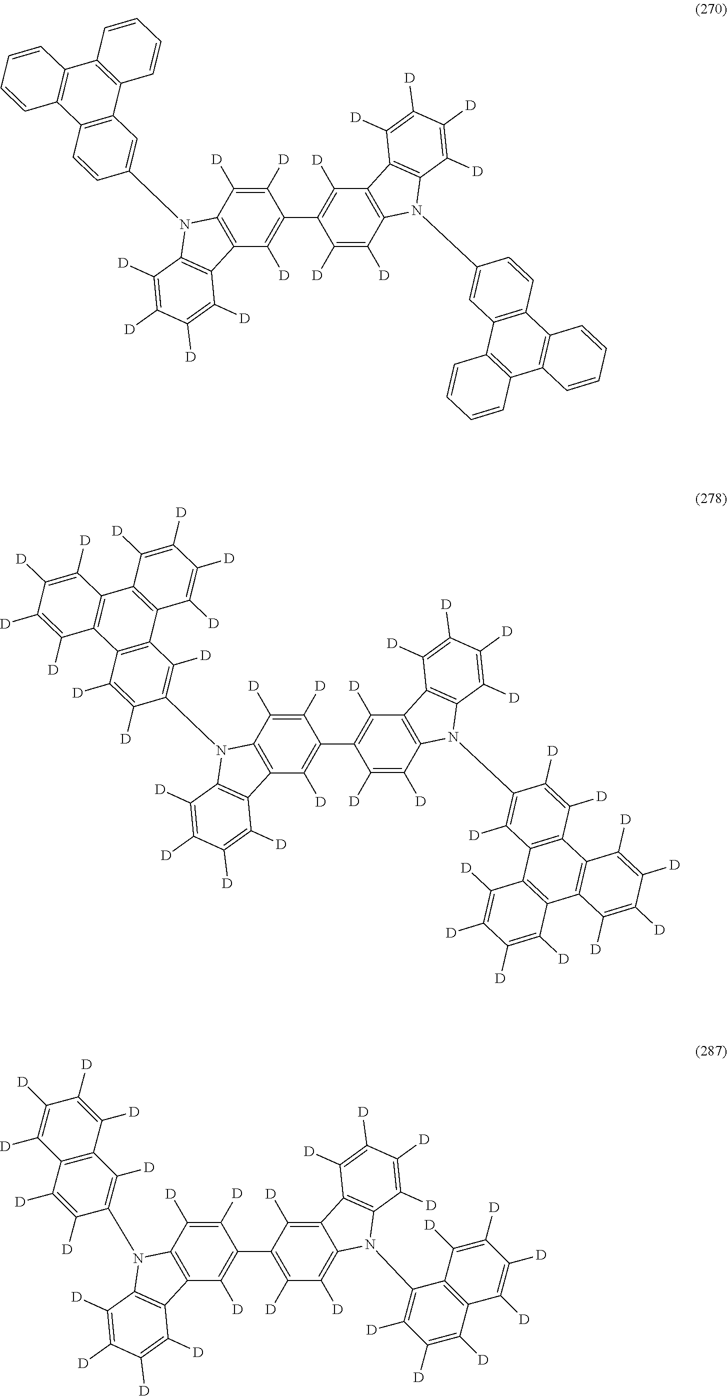

Another embodiment of the present invention is a light-emitting device including an organic compound layer between a pair of electrodes, and the organic compound layer contains an organic compound represented by General Formula (G1).

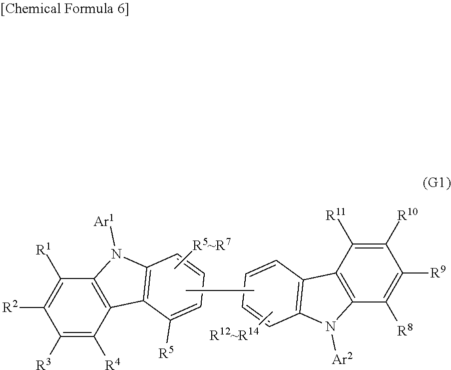

In General Formula (G1), Ar¹ and Ar² each independently represent a substituted or unsubstituted aryl group having 6 to 30 carbon atoms in a ring, and R¹ to R¹⁴ each independently represent hydrogen (including deuterium) or an alkyl group having 1 to 6 carbon atoms. Note that the organic compound represented by General Formula (G1) contains two or more deuteriums, and the sum of the number of the carbon atoms in the ring of Ar¹ and the number of the carbon atoms in the ring of Ar² is 18 or more.

Another embodiment of the present invention is a light-emitting device including an organic compound layer between a pair of electrodes, and the organic compound layer contains an organic compound represented by General Formula (G2).

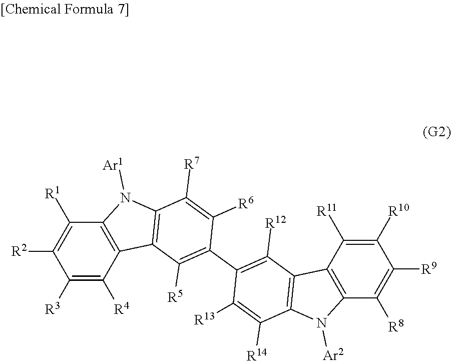

In General Formula (G2), Ar¹ and Ar² each independently represent a substituted or unsubstituted aryl group having 6 to 30 carbon atoms in a ring, and R¹ to R¹⁴ each independently represent hydrogen (including deuterium) or an alkyl group having 1 to 6 carbon atoms. Note that the organic compound represented by General Formula (G2) contains two or more deuteriums, and the sum of the number of the carbon atoms in the ring of Ar¹ and the number of the carbon atoms in the ring of Ar² is 18 or more.

In the light-emitting device with any of the above structures, at least one of Ar¹ and Ar² is preferably a group represented by any one of General Formulae (Ar-1) to (Ar-5).

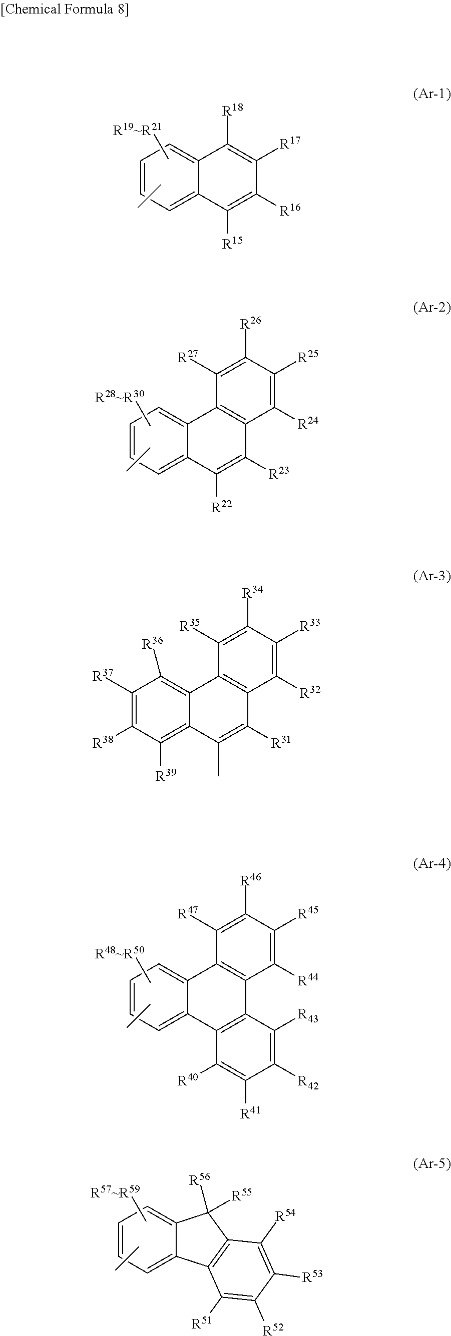

In General Formulae (Ar-1) to (Ar-5), R¹⁵ to R⁵⁹ each independently represent hydrogen (including deuterium) or an alkyl group having 1 to 6 carbon atoms.

Another embodiment of the present invention is a light-emitting device including an organic compound layer between a pair of electrodes, and the organic compound layer contains an organic compound represented by General Formula (G3).

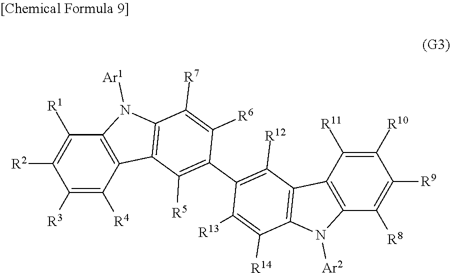

In General Formula (G3), Ar¹ and Ar² each independently represent a substituted or unsubstituted aryl group having 10 to 30 carbon atoms in a ring and having the same fused ring, and R¹ to R¹⁴ each independently represent hydrogen (including deuterium) or an alkyl group having 1 to 6 carbon atoms. Note that the organic compound represented by General Formula (G3) contains two or more deuteriums.

In the light-emitting device with the above structure, Ar¹ is preferably a group represented by any one of General Formulae (Ar-1) to (Ar-4).

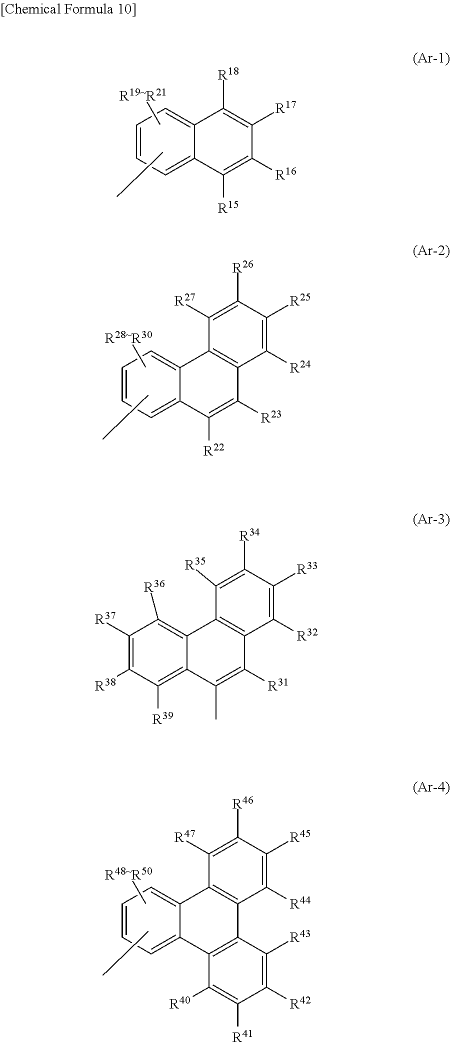

In General Formulae (Ar-1) to (Ar-4), R¹⁵ to R⁵⁰ each independently represent hydrogen (including deuterium) or an alkyl group having 1 to 6 carbon atoms.

Another embodiment of the present invention is a display device including the light-emitting device with the above structure, and a transistor or a substrate.

Another embodiment of the present invention is an electronic appliance including the light-emitting apparatus with the above structure, and a sensing portion, an input portion, or a communication portion.

One embodiment of the present invention can provide an organic compound containing deuterium with a low synthesis cost. Another embodiment of the present invention can provide an organic compound with high purity. Another embodiment of the present invention can provide a synthesis method suitable for providing a high-purity organic compound. Another embodiment of the present invention can provide an organic compound having a high hole-transport property. Another embodiment of the present invention can provide a light-emitting device with a long lifetime. Another embodiment of the present invention can provide a light-emitting device with high reliability.

Another embodiment of the present invention can provide a novel organic compound. Another embodiment of the present invention can provide a novel hole-transport material. Another embodiment of the present invention can provide a novel host material. Another embodiment of the present invention can provide a novel light-emitting device. Another embodiment of the present invention can provide a novel display device or a novel electronic appliance.

Note that the description of these effects does not preclude the existence of other effects. Note that one embodiment of the present invention does not need to have all the effects. Note that other effects will be apparent from the description of the specification, the drawings, the claims, and the like, and other effects can be derived from the description of the specification, the drawings, the claims, and the like.

### BRIEF DESCRIPTION OF THE DRAWINGS

In the accompanying drawings:

FIGS. 1A and 1B illustrate a structure of a light-emitting device in an embodiment;

FIGS. 2A to 2E each illustrate a structure of a light-emitting device in an embodiment;

FIGS. 3A and 3B are, respectively, a top view and a cross-sectional view of a light-emitting apparatus;

FIGS. 4A to 4E are cross-sectional views illustrating an example of a method for manufacturing a light-emitting apparatus;

FIGS. 5A and 5B are cross-sectional views illustrating the example of the method for manufacturing the light-emitting apparatus;

FIGS. 6A to 6D are cross-sectional views illustrating the example of the method for manufacturing the light-emitting apparatus;

FIGS. 7A to 7C are cross-sectional views illustrating the example of the method for manufacturing the light-emitting apparatus;

FIGS. 8A to 8C are cross-sectional views illustrating the example of the method for manufacturing the light-emitting apparatus;

FIGS. 9A to 9C are cross-sectional views illustrating the example of the method for manufacturing the light-emitting apparatus;

FIGS. 10A and 10B are perspective views illustrating a structure example of a display module;

FIGS. 11A and 11B are cross-sectional views each illustrating a structure example of a light-emitting apparatus;

FIG. 12 is a perspective view illustrating a structure example of a light-emitting apparatus;

FIG. 13A is a cross-sectional view illustrating a structure example of the light-emitting apparatus, and FIGS. 13B and 13C are cross-sectional views illustrating structure examples of transistors;

FIG. 14 is a cross-sectional view illustrating a structure example of a light-emitting apparatus;

FIGS. 15A to 15C are a cross-sectional view and top views illustrating a structure example of a light-emitting apparatus;

FIGS. 16A to 16D are cross-sectional views illustrating structure examples of a light-emitting apparatus;

FIGS. 17A to 17C are a cross-sectional view and top views illustrating a structure example of a light-emitting apparatus;

FIGS. 18A to 18D illustrate examples of electronic appliances;

FIGS. 19A to 19F illustrate examples of electronic appliances;

FIGS. 20A to 20G illustrate examples of electronic appliances;

FIG. 21 shows a result of electron diffraction crystal structure analysis;

FIG. 22 shows an absorption spectrum and a PL spectrum of a toluene solution of BisβNCz-d28;

FIG. 23 shows an absorption spectrum and a PL spectrum of a thin film of BisβNCz-d28;

FIG. 24 shows a result of analyzing spin density distribution of BisβNCz-d28 in the triplet excited state by molecular dynamics calculation;

FIG. 25 illustrates a structure of a device in Example.

FIG. 26 is a graph showing luminance-current density characteristics of a light-emitting device 1 and a comparative light-emitting device 2;

FIG. 27 is a graph showing luminance-voltage characteristics of the light-emitting device 1 and the comparative light-emitting device 2;

FIG. 28 is a graph showing current efficiency-luminance characteristics of the light-emitting device 1 and the comparative light-emitting device 2;

FIG. 29 is a graph showing current density-voltage characteristics of the light-emitting device 1 and the comparative light-emitting device 2;

FIG. 30 is a graph showing the external quantum efficiency-luminance characteristics of the light-emitting device 1 and the comparative light-emitting device 2;

FIG. 31 is a graph showing electroluminescence spectra of the light-emitting device 1 and the comparative light-emitting device 2;

FIG. 32 is a graph showing a luminance change over driving time of the light-emitting device 1 and the comparative light-emitting device 2;

FIG. 33 shows an absorption spectrum and a PL spectrum of a toluene solution of βNCCαN-d28;

FIG. 34 shows an absorption spectrum and a PL spectrum of a thin film of βNCCαN-d28;

FIG. 35 shows an absorption spectrum and a PL spectrum of a toluene solution of BisβNCz-d28;

FIG. 36 shows an absorption spectrum and a PL spectrum of a thin film of BisβNCz-d28;

FIG. 37 is a graph showing luminance-current density characteristics of a light-emitting device 3 and a light-emitting device 4;

FIG. 38 is a graph showing luminance-voltage characteristics of the light-emitting device 3 and the light-emitting device 4;

FIG. 39 is a graph showing current efficiency-luminance characteristics of the light-emitting device 3 and the light-emitting device 4;

FIG. 40 is a graph showing current density-voltage characteristics of the light-emitting device 3 and the light-emitting device 4;

FIG. 41 is a graph showing the external quantum efficiency-luminance characteristics of the light-emitting device 3 and the light-emitting device 4;

FIG. 42 is a graph showing electroluminescence spectra of the light-emitting device 3 and the light-emitting device 4; and

FIG. 43 shows luminance changes over driving time of the light-emitting device 3 and the light-emitting device 4.

### DETAILED DESCRIPTION OF THE INVENTION

### Embodiment 1

In this embodiment, a light-emitting device of one embodiment of the present invention and an organic compound of one embodiment of the present invention that can be used in the light-emitting device will be described.

### Structure Example of Light-Emitting Device

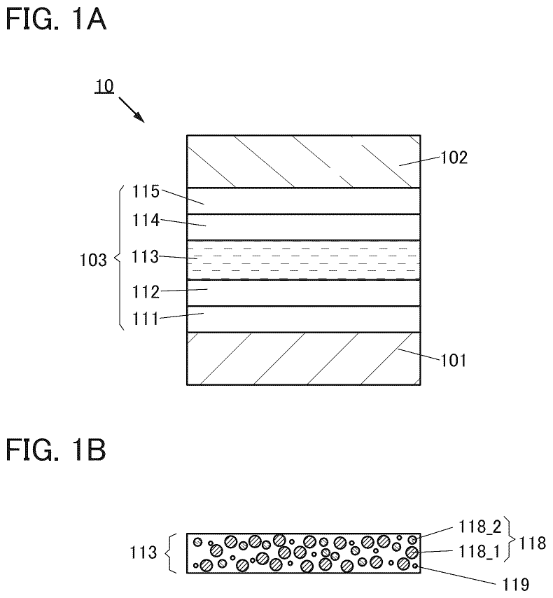

First, a structure of a light-emitting device of one embodiment of the present invention is described with reference to FIGS. 1A and 1B.

FIG. 1A is a schematic cross-sectional view illustrating a light-emitting device 10, which is an example of the light-emitting device of one embodiment of the present invention.

The light-emitting device 10 includes a pair of electrodes (a first electrode 101 and a second electrode 102) and an organic compound layer 103 between the pair of electrodes. The organic compound layer 103 includes at least a light-emitting layer 113. The organic compound layer 103 illustrated in FIG. 1A includes functional layers such as a hole-injection layer 111, a hole-transport layer 112, an electron-transport layer 114, and an electron-injection layer 115, in addition to the light-emitting layer 113.

Although description in this embodiment is made assuming that the first electrode 101 and the second electrode 102 of the pair of electrodes serve as an anode and a cathode, respectively, the structure of the light-emitting device 10 is not limited thereto. That is, the first electrode 101 may be a cathode, the second electrode 102 may be an anode, and the stacking order of the layers between the electrodes may be reversed. In other words, the hole-injection layer 111, the hole-transport layer 112, the light-emitting layer 113, the electron-transport layer 114, and the electron-injection layer 115 may be stacked in this order from the anode side.

The structure of the organic compound layer 103 is not limited to the structure illustrated in FIG. 1A, and a structure including, in addition to the light-emitting layer 113, at least one layer selected from the hole-injection layer 111, the hole-transport layer 112, the electron-transport layer 114, and the electron-injection layer 115 maybe employed. Alternatively, the organic compound layer 103 may include a functional layer which has a function of lowering a hole- or electron-injection barrier, improving a hole- or electron-transport property, inhibiting a hole- or electron-transport property, or reducing quenching by an electrode, for example. Note that the functional layer may be either a single layer or stacked layers.

FIG. 1B is a schematic cross-sectional view illustrating an example of the light-emitting layer 113 in FIG. 1A. The light-emitting layer 113 in FIG. 1B includes a host material 118 and a guest material 119. As the host material 118, one or more types of organic compounds can be used. FIG. 1B illustrates an example of the light-emitting layer 113 in which an organic compound 118_1 and an organic compound 118_2 are used as the host material 118.

In the light-emitting device of one embodiment of the present invention, it is preferable to use, as the host material 118, an organic compound having a structure where two carbazole rings in each of which an aryl group is bonded to N (nitrogen) at the 9-position are bonded to each other by a single bond. Such an organic compound has a high hole-transport property and thus can favorably function as the host material 118, enabling the light-emitting device to have a favorable carrier balance; thus, the organic compound can be suitably used for the light-emitting device.

The two carbazole rings or the aryl groups bonded to the carbazole rings of the organic compound preferably include deuterium. In that case, the thermal stability and electrochemical stability of the organic compound can be increased; thus, the use of the organic compound as the host material 118 can inhibit deterioration of the light-emitting device of one embodiment of the present invention and increase its reliability.

When the sum of the number of carbon atoms of the aryl group bonded to N at the 9-position of one carbazole ring of the organic compound and the aryl group bonded to N at the 9-position of the other carbazole ring is 18 or more, the organic compound can have a higher glass transition point than when the sum of the number of those carbon atoms is less than 18. Thus, the use of the organic compound as the host material 118 can prevent crystallization of the light-emitting layer 113 and increase the heat resistance and reliability of the light-emitting device of one embodiment of the present invention. Moreover, when the sum of the number of carbon atoms of the aryl group bonded to N at the 9-position of one carbazole ring and the aryl group bonded to N at the 9-position of the other carbazole ring is 18 or more, the organic compound can have a higher carrier-transport property than when the sum of the number of those carbon atoms is less than 18; thus, a highly efficient device that can be driven with a low voltage can be provided when the organic compound is used as the host material 118.

In the case where the host material 118 is the above organic compound that does not include a substituent such as an alkyl group, the intermolecular distance in the light-emitting layer 113 is shorter than in the case where the host material 118 includes an alkyl group. Thus, the efficiency of carrier conduction (hopping conduction) can be increased and the carrier transportation can be favorable. Accordingly, a highly efficient light-emitting device that can be driven with a low voltage can be provided. Meanwhile, in the case where the organic compound includes an alkyl group, the sublimation property of the material is improved; thus, the organic compound can be sublimated at a low temperature and is suitable for high purification by sublimation. Thus, in the case where the organic compound is deposited by evaporation, a high-purity film can be formed, so that deterioration due to impurities can be inhibited. For this reason, when the organic compound is used as the host material 118, a light-emitting device with a long lifetime can be provided.

Next, a more specific structure of the organic compound that can be used as the host material 118 is described with a general formula. In the light-emitting device of one embodiment of the present invention, an organic compound represented by General Formula (G1) can be used as the host material 118.

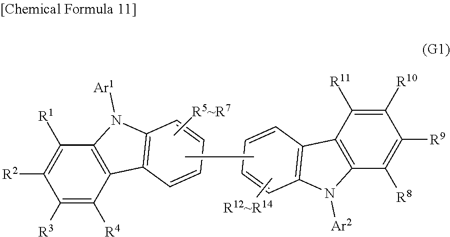

In General Formula (G1), Ar¹ and Ar² each independently represent a substituted or unsubstituted aryl group having 6 to 30 carbon atoms in a ring, and R¹ to R¹⁴ each independently represent hydrogen (including deuterium) or an alkyl group having 1 to 6 carbon atoms. Note that the organic compound represented by General Formula (G1) contains two or more deuteriums, and the sum of the number of the carbon atoms in the ring of Ar¹ and the number of the carbon atoms in the ring of Ar² is 18 or more.

The organic compound represented by General Formula (G1) has a structure where two carbazole rings in each of which an aryl group (Ar¹ and Ar²) is bonded to N at the 9-position are bonded to each other by a single bond; thus, the organic compound has a high hole-transport property and can favorably function as the host material 118.

Since the organic compound represented by General Formula (G1) contains two or more deuteriums, the organic compound has thermal stability and high electrochemical stability. Thus, the use of the organic compound as the host material 118 can inhibit deterioration of the light-emitting device 10 and increase its reliability.

The organic compound represented by General Formula (G1) has a high glass transition point because the sum of the number of carbon atoms of the aryl group (Ar¹) bonded to N at the 9-position of one carbazole ring and the aryl group (Ar²) bonded to N at the 9-position of the other carbazole ring is 18 or more. Thus, the use of the organic compound as the host material 118 can prevent crystallization of the light-emitting layer 113 and increase the reliability of the light-emitting device of one embodiment of the present invention. Moreover, when the sum of the number of carbon atoms of the aryl group bonded to N at the 9-position of one carbazole ring and the aryl group bonded to N at the 9-position of the other carbazole ring is 18 or more, the organic compound can have a higher carrier-transport property than when the sum of the number of those carbon atoms is less than 18; thus, a highly efficient device that can be driven with a low voltage can be provided.

In the light-emitting device of one embodiment of the present invention, an organic compound represented by General Formula (G2) can be used as the host material 118.

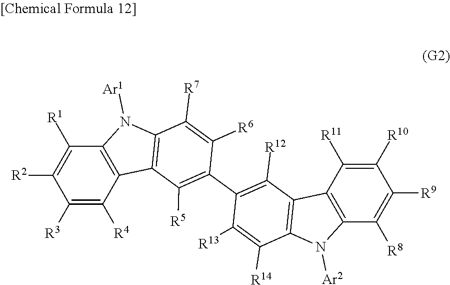

In General Formula (G2), Ar¹ and Ar² each independently represent a substituted or unsubstituted aryl group having 6 to 30 carbon atoms in a ring, and R¹ to R¹⁴ each independently represent hydrogen (including deuterium) or an alkyl group having 1 to 6 carbon atoms. Note that the organic compound represented by General Formula (G2) contains two or more deuteriums, and the sum of the number of the carbon atoms in the ring of Ar¹ and the number of the carbon atoms in the ring of Ar² is 18 or more.

The organic compound represented by General Formula (G2) is different from the organic compound represented by General Formula (G1) in that the bonding position of the single bond that connects the two carbazole rings is limited to C (carbon) at the 3-position of each of the carbazole rings. Since the bonding position of the single bond that connects the two carbazole rings is limited to C at the 3-position of each of the carbazole rings, in the benzene ring to which both the single bond and N of the carbazole ring are bonded, the single bond and N are at the para-positions of the benzene ring. The same applies to both of the two carbazole rings. This structure is preferable because the π-conjugated system in the molecule spreads to both of the two carbazole rings and the organic compound represented by General Formula (G2) can have a high HOMO level, increasing the hole-transport property of the organic compound.

An organic compound in which the bonding position of a single bond that connects two carbazole rings is limited to C at the 3-position of each of the carbazole rings, like the organic compound represented by General Formula (G2), is preferable because the synthesis cost can be reduced. In the synthesis of the organic compound, an intermediate in which a halogen element or the like is introduced to C at the 3-position of the carbazole ring needs to be used. Since a method for introducing a halogen element or the like into C at the 3-position of a carbazole ring is generally established, impurities are less likely to be generated in the synthesis reaction as compared with introduction of a halogen element or the like to C at another position of the carbazole ring. Thus, an organic compound that can be synthesized using the intermediate in which a halogen element or the like is introduced to C at the 3-position of the carbazole ring, like the organic compound represented by General Formula (G2), is easy to purify and easily has an increased purity, which is preferable because the synthesis cost can be reduced.

In the case where the organic compound represented by General Formula (G1) or the organic compound represented by General Formula (G2) is used as the host material 118 in the light-emitting device of one embodiment of the present invention, at least one of Ar¹ and Ar² is preferably a group represented by any one of General Formulae (Ar-1) to (Ar-5).

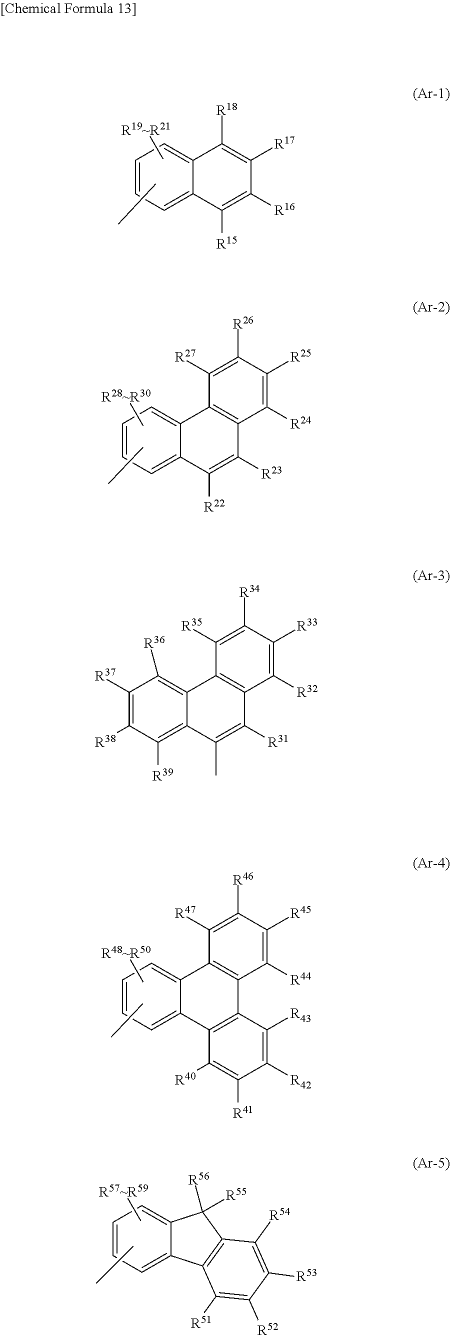

In General Formulae (Ar-1) to (Ar-5), R¹⁵ to R⁵⁹ each independently represent hydrogen (including deuterium) or an alkyl group having 1 to 6 carbon atoms.

The groups represented by General Formulae (Ar-1) to (Ar-5) each include a fused aromatic ring, and thus at least one of Ar¹ and Ar² in the organic compound represented by General Formula (G1) or (G2) is preferably any one of these groups, in which case an organic compound with high electrochemical stability can be obtained. For example, when a heteroaromatic ring or the like in which nitrogen (N) is substituted for carbon (C) of General Formulae (Ar-1) to (Ar-5) is used, light might not be emitted effectively by being affected by electrons or the like in the light-emitting device; thus, a fused aromatic ring is preferably used.

In the organic compound represented by General Formula (G2), Ar¹ and Ar² are each preferably independently a group represented by any one of the groups represented by General Formulae (Ar-1) to (Ar-5), in which case an organic compound with a high glass transition temperature (T_{g}) and high electrochemical stability is obtained.

In the light-emitting device of one embodiment of the present invention, an organic compound represented by General Formula (G3) can be used as the host material 118.

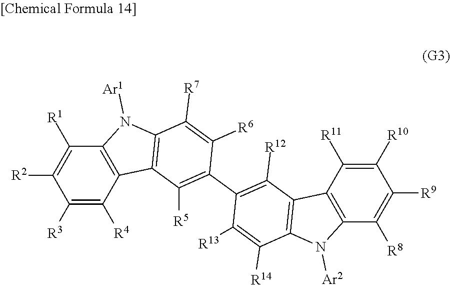

In General Formula (G3), Ar¹ and Ar² each independently represent a substituted or unsubstituted aryl group having 10 to 30 carbon atoms in a ring and include the same fused ring, and R¹ to R¹⁴ each independently represent hydrogen (including deuterium) or an alkyl group having 1 to 6 carbon atoms. Note that the organic compound represented by General Formula (G3) contains two or more deuteriums.

Note that in the organic compound represented by General Formula (G3), the expression “Ar¹ and Ar² include the same fused ring” means both Ar¹ and Ar² have a naphthalene ring, for example. Note that the position of a bond of the fused ring, the presence or absence of a substituent, or the like is not necessarily the same in Ar¹ and Ar² as long as Ar¹ and Ar² include the same fused ring. For example, Ar¹ can be a 1-naphthyl group and Ar² can be a 2-naphthyl group.

The organic compound represented by General Formula (G3) is different from the organic compound represented by General Formula (G2) in that the aryl groups bonded to N of the two carbazole rings include the same fused ring. Since the fused rings included in the aryl groups bonded to N of the two carbazole rings are the same, the symmetry of the organic compound and the carrier-transport property can be high.

In the light-emitting device of one embodiment of the present invention, in the case where the organic compound represented by General Formula (G3) is used as the host material 118, Ar¹ is preferably a group represented by any one of General Formulae (Ar-1) to (Ar-4).

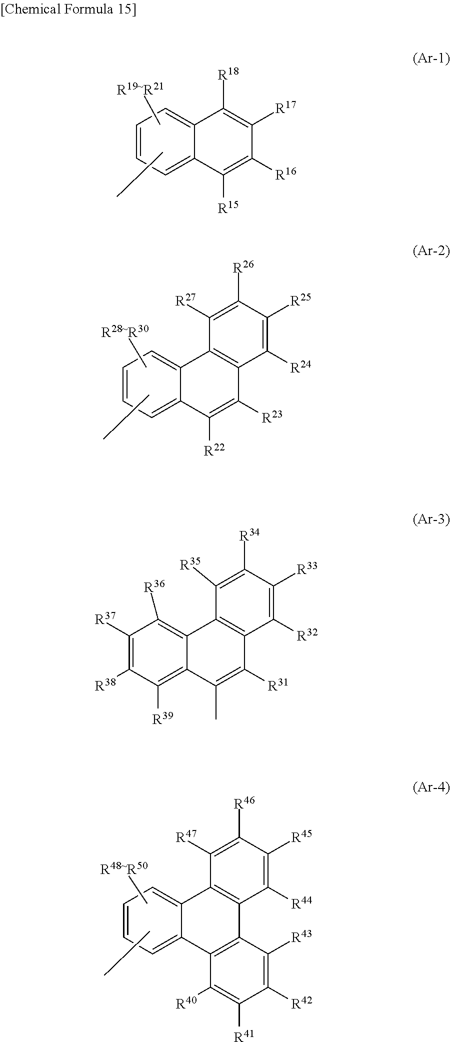

In General Formulae (Ar-1) to (Ar-4), R¹⁵ to R⁵⁰ each independently represent hydrogen (including deuterium) or an alkyl group having 1 to 6 carbon atoms.

The groups represented by General Formulae (Ar-1) to (Ar-4) each include a fused aromatic ring. In the organic compound represented by General Formula (G3), Ar¹ is preferably a group represented by any one of General Formulae (Ar-1) to (Ar-4), in which case the organic compound can have a high T_{g} and high electrochemical stability.

In the light-emitting device of one embodiment of the present invention, an organic compound represented by General Formula (G4) can be used as the host material 118. Another embodiment of the present invention is the organic compound represented by General Formula (G4).

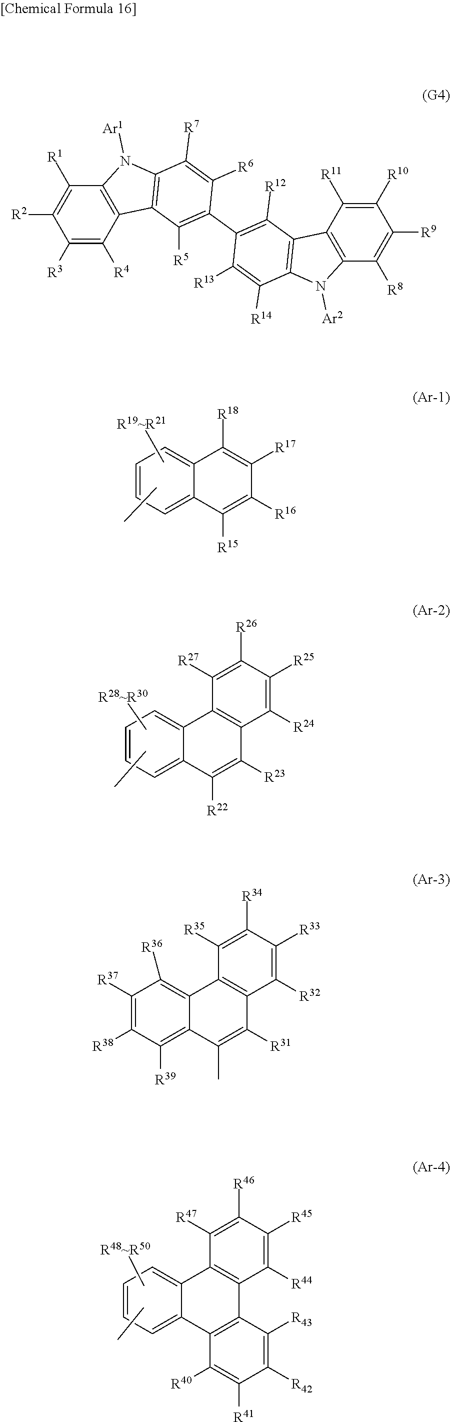

In General Formula (G4), Ar¹ and Ar² are each independently a group represented by any one of General Formulae (Ar-1) to (Ar-4) and include the same fused ring, and R¹ to R¹⁴ each independently represent hydrogen (including deuterium). In General Formulae (Ar-1) to (Ar-4), R¹⁵ to R⁵⁰ each independently represent hydrogen (including deuterium) or an alkyl group having 1 to 6 carbon atoms. Note that the organic compound represented by General Formula (G4) contains two or more deuteriums.

Note that in the organic compound represented by General Formula (G4), the expression “Ar¹ and Ar² include the same fused ring” means, for example, Ar² is also a group represented by General Formula (Ar-1) when Ar¹ is a group represented by General Formula (Ar-1). It can also be said that Ar¹ and Ar² are groups represented by the same general formula. Note that the substituents (R¹⁵ to R⁵⁰) included in the groups represented by the general formulae may be the same or different from each other.

The organic compound represented by General Formula (G4) is different from the organic compound represented by General Formula (G3) in that Ar¹ is limited to a group represented by any one of General Formulae (Ar-1) to (Ar-4). The groups represented by General Formulae (Ar-1) to (Ar-4) each have a fused aromatic ring, and thus Ar¹ is preferably limited to a group represented by any one of General Formulae (Ar-1) to (Ar-4), in which case an organic compound having a high sublimation property, a high T_{g}, and high electrochemical stability is obtained.

The organic compound represented by General Formula (G4) is different from the organic compound represented by General Formula (G3) also in that R¹ to R¹⁴ are limited to hydrogen (including deuterium). In the case where the host material 118 is the organic compound which is represented by General Formula (G4) and in which R¹ to R¹⁴ are hydrogen (including deuterium), the intermolecular distance in the light-emitting layer 113 is shorter than in the case where the host material 118 includes an alkyl group. Thus, the efficiency of carrier conduction (hopping conduction) can be increased and the carrier-transport property can be favorable. Accordingly, a highly efficient light-emitting device that can be driven with a low voltage can be provided. When two or more kinds of compounds are mixed to form an exciplex, the exciplex can be efficiently formed and energy transfer from the host material to a dopant is efficiently performed; thus, a highly efficient light-emitting device that can be driven with a low voltage can be provided.

In the light-emitting device of one embodiment of the present invention, an organic compound represented by General Formula (G5) can be used as the host material 118. Another embodiment of the present invention is the organic compound represented by General Formula (G5).

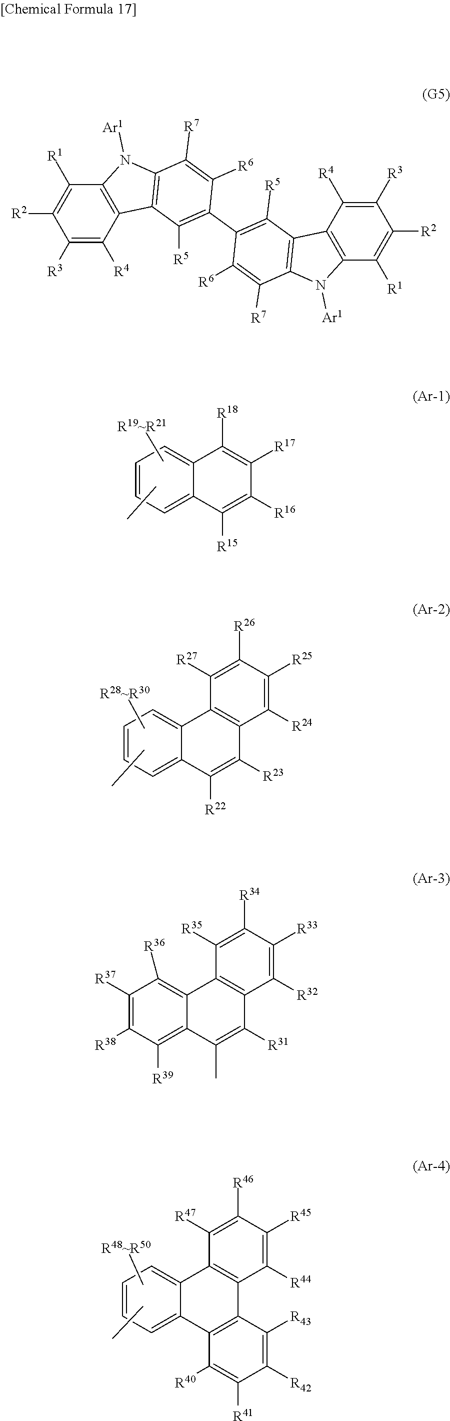

In General Formula (G5), Ar¹ is a group represented by any one of General Formulae (Ar-1) to (Ar-4), and R¹ to R⁷ each independently represent hydrogen (including deuterium). In General Formulae (Ar-1) to (Ar-4), R¹⁵ to R⁵⁰ each independently represent hydrogen (including deuterium) or an alkyl group having 1 to 6 carbon atoms. Note that the organic compound represented by General Formula (G5) contains two or more deuteriums.

In the organic compound represented by General Formula (G5), two Ar¹'s are represented by the same general formulae, any of General Formulae (Ar-1) to (Ar-4), one set of R¹ to R⁷ is the same as the other set of R¹ to R⁷ and one set of R¹⁵ to R⁵⁰ is the same as the other set of R¹⁵ to R⁵⁰.

The organic compound represented by General Formula (G5) is different from the organic compound represented by General Formula (G4) in that R⁸ is replaced with R¹, R⁹ is replaced with R², R¹⁰ is replaced with R³, R¹¹ is replaced with R⁴, R¹² is replaced with R⁵, R¹³ is replaced with R⁶, R¹⁴ is replaced with R⁷, and Ar² is replaced with Ar¹. In the case where the carbazole ring includes deuterium in the organic compound of one embodiment of the present invention, such a structure in which the two carbazole rings include deuterium at the same substitution site can achieve higher symmetry. Specifically, the organic compound represented by General Formula (G5) has a highly symmetric molecular structure with the C₂ axis in which the same two skeletons are bonded to each other by a single bond. Therefore, the two skeletons can be formed using a common source material and a common intermediate, which is preferable because the synthesis can be easily performed. Specifically, the organic compound represented by General Formula (G5) can be synthesized easily because a common source material and a common intermediate can be used for the two skeletons. In addition, a by-product of a homocoupling product that is easily formed has the same molecular structure as the target substance; thus, generation of impurities can be inhibited, which is preferable because the target substance can have high purity. In the organic compound represented by General Formula (G5), spin density is distributed over Ar¹ in the lowest triplet excited state (T₁) in molecular dynamics calculation. Therefore, the organic compound with high symmetry is preferably used as the host material 118, in which case the excitation energy is dispersed to two places and the excitation frequency per Ar¹ group is lowered when the light-emitting device is driven at the same current density (when the excitation frequency per molecule is the same), so that the molecule is less likely to deteriorate.

In the light-emitting device of one embodiment of the present invention, an organic compound represented by General Formula (G6) can be used as the host material 118. Another embodiment of the present invention is the organic compound represented by General Formula (G6).

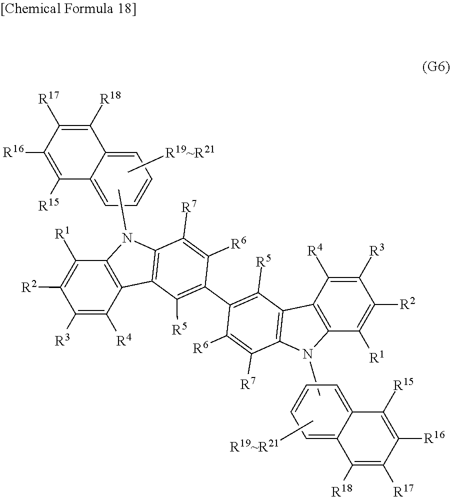

In General Formula (G6), R¹ to R⁷ each independently represent hydrogen (including deuterium), and R¹⁵ to R²¹ each independently represent hydrogen (including deuterium) or an alkyl group having 1 to 6 carbon atoms. Note that the organic compound represented by General Formula (G6) contains two or more deuteriums.

The organic compound represented by General Formula (G6) is different from the organic compounds represented by the above general formulae in that the aryl groups bonded to N of the two carbazole rings are each limited to a substituted or unsubstituted naphthyl group. It is preferable that both of the aryl groups bonded to N of the two carbazole rings be substituted or unsubstituted naphthyl groups, in which case the compound can have an increased sublimation property and high heat resistance at the same time. A naphthyl group is the smallest substituent among fused aromatic ring groups, and the absolute number of hydrogens contained in the substituent is small. Thus, in the case where protium of the substituent is substituted by deuterium, the absolute number of protiums for the substitution is small and the selection range for the substitution sites is narrow. Accordingly, a load and cost for synthesis can be reduced, which is preferable.

In the light-emitting device of one embodiment of the present invention, an organic compound represented by General Formula (G7) can be used as the host material 118. Another embodiment of the present invention is the organic compound represented by General Formula (G7).

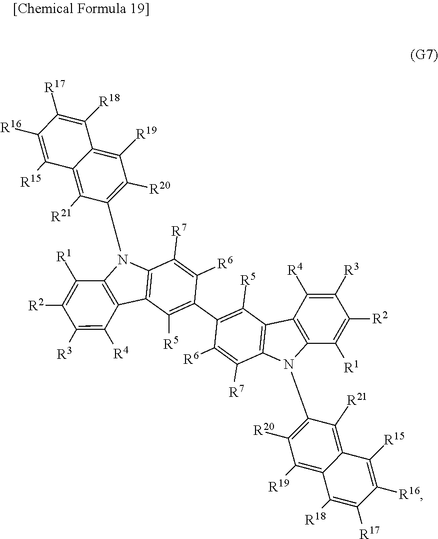

In General Formula (G7), R¹ to R⁷ each independently represent hydrogen (including deuterium), and R¹⁵ to R²¹ each independently represent hydrogen (including deuterium) or an alkyl group having 1 to 6 carbon atoms. Note that the organic compound represented by General Formula (G7) contains two or more deuteriums.

In the organic compound represented by General Formula (G7), two R¹'s are the same group, two R²'s are the same group, two R³'s are the same group, two R⁴'s are the same group, two R⁵'s are the same group, two R⁶'s are the same group, two R⁷'s are the same group, two R¹⁵'s are the same group, two R¹⁶'s are the same group, two R¹¹'s are the same group, two R¹¹'s are the same group, two R¹⁹'s are the same group, two R²⁰'s are the same group, and two R²¹'s are the same group.

The organic compound represented by General Formula (G7) is different from the organic compound represented by General Formula (G6) in that the aryl groups bonded to N of the two carbazole rings are limited to substituted or unsubstituted 2-naphthyl groups (also referred to as β-naphthyl groups). The aryl groups bonded to N of the two carbazole rings are the substituted or unsubstituted 2-naphthyl groups and thus the organic compound represented by General Formula (G7) can have a highly symmetric molecular structure with the C₂ axis in which the same two skeletons are bonded to each other by a single bond. The two skeletons included in the organic compound represented by General Formula (G7) can be formed using a common source material and a common intermediate, which is preferable because the organic compound can be synthesized easily. In the organic compound represented by General Formula (G7), spin density is distributed over the 2-naphthyl group in the T₁ level in molecular dynamics calculation. The organic compound is preferably used as the host material 118, in which case the excitation energy is dispersed to two places and the excitation frequency per 2-naphthyl group is lowered when the light-emitting device is driven at the same current density (when the excitation frequency per molecule is the same), so that the molecule is less likely to deteriorate. An organic compound including a 2-naphthyl group has a higher T₁ level than an organic compound including a 1-naphthyl group, and thus, the selection range of light-emitting materials in the light-emitting device can be widened, which is preferable.

In the light-emitting device of one embodiment of the present invention, an organic compound represented by General Formula (G8) can be used as the host material 118. Another embodiment of the present invention is the organic compound represented by General Formula (G8).

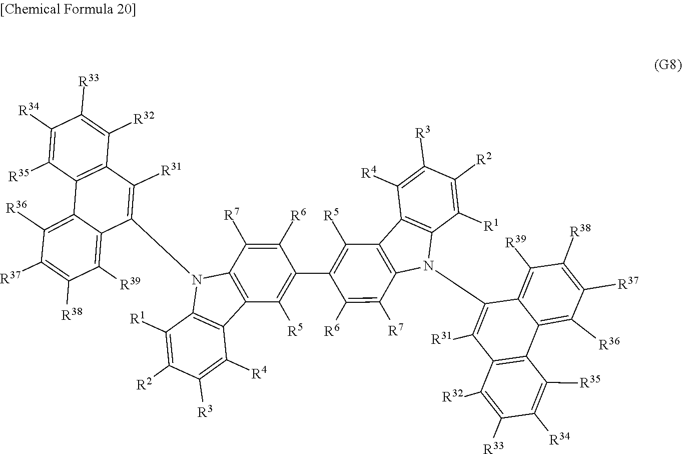

In General Formula (G8), R¹ to R⁷ each independently represent hydrogen (including deuterium), and R³¹ to R³⁹ each independently represent hydrogen (including deuterium) or an alkyl group having 1 to 6 carbon atoms. Note that the organic compound represented by General Formula (G8) contains two or more deuteriums.

In the organic compound represented by General Formula (G8), two R¹'s are the same group, two R²'s are the same group, two R³'s are the same group, two R⁴'s are the same group, two R⁵'s are the same group, two R⁶'s are the same group, two R⁷'s are the same group, two R³¹'s are the same group, two R³²'s are the same group, two R³³'s are the same group, two R³⁴'s are the same group, two R³⁵'s are the same group, two R³⁶'s are the same group, two R³⁷'s are the same group, two R³⁸'s are the same group, and two R³⁹'s are the same group.

The organic compound represented by General Formula (G8) has a bicarbazole skeleton with high symmetry, which is preferable because the number of kinds of source materials used for the synthesis can be reduced and thus the synthesis can be facilitated. The Suzuki coupling reaction can also be used for the final step of synthesizing the organic compound. Although the Suzuki coupling reaction might potentially cause a homocoupling product as a by-product, a product obtained through the homocoupling reaction for synthesizing the organic compound represented by General Formula (G8) is also the target substance, and thus, a load on a purification process is small, which is preferable. Since the organic compound represented by General Formula (G8) includes a phenanthryl group, the robustness of the organic compound can be increased while a high T₁ level is maintained, and the glass transition temperature can be thus increased, which is preferable.

In the light-emitting device of one embodiment of the present invention, an organic compound represented by General Formula (G9) can be used as the host material 118. Another embodiment of the present invention is the organic compound represented by General Formula (G9).

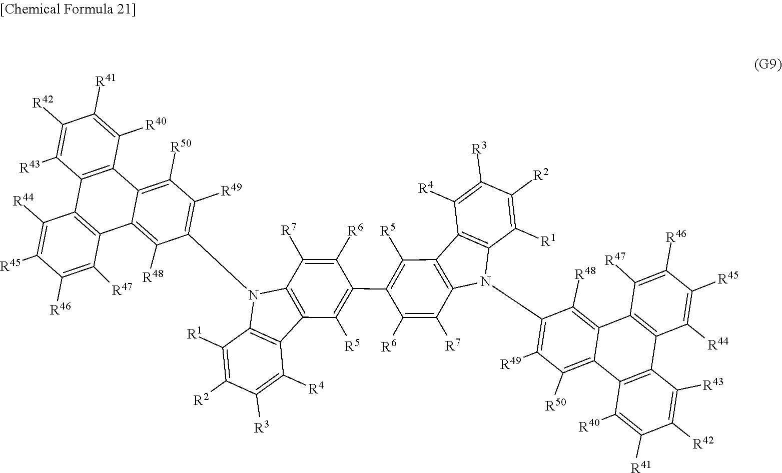

In General Formula (G9), R¹ to R⁷ each independently represent hydrogen (including deuterium), and R⁴¹ to R⁵⁰ each independently represent hydrogen (including deuterium) or an alkyl group having 1 to 6 carbon atoms. Note that the organic compound represented by General Formula (G9) contains two or more deuteriums.

In the organic compound represented by General Formula (G9), two R¹'s are the same group, two R²'s are the same group, two R³'s are the same group, two R⁴'s are the same group, two R⁵'s are the same group, two R⁶'s are the same group, two R⁷'s are the same group, two R⁴⁰'s are the same group, two R⁴¹'s are the same group, two R⁴²'s are the same group, two R⁴³'s are the same group, two R⁴⁴'s are the same group, two R⁴⁵'s are the same group, two R⁴⁶'s are the same group, two R⁴⁷'s are the same group, two R⁴⁸'s are the same group, two R⁴⁹'s are the same group, and two R⁵⁰'s are the same group.

The organic compound represented by General Formula (G9) has a bicarbazole skeleton with high symmetry, which is preferable because the number of kinds of source materials used for the synthesis can be reduced and thus the synthesis can be facilitated. Since the organic compound represented by General Formula (G9) includes a triphenylenyl group, the robustness of the organic compound can be increased while a high T₁ level is maintained, and the glass transition temperature can be thus increased, which is preferable.

Note that in the case where the host material 118 is the organic compound which is represented by any one of General Formulae (G1) to (G9) and does not include an alkyl group, the intermolecular distance in the light-emitting layer 113 is shorter than in the case where the host material 118 includes an alkyl group. Thus, the efficiency of carrier conduction (hopping conduction) can be increased and the carrier-transport property can be favorable. Accordingly, a highly efficient light-emitting device that can be driven with a low voltage can be provided.

### <Position of Deuterium>

Here, the preferred positions of deuterium in the organic compounds represented by General Formulae (G1) to (G9) above are described.

In the organic compounds represented by General Formulae (G1) to (G4), R³'s and R¹⁰'s are preferably deuterium. In the organic compounds represented by General Formulae (G5) to (G9), two R³'s are preferably deuterium. Since R³'s and R¹⁰'s in each of General Formulae (G1) to (G4) and two R³'s in each of General Formulae (G5) to (G9) are positioned at the para-position of nitrogen in the carbazole ring of the organic compound, the C—H bond at the para-position is weaker and the reactivity is higher than in the other positions. Thus, R³'s and R¹⁰'s in each of General Formulae (G1) to (G4) and two R³'s in each of General Formulae (G5) to (G9) are preferably deuterium, in which case the stability of the organic compounds can be increased by deuteration at a minimum number of positions. Thus, R³'s and R¹⁰'s in each of General Formulae (G1) to (G4) and two R³'s in each of General Formulae (G5) to (G9) are preferably deuterium, in which case the chemical reactivity of the organic compounds can be reduced most efficiently.

In the organic compounds represented by General Formulae (G1) to (G4), R⁵'s, R⁶'s, R⁷'s, R¹²'s, R¹³'s, and R¹⁴'s are further preferably deuterium. In the organic compounds represented by General Formulae (G5) to (G9), two R⁵'s, two R⁶'s, and two R⁷'s are further preferably deuterium. Since R⁵, R⁶, R⁷, R¹², R¹³, and R¹⁴ in each of General Formulae (G1) to (G4) and two R⁵'s, two R⁶'s, and two R⁷'s in each of General Formulae (G5) to (G9) are at positions where the C—H bond is easily dissociated due to a steric hindrance or the like of one carbazole ring with the other carbazole ring of the organic compound, R⁵, R⁶, R⁷, R¹², R¹³, and R¹⁴ in each of General Formulae (G1) to (G4) and two R⁵'s, two R⁶'s, and two R⁷'s in each of General Formulae (G5) to (G9) are preferably deuterium to increase the stability of the organic compounds. Thus, six or more of hydrogens contained in each of the organic compounds represented by General Formulae (G1) to (G9) are further preferably deuterium.

In the organic compounds represented by General Formulae (G1) to (G4), R¹ to R⁷ are preferably deuterium, and R⁸ to R¹⁴ are further preferably deuterium. It is further preferable that two sets of R¹ to R⁷ in each of the organic compounds represented by General Formulae (G5) to (G9) be deuterium. In that case, all hydrogens contained in the carbazole rings of the organic compounds are deuterium; thus, a degradation reaction accompanied by, for example, release of hydrogen from the entire carbazole rings can be inhibited. This can increase the stability of the organic compounds. Thus, the organic compounds represented by General Formulae (G1) to (G4) each preferably contain 7 or more deuteriums, further preferably contain 14 or more deuteriums. It is further preferable that the organic compounds represented by General Formulae (G5) to (G9) each contain 14 or more deuteriums.

In each of the organic compounds represented by General Formulae (G5) to (G9), two sets of R¹⁵ to R²¹ are further preferably deuterium. This can increase the stability of the naphthyl group and the stability of the organic compound. Furthermore, the results of the molecular dynamics calculation show that spin density is distributed over the naphthyl group in the T₁ level. In the T₁ level, the thermal and electrochemical stability in an excited state is also improved when the skeleton over which spin density is distributed contains deuterium. Thus, it is further preferable that the organic compounds represented by General Formulae (G5) to (G9) each contain 14 or more deuteriums.

All hydrogens contained in the organic compounds represented by General Formulae (G1) to (G9) are preferably deuterium, in which case the thermal and electrochemical stability can be maximized.

### Specific Examples of Substituent

Next, specific examples of substituents that can be applied to the organic compounds represented by General Formulae (G1) to (G9) above are described. The substituent that can be used in any of the organic compounds represented by General Formulae (G1) to (G9) above is not limited to the following specific examples of substituents.

An aryl group having 6 to 30 carbon atoms in a ring refers to a monovalent group obtained by removing one hydrogen atom from a monocyclic or polycyclic aromatic hydrocarbon in which the sum of the number of carbon atoms in a ring is greater than or equal to 6 and less than or equal to 30. Here, the polycyclic aromatic hydrocarbon refers to both an aromatic hydrocarbon in which a plurality of rings are connected through a bond, such as biphenyl, and a fused polycyclic aromatic hydrocarbon in which a plurality of rings are fused, such as naphthalene. Specific examples of the aryl group having 6 to 30 carbon atoms in a ring include a phenyl group, a naphthyl group (a 1-naphthyl group or a 2-naphthyl group), a biphenylyl group (a biphenyl-2-yl group, a biphenyl-3-yl group, or a biphenyl-4-yl group), a fluorenyl group, a phenanthryl group, an anthryl group, a phenylnaphthyl group, a naphthylphenyl group, a fluoranthenyl group, a terphenylyl group, a quaterphenylyl group, a 9,9′-diphenylfluorenyl group, a 9,9′-spirobifluorenyl group, and a binaphthylphenyl group. In the case where the aryl group having 6 to 30 carbon atoms in a ring has a substituent, specific examples of the substituent include an alkyl group having 1 to 6 carbon atoms and an alkyl group having 2 to 6 carbon atoms. The aryl group having 6 to 30 carbon atoms in a ring preferably includes deuterium. In the case where the aryl group having 6 to 30 carbon atoms in a ring includes a substituent, the substituent preferably includes deuterium.

An alkyl group having 1 to 6 carbon atoms refers to a monovalent group formed by removing one hydrogen atom from an alkane having 1 to 6 carbon atoms. Specific examples of the alkyl group having 1 to 6 carbon atoms include a methyl group, an ethyl group, an n-propyl group, an isopropyl group, an n-butyl group, a sec-butyl group, an isobutyl group, a tert-butyl group, an n-pentyl group, an isopentyl group, a sec-pentyl group, a tert-pentyl group, a neopentyl group, an n-hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, and a 2,3-dimethylbutyl group. Note that the alkyl group having 1 to 6 carbon atoms preferably includes deuterium.

### Specific Examples of Organic Compound

Next, specific examples of the organic compounds represented by the above general formulae are shown below. Note that the organic compounds represented by the above general formulae are not limited to the following specific examples.

Specific examples of the organic compounds represented by General Formulae (G1) to (G3) include organic compounds represented by Structural Formulae (100) to (136). Note that the organic compounds represented by General Formulae (G1) to (G3) are not limited to the organic compounds represented by Structural Formulae (100) to (136).

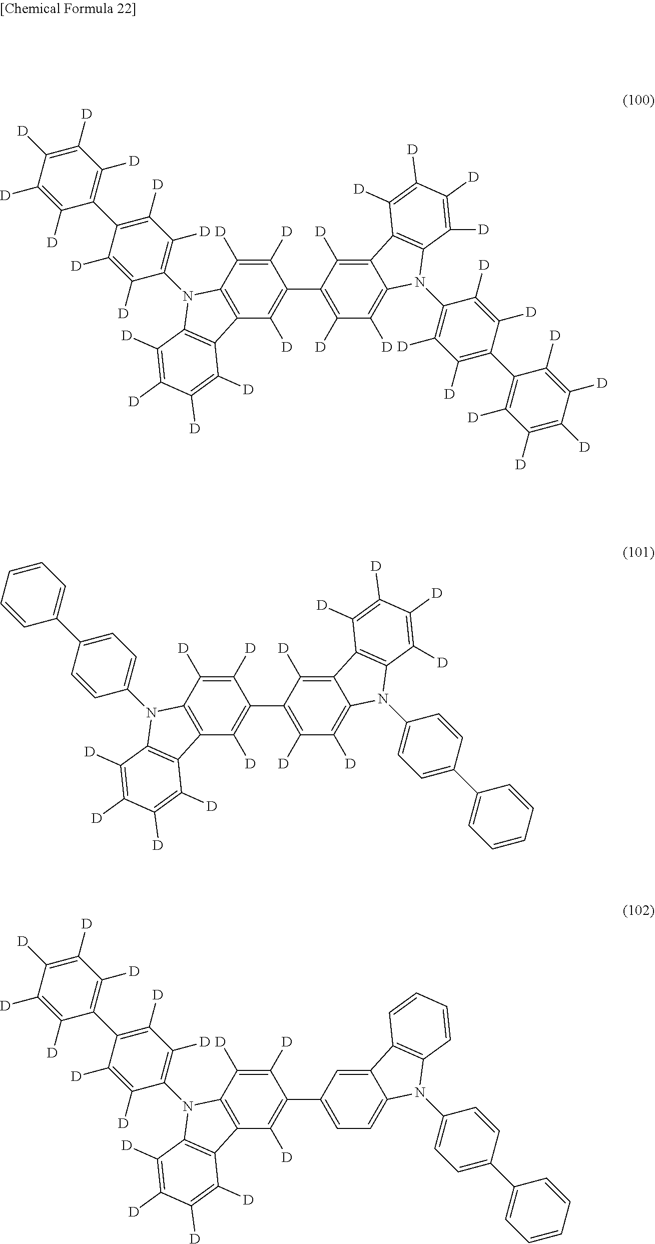

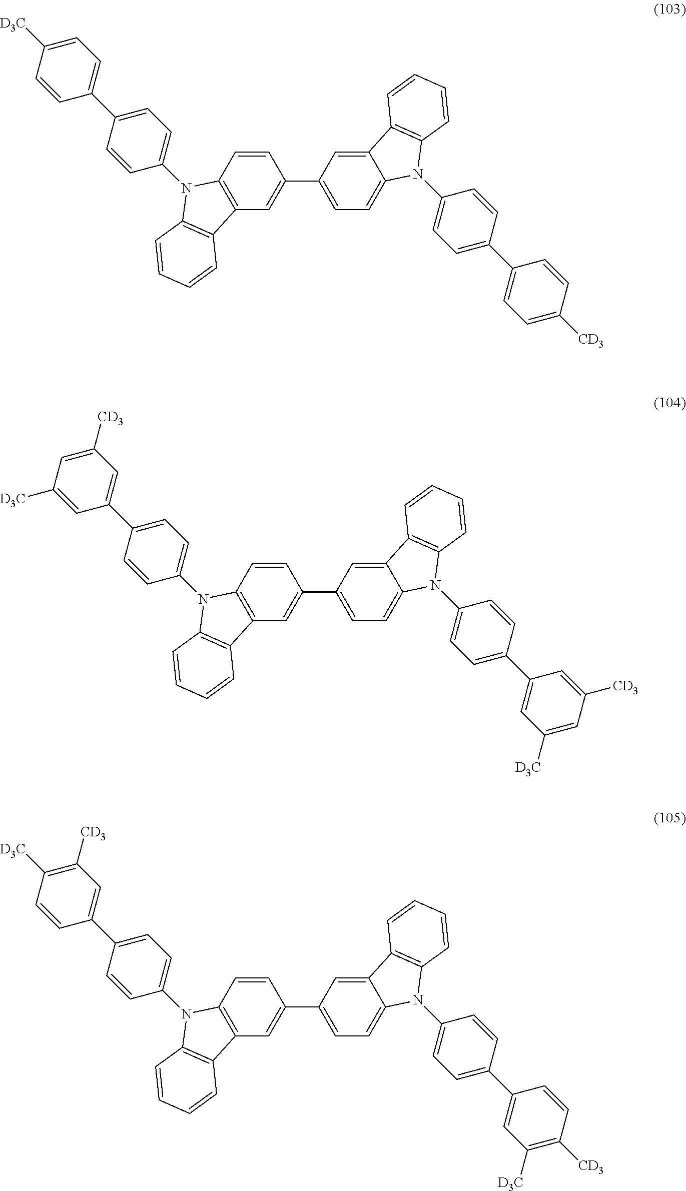

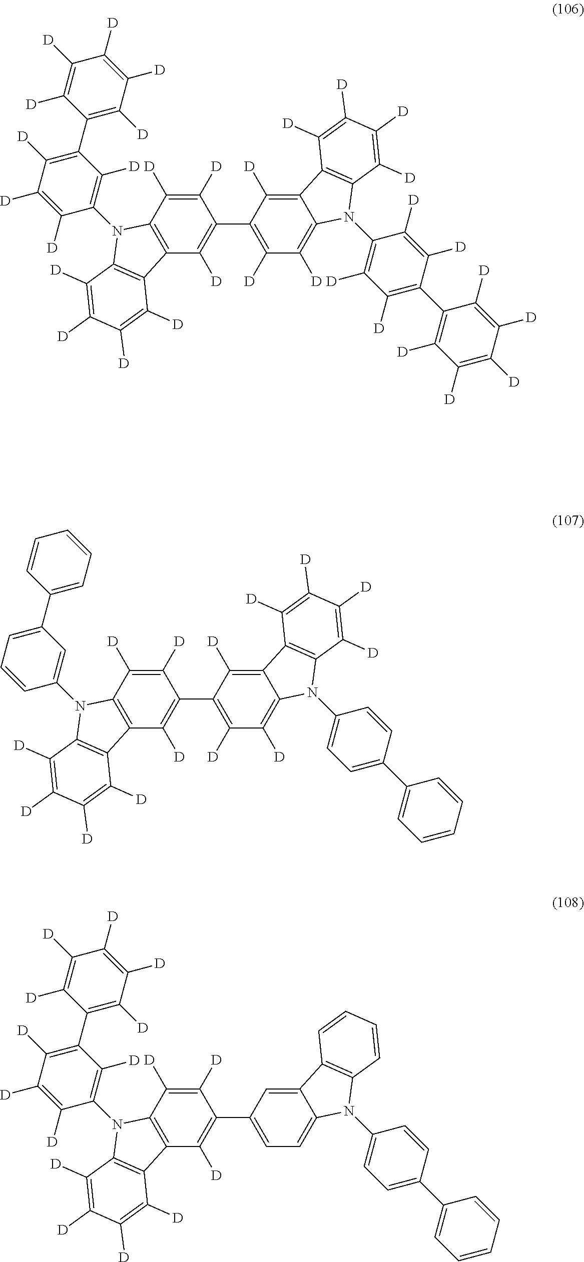

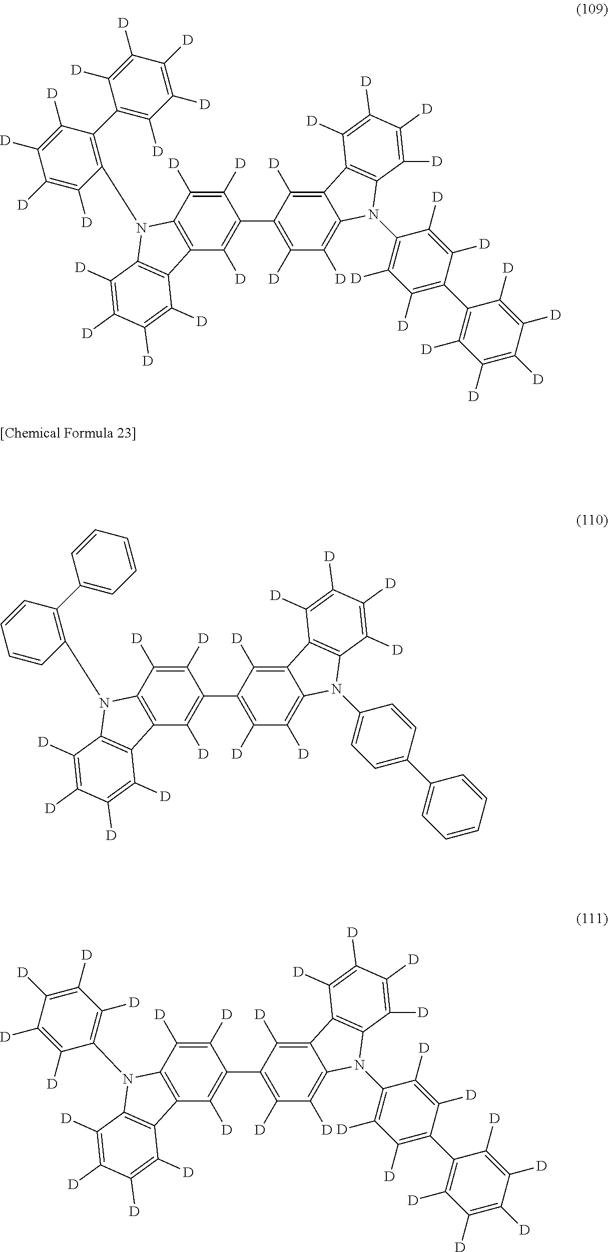

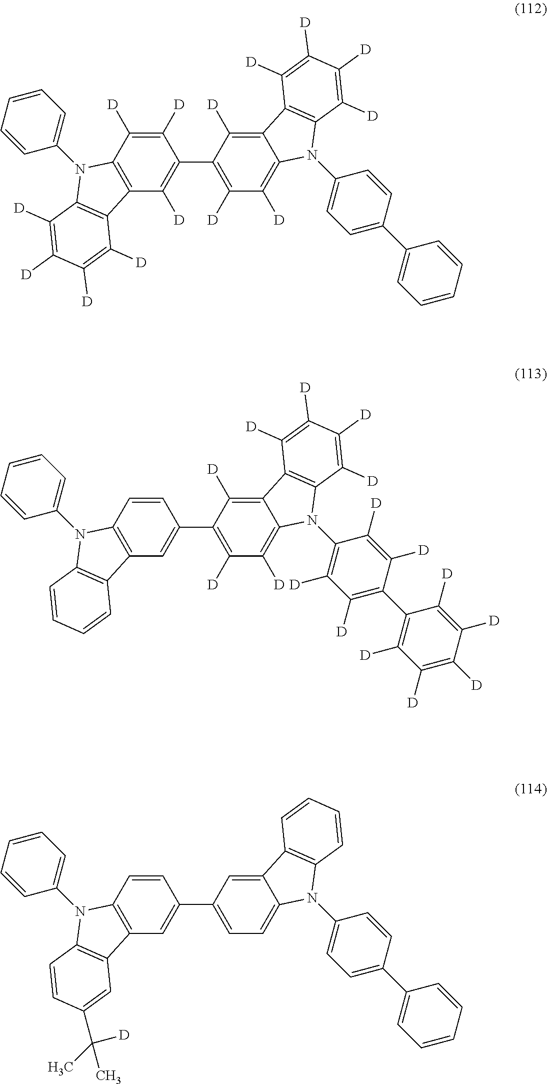

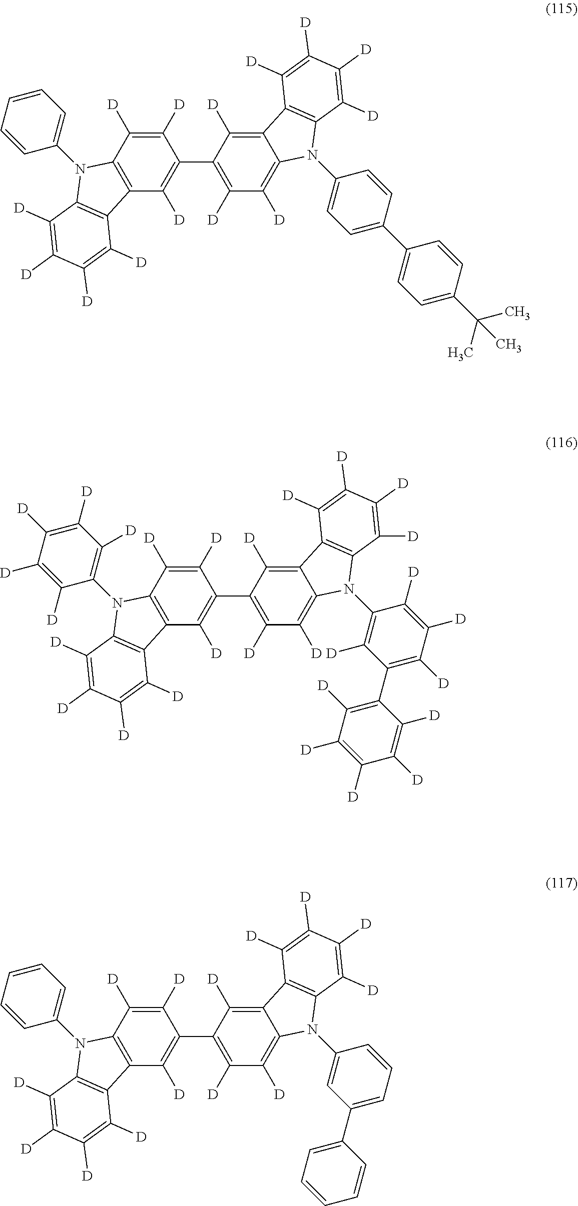

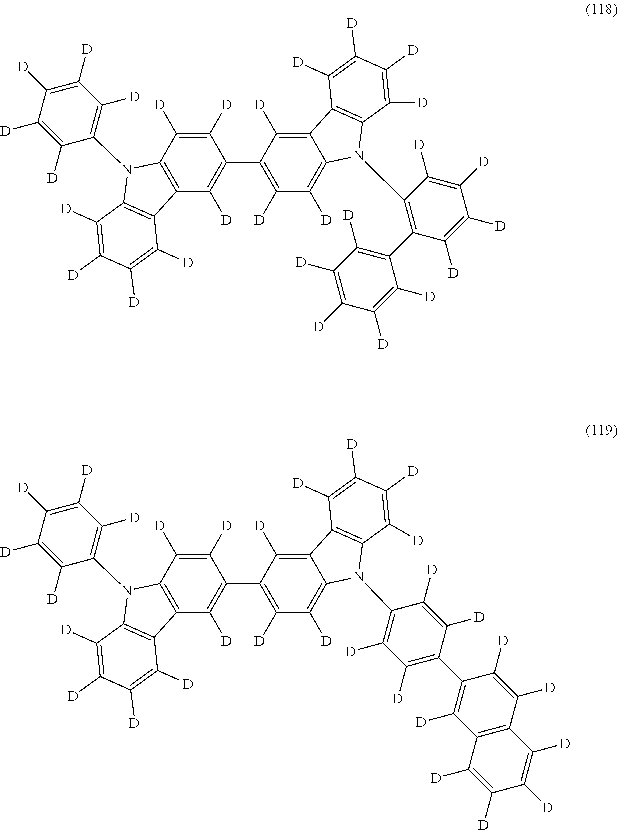

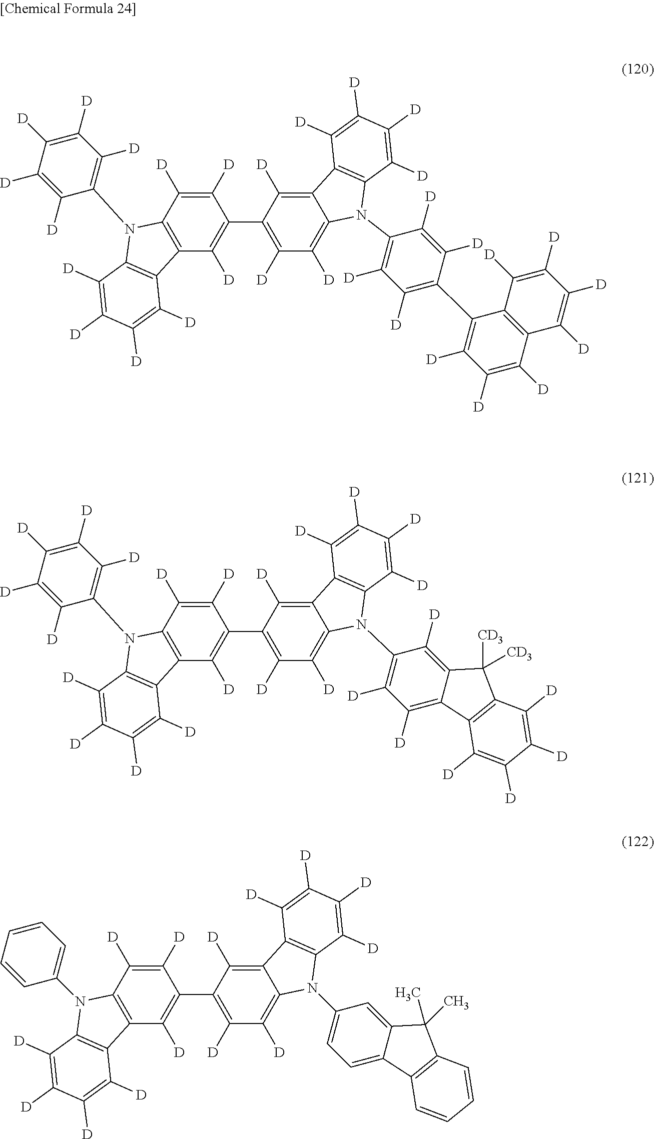

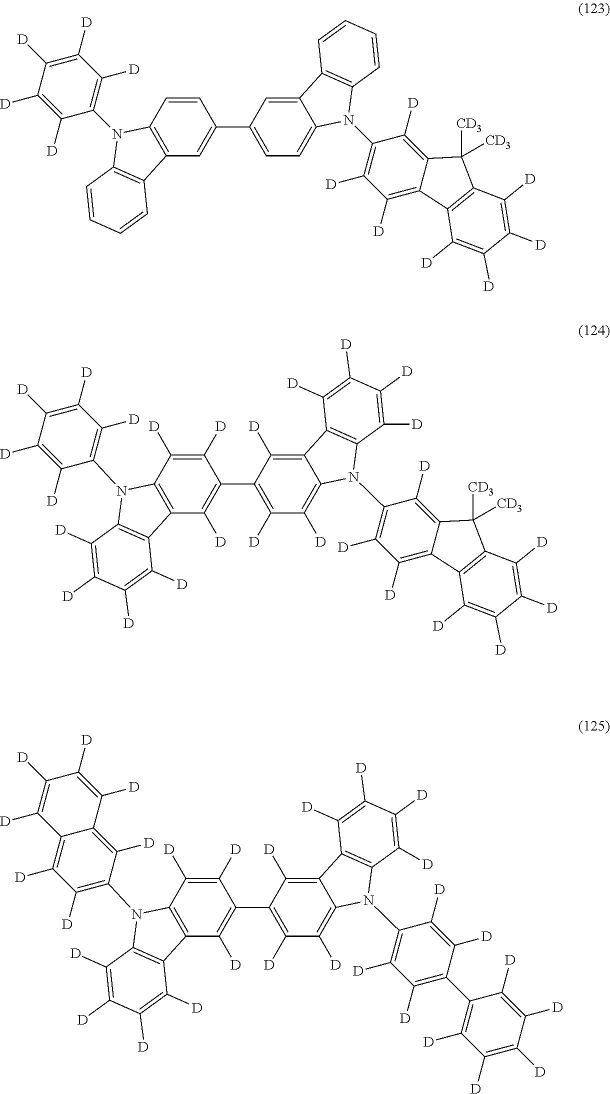

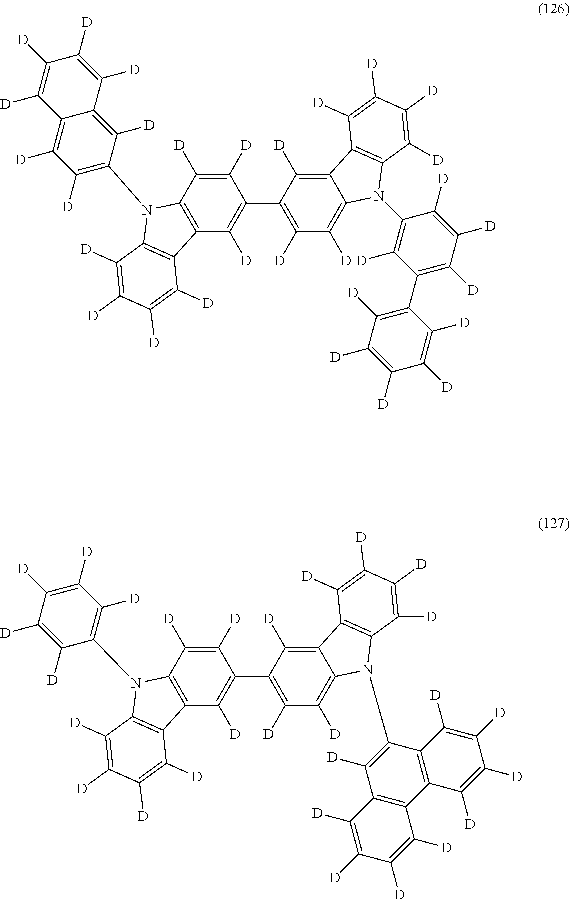

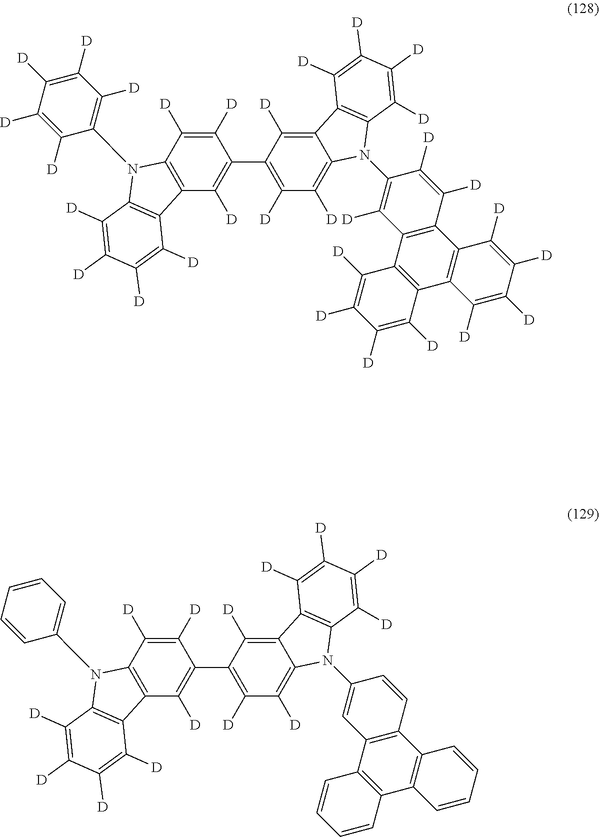

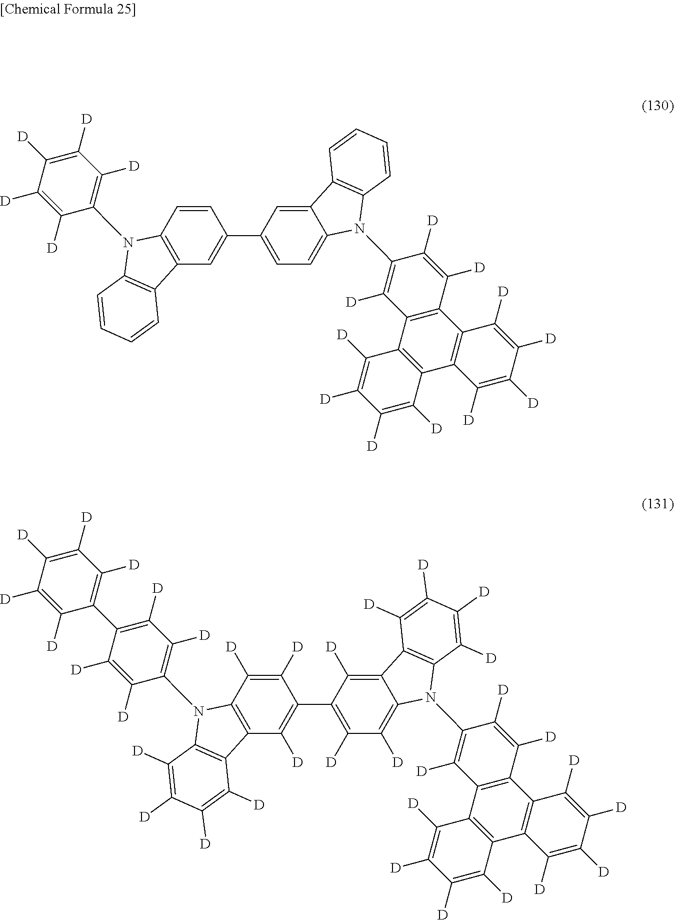

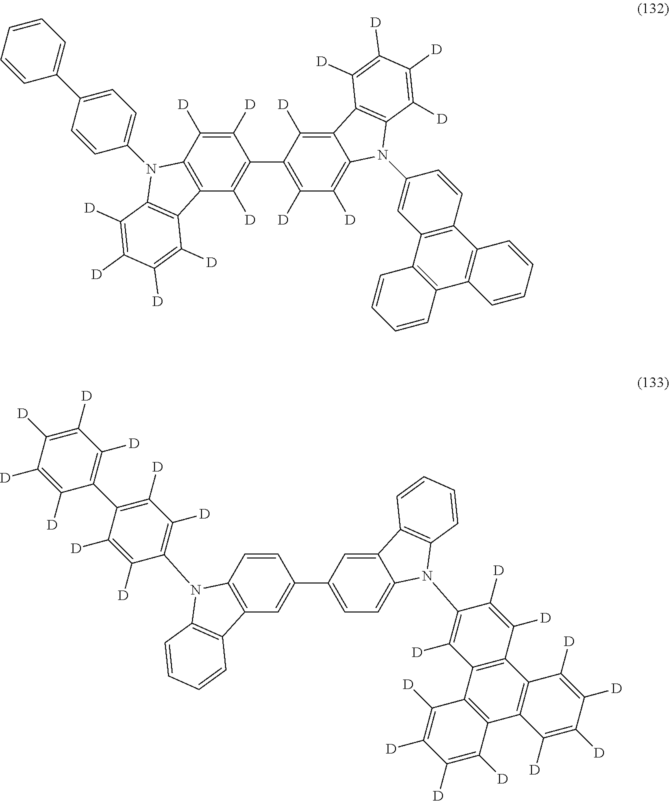

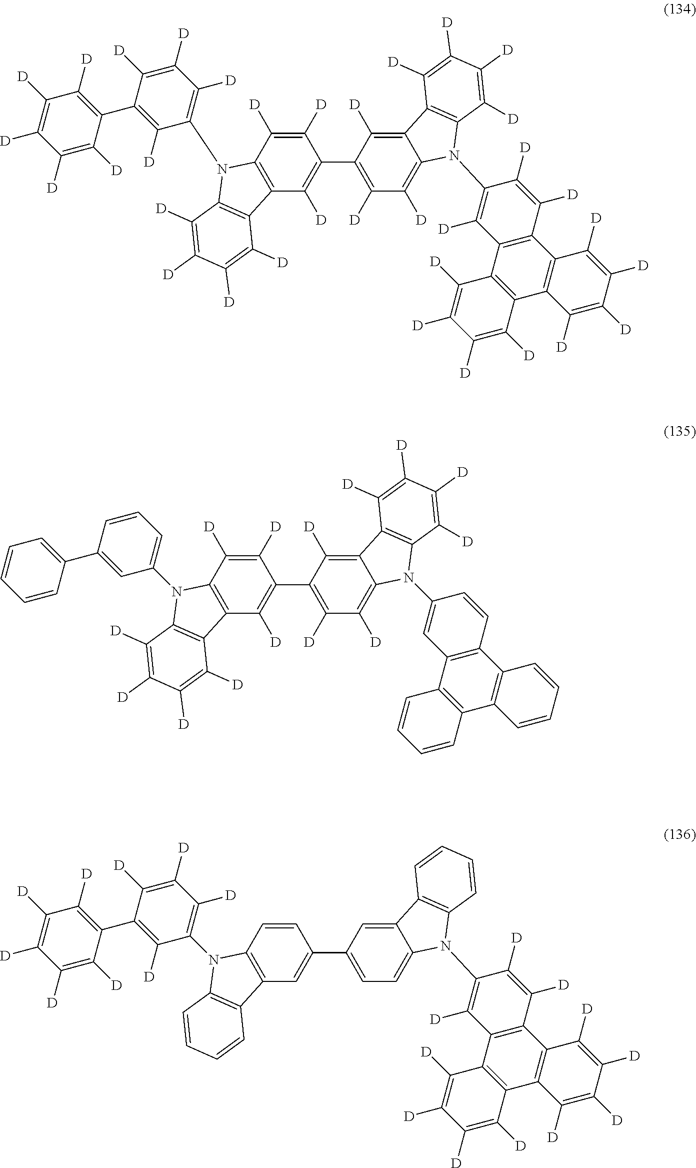

Specific examples of the organic compounds represented by General Formulae (G4) to (G9) include organic compounds represented by Structural Formulae (200) to (287). Note that the organic compounds represented by General Formulae (G4) to (G9) are not limited to the organic compounds represented by Structural Formulae (200) to (287). The organic compounds represented by Structural Formulae (200) to (287) are also specific examples of the organic compounds represented by General Formulae (G1) to (G3) above.

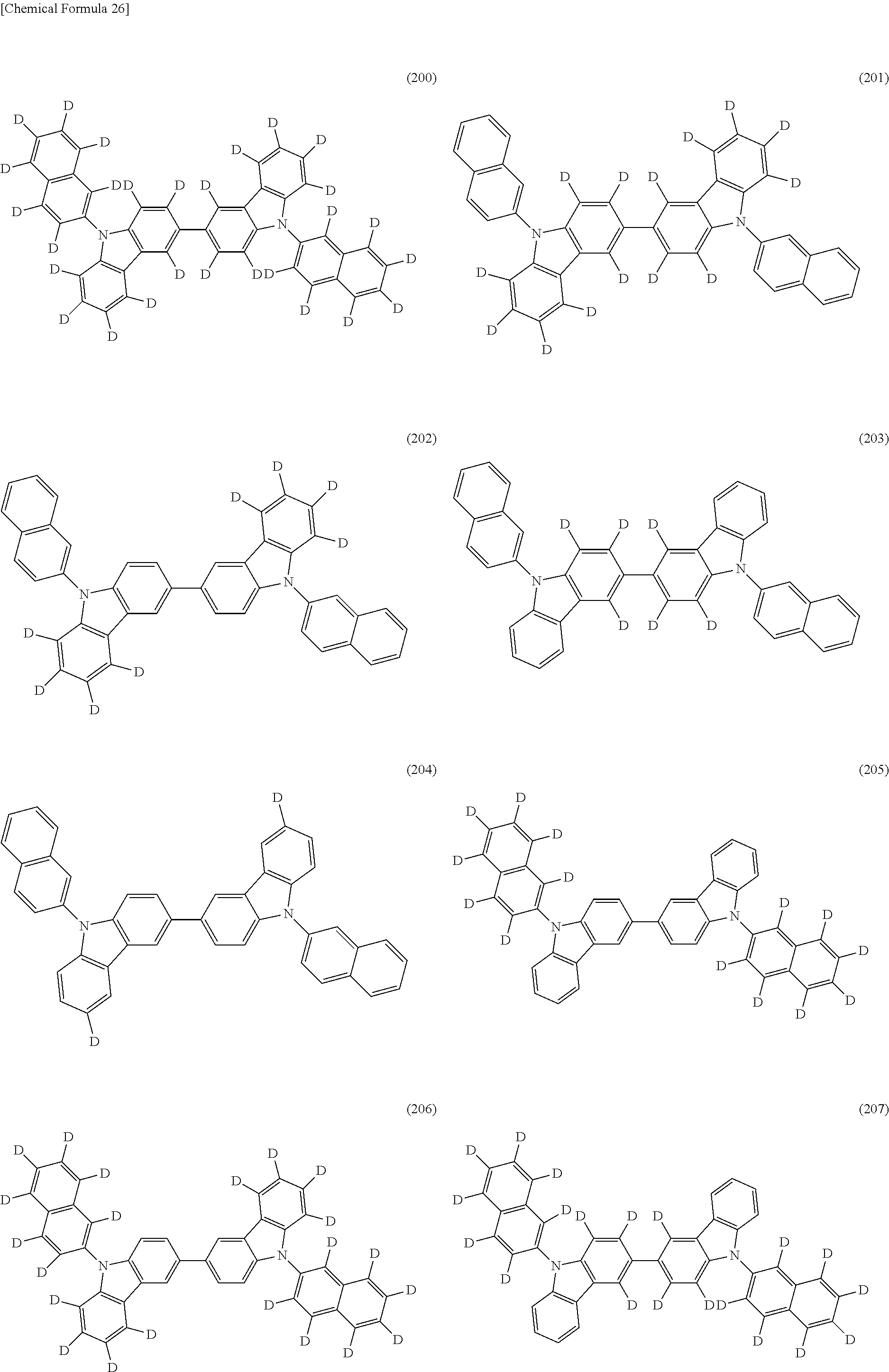

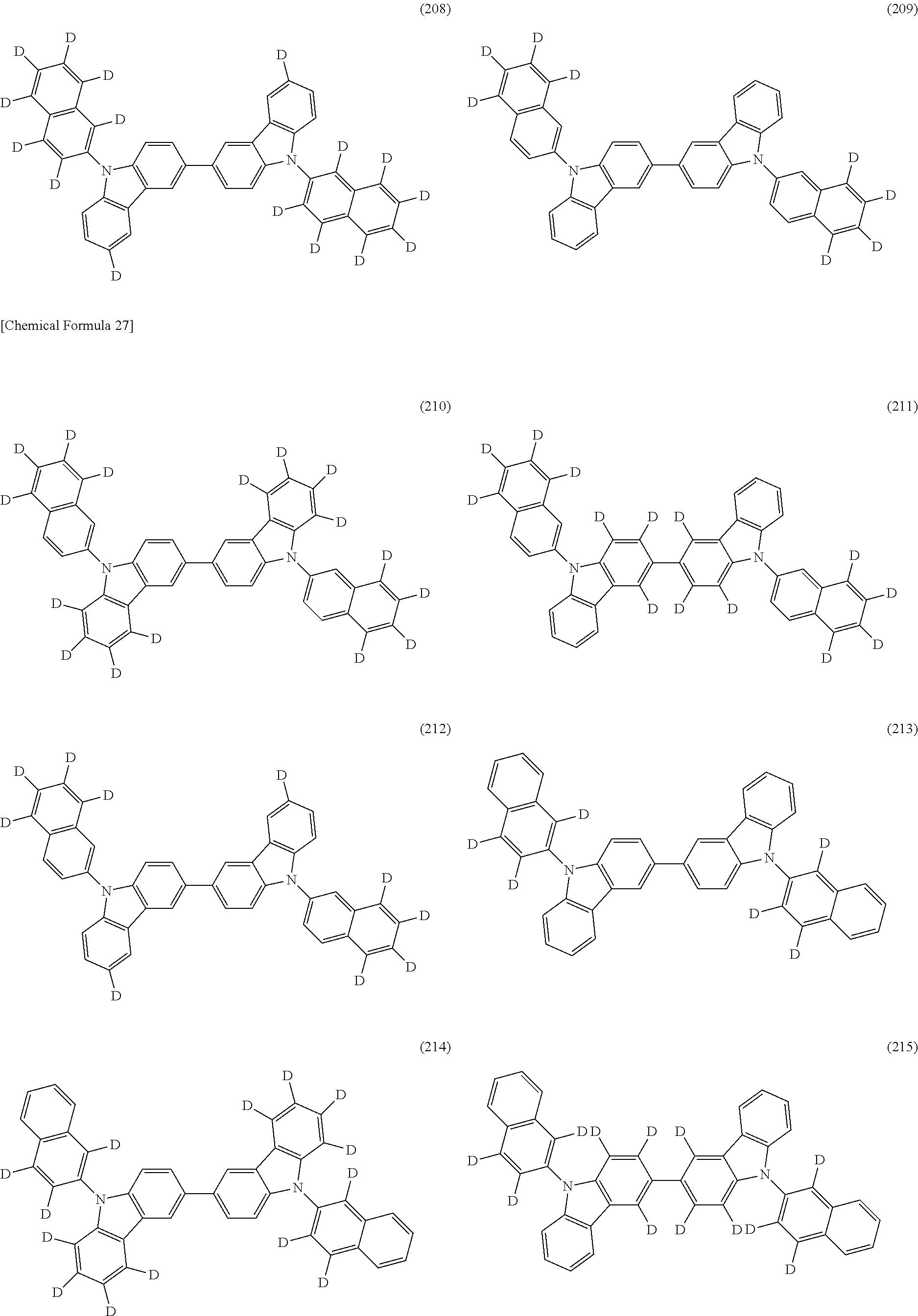

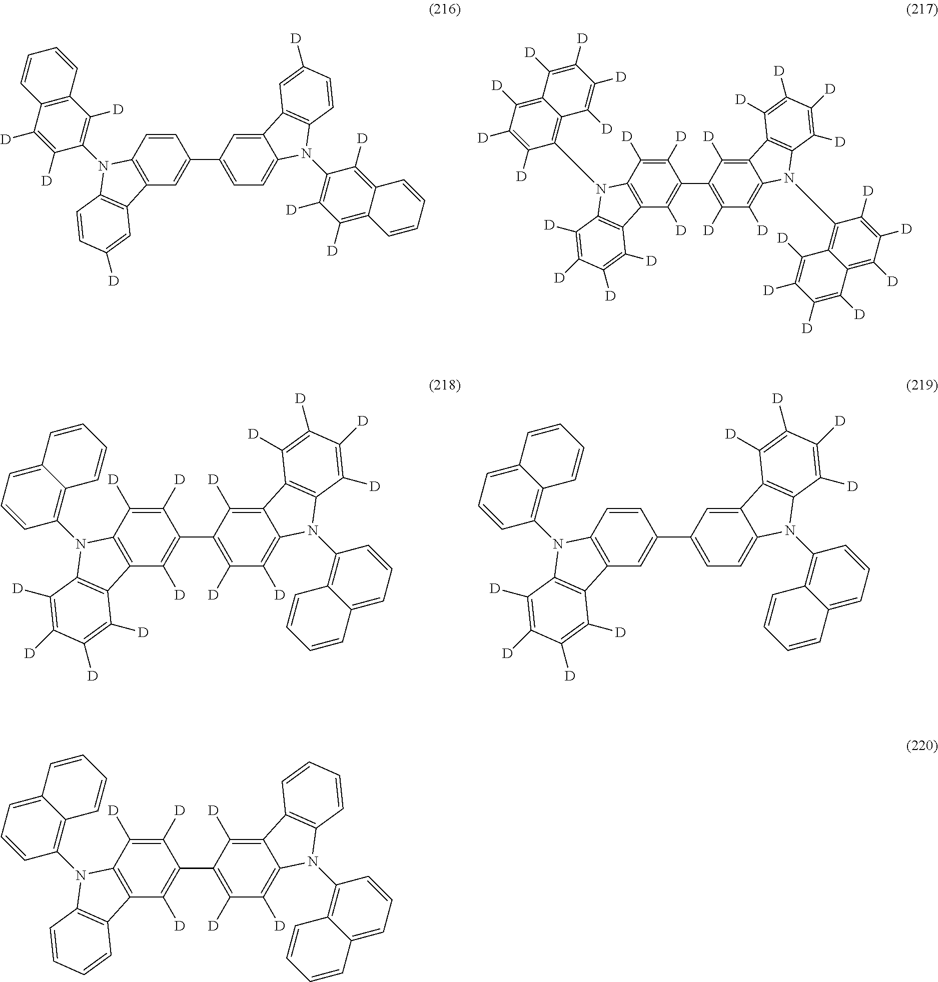

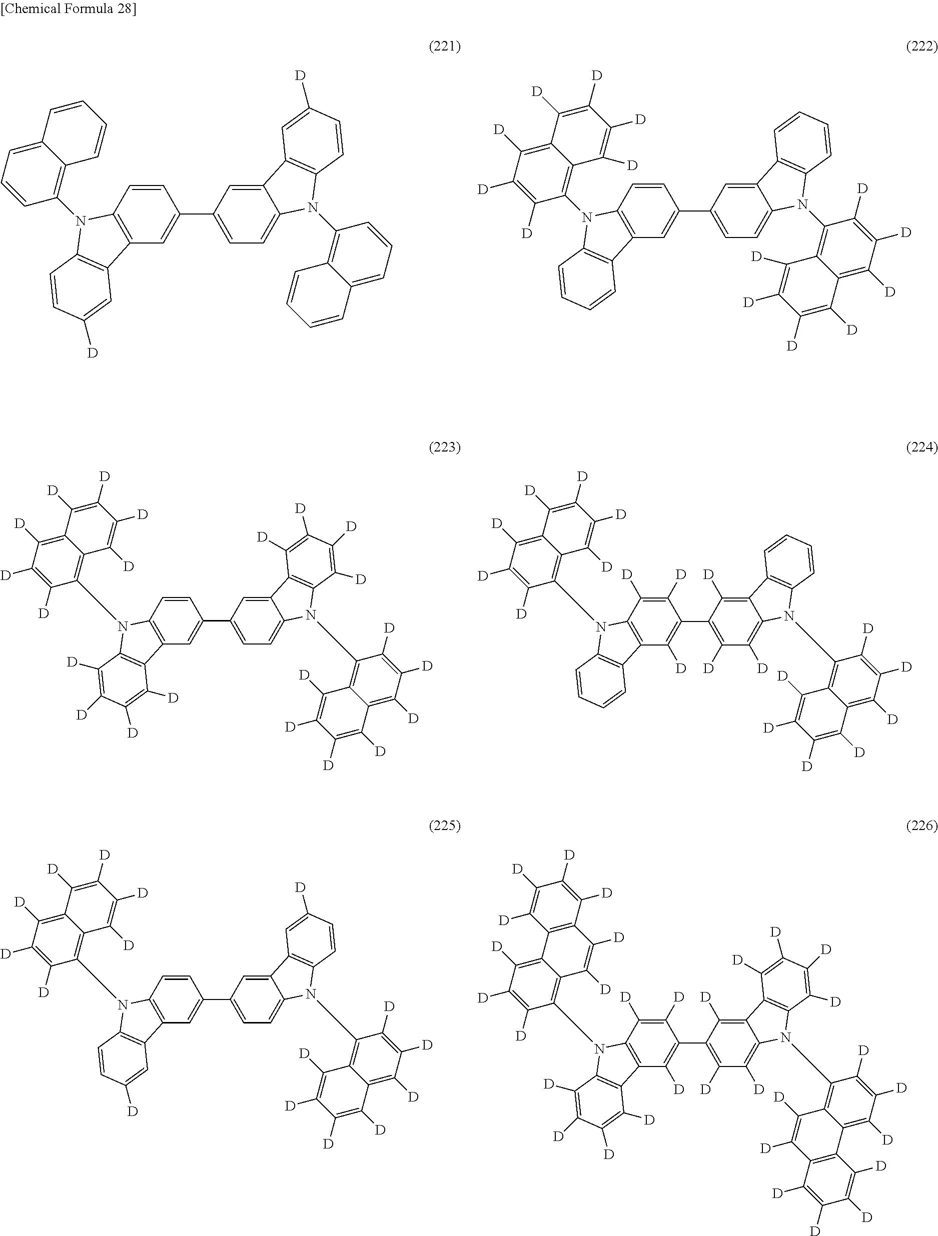

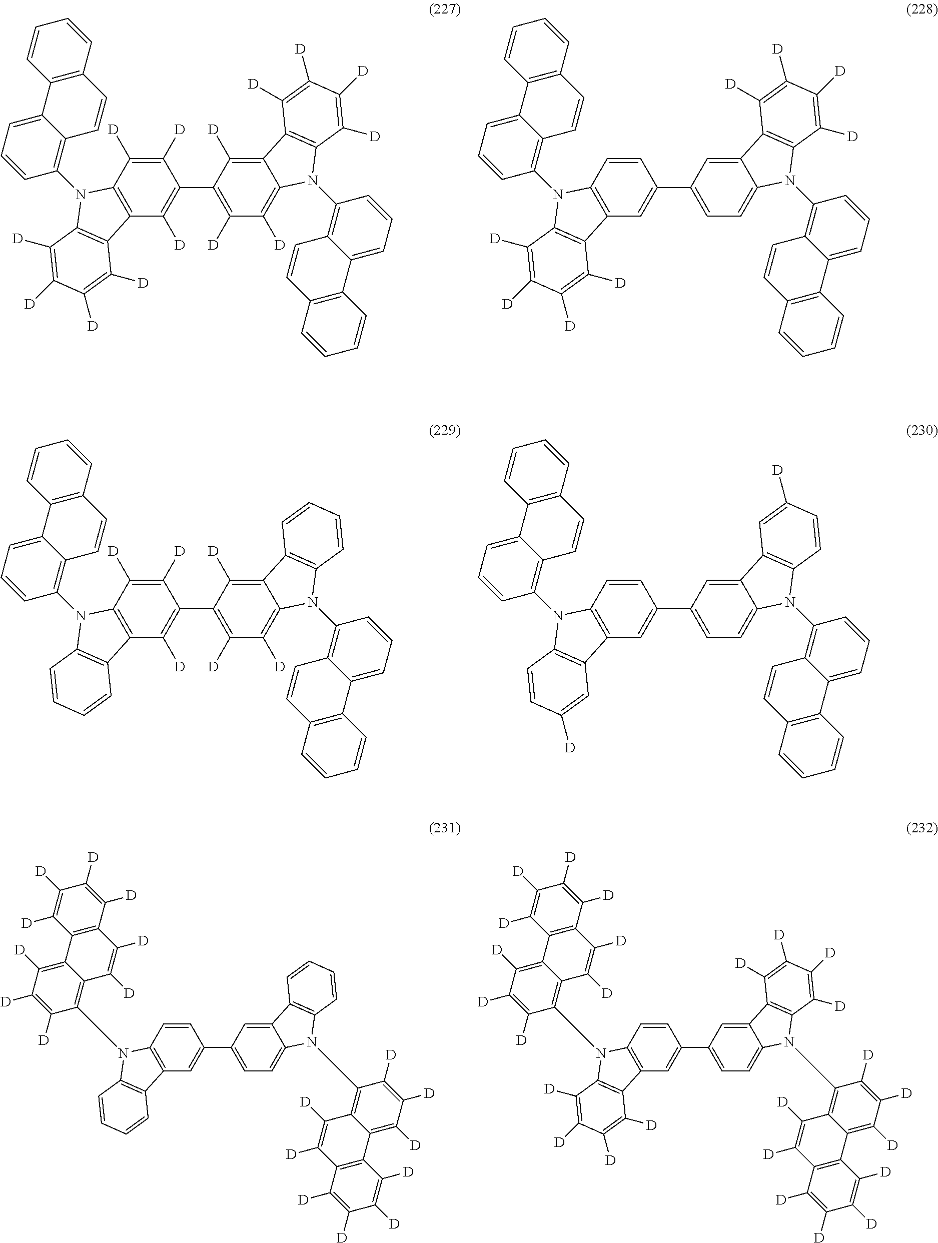

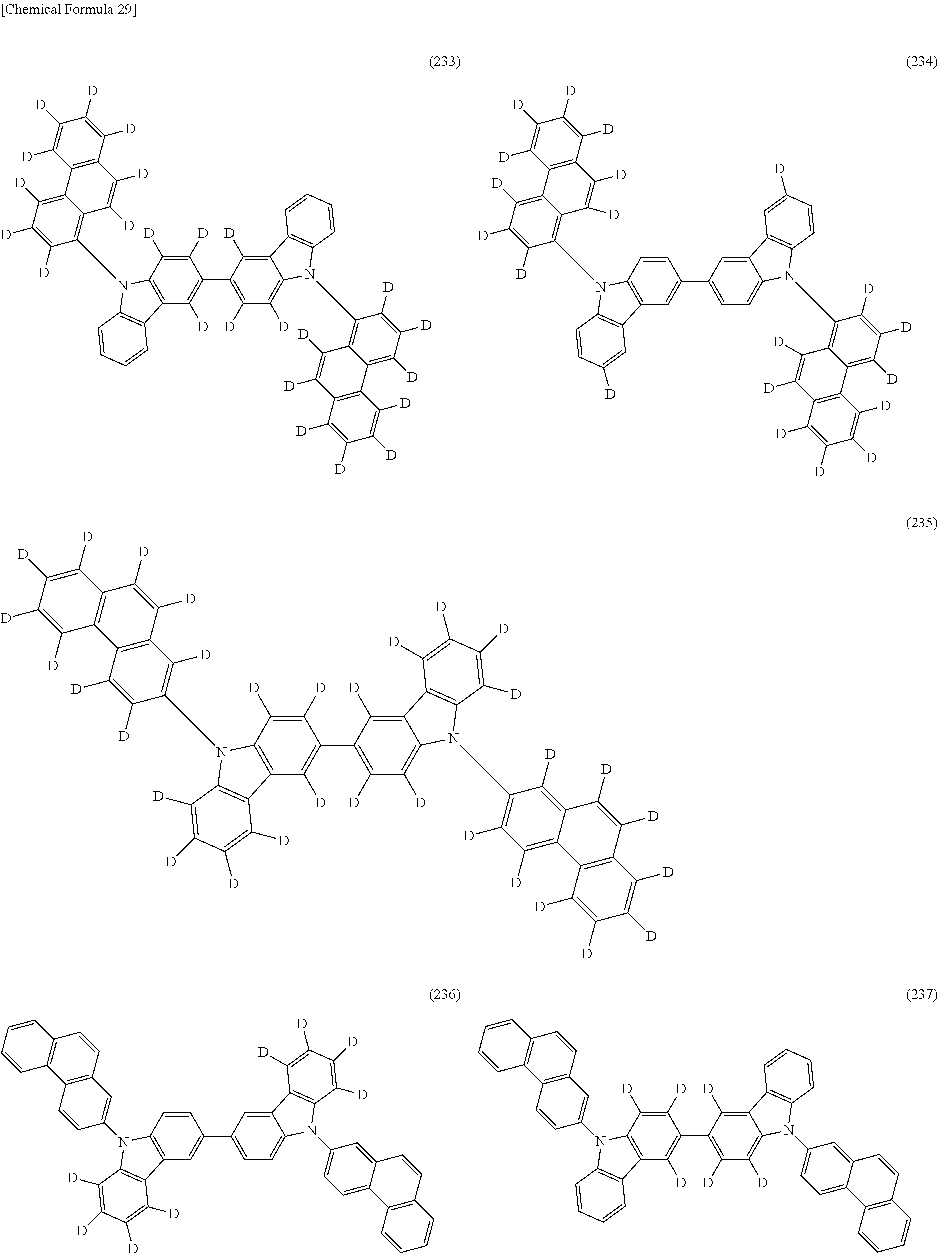

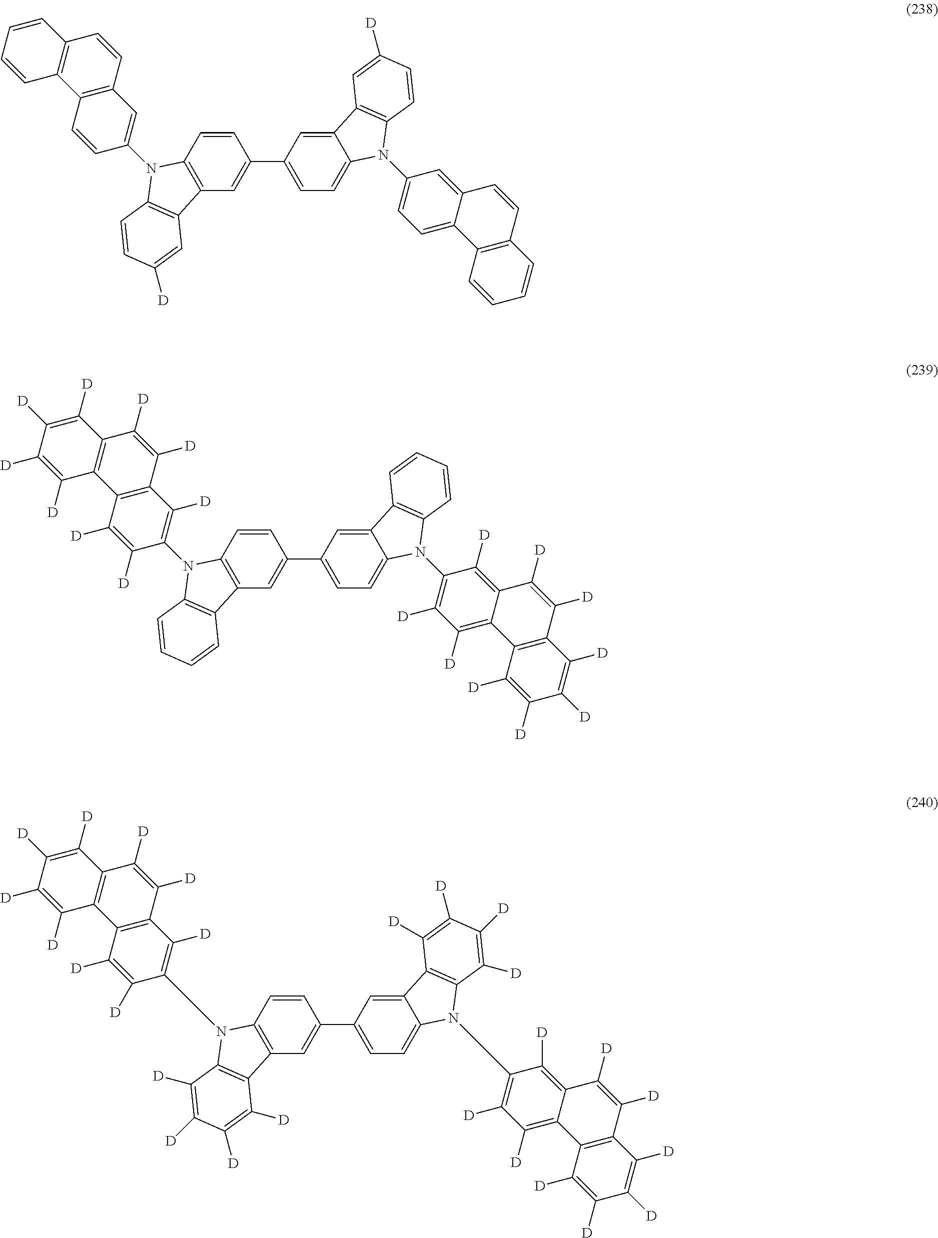

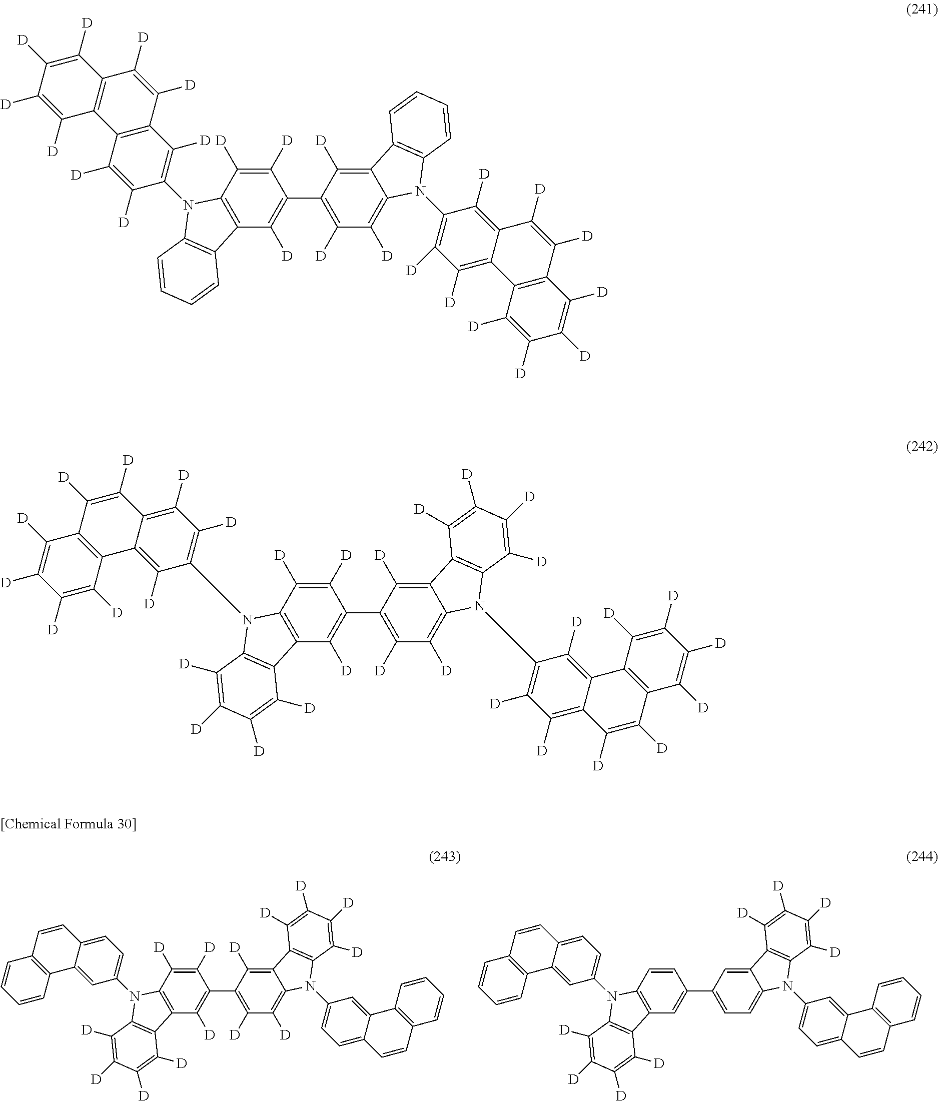

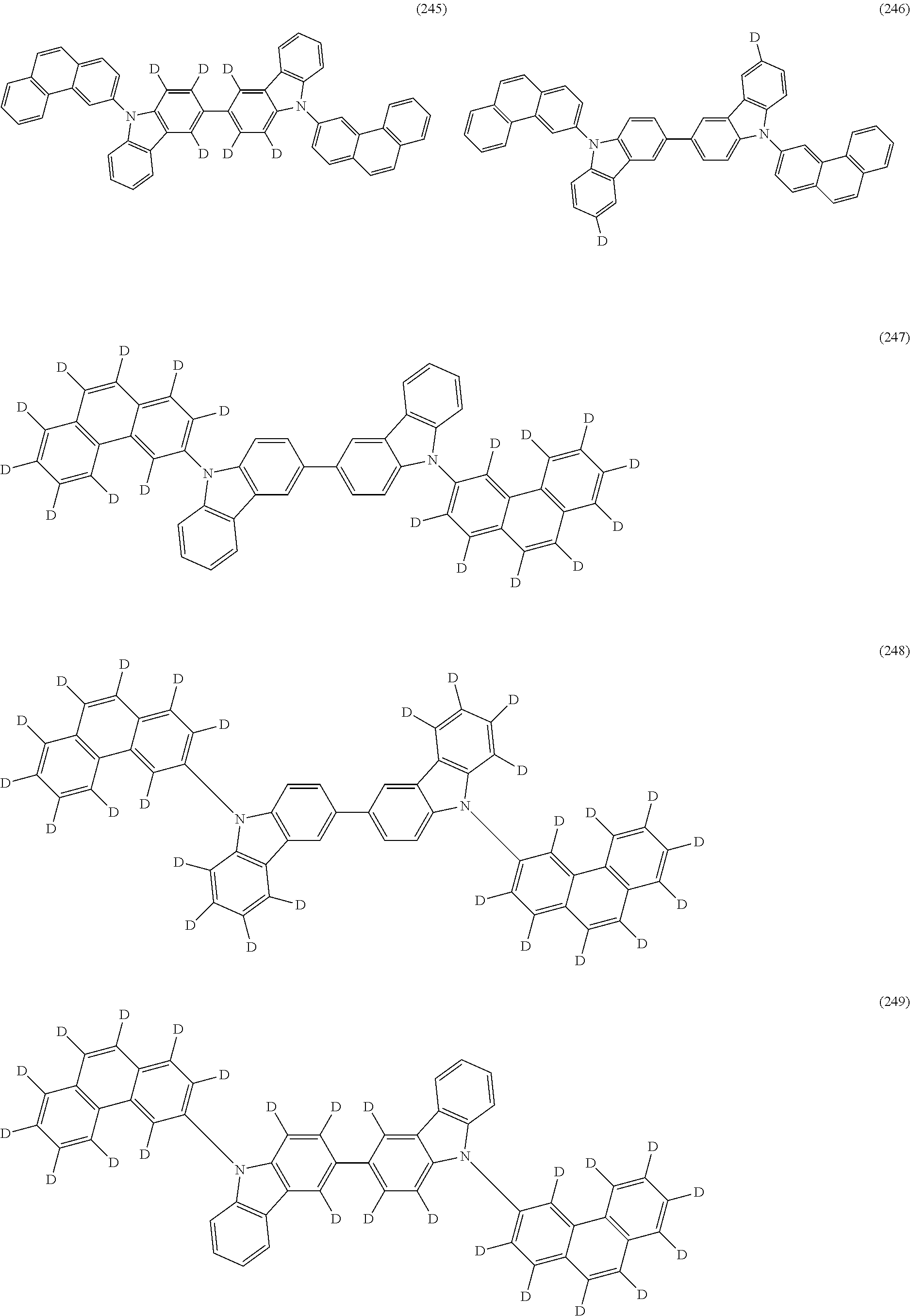

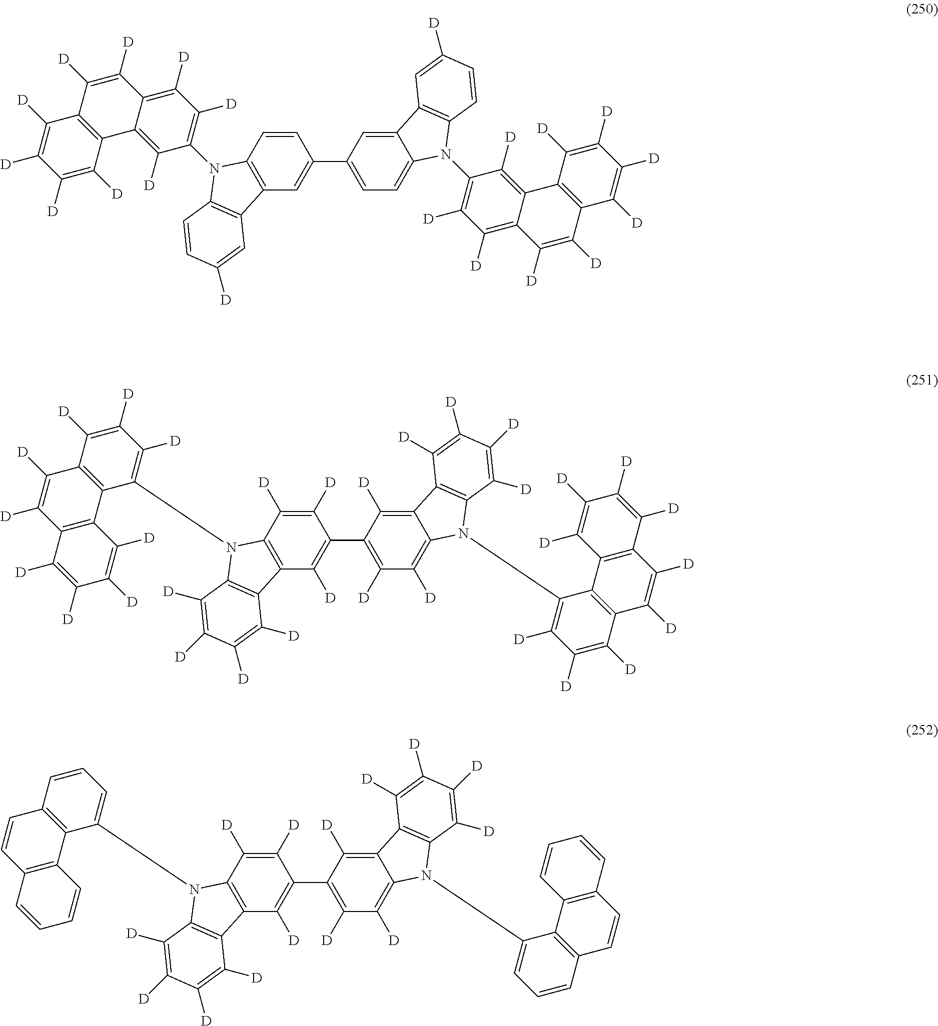

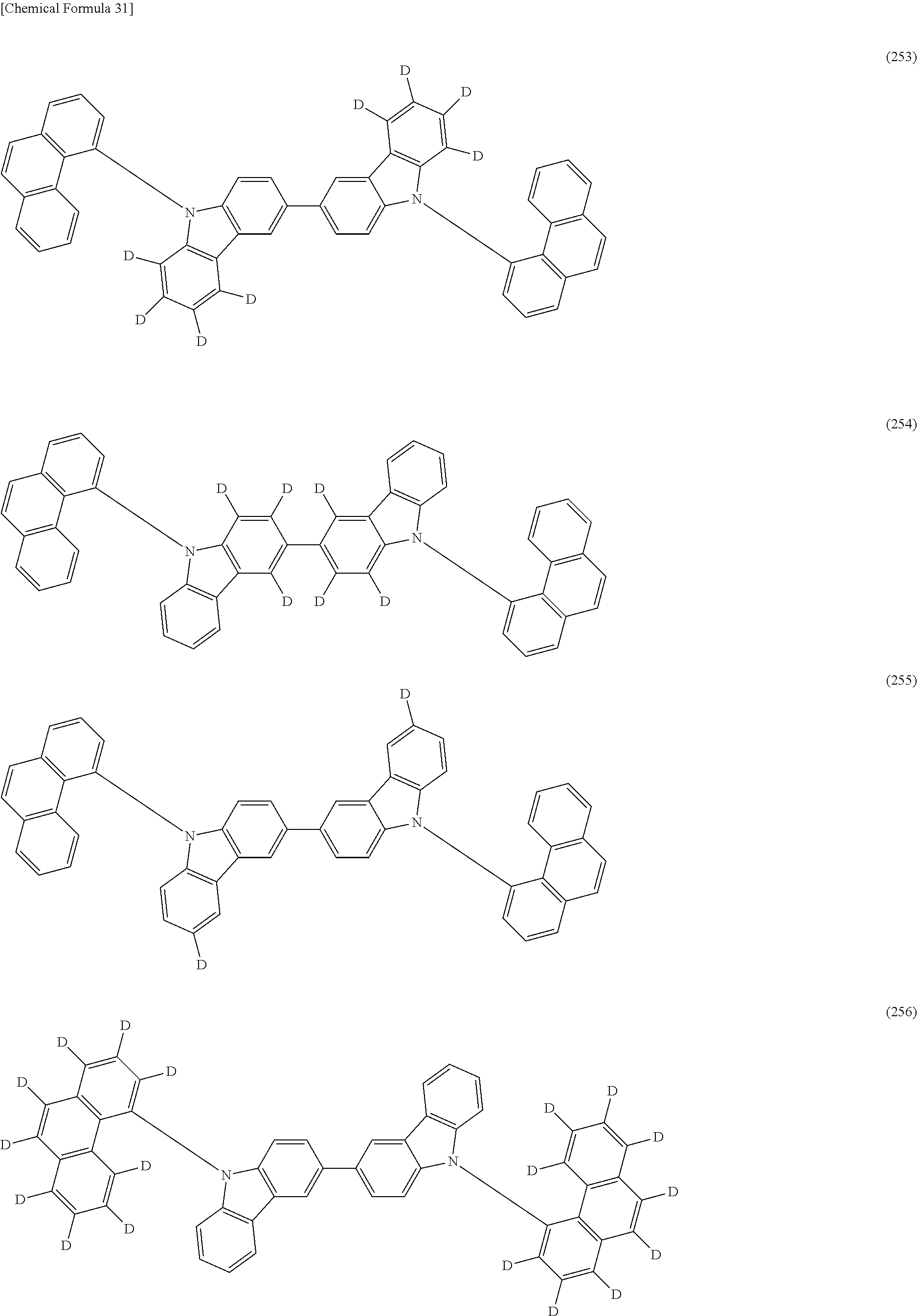

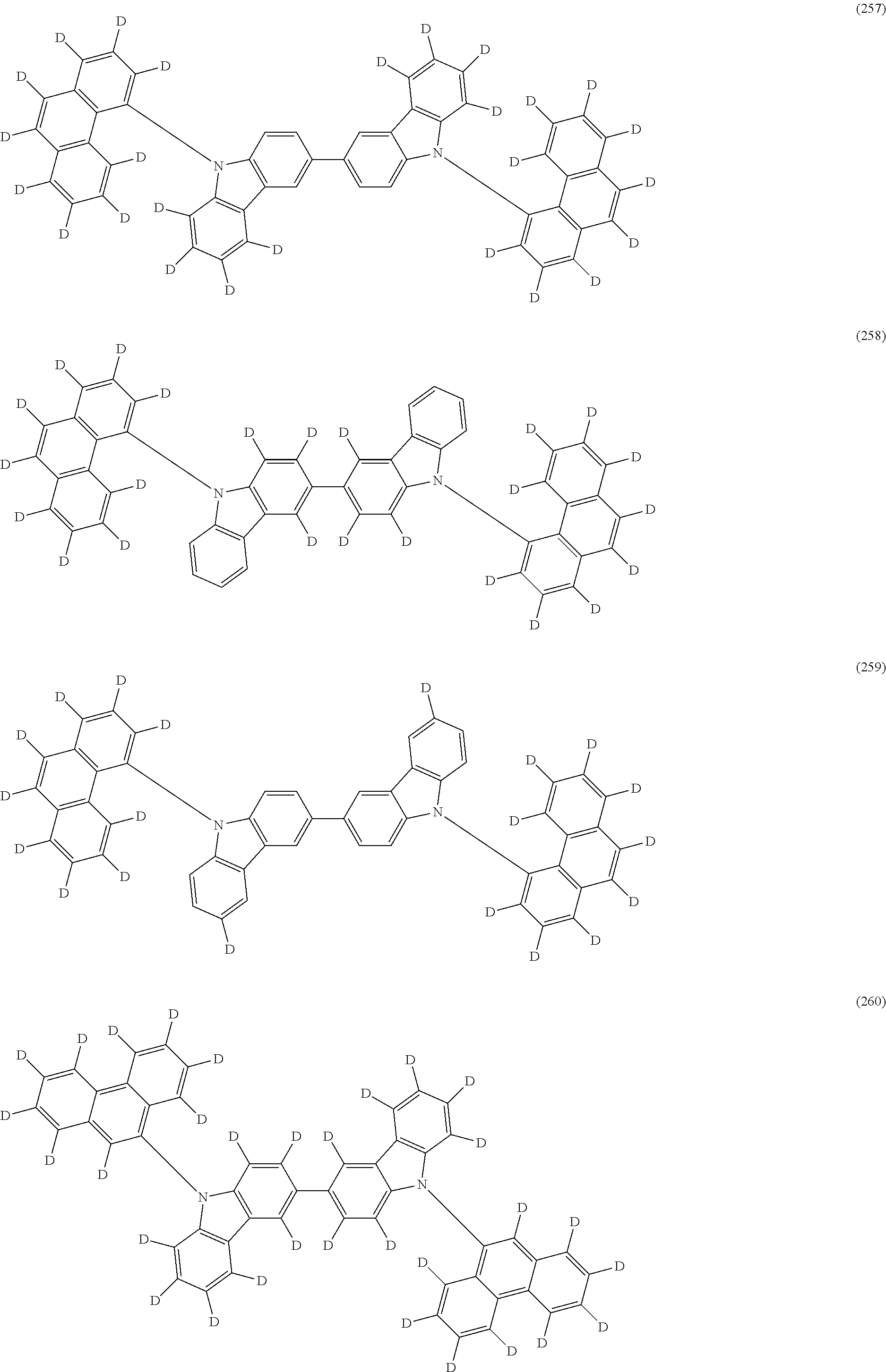

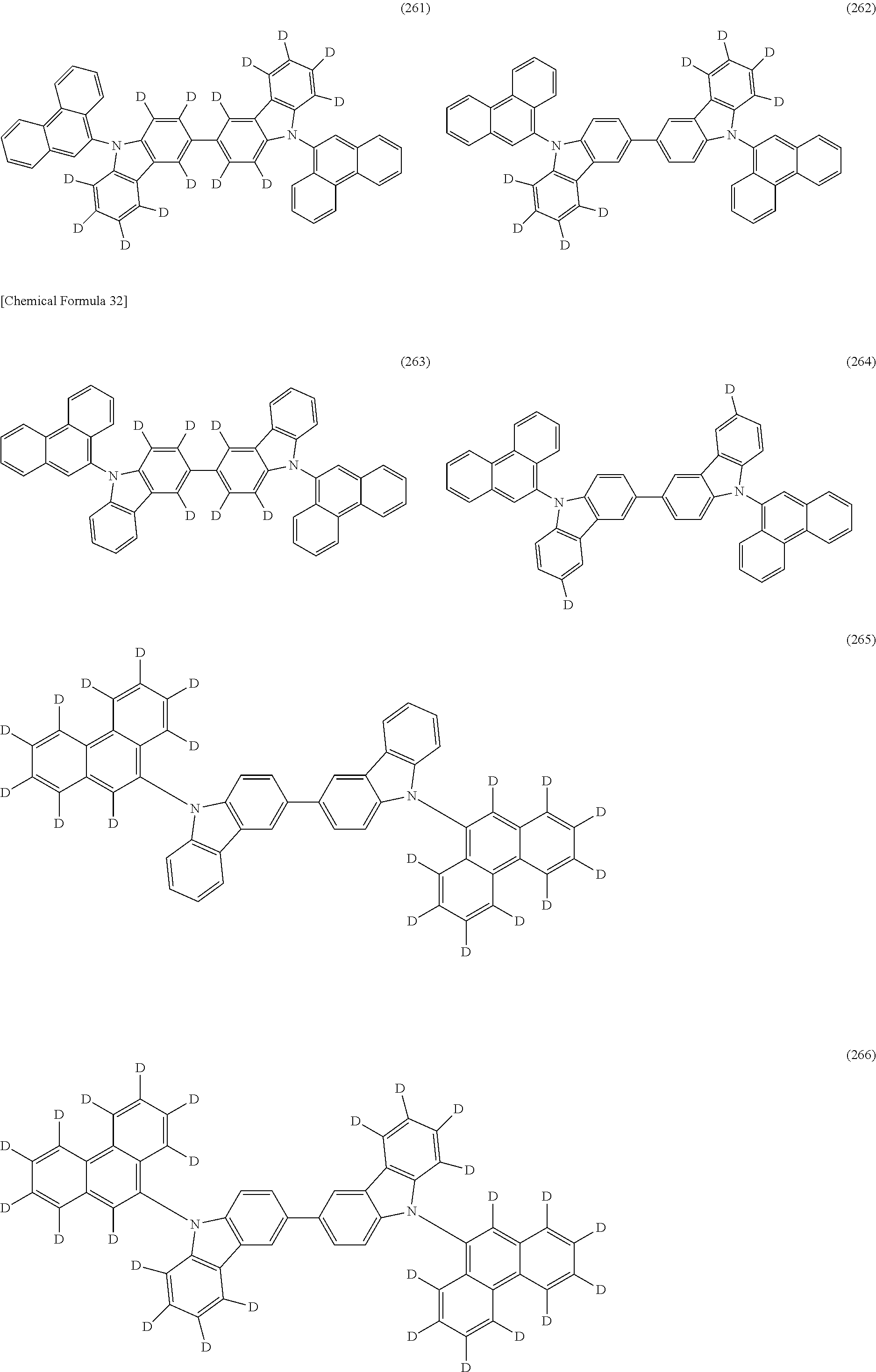

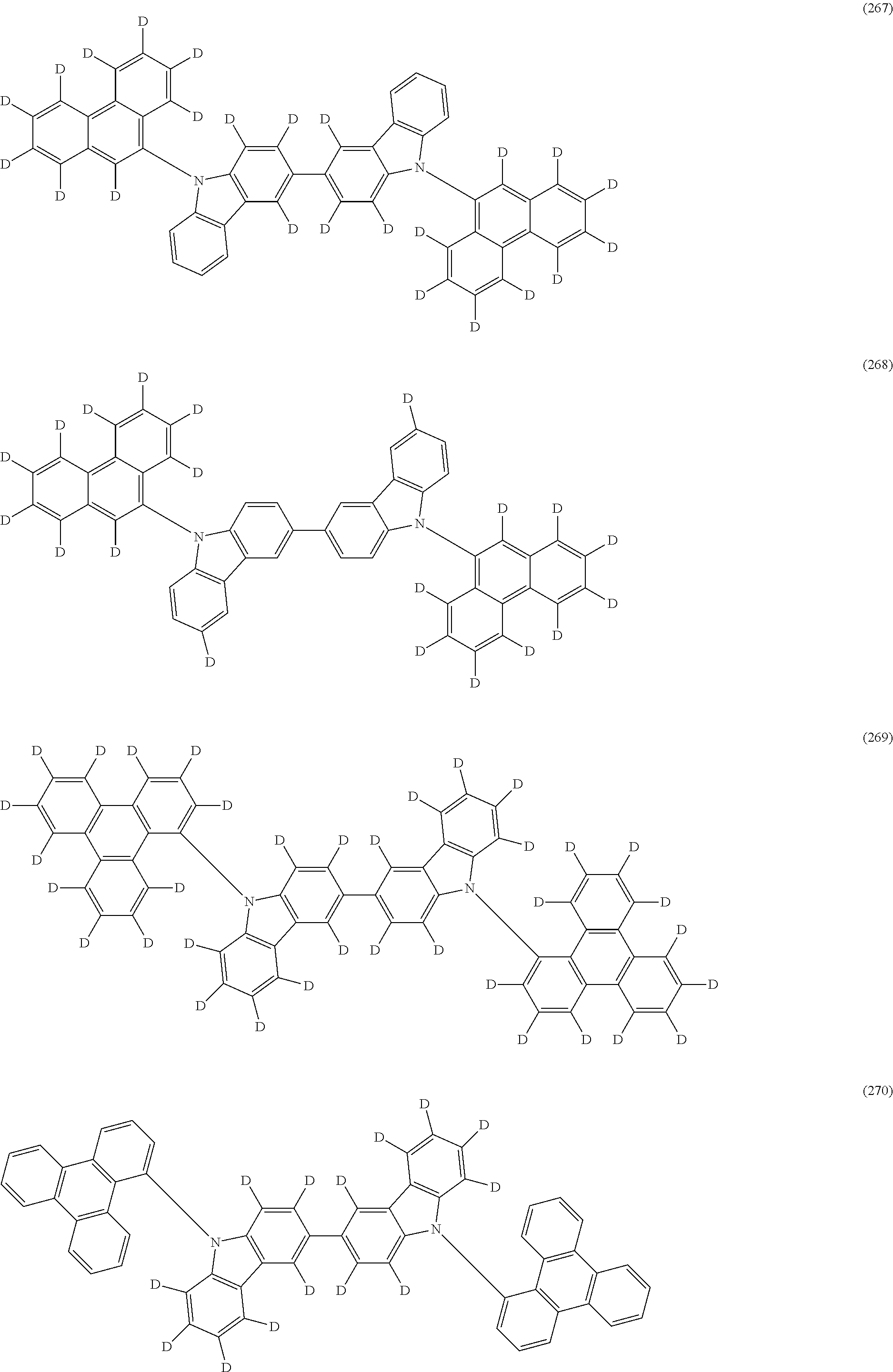

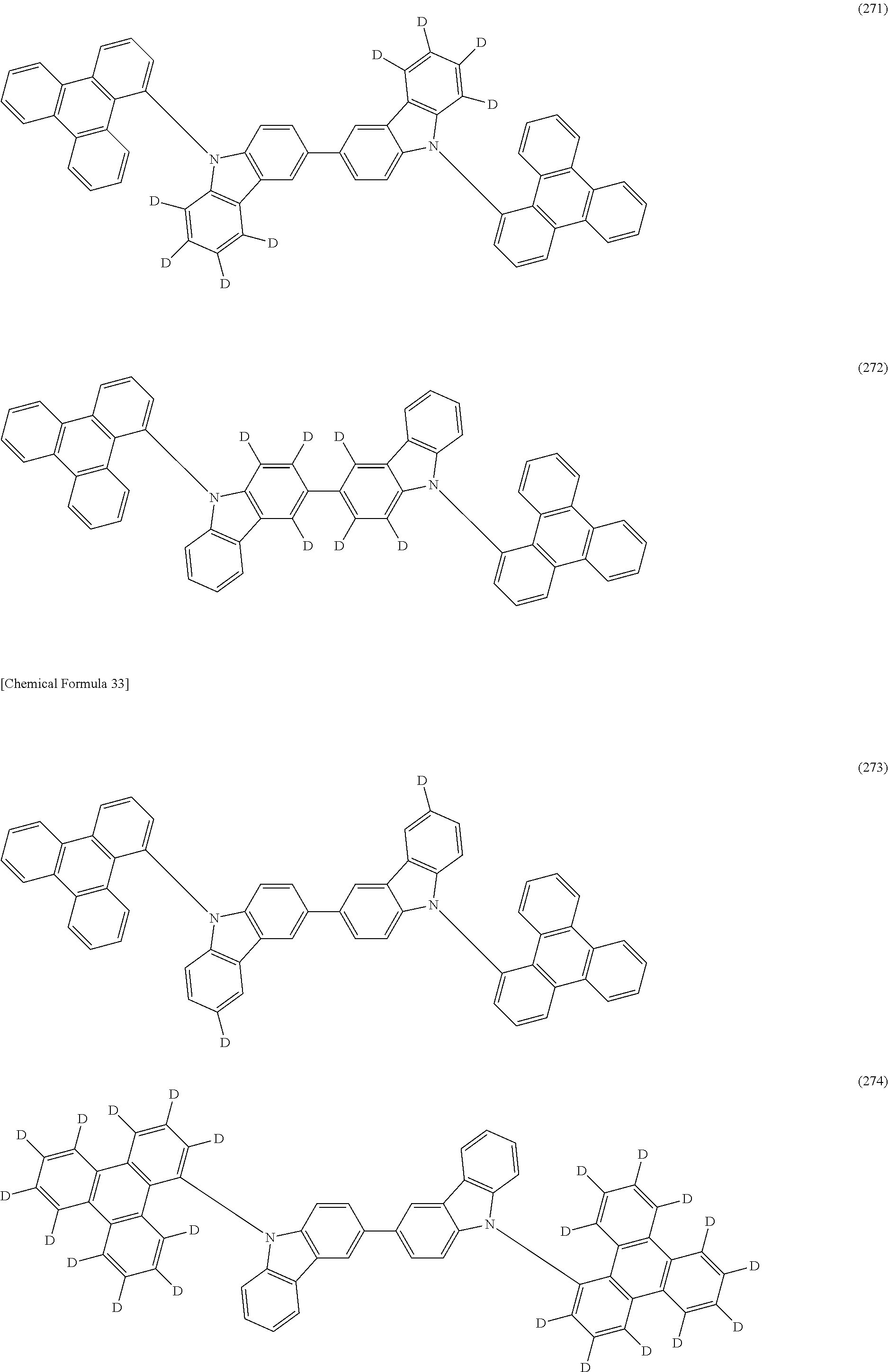

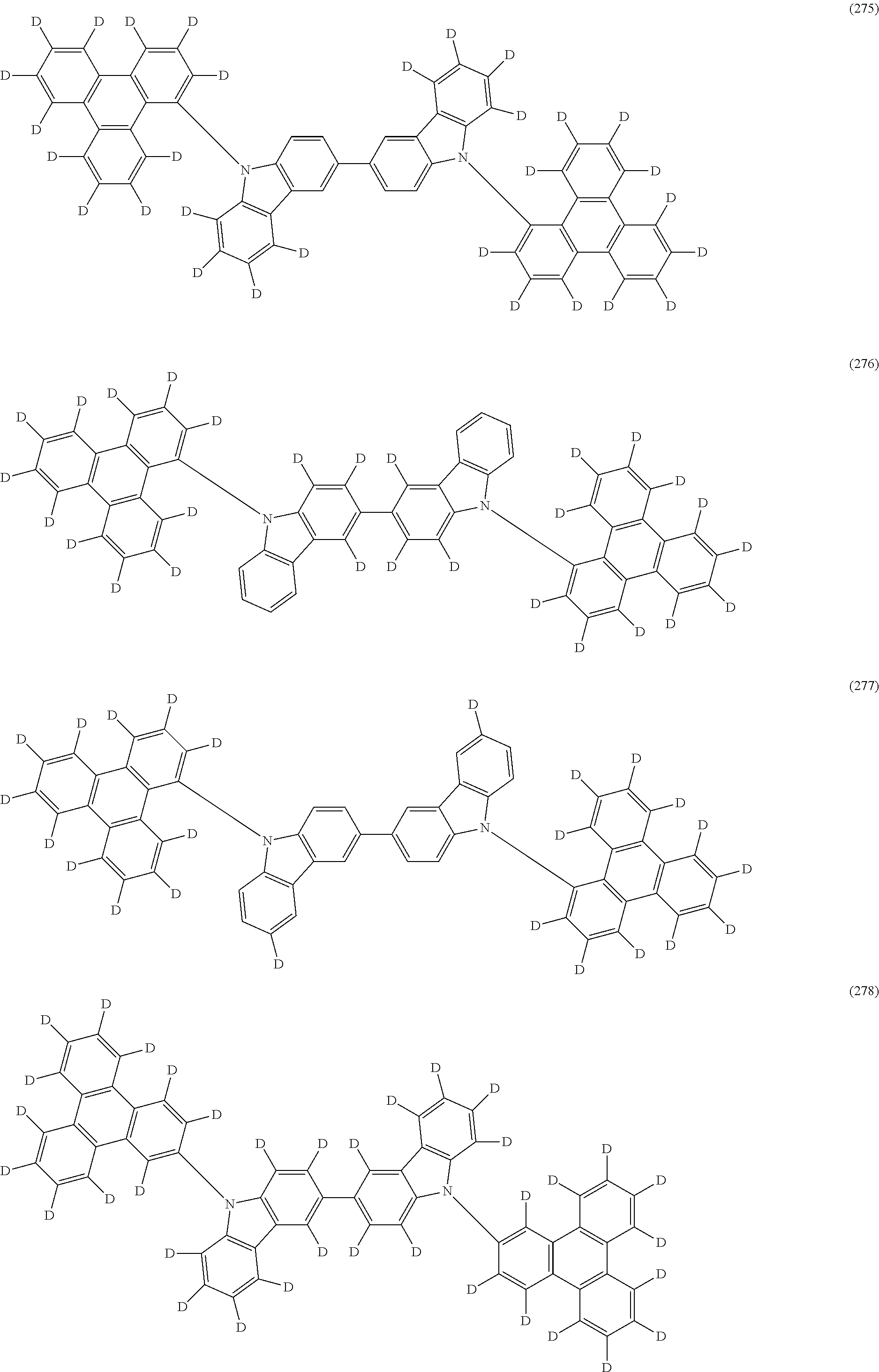

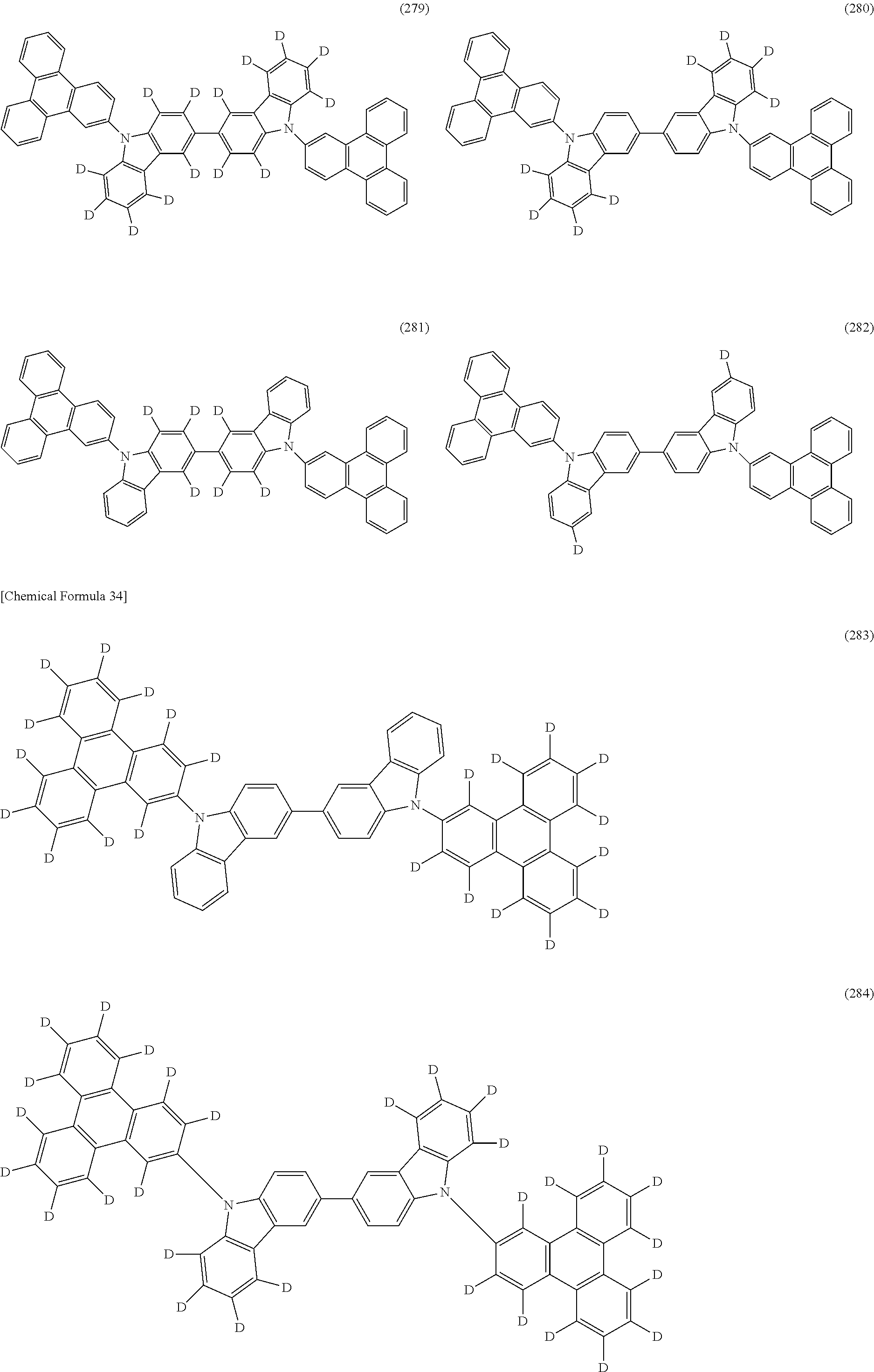

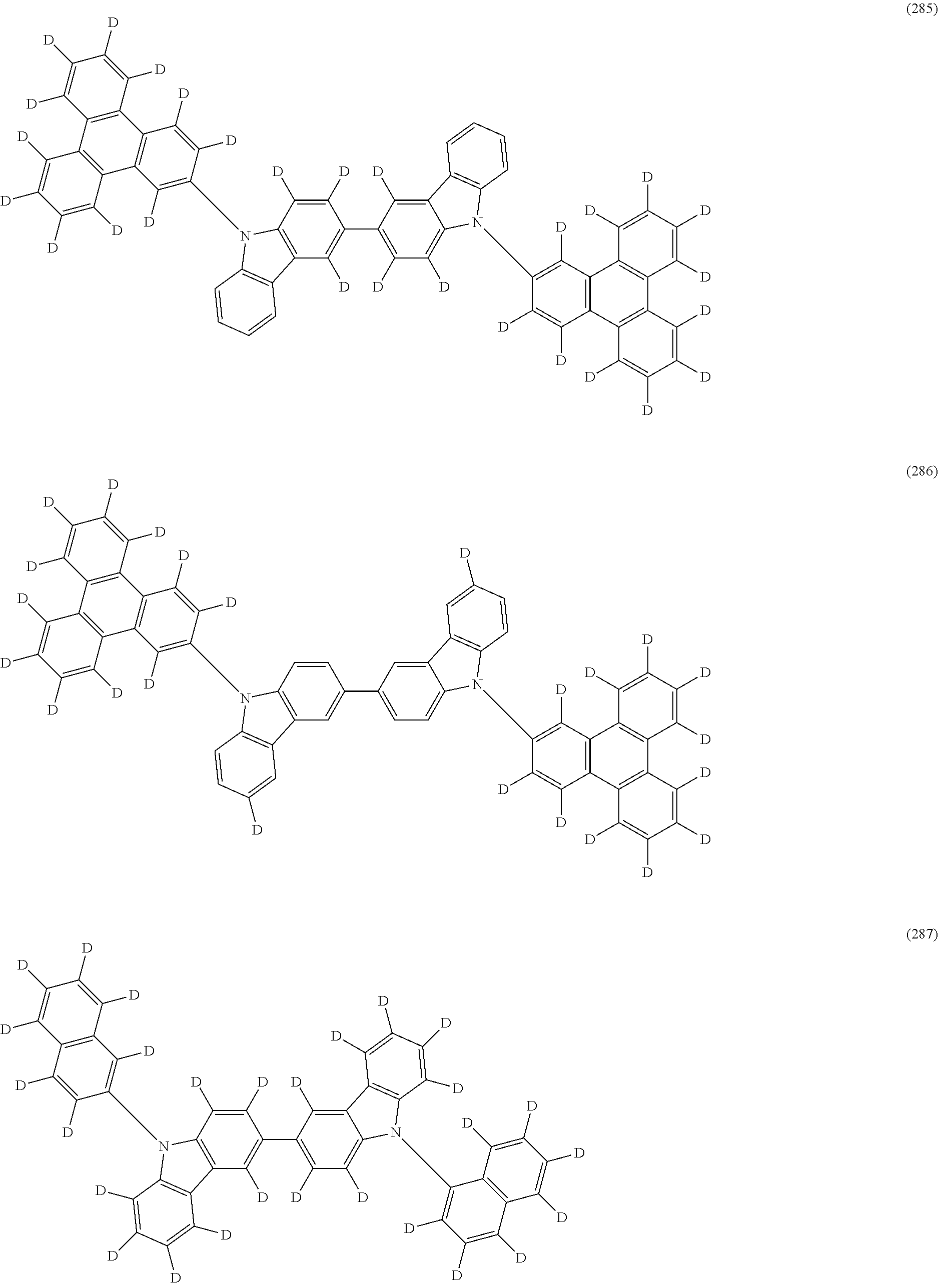

### <Synthesis Method>

In the following, a synthesis method of the organic compound represented by General Formula (G5) below is described.

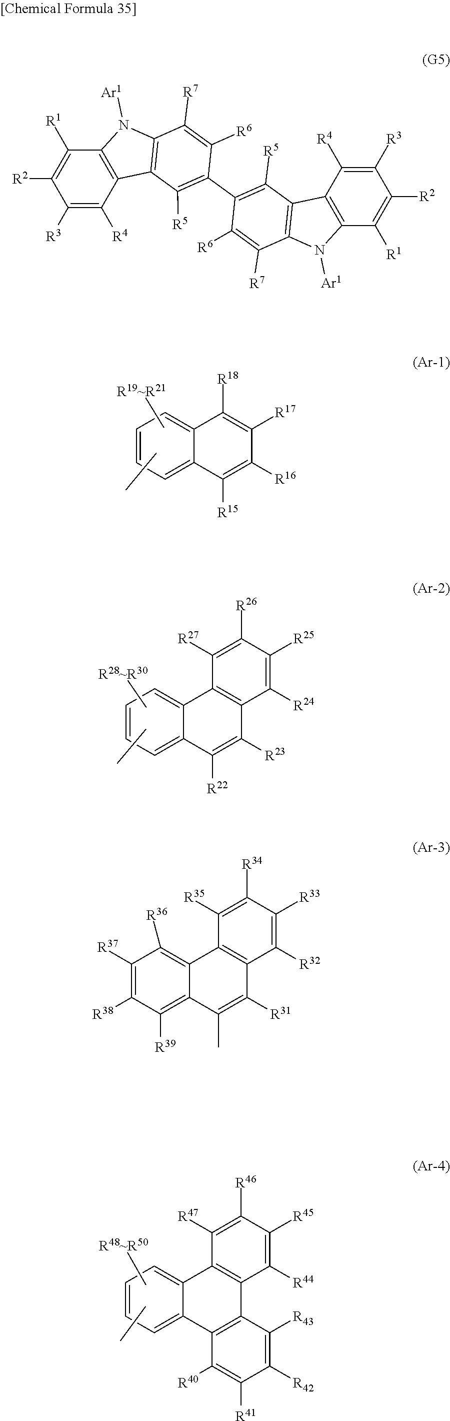

In General Formula (G5), Ar¹ is a group represented by any one of General Formulae (Ar-1) to (Ar-4), and R¹ to R⁷ each independently represent hydrogen (including deuterium). In General Formulae (Ar-1) to (Ar-4), R¹⁵ to R⁵⁰ each independently represent hydrogen (including deuterium) or an alkyl group having 1 to 6 carbon atoms. Note that the organic compound represented by General Formula (G5) contains two or more deuteriums.

In the organic compound represented by General Formula (G5), two Ar¹'s are the same group, two R¹'s are the same group, two R²'s are the same group, two R³'s are the same group, two R⁴'s are the same group, two R⁵'s are the same group, two R⁶'s are the same group, and two R⁷'s are the same group.

First, in accordance with Synthesis Scheme (a-1), an aryl halide compound (Compound 1) and a 9H-carbazole compound (Compound 2) are coupled, whereby a 9-aryl-9H-carbazole compound (Compound 3) can be obtained.

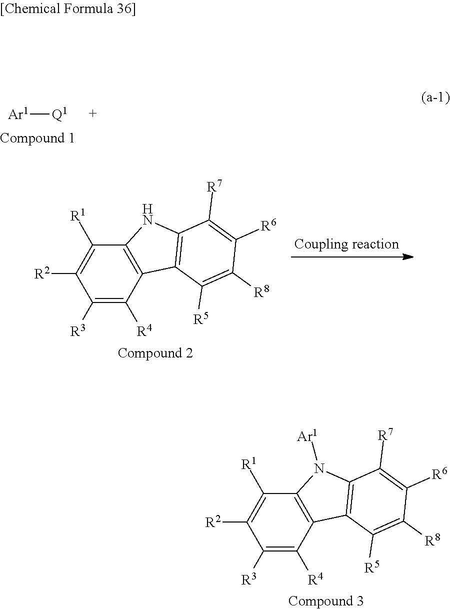

In Synthesis Scheme (a-1), Ar¹ and R¹ to R⁷ are the same as those described above and are not described here. In Synthesis Scheme (a-1), Q¹ represents chlorine, bromine, iodine, or a trifluoromethanesulfonyl group.

In the case where the Buchwald-Hartwig reaction using a palladium catalyst is employed in Synthesis Scheme (a-1), a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride (dimer) and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, or di(tert-butyl)(1-methyl-2,2-diphenylcyclopropyl)phosphine (abbreviation: cBRIDP), can be used. In the reaction, an organic base such as sodium-tert-butoxide, an inorganic base such as potassium carbonate, cesium carbonate, or sodium carbonate, or the like can be used. In the reaction, an interphase transfer catalyst such as 18-crown-6 can also be used. In the reaction, toluene, xylene, benzene, tetrahydrofuran, dioxane, or the like can be used as a solvent. Reagents that can be used in the reaction are not limited to the above-described reagents.

In Synthesis Scheme (a-1), an Ullmann reaction using copper or a copper compound and nickel or a nickel compound can also be performed. As an example of the base to be used in the reaction, an inorganic base such as potassium carbonate can be given. As a solvent that can be used in the reaction, 1,3-dimethyl-3,4,5,6-tetrahydro-2(1H)-pyrimidinone (DMPU), N-methylpyrrolidone, toluene, xylene, benzene, and the like can be given. In the Ullmann reaction, when the reaction temperature is higher than or equal to 100° C., the target substance can be obtained in a shorter time in a higher yield; thus, it is preferable to use DMPU or xylene having a high boiling point. A reaction temperature of 150° C. or higher is further preferable, and accordingly, DMPU is further preferably used. Reagents that can be used in the reaction are not limited to the above-described reagents.

Next, in accordance with Synthesis Scheme (a-2), the 9-aryl-9H-carbazole compound (Compound 3) and a halogenating reagent (Compound 4) are reacted, whereby a 9-aryl-9H-carbazole halide compound (Compound 5) can be obtained.

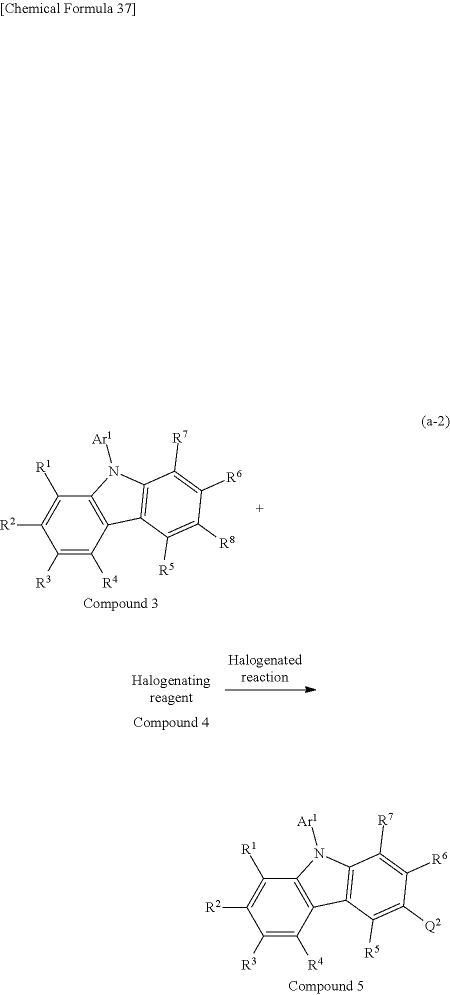

In Synthesis Scheme (a-2), Ar¹ and R¹ to R⁷ are the same as those described above and are not described here. In the synthesis scheme (a-2), Q² represents chlorine, bromine, or iodine. A trifluoromethanesulfonyl group can also be used as Q².

In Synthesis Scheme (a-2), for example, N-chlorosuccinimide, N-bromosuccinimide, N-iodosuccinimide, or bromine can be used as a halogenating reagent. Reagents that can be used in the reaction are not limited to the above-described reagents.

Next, the target bicarbazole compound (G5) can be obtained also by a method in which 9-aryl-9H-carbazole compounds (Compounds 3) are homocoupled in accordance with Synthesis Scheme (a-3).

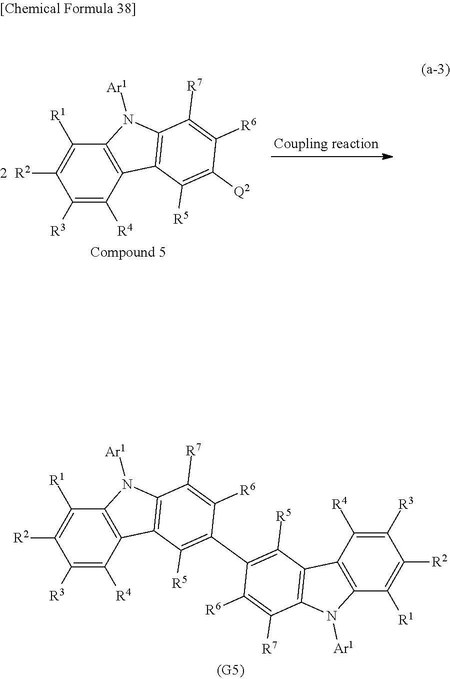

In Synthesis Scheme (a-3), Ar¹, R¹ to R⁷, and Q² are the same as those described above and are not described here.

In the case where the Buchwald-Hartwig reaction using a palladium catalyst or the Ullmann reaction using copper or copper oxide is performed in Synthesis Scheme (a-3), the same reaction conditions as those in Synthesis Schemes (a-1) and (a-2) can be used.

Then, in accordance with Synthesis Scheme (a-4), a 9-aryl-9H-carbazole halide compound (Compound 5) and a borylation reagent (Compound 6) are reacted, whereby a 9-aryl-9H-carbazole boryl compound (Compound 7) can be obtained.

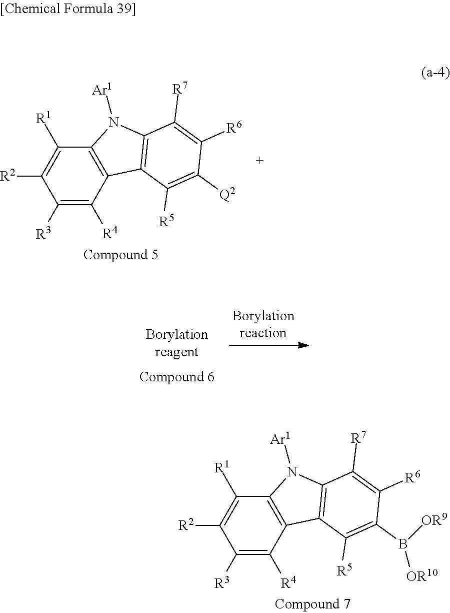

In Synthesis Scheme (a-5), Ar¹, R¹ to R⁷, and Q² are the same as those described above and are not described here.

In Synthesis Scheme (a-5), R⁹ and R¹⁰ each independently represent hydrogen or an alkyl group having 1 to 6 carbon atoms, R⁹ and R¹⁰ may be bonded to each other to form a ring, and examples of a boron compound in that case include pinacol borane.

In Synthesis Scheme (a-5), Compound 7 may be an organoaluminum compound, an organozirconium compound, an organozinc compound, or an organotin compound using an organometallic reagent or the like instead of a borylation reagent.

Next, in accordance with Synthesis Scheme (a-5), a 9-aryl-9H-carbazole boryl compound (Compound 7) and a 9-aryl-9H-carbazole halide compound (Compound 5) are cross-coupled, whereby the target bicarbazole compound (G5) can be obtained. When the synthesis is performed in accordance with Synthesis Scheme (a-5), a homocoupling product of Compound 7 and a homocoupling product of Compound 5 that are slightly generated in the coupling reaction are the target compound (G5). Accordingly, by performing the synthesis in accordance with Synthesis Scheme (a-5), the target substance can be obtained with high purity even when a by-product that is a homocoupling product of each of the two source materials is generated.

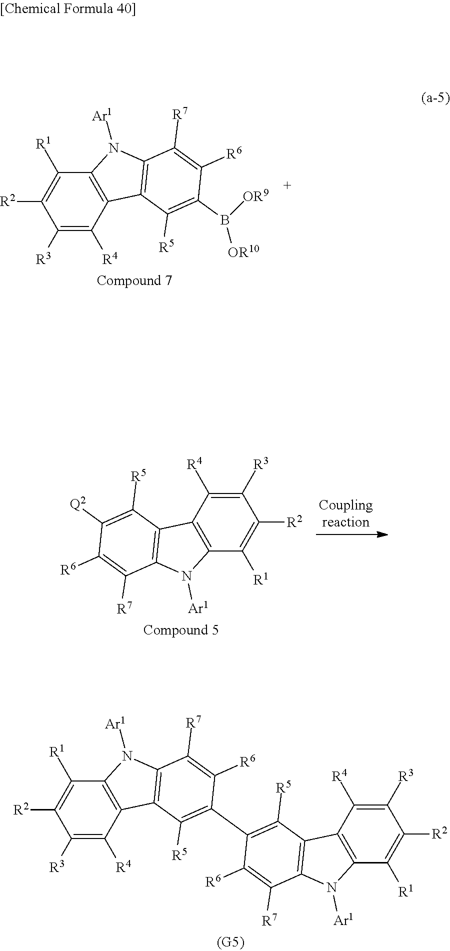

In Synthesis Scheme (a-5), Ar¹, R¹ to R⁷, R⁹, R¹⁰, and Q² are the same as those described above and are not described here.

In Synthesis Scheme (a-5), for example, palladium(II) acetate, tetrakis(triphenylphosphine)palladium(0), or bis(triphenylphosphine)palladium(II) dichloride can be used as a palladium catalyst. In addition, for example, tri(ortho-tolyl)phosphine, triphenylphosphine, or tricyclohexylphosphine can be used as a ligand of the palladium catalyst.

In Synthesis Scheme (a-5), as a base, an organic base such as sodium-tert-butoxide, an inorganic base such as potassium carbonate or sodium carbonate, or the like can be used.

In Synthesis Scheme (a-5), as a reaction solvent, a mixed solvent of toluene and water, a mixed solvent of xylene and water, a mixed solvent of benzene and water, a mixed solvent of water and an ether such as ethylene glycol dimethyl ether or 1,4-dioxane, or the like can be used. A boronic acid and a boryl compound react at a higher rate and potentially bring about an effect of increasing the yield when having higher solubility in an aqueous phase; thus, it is preferable to add water. However, in the case where an ether is used for the solvent, a similar effect can be potentially brought about even when water is not added.

In Synthesis Scheme (a-5), as a reaction solvent, a mixed solvent of toluene, water, and alcohol such as ethanol, a mixed solvent of xylene, water, and alcohol such as ethanol, a mixed solvent of benzene, water, and alcohol such as ethanol, or the like can be used. In particular, toluene and water, a mixed solvent of toluene, water, and ethanol, or a mixed solvent of water and an ether such as ethylene glycol dimethyl ether is preferable.

Compound (G5) can also be synthesized according to Synthesis Scheme (a-6). That is, in accordance with Synthesis Scheme (a-6), an aryl halide compound (Compound 1) and a bicarbazole compound (Compound 8) are coupled, whereby the target bicarbazole compound (G5) can be obtained.

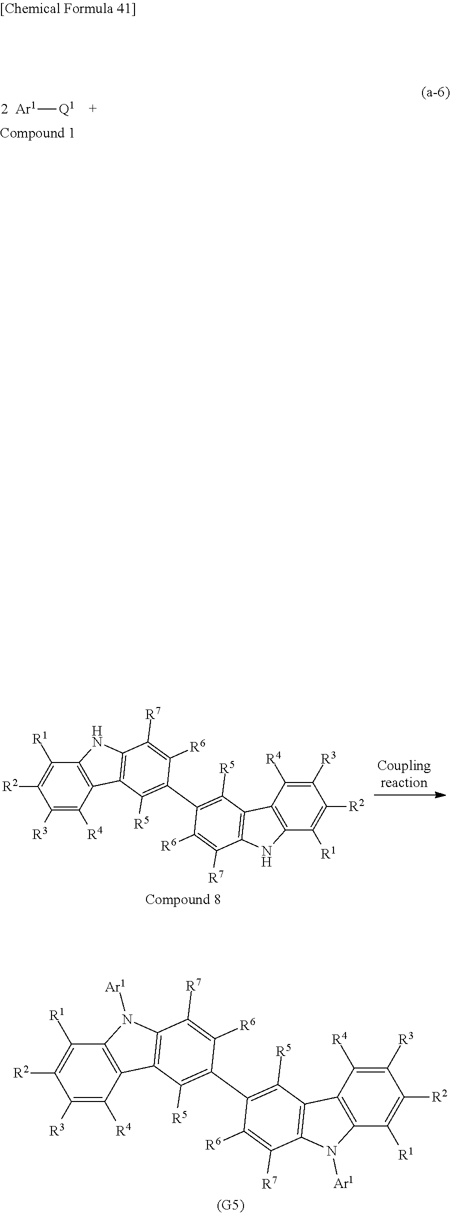

In Synthesis Scheme (a-6), Ar¹, R¹ to R⁷, and Q¹ are the same as those described above and are not described here.

In the case where the Buchwald-Hartwig reaction using a palladium catalyst or the Ullmann reaction using copper or copper oxide is performed in Synthesis Scheme (a-6), the same reaction condition as that in Synthesis Scheme (a-1) can be used.

The above is the description of the synthesis method of General Formula (G5). Note that the method for synthesizing the organic compound represented by General Formula (G5) is not limited to Synthesis Schemes (a-1) to (a-6) above.

In the case where two or more kinds of organic compounds are used as the host material 118 in the light-emitting device of one embodiment of the present invention, the above-described organic compound represented by any one of General Formulae (G1) to (G9) and an organic compound that can form an exciplex with the organic compound are preferably used as the host material 118. For example, in the case where the light-emitting layer 113 contains two kinds of organic compounds (the organic compounds 118_1 and 118_2) as the host material 118 as illustrated in FIG. 1B, the organic compound represented by any one of General Formulae (G1) to (G9) is preferably used as the organic compound 118_1, and the organic compound that can form an exciplex with the organic compound is preferably used as the organic compound 118_2.

As the organic compound that can form an exciplex with the organic compound represented by any one of General Formulae (G1) to (G9), an electron-transport material is preferably used. A compound having a π-electron deficient heteroaromatic ring skeleton such as a nitrogen-containing heteroaromatic compound, or a zinc- or aluminum-based metal complex can be used, for example, as an electron-transport material. Examples of the compound having a π-electron deficient heteroaromatic ring skeleton include compounds such as an oxadiazole derivative, a triazole derivative, a benzimidazole derivative, a quinoxaline derivative, a dibenzoquinoxaline derivative, a phenanthroline derivative, a pyridine derivative, a bipyridine derivative, a pyrimidine derivative, and a triazine derivative. Examples of the zinc- or aluminum-based metal complex include a metal complex having a quinoline ligand, a metal complex having a benzoquinoline ligand, a metal complex having an oxazole ligand, and a metal complex having a thiazole ligand. Details of the compound having a it-electron deficient heteroaromatic ring skeleton and the zinc- or aluminum-based metal complex are described later.

Although the example where the organic compound represented by any of General Formulae (G1) to (G9) is used as the host material in the light-emitting layer of the light-emitting device is described, the organic compound can also be used for layers other than the light-emitting layer 113 in the light-emitting device. Since the organic compound represented by any of General Formulae (G1) to (G9) has a high hole-transport property, the light-emitting device including the organic compound can have high emission efficiency and be driven with a low-voltage. Since the organic compound represented by any of General Formulae (G1) to (G9) has high thermal stability and high electrochemical stability, the light-emitting device including the organic compound can be inhibited from deteriorating and have increased reliability. Specifically, the organic compound represented by any of General Formulae (G1) to (G9) can be used for a hole-transport layer, a cap layer, or the like in addition to the light-emitting layer.

Note that the compounds described in this embodiment can be used in combination with any of the structures described in the other embodiments as appropriate.

### Embodiment 2

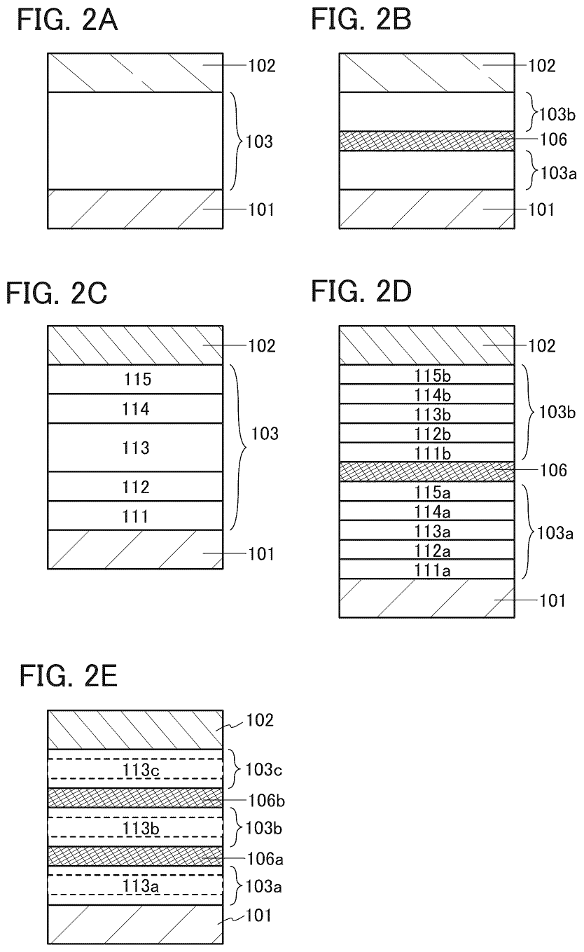

In this embodiment, structures of the light-emitting device including any of the organometallic compounds described in Embodiment 1 will be described with reference to FIGS. 2A to 2E.

### <Basic Structure of Light-Emitting Device>

A basic structure of the light-emitting device is described. FIG. 2A illustrates a light-emitting device having a structure (single structure) in which an organic compound layer including a light-emitting layer is provided between a pair of electrodes. Specifically, the organic compound layer 103 is positioned between the first electrode 101 and the second electrode 102.

FIG. 2B illustrates a light-emitting device that has a stacked-layer structure (tandem structure) in which a plurality of organic compound layers (two organic compound layers 103a and 103b in FIG. 2B) are provided between a pair of electrodes and a charge-generation layer 106 is provided between the organic compound layers. A light-emitting device having the tandem structure enables manufacture of a light-emitting apparatus that has high efficiency without changing the amount of current.

The charge-generation layer 106 has a function of injecting electrons into one of the organic compound layers 103a and 103b and injecting holes into the other of the organic compound layers 103a and 103b when a potential difference is caused between the first electrode 101 and the second electrode 102. Thus, when voltage is applied in FIG. 2B such that the potential of the first electrode 101 is higher than that of the second electrode 102, the charge-generation layer 106 injects electrons into the organic compound layer 103a and injects holes into the organic compound layer 103b.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 or the second electrode 102.

FIG. 2C illustrates a stacked-layer structure of the organic compound layer 103 in the light-emitting device of one embodiment of the present invention. In this case, the first electrode 101 is regarded as functioning as an anode and the second electrode 102 is regarded as functioning as a cathode. The organic compound layer 103 has a structure in which the hole-injection layer 111, the hole-transport layer 112, the light-emitting layer 113, the electron-transport layer 114, and the electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can achieve higher reliability than a single-layer structure in some cases. In the case where a plurality of organic compound layers are provided as in the tandem structure illustrated in FIG. 2B, the layers in each organic compound layer are sequentially stacked from the anode side as described above. When the first electrode 101 is the cathode and the second electrode 102 is the anode, the stacking order of the layers in the organic compound layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the organic compound layers (103, 103a, and 103b) contains an appropriate combination of a light-emitting substance and a plurality of substances, so that fluorescent light of a desired color or phosphorescent light of a desired color can be obtained. The light-emitting layer 113 may have a stacked-layer structure of layers having different emission colors. In that case, light-emitting substances and other substances are different between the stacked light-emitting layers. Alternatively, the plurality of organic compound layers (103a and 103b) in FIG. 2B may exhibit their respective emission colors. Also in that case, the light-emitting substances and other substances are different between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 2C. Thus, light from the light-emitting layer 113 in the organic compound layer 103 can be resonated between the electrodes and light emitted through the second electrode 102 can be intensified. This makes it easy to achieve high resolution. In addition, emission intensity with a predetermined wavelength in the front direction can be increased, whereby power consumption can be reduced.

Note that in the case where the first electrode 101 of the light-emitting device is a reflective electrode that has a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is k, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: k) obtained from the light-emitting layer 113, each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) is preferably adjusted to (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

The light-emitting device illustrated in FIG. 2D is a light-emitting device having the tandem structure. The tandem structure enables a light-emitting device to emit light with high luminance. Furthermore, the amount of current needed for obtaining a predetermined luminance can be smaller in the tandem structure than in the single structure; thus, the tandem structure enables higher reliability. In addition, power consumption can be reduced.

The light-emitting device illustrated in FIG. 2E is an example of the light-emitting device having the tandem structure illustrated in FIG. 2B, and includes three organic compound layers (103a, 103b, and 103c) stacked with charge-generation layers (106a and 106b) positioned therebetween, as illustrated in FIG. 2E. The three organic compound layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light, or the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity lower than or equal to 1×10⁻² Ωcm.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity lower than or equal to 1×10⁻² Ωcm.

### <Specific Structure of Light-Emitting Device>

Next, a specific structure of the light-emitting device of one embodiment of the present invention will be described. Here, the description is made using FIG. 2D illustrating the tandem structure. Note that the structure of the organic compound layer applies also to the structure of the light-emitting devices having the single structure in FIGS. 2A and 2C. When the light-emitting device in FIG. 2D has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the organic compound layer 103b, with the use of a material selected as appropriate.

### <Materials of Light-Emitting Device>

### <<Light-Emitting Layer>>

The light-emitting layers (113, 113a, and 113b) contain a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables a structure that exhibits different emission colors (e.g., white light emission obtained by a combination of complementary emission colors). Furthermore, one light-emitting layer may have a stacked-layer structure including different light-emitting substances.

The light-emitting layers (113, 113a, and 113b) may each contain one or more kinds of organic compounds (e.g., a host material) in addition to a light-emitting substance (a guest material). Specifically, the organic compound described in Embodiment 1 is preferably used. Thus, the reliability of the light-emitting device of one embodiment of the present invention can be increased.

In the case where a plurality of host materials are used in the light-emitting layers (113, 113a, and 113b), the structure described with reference to FIG. 1B in Embodiment 1 can be used for the light-emitting layer 113, for example. In the light-emitting layer 113, the host materials 118 are present in the largest proportion by weight, and the guest material 119 is dispersed in the host materials 118. The T₁ level of the host material 118 (the organic compound 118_1 and the organic compound 118_2) in the light-emitting layer 113 is preferably higher than the T₁ level of the guest material (the guest material 119) in the light-emitting layer 113.

As the organic compound 118_1, a material having an electron-transport property higher than a hole-transport property can be used, and a material having an electron mobility higher than or equal to 1×10⁻⁶ cm²/Vs is preferable. A compound having a π-electron deficient heteroaromatic ring skeleton such as a nitrogen-containing heteroaromatic compound, or a zinc- or aluminum-based metal complex can be used, for example, as a material which easily accepts electrons (a material having an electron-transport property). Examples of the compound having a π-electron deficient heteroaromatic ring skeleton include compounds such as an oxadiazole derivative, a triazole derivative, a benzimidazole derivative, a quinoxaline derivative, a dibenzoquinoxaline derivative, a phenanthroline derivative, a pyridine derivative, a bipyridine derivative, a pyrimidine derivative, and a triazine derivative. Examples of the zinc- or aluminum-based metal complex include a metal complex having a quinoline ligand, a metal complex having a benzoquinoline ligand, a metal complex having an oxazole ligand, and a metal complex having a thiazole ligand.

Specific examples thereof include metal complexes having a quinoline or benzoquinoline skeleton, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq). Alternatively, a metal complex having an oxazole-based or thiazole-based ligand, such as bis[2-(2-benzoxazolyl)phenolato]zinc(II) (abbreviation: ZnPBO) or bis[2-(2-benzothiazolyl)phenolato]zinc(II) (abbreviation: ZnBTZ), can be used. Other than such metal complexes, any of the following can be used: heterocyclic compounds such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: COi1), 3-(4-biphenylyl)-4-phenyl-5-(4-tert-butylphenyl)-1,2,4-triazole (abbreviation: TAZ), 9-[4-(4,5-diphenyl-4H-1,2,4-triazol-3-yl)phenyl]-9H-carbazole (abbreviation: CzTAZ1), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), bathophenanthroline (abbreviation: BPhen), bathocuproine (abbreviation: BCP), and 2,2′-(1,3-phenylene)bis(9-phenyl-1,10-phenanthroline) (abbreviation: mPPhen2P); heterocyclic compounds having a diazine skeleton such as 2-{3-[3-(N-phenyl-9H-carbazol-3-yl)-9H-carbazol-9-yl]phenyl}dibenzo[f,h]quinoxaline (abbreviation: 2mPCCzPDBq), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 2-[3-(3,9′-bi-9H-carbazol-9-yl)phenyl]dibenzo[f,h]quinoxaline (abbreviation: 2mCzCzPDBq), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), and 8-(p-terphenyl-3-yl)-4-[3-(dibenzothiophen-4-yl)phenyl]-[1]benzofuro[3,2-d]pyrimidine (abbreviation: 8mpTP-4mDBtPBfpm); heterocyclic compounds having a triazine skeleton such as 2-{4-[3-(N-phenyl-9H-carbazol-3-yl)-9H-carbazol-9-yl]phenyl}-4,6-diphenyl-1,3,5-triazine (abbreviation: PCCzPTzn); heterocyclic compounds having a pyridine skeleton such as 3,5-bis[3-(9H-carbazol-9-yl)phenyl]pyridine (abbreviation: 35DCzPPy) and 1,3,5-tri[3-(3-pyridyl)phenyl]benzene (abbreviation: TmPyPB); and heteroaromatic compounds such as 4,4′-bis(5-methylbenzoxazol-2-yl)stilbene (abbreviation: BzOs). Among the heterocyclic compounds, the heterocyclic compounds having a triazine skeleton, a diazine (pyrimidine, pyrazine, or pyridazine) skeleton, or a pyridine skeleton are highly reliable and stable and are thus preferably used. In addition, the heterocyclic compounds having any of these skeletons have a high electron-transport property to contribute to a reduction in driving voltage. Further alternatively, a high-molecular compound such as poly(2,5-pyridinediyl) (abbreviation: PPy), poly[(9,9-dihexylfluorene-2,7-diyl)-co-(pyridine-3,5-diyl)](abbreviation: PF-Py), or poly[(9,9-dioctylfluorene-2,7-diyl)-co-(2,2′-bipyridine-6,6′-diyl)](abbreviation: PF-BPy) can be used. The substances described here are mainly substances having an electron mobility higher than or equal to 1×10⁻⁶ cm²/Vs. Note that other substances may also be used as long as their electron-transport properties are higher than their hole-transport properties.

As the organic compound 118_2, a substance which can form an exciplex together with the organic compound 118_1 is preferably used. Specifically, the organic compound 118_2 preferably includes a skeleton having a high donor property, such as a π-electron rich heteroaromatic ring skeleton or an aromatic amine skeleton. Examples of the compound having a π-electron rich heteroaromatic ring skeleton include heteroaromatic compounds such as a dibenzothiophene derivative, a dibenzofuran derivative, and a carbazole derivative. In that case, it is preferable that the organic compound 118_1, the organic compound 118_2, and the guest material 119 (a phosphorescent compound) be selected such that the emission peak of the exciplex formed by the organic compounds 118_1 and 118_2 overlaps with an absorption band, specifically the longest-wavelength absorption band, of a triplet metal to ligand charge transfer (MLCT) transition of the guest material 119 (the phosphorescent compound). This makes it possible to provide a light-emitting device with drastically improved emission efficiency. Note that in the case where a thermally activated delayed fluorescence material is used instead of the phosphorescent compound, it is preferable that the longest-wavelength absorption band be a singlet absorption band.

The organic compound described in Embodiment 1 is preferably used as the organic compound 118_2. Thus, the reliability of the light-emitting device of one embodiment of the present invention can be increased. As the organic compound 118_2, any of the hole-transport materials given below can be used.

A material having a hole-transport property higher than an electron-transport property can be used as a hole-transport material, and a material having a hole mobility higher than or equal to 1×10⁻⁶ cm²/Vs is preferable. Specifically, an aromatic amine, a carbazole derivative, an aromatic hydrocarbon, a stilbene derivative, or the like can be used. The hole-transport material may be a high molecular compound.

Examples of the aromatic amine compounds that can be used as the material having a high hole-transport property are N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis[4-bis(3-methylphenyl)aminophenyl]-N,N′-diphenyl-4,4′-diaminobiphenyl (abbreviation: DNTPD), 1,3,5-tris[N-(4-diphenylaminophenyl)-N-phenylamino]benzene (abbreviation: DPA3B), and the like.

Specific examples of the carbazole derivative include 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples of the carbazole derivative are 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthryl)phenyl]-9H-carbazole (abbreviation: CzPA), 1,4-bis[4-(N-carbazolyl)phenyl]-2,3,5,6-tetraphenylbenzene, and the like.

Examples of the aromatic hydrocarbon include 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, and 2,5,8,11-tetra(tert-butyl)perylene. Other examples include pentacene and coronene. The aromatic hydrocarbon having a hole mobility higher than or equal to 1×10⁻⁶ cm²/Vs and having 14 to 42 carbon atoms is particularly preferable.

The aromatic hydrocarbon may have a vinyl skeleton. Examples of the aromatic hydrocarbon having a vinyl skeleton include 4,4′-bis(2,2-diphenylvinyl)biphenyl (abbreviation: DPVBi) and 9,10-bis[4-(2,2-diphenylvinyl)phenyl]anthracene (abbreviation: DPVPA).

A high molecular compound such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N-phenylamino}phenyl)methacrylamide](abbreviation: PTPDMA), or poly[N,N′-bis(4-butylphenyl)-N,N′-bis(phenyl)benzidine](abbreviation: Poly-TPD) can also be used.

Examples of the material having a high hole-transport property include aromatic amine compounds such as 4,4′-bis[N-(1-naphthyl)-N-phenylamino]biphenyl (abbreviation: NPB or α-NPD), N,N′-diphenyl-N,N′-bis(3-methylphenyl)-4,4′-diaminobiphenyl (abbreviation: TPD), 4,4′,4″-tris(carbazol-9-yl)triphenylamine (abbreviation: TCTA), 4,4′,4″-tris[N-(1-naphthyl)-N-phenylamino]triphenylamine (abbreviation: 1′-TNATA), 4,4′,4″-tris(N,N′-diphenylamino)triphenylamine (abbreviation: TDATA), 4,4′,4″-tris[N-(3-methylphenyl)-N-phenylamino]triphenylamine (abbreviation: m-MTDATA), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), N-(9,9-spirobi[9H-fluoren]-2-yl)-N,N′,N-triphenyl-1,4-phenylenediamine (abbreviation: DPASF), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N,N″-triphenyl-N,N,N″-tris(9-phenylcarbazol-3-yl)benzene-1,3,5-triamine (abbreviation: PCA3B), N-(9,9-diphenyl-9H-fluoren-2-yl)-N,9-diphenyl-9H-carbazol-3-amine (abbreviation: PCAFLP(2)), N-(9,9-diphenyl-9H-fluoren-2-yl)-N,9-diphenyl-9H-carbazol-2-amine (abbreviation: PCAFLP(2)-02), N-(biphenyl-4-yl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), N-(9,9-spirobi[9H-fluoren]-2-yl)-N,9-diphenylcarbazol-3-amine (abbreviation: PCASF), N,N′-diphenyl-N,N′-bis(4-diphenylaminophenyl)spirobi[9H-fluorene]-2,7-diamine (abbreviation: DPA2SF), N-[4-(9H-carbazol-9-yl)phenyl]-N-(4-phenyl)phenylaniline (abbreviation: YGA1BP), and N,N′-bis[4-(carbazol-9-yl)phenyl]-N,N′-diphenyl-9,9-dimethylfluorene-2,7-diamine (abbreviation: YGA2F). Other examples are amine compounds, carbazole compounds, thiophene compounds, furan compounds, fluorene compounds, triphenylene compounds, phenanthrene compounds, and the like such as 3-[4-(1-naphthyl)phenyl]-9-phenyl-9H-carbazole (abbreviation: PCPN), 9-[4-(9-phenyl-9H-carbazol-3-yl)phenyl]phenanthrene (abbreviation: PCPPn), 3,3′-bis(9-phenyl-9H-carbazole) (abbreviation: PCCP), 1,3-bis(N-carbazolyl)benzene (abbreviation: mCP), 3,6-bis(3,5-diphenylphenyl)-9-phenylcarbazole (abbreviation: CzTP), 3,6-di(9H-carbazol-9-yl)-9-phenyl-9H-carbazole (abbreviation: PhCzGI), 2,8-di(9H-carbazol-9-yl)dibenzothiophene (abbreviation: Cz2DBT), 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Among the above compounds, compounds having a pyrrole skeleton, a furan skeleton, a thiophene skeleton, or an aromatic amine skeleton are preferable because of their high stability and high reliability. In addition, the compounds having any of these skeletons have a high hole-transport property to contribute to a reduction in driving voltage.

Note that in the case where an organic compound having an electron-transport property is used as the organic compound 118_1 and an organic compound having a hole-transport property is used as the organic compound 118_2, the HOMO level of the organic compound having a hole-transport property is preferably higher than or equal to the HOMO level of the organic compound having an electron-transport property. The LUMO level of the organic compound having a hole-transport property is preferably higher than or equal to that of the organic compound having an electron-transport property, in which case an exciplex can be formed more efficiently.

The values of the HOMO and LUMO levels can be obtained through a cyclic voltammetry (CV) measurement.

In the cyclic voltammetry (CV) measurement, the values (E) of HOMO and LUMO levels can be calculated on the basis of an oxidation peak potential (Eₚₐ) and a reduction peak potential (E_{pc}), which are obtained by changing the potential of a working electrode with respect to a reference electrode. In the measurement, a HOMO level and a LUMO level can be obtained by potential scanning in the positive direction and potential scanning in the negative direction, respectively. The scanning speed in the measurement is 0.1 V/s.

Specifically, a standard oxidation-reduction potential (Eₒ) (=Eₚₐ+E_{pc})/2) is calculated from an oxidation peak potential (Eₚₐ) and a reduction peak potential (E_{pc}), which are obtained by the cyclic voltammogram of a material. Then, the standard oxidation-reduction potential (Eₒ) is subtracted from the potential energy (Eₓ) of the reference electrode with respect to a vacuum level, whereby each of the values (E) (=Eₓ−Eₒ) of HOMO and LUMO levels can be obtained.

Note that the reversible oxidation-reduction wave was obtained in the above case; in the case where an irreversible oxidation-reduction wave is obtained, the HOMO level is calculated as follows: a value obtained by subtracting a predetermined value (0.1 eV) from an oxidation peak potential (Eₚₐ) is assumed to be a reduction peak potential (E_{pc}), and a standard oxidation-reduction potential (Eₒ) is calculated to one decimal place. To calculate the LUMO level, a value obtained by adding a predetermined value (0.1 eV) to a reduction peak potential (E_{pc}) is assumed to be an oxidation peak potential (Eₚₐ), and a standard oxidation-reduction potential (Eₒ) is calculated to one decimal place.

There is no particular limitation on the guest material 119 that can be used for the light-emitting layers (113, 113a, and 113b), and a light-emitting substance that converts singlet excitation energy into light in the visible light range or a light-emitting substance that converts triplet excitation energy into light in the visible light range can be used.

<<Light-Emitting Substance that Converts Singlet Excitation Energy into Light Emission>>

The following substances that emit fluorescent light (fluorescent substances) can be given as examples of the light-emitting substance that converts singlet excitation energy into light emission and can be used in the light-emitting layers (113, 113a, and 113b): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPrn), N,N′-(pyrene-1,6-diyl)bis[(N-phenylbenzo[b]naphtho[1,2-d]furan)-6-amine](abbreviation: 1,6BnfAPrn), N,N′-(pyrene-1,6-diyl)bis[(N-phenylbenzo[b]naphtho[1,2-d]furan)-8-amine](abbreviation: 1,6BnfAPrn-02), and N,N′-(pyrene-1,6-diyl)bis[(6,N-diphenylbenzo[b]naphtho[1,2-d]furan)-8-amine](abbreviation: 1,6BnfAPrn-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenyl-4,4′-stilbenediamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N′-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), or N-[4-(9,10-diphenyl-2-anthryl)phenyl]-N,N,N′-triphenyl-1,4-phenylenediamine (abbreviation: 2DPAPPA).

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N,N′-tetrakis(4-methylphenyl)tetracene-5,11-diamine (abbreviation: p-mPhTD), 7,14-diphenyl-N,N,N,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02), or 3,10-bis[N-(dibenzofuran-3-yl)-N-phenylamino]naphtho[2,3-b;6,7-b′]bisbenzofuran (abbreviation: 3,10FrA2Nbf(IV)-02). In particular, a pyrenediamine compound such as 1,6FLPAPrn, 1,6mMemFLPAPrn, or 1,6BnfAPrn-03 can be used, for example.

<<Light-Emitting Substance that Converts Triplet Excitation Energy into Light Emission>>

Next, examples of the light-emitting substance that converts triplet excitation energy into light emission and can be used in the light-emitting layer 113 include substances that emit phosphorescent light (phosphorescent substances) and thermally activated delayed fluorescent (TADF) materials that exhibit thermally activated delayed fluorescence.

A phosphorescent substance is a compound that emits phosphorescent light but does not emit fluorescent light at a temperature higher than or equal to a low temperature (e.g., 77 K) and lower than or equal to room temperature (i.e., higher than or equal to 77 K and lower than or equal to 313 K). The phosphorescent substance preferably includes a metal element with large spin-orbit interaction, and can be an organometallic complex, a metal complex (platinum complex), or a rare earth metal complex, for example. Specifically, the phosphorescent substance preferably includes a transition metal element. It is preferable that the phosphorescent substance include a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

<<Phosphorescent Substance (Greater than or Equal to 400 nm and Less than 580 nm: Blue or Green)>>

As examples of a phosphorescent substance which emits blue or green light and whose emission spectrum has a peak wavelength greater than or equal to 400 nm and less than 580 nm, the following substances can be given.

Examples of the phosphorescent substance include organometallic complexes having a 4H-triazole ring, such as tris{2-[5-(2-methylphenyl)-4-(2,6-dimethylphenyl)-4H-1,2,4-triazol-3-yl-κN²]phenyl-KC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) andtris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[l-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a phenylpyridine derivative having an electron-withdrawing group is a ligand, such as bis[2-(4′,6′-difluorophenyl)pyridinato-N,C^{2′}]iridium(III)tetrakis(1-pyrazolyl)borate (abbreviation: FIr6), bis[2-(4′,6′-difluorophenyl)pyridinato-N,C^{2′}]iridium(III)picolinate (abbreviation: FIrpic), bis{2-[3′,5′-bis(trifluoromethyl)phenyl]pyridinato-N,C^{2′}′}iridium(III)picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

<<Phosphorescent Substance (Greater than or Equal to 490 nm and Less than 590 nm: Green or Yellow)>>

As examples of a phosphorescent substance which emits green or yellow light and whose emission spectrum has a peak wavelength greater than or equal to 490 nm and less than 590 nm, the following substances can be given.

Examples of the phosphorescent substance include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-KC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C²′)iridium(III)acetylacetonate (abbreviation: [Ir(ppy)₂(acac)]), bis(benzo[h]quinolinato)iridium(III)acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′}′)iridium(III) (abbreviation: [Ir(pq)₃]), bis(2-phenylquinolinato-N,C²′)iridium(III)acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-KC][2-(4-methyl-5-phenyl-2-pyridinyl-κN)phenyl-κC], [2-d₃-methyl-8-(2-pyridinyl-κN)benzofuro[2,3-b]pyridine-KC]bis[2-(5-d₃-methyl-2-pyridinyl-κN²)phenyl-κC]iridium(III) (abbreviation: [Ir(5mppy-d₃)₂(mbfpypy-d₃)]), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d₆)₂(mbfpypy-iPr-d₄)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)), and tris{2-[5-(methyl-d₃)-4-phenyl-2-pyridinyl-N]phenyl-κC}iridium(III) (abbreviation: Ir(5m4dppy-d₃)₃; organometallic complexes such as bis(2,4-diphenyl-1,3-oxazolato-N,C^{2′})iridium(III)acetylacetonate (abbreviation: [Ir(dpo)₂(acac)]), bis{2-[4′-(perfluorophenyl)phenyl]pyridinato-N,C^{2′}}iridium(III)acetylacetonate (abbreviation: [Ir(p-PF-ph)₂(acac)]), and bis(2-phenylbenzothiazolato-N,C^{2′})iridium(III)acetylacetonate (abbreviation: [Ir(bt)₂(acac)]); and a rare earth metal complex such as tris(acetylacetonato) (monophenanthroline)terbium(III) (abbreviation: [Tb(acac)₃(Phen)]).

<<Phosphorescent Substance (Greater than or Equal to 570 nm and Less than 750 nm: Yellow or Red)>>

As examples of a phosphorescent substance which emits yellow or red light and whose emission spectrum has a peak wavelength greater than or equal to 570 nm and less than 750 nm, the following substances can be given.

Examples of the phosphorescent substance include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis{2-[5-(2,6-dimethylphenyl)-3-(3,5-dimethylphenyl)-2-pyrazinyl-κN]-4,6-dimethylphenyl-κC}(2,2′,6,6′-tetramethyl-3,5-heptanedionato-κ²O,O′)iridium(III) (abbreviation: [Ir(dmdppr-dmp)₂(dpm)]), (acetylacetonato)bis(2-methyl-3-phenylquinoxalinato-N,C^{2′})iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C^{2′})iridium(III) (abbreviation: [Ir(piq)₃]), bis(1-phenylisoquinolinato-N,C^{2′})iridium(III)acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### <<TADF Material>>

Any of materials described below can be used as the TADF material. The TADF material is a material that has a small difference between its S₁ and T₁ levels (preferably less than or equal to 0.20 eV), enables up-conversion of a triplet excited state into a singlet excited state (i.e., reverse intersystem crossing) using a little thermal energy, and efficiently exhibits light (fluorescent light) from the singlet excited state. The thermally activated delayed fluorescence is efficiently obtained under the condition where the difference in energy between the triplet excitation energy level and the singlet excitation energy level is greater than or equal to 0.00 eV and less than or equal to 0.20 eV, preferably greater than or equal to 0.00 eV and less than or equal to 0.10 eV. Delayed fluorescent light by the TADF material refers to light emission having a spectrum similar to that of normal fluorescent light and an extremely long lifetime. The lifetime is longer than or equal to 1×10⁻⁶ seconds, or longer than or equal to 1×10⁻³ seconds.

Note that the TADF material can be also used as an electron-transport material, a hole-transport material, or a host material.

Examples of the TADF material include fullerene, a derivative thereof, an acridine derivative such as proflavine, and eosin. Other examples include a metal-containing porphyrin, such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

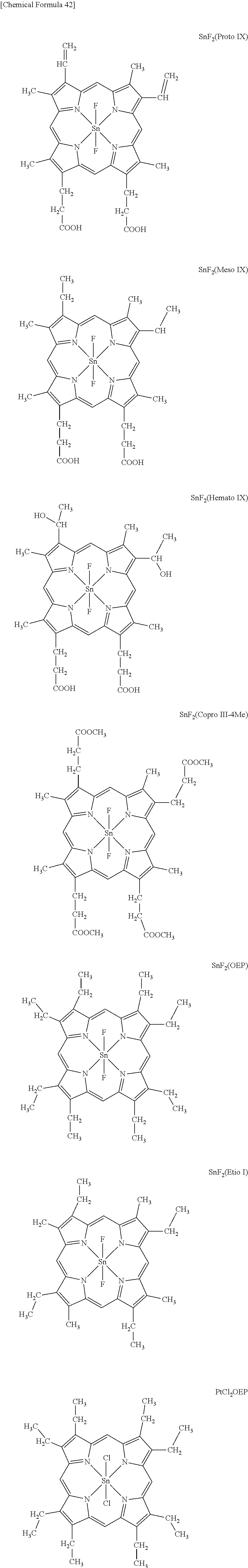

Additionally, a π-electron rich heteroaromatic compound and a π-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02) may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are enhanced and the energy difference between the singlet excited state and the triplet excited state becomes small. As the TADF material, a TADF material in which the singlet and triplet excited states are in thermal equilibrium (TADF100) may be used. Since such a TADF material enables a short emission lifetime (excitation lifetime), the efficiency of a light-emitting device in a high-luminance region can be less likely to decrease.

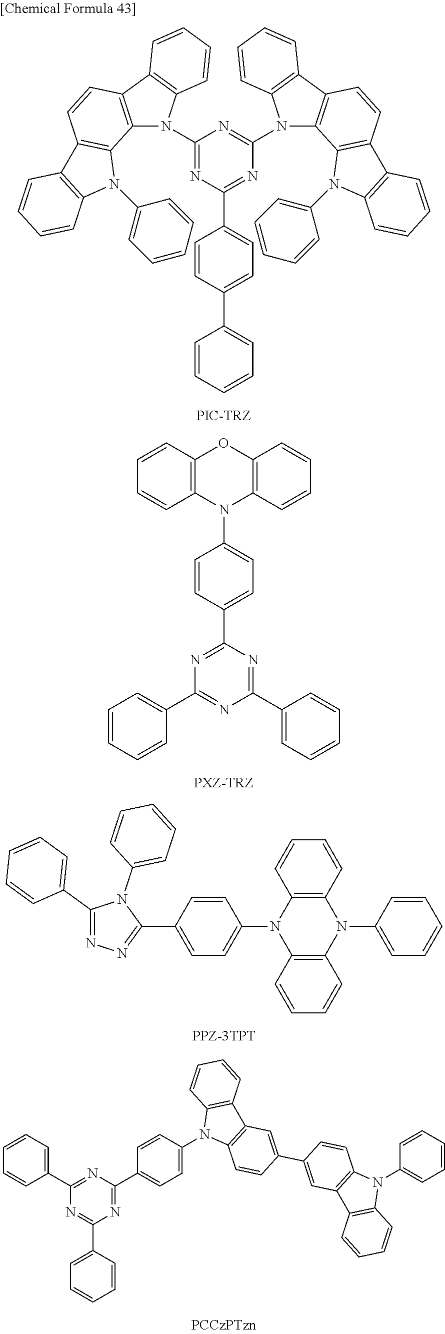 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

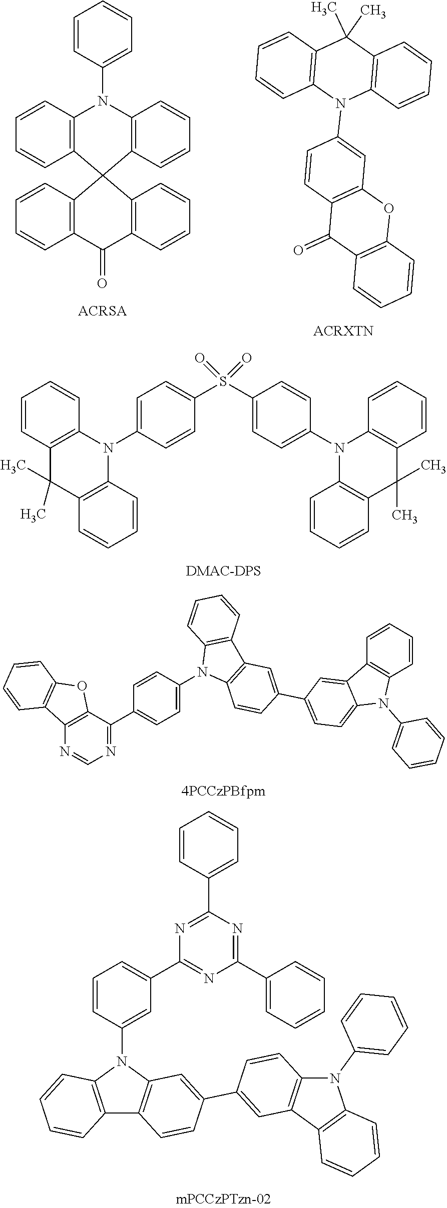 [SMILES: O=C1c2ccccc2C2(c3ccccc31)c1ccccc1N(c1ccccc1)c1ccccc12 ; CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21 ; c1ccc(-n2c3ccccc3c3cc(-c4ccc5c(c4)c4ccccc4n5-c4ccc(-c5ncnc6c5oc5ccccc56)cc4)ccc32)cc1 ; c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

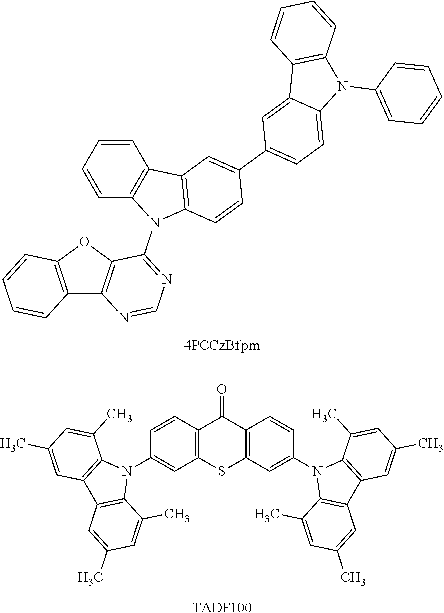

In addition to the above, another example of a material having a function of converting triplet excitation energy into light emission is a nano-structure of a transition metal compound having a perovskite structure. In particular, a nano-structure of a metal halide perovskite material is preferable. The nano-structure is preferably a nanoparticle or a nanorod.

The light-emitting layer 113 can include two or more layers. For example, in the case where the light-emitting layer 113 is formed by stacking a first light-emitting layer and a second light-emitting layer in this order from the hole-transport layer side, the first light-emitting layer is formed using a substance having a hole-transport property as the host material and the second light-emitting layer is formed using a substance having an electron-transport property as the host material. A light-emitting material included in the first light-emitting layer may be the same as or different from a light-emitting material included in the second light-emitting layer. In addition, the materials may have functions of emitting light of the same color or light of different colors. When light-emitting materials having functions of emitting light of different colors are used for the two light-emitting layers, light of a plurality of emission colors can be obtained at the same time. It is particularly preferable to select light-emitting materials of the light-emitting layers so that white light can be obtained by combining light emission from the two light-emitting layers.

The light-emitting layer 113 may include a material other than the host material 118 and the guest material 119.

Note that the light-emitting layer 113 can be formed by an evaporation method (including a vacuum evaporation method), an ink-jet method, a coating method, gravure printing, or the like. Besides the above-mentioned materials, an inorganic compound such as a quantum dot or a high molecular compound (e.g., an oligomer, a dendrimer, or a polymer) may be used.

### <<Hole-Injection Layer>>

The hole-injection layers (111, 111a, and 111b) inject holes from the first electrode 101 serving as the anode and the charge-generation layers (106, 106a, and 106b) to the organic compound layers (103, 103a, and 103b) and contain an organic acceptor material and a material having a high hole-injection property.

The hole-injection layers (111, 111a, and 111b) have a function of lowering a barrier for hole injection from one of the pair of electrodes (the first electrode 101 or the second electrode 102) to promote hole injection and is formed using a transition metal oxide, a phthalocyanine derivative, or an aromatic amine, for example. As examples of the transition metal oxide, molybdenum oxide, vanadium oxide, ruthenium oxide, tungsten oxide, manganese oxide, and the like can be given. As examples of the phthalocyanine derivative, phthalocyanine, metal phthalocyanine, and the like can be given. As examples of the aromatic amine, a benzidine derivative, a phenylenediamine derivative, and the like can be given. It is also possible to use a high molecular compound such as polythiophene or polyaniline; a typical example thereof is poly(ethylenedioxythiophene)/poly(styrenesulfonic acid), which is self-doped polythiophene.

As each of the hole-injection layers (111, 111a, and 111b), a layer containing a composite material of a hole-transport material and a material having a property of accepting electrons from the hole-transport material can also be used. Alternatively, a stack of a layer containing a material having an electron-accepting property and a layer containing a hole-transport material may also be used. In a steady state or in the presence of an electric field, electric charge can be transferred between these materials. As examples of the material having an electron-accepting property, organic acceptors such as a quinodimethane derivative, a chloranil derivative, and a hexaazatriphenylene derivative can be given. A specific example is a compound having an electron-withdrawing group (a halogen group or a cyano group), such as 7,7,8,8-tetracyano-2,3,5,6-tetrafluoroquinodimethane (abbreviation: F₄-TCNQ), chloranil, or 2,3,6,7,10,11-hexacyano-1,4,5,8,9,12-hexaazatriphenylene (abbreviation: HAT-CN). Alternatively, a transition metal oxide such as an oxide of a metal from Group 4 to Group 8 can be used. Specifically, vanadium oxide, niobium oxide, tantalum oxide, chromium oxide, molybdenum oxide, tungsten oxide, manganese oxide, rhenium oxide, or the like can be used. In particular, molybdenum oxide is preferable because it is stable in the air, has a low hygroscopic property, and is easy to handle.

A material having a hole-transport property higher than an electron-transport property can be used as a hole-transport material, and a material having a hole mobility higher than or equal to 1×10⁻⁶ cm²/Vs is preferable. Specifically, any of the aromatic amine, carbazole derivative, aromatic hydrocarbon, stilbene derivative, and the like described as examples of the hole-transport material that can be used in the light-emitting layer 113 can be used. Furthermore, the hole-transport material may be a high molecular compound.

### <<Hole-Transport Layer>>

The hole-transport layers (112, 112a, and 112b) contain a hole-transport material and can be formed using any of the hole-transport materials given as examples of the material of the hole-injection layers (111, 111a, and 111b). In order that the hole-transport layers (112, 112a, and 112b) can have a function of transporting holes injected into the hole-injection layers (111, 111a, and 111b) to the light-emitting layers (113, 113a, and 113b), the HOMO level of the hole-transport layers (112, 112a, and 112b) is preferably equal or close to the HOMO level of the hole-injection layers (111, 111a, and 111b).

As the hole-transport material, a substance having a hole mobility higher than or equal to 1×10⁻⁶ cm²/Vs is preferably used. Note that other substances may also be used as long as their hole-transport properties are higher than their electron-transport properties. The layer including a substance having a high hole-transport property is not limited to a single layer and may be a stack of two or more layers each including any of the above substances. The organic compound described in Embodiment 1 has a high hole-transport property, and thus can also be used for the hole-transport layer. Thus, the reliability of the light-emitting device can be increased.

### <<Electron-Transport Layer>>

The electron-transport layers (114, 114a, and 114b) have a function of transporting, to the light-emitting layer 113, electrons injected from the other of the pair of electrodes (the first electrode 101 or the second electrode 102) through the electron-injection layers (115, 115a, and 115b). As an electron-transport material, a material having an electron-transport property higher than a hole-transport property can be used, and a material having an electron mobility higher than or equal to 1×10⁻⁶ cm²/Vs is preferable. A compound having a π-electron deficient heteroaromatic ring skeleton such as a nitrogen-containing heteroaromatic compound or a metal complex can be used, for example, as a compound which easily accepts electrons (a material having an electron-transport property). Specific examples include a metal complex having a quinoline ligand, a benzoquinoline ligand, an oxazole ligand, or a thiazole ligand, which is described as the electron-transport material usable for the light-emitting layer 113. In addition, an oxadiazole derivative, a triazole derivative, a benzimidazole derivative, a quinoxaline derivative, a dibenzoquinoxaline derivative, a phenanthroline derivative, a pyridine derivative, a bipyridine derivative, a pyrimidine derivative, a triazine derivative, or the like can be used. As the electron-transport material, a substance having an electron mobility higher than or equal to 1×10⁻⁶ cm²/Vs is preferably used. Note that other substances may also be used for the electron-transport layer as long as their electron-transport properties are higher than their hole-transport properties. Each of the electron-transport layers (114, 114a, and 114b) is not limited to a single layer and may be a stack of two or more layers each containing any of the above substances.

Between the electron-transport layer (114, 114a, or 114b) and the light-emitting layer (113, 113a, or 113b), a layer that controls transfer of electron carriers may be provided. This is a layer formed by addition of a small amount of a substance having a high electron-trapping property to a material having a high electron-transport property as described above, and the layer is capable of adjusting carrier balance by suppressing transport of electron carriers. Such a structure is very effective in inhibiting a problem (such as a reduction in element lifetime) caused when electrons pass through the light-emitting layer.

### <<Electron-Injection Layer>>

The electron-injection layers (115, 115a, and 115b) have a function of reducing a barrier for electron injection from the second electrode 102 to promote electron injection and can be formed using a Group 1 metal or a Group 2 metal, or an oxide, a halide, or a carbonate of any of these metals, for example. Alternatively, a composite material containing an electron-transport material described above and a material having a property of donating electrons to the electron-transport material can also be used. As examples of the material having an electron-donating property, a Group 1 metal, a Group 2 metal, an oxide of any of these metals, and the like can be given. Specifically, an alkali metal, an alkaline earth metal, or a compound thereof, such as lithium fluoride (LiF), sodium fluoride (NaF), cesium fluoride (CsF), calcium fluoride (CaF₂), or lithium oxide (LiOx), can be used. Alternatively, a rare earth metal compound like erbium fluoride (ErF₃) can be used. Electrode may also be used for the electron-injection layer 115. Examples of the electrode include a substance in which electrons are added at high concentration to calcium oxide-aluminum oxide. The electron-injection layers (115, 115a, and 115b) can be formed using the substance that can be used for the electron-transport layers (114, 114a, and 114b).

A composite material in which an organic compound and an electron donor (donor) are mixed may also be used for the electron-injection layers (115, 115a, and 115b). Such a composite material is excellent in an electron-injection property and an electron-transport property because electrons are generated in the organic compound by the electron donor. In this case, the organic compound is preferably a material that is excellent in transporting the generated electrons. Specifically, the above-described substances for forming the electron-transport layer 114 (e.g., a metal complex or a heteroaromatic compound) can be used, for example. As the electron donor, a substance having an electron-donating property with respect to the organic compound is used. Specifically, it is preferable to use an alkali metal, an alkaline earth metal, or a rare earth metal, such as lithium, sodium, cesium, magnesium, calcium, erbium, or ytterbium. It is also preferable to use an alkali metal oxide or an alkaline earth metal oxide, such as lithium oxide, calcium oxide, or barium oxide. Alternatively, a Lewis base such as magnesium oxide can be used. Further alternatively, an organic compound such as tetrathiafulvalene (abbreviation: TTF) can be used.

Note that the light-emitting layer, the hole-injection layer, the hole-transport layer, the electron-transport layer, and the electron-injection layer described above can each be formed by an evaporation method (including a vacuum evaporation method), an ink-jet method, a coating method, a gravure printing method, or the like. Besides the above-mentioned materials, an inorganic compound such as a quantum dot or a high molecular compound (e.g., an oligomer, a dendrimer, or a polymer) may be used in the light-emitting layer, the hole-injection layer, the hole-transport layer, the electron-transport layer, and the electron-injection layer.

The quantum dot may be a colloidal quantum dot, an alloyed quantum dot, a core-shell quantum dot, or a core quantum dot, for example. The quantum dot containing elements belonging to Groups 2 and 16, elements belonging to Groups 13 and 15, elements belonging to Groups 13 and 17, elements belonging to Groups 11 and 17, or elements belonging to Groups 14 and 15 may be used. Alternatively, the quantum dot containing an element such as cadmium (Cd), selenium (Se), zinc (Zn), sulfur (S), phosphorus (P), indium (In), tellurium (Te), lead (Pb), gallium (Ga), arsenic (As), or aluminum (Al) may be used.

### <<Pair of Electrodes>>

The first electrode 101 and the second electrode 102 function as an anode and a cathode of the light-emitting device. The first electrode 101 and the second electrode 102 can be formed using a metal, an alloy, or a conductive compound, a mixture or a stack thereof, or the like.

One of the first electrode 101 and the second electrode 102 is preferably formed using a conductive material having a function of reflecting light. Examples of the conductive material include aluminum (Al), an alloy containing Al, and the like. Examples of the alloy containing Al include an alloy containing Al and L (L represents one or more of titanium (Ti), neodymium (Nd), nickel (Ni), and lanthanum (La)), such as an alloy containing Al and Ti and an alloy containing Al, Ni, and La. Aluminum has low resistance and high light reflectivity. Aluminum is included in earth's crust in large amount and is inexpensive; therefore, it is possible to reduce costs for manufacturing a light-emitting device with aluminum. Alternatively, silver (Ag), an alloy of Ag andN (N represents one or more of yttrium (Y), Nd, magnesium (Mg), ytterbium (Yb), Al, Ti, gallium (Ga), zinc (Zn), indium (In), tungsten (W), manganese (Mn), tin (Sn), iron (Fe), Ni, copper (Cu), palladium (Pd), iridium (Ir), and gold (Au)), or the like may be used. Examples of the alloy containing silver include an alloy containing silver, palladium, and copper, an alloy containing silver and copper, an alloy containing silver and magnesium, an alloy containing silver and nickel, an alloy containing silver and gold, an alloy containing silver and ytterbium, and the like. Besides, a transition metal such as tungsten, chromium (Cr), molybdenum (Mo), copper, or titanium can be used.

Light emitted from the light-emitting layer is extracted through the first electrode 101 and/or the second electrode 102. Thus, at least one of the first electrode 101 and the second electrode 102 is preferably formed using a conductive material having a function of transmitting light. As the conductive material, a conductive material having a visible light transmittance higher than or equal to 40% and lower than or equal to 100%, preferably higher than or equal to 60% and lower than or equal to 100%, and a resistivity lower than or equal to 1×10⁻² Ω·cm can be used.

Each of the first electrode 101 and the second electrode 102 may be formed using a conductive material having functions of transmitting light and reflecting light. As the conductive material, a conductive material having a visible light reflectance higher than or equal to 20% and lower than or equal to 80%, preferably higher than or equal to 40% and lower than or equal to 70%, and a resistivity lower than or equal to 1×10⁻² Ω·cm can be used. For example, one or more kinds of conductive metals and alloys, conductive compounds, and the like can be used. Specifically, a metal oxide such as indium tin oxide (hereinafter, referred to as ITO), indium tin oxide containing silicon or silicon oxide (ITSO), indium oxide-zinc oxide (indium zinc oxide), indium oxide-tin oxide containing titanium, indium titanium oxide, or indium oxide containing tungsten oxide and zinc oxide can be used. A metal thin film having a thickness that allows transmission of light (preferably, a thickness greater than or equal to 1 nm and less than or equal to 30 nm) can also be used. As the metal, Ag, an alloy of Ag and Al, an alloy of Ag and Mg, an alloy of Ag and Au, an alloy of Ag and Yb, or the like can be used.

In this specification and the like, as the material having a function of transmitting light, a material having a function of transmitting visible light and having conductivity is used. Examples of the material include, in addition to the above-described oxide conductor typified by ITO, an oxide semiconductor and an organic conductor containing an organic substance. Examples of the organic conductor containing an organic substance include a composite material in which an organic compound and an electron donor (donor) are mixed and a composite material in which an organic compound and an electron acceptor (acceptor) are mixed. Alternatively, an inorganic carbon-based material such as graphene may be used. The resistivity of the material is preferably lower than or equal to 1×10⁵ Ω·cm, further preferably lower than or equal to 1×10⁴ Ω·cm.

Alternatively, the first electrode 101 and/or the second electrode 102 may be formed by stacking two or more of these materials.

In order to improve the light extraction efficiency, a material whose refractive index is higher than that of an electrode having a function of transmitting light may be formed in contact with the electrode. The material may be electrically conductive or non-conductive as long as it has a function of transmitting visible light. In addition to the oxide conductors described above, an oxide semiconductor and an organic substance are given as the examples of the material. Examples of the organic substance include the materials for the light-emitting layer, the hole-injection layer, the hole-transport layer, the electron-transport layer, and the electron-injection layer. Alternatively, an inorganic carbon-based material or a metal film thin enough to transmit light can be used. Further alternatively, stacked layers with a thickness of several nanometers to several tens of nanometers may be used.

In the case where the first electrode 101 or the second electrode 102 functions as the cathode, the electrode preferably contains a material having a low work function (lower than or equal to 3.8 eV). For example, it is possible to use an element belonging to Group 1 or 2 of the periodic table (e.g., an alkali metal such as lithium, sodium, or cesium, an alkaline earth metal such as calcium or strontium, or magnesium), an alloy containing any of these elements (e.g., Ag—Mg or Al—Li), a rare earth metal such as europium (Eu) or Yb, an alloy containing any of these rare earth metals, an alloy containing aluminum or silver, or the like.

When the first electrode 101 or the second electrode 102 is used as the anode, a material with a high work function (4.0 eV or higher) is preferably used.

The first electrode 101 and the second electrode 102 may be a stacked layer of a conductive material having a function of reflecting light and a conductive material having a function of transmitting light. This structure is preferably employed, in which case the first electrode 101 and the second electrode 102 can have a function of adjusting the optical path length so that light with a desired wavelength emitted from each light-emitting layer resonates and is intensified.

As a method for forming the first electrode 101 and the second electrode 102, a sputtering method, an evaporation method, a printing method, a coating method, a molecular beam epitaxy (MBE) method, a CVD method, a pulsed laser deposition method, an atomic layer deposition (ALD) method, or the like can be used as appropriate.

### <<Charge-Generation Layer>>

The charge-generation layer 106 has a function of injecting electrons into the organic compound layer 103a and injecting holes into the organic compound layer 103b when a voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these layers may be stacked. Furthermore, an electron-relay layer may be provided between the p-type layer and the electron-injection buffer layer. Forming the charge-generation layer 106 with the use of any of the above materials can inhibit an increase in driving voltage caused by the stack of the organic compound layers.

In the case where the charge-generation layer 106 is a p-type layer in which an electron acceptor is added to a hole-transport material, which is an organic compound, any of the materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films containing the respective materials may be used.

In the case where the charge-generation layer 106 is an electron-injection buffer layer in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An organic compound such as tetrathianaphthacene may be used as the electron donor.

When an electron-relay layer is provided between a p-type layer and an electron-injection buffer layer in the charge-generation layer 106, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer is preferably higher than or equal to −5.0 eV, further preferably higher than or equal to −5.0 eV and lower than or equal to −3.0 eV. Note that as the substance having an electron-transport property in the electron-relay layer, a phthalocyanine-based material or a metal complex having a metal-oxygen bond and an aromatic ligand is preferably used.

Although FIG. 2D illustrates the structure in which two of the organic compound layers 103 are stacked, three or more organic compound layers may be stacked with charge-generation layers each provided between two adjacent organic compound layers.

### <<Cap Layer>>

Although not illustrated in FIGS. 2A to 2E, a cap layer may be provided over the second electrode 102 of the light-emitting device. For example, a material with a high refractive index can be used for the cap layer. When the cap layer is provided over the second electrode 102, extraction efficiency of light emitted from the second electrode 102 can be improved.

Specific examples of a material that can be used for the cap layer include 5,5′-diphenyl-2,2′-di-5H-[1]benzothieno[3,2-c]carbazole (abbreviation: BisBTc) and 4,4′,4″-(benzene-1,3,5-triyl)tri(dibenzothiophene) (abbreviation: DBT3P-II). In addition, the organic compound described in Embodiment 1 can also be used for the cap layer. Thus, the reliability of the light-emitting device can be increased.

### <<Substrate>>

A light-emitting device of one embodiment of the present invention may be formed over a substrate of glass, plastic, or the like. As the way of stacking layers over the substrate, layers may be sequentially stacked from the first electrode 101 side or sequentially stacked from the second electrode 102 side.

For the substrate over which the light-emitting device of one embodiment of the present invention can be formed, glass, quartz, plastic, or the like can be used, for example. Alternatively, a flexible substrate may be used. The flexible substrate means a substrate that can be bent, such as a plastic substrate made of polycarbonate or polyarylate, for example. Alternatively, a film, an inorganic vapor deposition film, or the like can be used. Another material may be used as long as the substrate functions as a support in a manufacturing process of the light-emitting device or an optical element or as long as it has a function of protecting the light-emitting device or an optical element.

In this specification and the like, a light-emitting device can be formed using any of a variety of substrates, for example. There is no particular limitation on the type of the substrate. Examples of the substrate include a semiconductor substrate (e.g., a single crystal substrate or a silicon substrate); an SOI substrate; a glass substrate; a quartz substrate; a plastic substrate; a metal substrate; a stainless steel substrate; a substrate including stainless steel foil; a tungsten substrate; a substrate including tungsten foil; a flexible substrate; an attachment film; and cellulose nanofiber (CNF), paper, and a base material film that include a fibrous material. As examples of a glass substrate, a barium borosilicate glass substrate, an aluminoborosilicate glass substrate, a soda lime glass substrate, and the like can be given. Examples of the flexible substrate, the attachment film, the base material film, and the like are substrates of plastics typified by polyethylene terephthalate (PET), polyethylene naphthalate (PEN), polyether sulfone (PES), and polytetrafluoroethylene (PTFE). Another example is an acrylic resin. Furthermore, polypropylene, polyester, polyvinyl fluoride, and polyvinyl chloride can be given as examples. Other examples include a resin such as a polyamide resin, a polyimide resin, an aramid resin, or an epoxy resin, an inorganic vapor deposition film, and paper.

Alternatively, a flexible substrate may be used as the substrate, and a light-emitting device maybe provided directly on the flexible substrate. Further alternatively, a separation layer may be provided between the substrate and the light-emitting device. The separation layer can be used to separate part or the whole of the light-emitting device, which is formed over the separation layer, from the substrate and transfer the separated component onto another substrate. In that case, the light-emitting device can be transferred to a substrate having low heat resistance or a flexible substrate as well. For the above separation layer, a stack including inorganic films, which are a tungsten film and a silicon oxide film, or a structure in which a resin film of polyimide or the like is formed over a substrate can be used, for example.

In other words, after the light-emitting device is formed using a substrate, the light-emitting device may be transferred to another substrate. Examples of the substrate to which the light-emitting device is transferred are, in addition to the above substrates, a cellophane substrate, a stone substrate, a wood substrate, a cloth substrate (including a natural fiber (e.g., silk, cotton, or hemp), a synthetic fiber (e.g., nylon, polyurethane, or polyester), a regenerated fiber (e.g., acetate, cupro, rayon, or regenerated polyester), and the like), a leather substrate, a rubber substrate, and the like. When such a substrate is used, a light-emitting device with high durability, high heat resistance, reduced weight, or reduced thickness can be formed.

The light-emitting device may be formed over an electrode electrically connected to, for example, a field-effect transistor (FET) formed over any of the above-described substrates. In that case, an active matrix display device in which the FET controls the driving of the light-emitting device can be manufactured.

The structure described above in this embodiment can be combined as appropriate with any of the structures described in the other embodiments.

### Embodiment 3

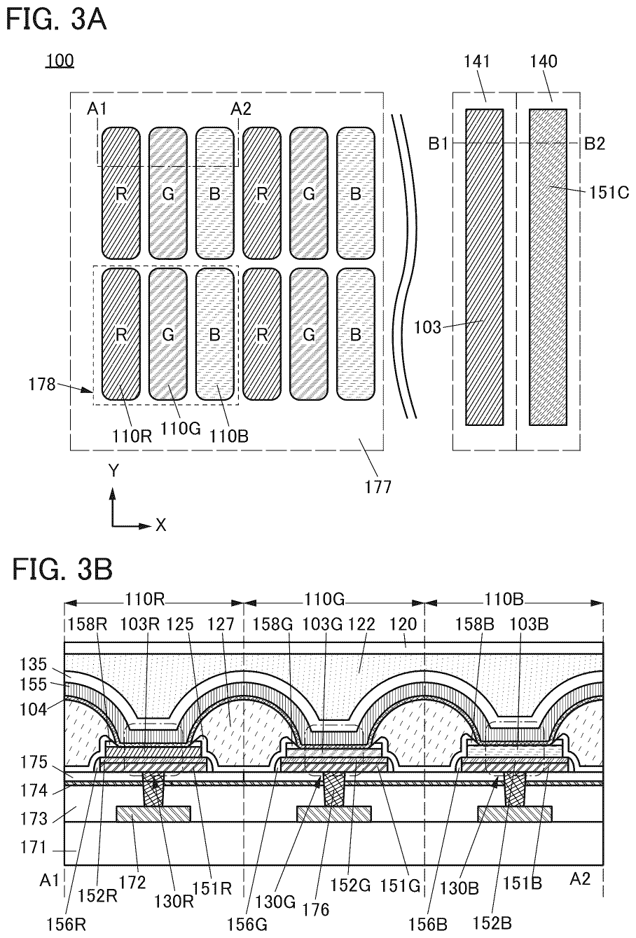

As illustrated in FIG. 3B, a plurality of light-emitting devices 130 are formed over an insulating layer 175 to constitute a display device. In this embodiment, the display device of one embodiment of the present invention will be described in detail.

A display device 100 includes a pixel portion 177 in which a plurality of pixels 178 are arranged in matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and yellow (Y), and four subpixels emitting light of R, G, and B and infrared (IR) light.

In this specification and the like, the row direction and the column direction are sometimes referred to as the X direction and the Y direction, respectively. The X direction and the Y direction intersect with each other and are perpendicular to each other, for example.

FIG. 3A illustrates an example where subpixels of different colors are arranged in the X direction and subpixels of the same color are arranged in the Y direction. Note that subpixels of different colors may be arranged in the Y direction, and subpixels of the same color may be arranged in the X direction.

Outside the pixel portion 177, a connection portion 140 is provided and a region 141 may also be provided. The region 141 is provided between the pixel portion 177 and the connection portion 140. The organic compound layer 103 is provided in the region 141. A conductive layer 151C is provided in the connection portion 140.

Although FIG. 3A illustrates an example where the region 141 and the connection portion 140 are located on the right side of the pixel portion 177, there is no particular limitation on the positions of the region 141 and the connection portion 140. The number of regions 141 and the number of connection portions 140 can each be one or more.

FIG. 3B is an example of a cross-sectional view along the dashed-dotted line A1-A2 in FIG. 3A. As illustrated in FIG. 3A, the display device 100 includes an insulating layer 171, a conductive layer 172 over the insulating layer 171, an insulating layer 173 over the insulating layer 171 and the conductive layer 172, an insulating layer 174 over the insulating layer 173, and an insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 135 is provided to cover the light-emitting device 130. A substrate 120 is attached onto the protective layer 135 with a resin layer 122. An inorganic insulating layer 125 and an insulating layer 127 over the inorganic insulating layer 125 are preferably provided between the adjacent light-emitting devices 130.

Although each of the inorganic insulating layer 125 and the insulating layer 127 looks like a plurality of layers in the cross-sectional view in FIG. 3B, each of the inorganic insulating layer 125 and the insulating layer 127 is preferably one continuous layer when the display device 100 is seen from above. In other words, the inorganic insulating layer 125 and the insulating layer 127 preferably include opening portions over a first electrode.

In FIG. 3B, a light-emitting device 130R, a light-emitting device 130G, and a light-emitting device 130B are each illustrated as the light-emitting device 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, 130G, or 130B may emit visible light of another color or infrared light.

The display device of one embodiment of the present invention can be, for example, a top-emission display device where light is emitted in the direction opposite to a substrate over which light-emitting devices are formed. Note that the display device of one embodiment of the present invention may be of a bottom-emission type.

Examples of a light-emitting substance included in the light-emitting device 130 include an organometallic complex and organic compounds such as a substance emitting fluorescent light (a fluorescent material), a substance emitting phosphorescent light (a phosphorescent material), and a substance exhibiting thermally activated delayed fluorescence (a thermally activated delayed fluorescent (TADF) material). Other examples include inorganic compounds (e.g., a quantum dot material).

The light-emitting device 130R has a structure as described in Embodiment 1. The light-emitting device 130R includes the first electrode (pixel electrode) including a conductive layer 151R and a conductive layer 152R, an organic compound layer 103R over the first electrode, a common layer 104 over the organic compound layer 103R, and a common electrode 155 over the common layer 104. Note that the common electrode 155 corresponds to the second electrode 102 in Embodiments 1 and 2. Although the common layer 104 is not necessarily provided, it is preferable to provide the common layer 104 to reduce damage to the organic compound layer 103R during processing. In the case where the common layer 104 is provided, the common layer 104 is preferably an electron-injection layer. Furthermore, in the case where the common layer 104 is not provided, the organic compound layer 103R corresponds to the organic compound layer 103 described in Embodiments 1 and 2. In the case where the common layer 104 is provided, a stack of the organic compound layer 103R and the common layer 104 corresponds to the organic compound layer 103 described in Embodiments 1 and 2.

The light-emitting device 130G has a structure as described in Embodiment 1. The light-emitting device 130G includes the first electrode (pixel electrode) including a conductive layer 151G and a conductive layer 152G, an organic compound layer 103G over the first electrode, the common layer 104 over the organic compound layer 103G, and the common electrode 155 over the common layer 104. Note that the common electrode 155 corresponds to the second electrode 102 in Embodiments 1 and 2. Although the common layer 104 is not necessarily provided, it is preferable to provide the common layer 104 to reduce damage to the organic compound layer 103G during processing. Furthermore, in the case where the common layer 104 is not provided, the organic compound layer 103G corresponds to the organic compound layer 103 described in Embodiments 1 and 2. In the case where the common layer 104 is provided, a stack of the organic compound layer 103G and the common layer 104 corresponds to the organic compound layer 103 described in Embodiments 1 and 2.

The light-emitting device 130B has a structure as described in Embodiment 1. The light-emitting device 130B includes the first electrode (pixel electrode) including a conductive layer 151B and a conductive layer 152B, an organic compound layer 103B over the first electrode, the common layer 104 over the organic compound layer 103B, and the common electrode 155 over the common layer 104. Note that the common electrode 155 corresponds to the second electrode 102 in Embodiments 1 and 2. Although the common layer 104 is not necessarily provided, it is preferable to provide the common layer 104 to reduce damage to the organic compound layer 103B during processing. Furthermore, in the case where the common layer 104 is not provided, the organic compound layer 103B corresponds to the organic compound layer 103 described in Embodiments 1 and 2. In the case where the common layer 104 is provided, a stack of the organic compound layer 103B and the common layer 104 corresponds to the organic compound layer 103 described in Embodiments 1 and 2.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layers 103R, 103G, and 103B are island-shaped layers that are independent of each other on a light-emitting device basis or on an emission color basis. Providing the island-shaped organic compound layer 103 in each of the light-emitting devices 130 can inhibit leakage current between the adjacent light-emitting devices 130 even in a high-resolution display device. This can prevent crosstalk, so that a display device with extremely high contrast can be obtained. Specifically, a display device having high current efficiency at low luminance can be obtained.

The island-shaped organic compound layer 103 is formed by forming an EL film and processing the EL film by a lithography method.

In the display device of one embodiment of the present invention, the first electrode (pixel electrode) of the light-emitting device preferably has a stacked-layer structure. For example, in the example illustrated in FIG. 3B, the first electrode of the light-emitting device 130 is a stack of the conductive layer 151 (151R, 151G, and 151B) and the conductive layer 152 (152R, 152G, and 152B). In the case where the display device 100 is of a top-emission type and the pixel electrode of the light-emitting device 130 functions as the anode, for example, the conductive layer 151 preferably has high visible light reflectance, and the conductive layer 152 preferably has a visible-light-transmitting property and a high work function. In the case where the display device 100 is of a top-emission type, the higher the visible light reflectance of the pixel electrode is, the higher the efficiency of extraction of the light emitted by the organic compound layer 103 is. In the case where the pixel electrode functions as the anode, the higher the work function of the pixel electrode is, the easier hole injection into the organic compound layer 103 is. Accordingly, when the pixel electrode of the light-emitting device 130 is a stacked of the conductive layer 151 with high visible light reflectance and the conductive layer 152 with a high work function, the light-emitting device 130 can have high light extraction efficiency and a low driving voltage. In this specification and the like, for example, description common to the conductive layers 151R, 151G, and 151B is sometimes made using the collective term “conductive layer 151”.

In the case where the conductive layer 151 has high visible light reflectance, the visible light reflectance of the conductive layer 151 is preferably higher than or equal to 40% and lower than or equal to 100% or higher than or equal to 70% and lower than or equal to 100%, for example. When used as an electrode having a visible-light-transmitting property, the conductive layer 152 preferably has a visible light transmittance higher than or equal to 40%, for example.

Here, such a pixel electrode being a stack composed of a plurality of layers might change in quality as a result of, for example, a reaction between the plurality of layers. For example, when a film formed after the formation of the pixel electrode is removed by a wet etching method, contact of a chemical solution with the pixel electrode might cause galvanic corrosion.

Thus, in the display device 100 of this embodiment, an insulating layer 156 (156R, 156G, and 156B) is formed on side surfaces of the conductive layers 151 and 152. This can inhibit a chemical solution from coming into contact with the conductive layer 151 even when a film that is formed after formation of the pixel electrode including the conductive layer 151 and the conductive layer 152 is removed by a wet etching method, for example. Accordingly, occurrence of galvanic corrosion in the pixel electrode can be inhibited, for example. This allows the display device 100 to be manufactured by a high-yield method and to be accordingly inexpensive. In addition, generation of a defect in the display device 100 can be inhibited, which makes the display device 100 highly reliable. In this specification and the like, description common to the insulating layers 156R, 156G, and 156B is sometimes made using the collective term “insulating layer 156”.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, indium tin oxide containing silicon can be suitably used for the conductive layer 152 because of having a high work function, for example, a work function higher than or equal to 4.0 eV.

The conductive layer 151 and the conductive layer 152 may each be a stack of a plurality of layers that include different materials. In that case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

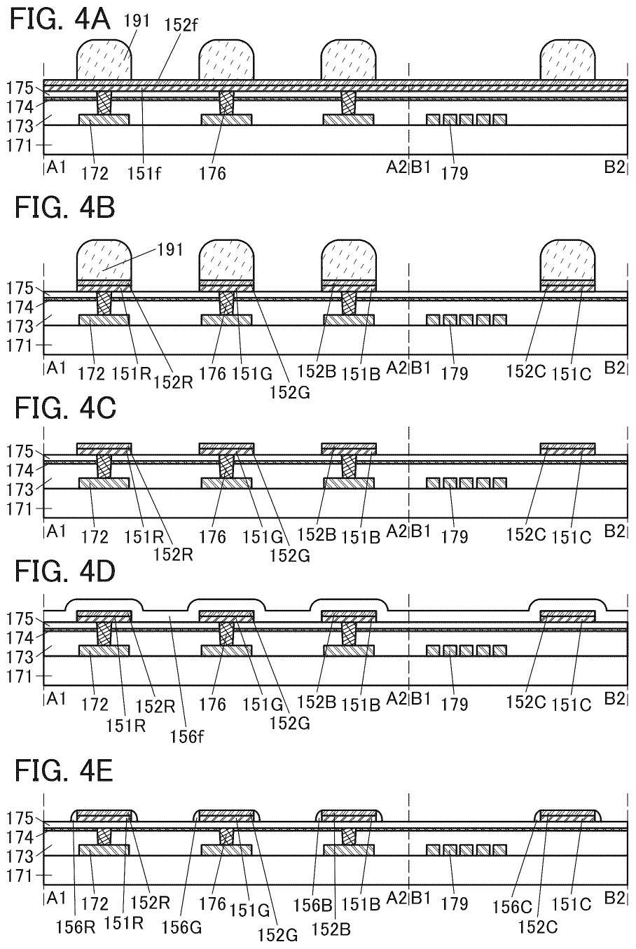

Next, a method for manufacturing the display device 100 having the structure illustrated in FIG. 3A is described with reference to FIGS. 4A to 4E, FIGS. 5A and 5B, FIGS. 6A to 6D, FIGS. 7A to 7C, FIGS. 8A to 8C, and FIGS. 9A to 9C. The organic compound layer of the light-emitting device included in the display device 100 is formed by manufacturing steps including treatment using water. When the light-emitting device of one embodiment of the present invention is used as the light-emitting device included in the display device of one embodiment of the present invention, the display device including the light-emitting device with reduced driving voltage and high emission efficiency can be provided.

### Manufacturing Method Example

Thin films included in the display device (e.g., insulating films, semiconductor films, and conductive films) can be formed by a sputtering method, a chemical vapor deposition (CVD) method, a vacuum evaporation method, a pulsed laser deposition (PLD) method, an ALD method, or the like. Examples of a CVD method include a plasma-enhanced CVD (PECVD) method and a thermal CVD method. An example of a thermal CVD method is a metal organic CVD (MOCVD) method.

Thin films included in the display device (e.g., insulating films, semiconductor films, and conductive films) can also be formed by a wet film-formation method such as spin coating, dipping, spray coating, ink-jetting, dispensing, screen printing, offset printing, doctor blade coating, slit coating, roll coating, curtain coating, or knife coating.

Specifically, for manufacture of the light-emitting device, a vacuum process such as an evaporation method and a solution process such as a spin coating method or an ink-jet method can be used. Examples of an evaporation method include physical vapor deposition methods (PVD methods) such as a sputtering method, an ion plating method, an ion beam evaporation method, a molecular beam evaporation method, and a vacuum evaporation method, and a chemical vapor deposition method (CVD method). Specifically, the functional layers (e.g., the hole-injection layer, the hole-transport layer, the hole-blocking layer, the light-emitting layer, the electron-blocking layer, the electron-transport layer, and the electron-injection layer) included in the organic compound layer can be formed by an evaporation method (e.g., a vacuum evaporation method), a coating method (e.g., a dip coating method, a die coating method, a bar coating method, a spin coating method, or a spray coating method), a printing method (e.g., ink-jetting, screen printing (stencil), offset printing (planography), flexography (relief printing), gravure printing, or micro-contact printing), or the like.

Thin films included in the display device can be processed by a lithography method, for example. Alternatively, a nanoimprinting method, a sandblasting method, a lift-off method, or the like maybe used to process thin films. Alternatively, island-shaped thin films maybe directly formed by a film formation method using a shielding mask such as a metal mask.

As a lithography method, for example, a photolithography method can be used. There are two typical examples of photolithography methods. In one of the methods, a resist mask is formed over a thin film that is to be processed, the thin film is processed by etching, for example, and then the resist mask is removed. In the other method, a photosensitive thin film is formed and then processed into a desired shape by light exposure and development.

As light used for exposure in the photolithography method, for example, light with an i-line (wavelength: 365 nm), light with a g-line (wavelength: 436 nm), light with an h-line (wavelength: 405 nm), or light in which the i-line, the g-line, and the h-line are mixed can be used. Alternatively, ultraviolet rays, KrF laser light, ArF laser light, or the like can be used. Exposure may be performed by liquid immersion exposure technique. As the light for exposure, extreme ultraviolet (EUV) light or X-rays may also be used. Furthermore, instead of the light used for exposure, an electron beam can be used. It is preferable to use EUV light, X-rays, or an electron beam to perform extremely minute processing. Note that when exposure is performed by scanning of a beam such as an electron beam, a photomask is not needed.

For etching of thin films, a dry etching method, a wet etching method, a sandblasting method, or the like can be used.

In a manufacturing process of the light-emitting device, an organic compound that is excited by absorbing light is used. The excited organic compound is highly likely to react with oxygen or water in the air in some cases. In other words, when the organic compound is irradiated with light having a wavelength that is absorbed by the organic compound while oxygen exists, a deterioration product might be generated in the organic compound.

In view of the above, in the case where a substrate over which the organic compound is formed is exposed to the air when processed by a photolithography method, the processing is preferably performed in an environment where lighting is controlled appropriately. The substrate over which the organic compound that is excited by absorbing light is formed is ideally processed under lighting with a wavelength that does not cause excitation of the organic compound; to ensure illuminance or color rendering properties with which work efficiency is not reduced, lighting with the shortest-wavelength emission edge among emission edges in the emission spectrum of a light source of less than or equal to 600 nm, preferably less than or equal to 580 nm is preferably used.

It is preferred to use yellow light (light of a fluorescent lamp or light of a light-emitting diode (LED)) which does not include light with a wavelength shorter than 500 nm for the lighting, for example. It is further preferred to use orange light (light of a fluorescent lamp or light of a light-emitting diode (LED)) which does not include light with a wavelength shorter than 530 nm. Light of a low-pressure sodium lamp can also be used. Light of an incandescent lamp, light of a fluorescent lamp, light of a light-emitting diode (LED), light of a halogen lamp, or sunlight can be used, for example, as long as an optical filter that can shield light with a short wavelength is used. As the optical filter that can shield light with a short wavelength, for example, a band-pass filter or a long-pass filter (short-wavelength cut filter) can be used. The above lighting can result in low illuminance.

First, as illustrated in FIG. 4A, the insulating layer 171 is formed over a substrate (not illustrated). Next, the conductive layer 172 and a conductive layer 179 are formed over the insulating layer 171, and the insulating layer 173 is formed over the insulating layer 171 so as to cover the conductive layer 172 and the conductive layer 179. Then, the insulating layer 174 is formed over the insulating layer 173, and the insulating layer 175 is formed over the insulating layer 174.

As the substrate, a substrate having heat resistance high enough to withstand at least heat treatment performed later can be used. When an insulating substrate is used, it is possible to use a glass substrate, a quartz substrate, a sapphire substrate, a ceramic substrate, an organic resin substrate, or the like. Alternatively, it is possible to use a semiconductor substrate such as a single crystal semiconductor substrate or a polycrystalline semiconductor substrate of silicon, silicon carbide, or the like, a compound semiconductor substrate of silicon germanium or the like, or an SOI substrate.

Next, as illustrated in FIG. 4A, openings reaching the conductive layer 172 are formed in the insulating layers 175, 174, and 173. Then, the plugs 176 are formed to fill the openings.

Next, as illustrated in FIG. 4A, a conductive film 151f to be the conductive layers 151R, 151G, 151B, and 151C is formed over the plugs 176 and the insulating layer 175. The conductive film 151f can be formed by a sputtering method or a vacuum evaporation method, for example. A metal material can be used for the conductive film 151f, for example.

Next, as illustrated in FIG. 4A, a conductive film 152f to be the conductive layers 152R, 152G, and 152B and a conductive layer 152C is formed over the conductive film 151f. The conductive film 152f can be formed by a sputtering method or a vacuum evaporation method, for example. A conductive oxide can be used for the conductive film 152f, for example. The conductive film 152f can be a stack of a film formed using a metal material and a film formed thereover using a conductive oxide. For example, the conductive film 152f can be a stack of a film formed using titanium, silver, or an alloy containing silver and a film formed thereover using a conductive oxide.

The conductive film 152f can be formed by an ALD method. In this case, for the conductive film 152f, an oxide containing one or more selected from indium, tin, zinc, gallium, titanium, aluminum, and silicon can be used. In this case, the conductive film 152f can be formed by repeating a cycle of introduction of a precursor (generally referred to as a metal precursor or the like in some cases), purge of the precursor, introduction of an oxidizer (generally referred to as a reactant, a non-metal precursor, or the like in some cases), and purge of the oxidizer. Here, in the case where an oxide film including a plurality of kinds of metals (e.g., an indium tin oxide film) is formed as the conductive film 152f, the composition of the metals can be controlled by varying the number of cycles for different kinds of precursors.

For example, in the case where an indium tin oxide film is formed as the conductive film 152f, after a precursor containing indium is introduced, the precursor is purged, and an oxidizer is introduced to form an In—O film, and then a precursor containing tin is introduced, the precursor is purged, and an oxidizer is introduced to form a Sn—O film. Here, when the number of cycles of forming an In—O film is larger than the number of cycles of forming a Sn—O film, the number of In atoms included in the conductive film 152f can be larger than the number of Sn atoms included therein.

For example, to form a zinc oxide film as the conductive film 152f, a Zn—O film is formed in the above procedure. For another example, to form an aluminum zinc oxide film as the conductive film 152f, a Zn—O film and an Al—O film are formed in the above procedure. For another example, to form a titanium oxide film as the conductive film 152f, a Ti—O film is formed in the above procedure. For another example, to form an indium tin oxide film including silicon as the conductive film 152f, an In—O film, a Sn—O film, and a Si—O film are formed in the above procedure. For another example, to form a zinc oxide film including gallium, a Ga—O film and a Zn—O film are formed in the above procedure.

As a precursor containing indium, it is possible to use, for example, triethylindium, trimethylindium, or [1,1,1-trimethyl-N-(trimethylsilyl)amide]-indium. As a precursor containing tin, it is possible to use, for example, tin chloride or tetrakis(dimethylamido)tin. As a precursor containing zinc, it is possible to use, for example, diethylzinc or dimethylzinc. As a precursor containing gallium, it is possible to use, for example, triethylgallium. As a precursor containing titanium, it is possible to use, for example, titanium chloride, tetrakis(dimethylamido)titanium, or tetraisopropyl titanate. As a precursor containing aluminum, it is possible to use, for example, aluminum chloride or trimethylaluminum. As a precursor containing silicon, it is possible to use, for example, trisilylamine, bis(diethylamino)silane, tris(dimethylamino)silane, bis(tert-butylamino)silane, or bis(ethylmethylamino)silane. As the oxidizer, water vapor, oxygen plasma, or an ozone gas can be used.

Subsequently, a resist mask 191 is formed over the conductive films 151f and 152f as illustrated in FIG. 4A. The resist mask 191 can be formed by application of a photosensitive material (photoresist), light exposure, and development.

Subsequently, as illustrated in FIG. 4B, the conductive films 151f and 152f in a region not overlapping with the resist mask 191, for example, are removed by an etching method, specifically, a dry etching method, for instance, so that the pixel electrodes each including the conductive layers 151 and 152 are formed. Note that in the case where the conductive film 151f includes a layer formed using a conductive oxide such as indium tin oxide, for example, the layer may be removed by a wet etching method. Thus, the conductive layers 151 and 152 are formed. In the case where part of the conductive film 151f is removed by a dry etching method, for example, a depressed portion may be formed in a region of the insulating layer 175 not overlapping with the conductive layer 151.

Note that the following process may be employed: the conductive film 152f is processed by a lithography method to form the conductive layers 152R, 152G, 152B, and 152C, and then, the conductive film 151f is processed using the conductive layers 152R, 152G, 152B, and 152C as masks. Specifically, after a resist mask is formed, part of the conductive film 152f is removed by an etching method, for example. The conductive film 152f can be removed by a wet etching method, for example. The conductive film 152f may be removed by a dry etching method. After that, the conductive film 151f is preferably removed by a wet etching method.

Here, hydrophobization treatment is preferably performed on the conductive layer 152. The hydrophobization treatment can change the hydrophilic properties of the subject surface to hydrophobic properties or increase the hydrophobic properties of the subject surface. The hydrophobization treatment for the conductive layer 152 can increase the adhesion between the conductive layer 152 and the organic compound layer 103 formed in a later step and inhibit film peeling. Note that the hydrophobization treatment is not necessarily performed.

Next, the resist mask 191 is removed as illustrated in FIG. 4C. The resist mask 191 can be removed by ashing using oxygen plasma, for example. Alternatively, an oxygen gas and any of CF₄, C₄F₈, SF₆, CHF₃, Cl₂, H₂O, BCl₃, and a Group 18 element such as He may be used. Alternatively, the resist mask 191 may be removed by wet etching.

Then, as illustrated in FIG. 4D, an insulating film 156f to be the insulating layers 156R, 156G, and 156B, and an insulating layer 156C is formed over the conductive layers 151R and 152R, the conductive layers 151G and 152G, the conductive layers 151B and 152B, the conductive layers 151C and 152C, and the insulating layer 175. The insulating film 156f can be formed by a CVD method, an ALD method, a sputtering method, or a vacuum evaporation method, for example.

For the insulating film 156f, an inorganic material can be used. As the insulating film 156f, an inorganic insulating film such as an oxide insulating film, a nitride insulating film, an oxynitride insulating film, or a nitride oxide insulating film can be used, for example. For example, an oxide insulating film including silicon, a nitride insulating film including silicon, an oxynitride insulating film including silicon, a nitride oxide insulating film including silicon, or the like can be used as the insulating film 156f. For the insulating film 156f, silicon oxynitride can be used, for example.

Subsequently, as illustrated in FIG. 4E, the insulating film 156f is processed to form the insulating layers 156R, 156G, 156B, and 156C. The insulating layer 156 can be formed by performing etching substantially uniformly on the top surface of the insulating film 156f, for example. Such uniform etching for planarization is also referred to as etch back treatment. Note that the insulating layer 156 may be formed by a lithography method.

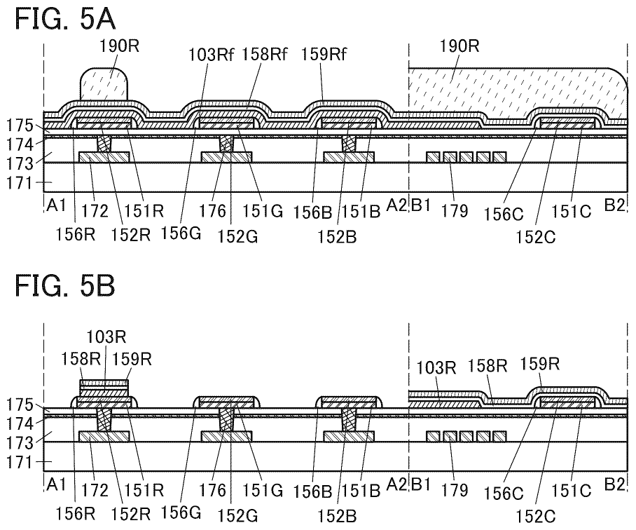

Next, as illustrated in FIG. 5A, an organic compound film 103Rf to be the organic compound layer 103R is formed over the conductive layers 152R, 152G, and 152B, the insulating layers 156R, 156G, and 156B, and the insulating layer 175.

As illustrated in FIG. 5A, the organic compound film 103Rf is not formed over the conductive layer 152C. For example, a mask for defining a film formation area (also referred to as an area mask, a rough metal mask, or the like to be distinguished from a fine metal mask) is used, so that the organic compound film 103Rf can be formed only in a desired region. Employing a film formation step using an area mask and a processing step using a resist mask enables a light-emitting device to be manufactured by a relatively easy process.

The organic compound film 103Rf can be formed by an evaporation method, specifically a vacuum evaporation method, for example. The organic compound film 103Rf may be formed by a transfer method, a printing method, an ink-jet method, a coating method, or the like.

Next, as illustrated in FIG. 5A, a sacrificial film 158Rf to be a sacrificial layer 158R and a mask film 159Rf to be a mask layer 159R are sequentially formed over the organic compound film 103Rf, the conductive layer 152C, and the insulating layer 175.

Although this embodiment shows an example where a mask film having a two-layer structure of the sacrificial film 158Rf and the mask film 159Rf is formed, a mask film may have a single-layer structure or a stacked-layer structure of three or more layers. In this specification and the like, a mask layer may be referred to as a sacrificial layer.

Providing the sacrificial layer over the organic compound film 103Rf can reduce damage to the organic compound film 103Rf in the manufacturing process of the display device, resulting in an increase in reliability of the light-emitting device.

As the sacrificial film 158Rf, a film that is highly resistant to the process conditions for the organic compound film 103Rf, specifically, a film having high etching selectivity with respect to the organic compound film 103Rf is used. For the mask film 159Rf, a film having high etching selectivity with respect to the sacrificial film 158Rf is used.

The sacrificial film 158Rf and the mask film 159Rf are formed at a temperature lower than the upper temperature limit of the organic compound film 103Rf. The typical substrate temperatures in formation of the sacrificial film 158Rf and the mask film 159Rf are each lower than or equal to 200° C., preferably lower than or equal to 150° C., further preferably lower than or equal to 120° C., still further preferably lower than or equal to 100° C., yet still further preferably lower than or equal to 80° C.

The sacrificial film 158Rf and the mask film 159Rf are preferably films that can be removed by a wet etching method. The use of a wet etching method can reduce damage to the organic compound film 103Rf in processing of the sacrificial film 158Rf and the mask film 159Rf, as compared to the case of using a dry etching method.

The sacrificial film 158Rf and the mask film 159Rf can be formed by a sputtering method, an ALD method (including a thermal ALD method and a PEALD method), a CVD method, or a vacuum evaporation method, for example. Alternatively, the sacrificial film 158Rf and the mask film 159Rf may be formed by the above-described wet film-formation method.

Note that the sacrificial film 158Rf that is formed over and in contact with the organic compound film 103Rf is preferably formed by a formation method that is less likely to damage the organic compound film 103Rf than a formation method of the mask film 159Rf. For example, the sacrificial film 158Rf is preferably formed by an ALD method or a vacuum evaporation method rather than a sputtering method.

As each of the sacrificial film 158Rf and the mask film 159Rf, one or more of a metal film, an alloy film, a metal oxide film, a semiconductor film, an organic insulating film, and an inorganic insulating film, for example, can be used.

For each of the sacrificial film 158Rf and the mask film 159Rf, it is possible to use a metal material such as gold, silver, platinum, magnesium, nickel, tungsten, chromium, molybdenum, iron, cobalt, copper, palladium, titanium, aluminum, yttrium, zirconium, or tantalum or an alloy material containing any of the metal materials, for example. It is particularly preferable to use a low-melting-point material such as aluminum or silver. A metal material that can block ultraviolet rays is preferably used for one or both of the sacrificial film 158Rf and the mask film 159Rf, in which case the organic compound film 103Rf can be inhibited from being irradiated with ultraviolet rays and deteriorating.

The sacrificial film 158Rf and the mask film 159Rf can each be formed using a metal oxide such as In—Ga—Zn oxide, indium oxide, In—Zn oxide, In—Sn oxide, indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or indium tin oxide containing silicon.

In place of gallium described above, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used.

As each of the sacrificial film and the mask film, a film including a material having a light-blocking property, particularly with respect to ultraviolet rays, is preferably used. Although a variety of materials such as a metal, an insulator, a semiconductor, and a metalloid that have a property of blocking ultraviolet rays can be used as a light-blocking material, each of the sacrificial film and the mask film is preferably a film capable of being processed by etching and is particularly preferably a film having good processability because part or the whole of each of the sacrificial film and the mask film is removed in a later step.

The sacrificial film and the mask film are preferably formed using a semiconductor material such as silicon or germanium, for example, for excellent compatibility with a semiconductor manufacturing process. Alternatively, an oxide or a nitride of the semiconductor material can be used. A non-metallic material such as carbon or a compound thereof can be used. A metal such as titanium, tantalum, tungsten, chromium, or aluminum or an alloy containing at least one of these metals can be used. Alternatively, an oxide containing the above-described metal, such as titanium oxide or chromium oxide, or a nitride such as titanium nitride, chromium nitride, or tantalum nitride can be used.

When a film including a material having a property of blocking ultraviolet rays is used as each of the sacrificial film and the mask film, the organic compound layer can be inhibited from being irradiated with ultraviolet rays in a light exposure step, for example. The organic compound layer is inhibited from being damaged by ultraviolet rays, so that the reliability of the light-emitting device can be improved.

Note that the same effect is obtained when a film including a material having a property of blocking ultraviolet rays is used for an after-mentioned inorganic insulating film 125f.

As each of the sacrificial film 158Rf and the mask film 159Rf, any of a variety of inorganic insulating films can be used. In particular, an oxide insulating film is preferable because its adhesion to the organic compound film 103Rf is higher than that of a nitride insulating film. For example, an inorganic insulating material such as aluminum oxide, hafnium oxide, or silicon oxide can be used for the sacrificial film 158Rf and the mask film 159Rf. As the sacrificial film 158Rf and the mask film 159Rf, aluminum oxide films can be formed by an ALD method, for example. An ALD method is preferably used, in which case damage to a base (in particular, the organic compound layer) can be reduced.

For example, an inorganic insulating film (e.g., an aluminum oxide film) formed by an ALD method can be used as the sacrificial film 158Rf, and an inorganic film (e.g., an In—Ga—Zn oxide film, an aluminum film, or a tungsten film) formed by a sputtering method can be used as the mask film 159Rf.

Note that the same inorganic insulating film can be used for both the sacrificial film 158Rf and the inorganic insulating layer 125 that is to be formed later. For example, an aluminum oxide film formed by an ALD method can be used for both the sacrificial film 158Rf and the inorganic insulating layer 125. For the sacrificial film 158Rf and the inorganic insulating layer 125, the same film formation conditions may be used or different film formation conditions may be used. For example, when the sacrificial film 158Rf is formed under conditions similar to those of the inorganic insulating layer 125, the sacrificial film 158Rf can be an insulating layer having a high barrier property against at least one of water and oxygen. Meanwhile, since the sacrificial film 158Rf is a layer a large part or the whole of which is to be removed in a later step, it is preferable that the processing of the sacrificial film 158Rf be easy. Therefore, the sacrificial film 158Rf is preferably formed with a substrate temperature lower than that for formation of the inorganic insulating layer 125.

One or both of the sacrificial film 158Rf and the mask film 159Rf may be formed using an organic material. For example, as the organic material, a material that can be dissolved in a solvent chemically stable with respect to at least the uppermost film of the organic compound film 103Rf may be used. Specifically, a material that will be dissolved in water or an alcohol can be suitably used. In forming a film of such a material, it is preferable to apply the material dissolved in a solvent such as water or an alcohol by a wet film-formation method and then perform heat treatment for evaporating the solvent. At this time, the heat treatment is preferably performed in a reduced-pressure atmosphere, in which case the solvent can be removed at a low temperature in a short time and thermal damage to the organic compound film 103Rf can be reduced accordingly.

The sacrificial film 158Rf and the mask film 159Rf may be formed using an organic resin such as polyvinyl alcohol (PVA), polyvinyl butyral, polyvinylpyrrolidone, polyethylene glycol, polyglycerin, pullulan, water-soluble cellulose, an alcohol-soluble polyamide resin, or a fluorine resin like perfluoropolymer.

For example, an organic film (e.g., a PVA film) formed by an evaporation method or any of the above wet film-formation methods can be used as the sacrificial film 158Rf, and an inorganic film (e.g., a silicon nitride film) formed by a sputtering method can be used as the mask film 159Rf.

Subsequently, a resist mask 190R is formed over the mask film 159Rf as illustrated in FIG. 5A. The resist mask 190R can be formed by application of a photosensitive material (photoresist), light exposure, and development.

The resist mask 190R may be formed using either a positive resist material or a negative resist material.

The resist mask 190R is provided at a position overlapping with the conductive layer 152R. The resist mask 190R is preferably provided also at a position overlapping with the conductive layer 152C. This can inhibit the conductive layer 152C from being damaged during the process of manufacturing the display device. Note that the resist mask 190R is not necessarily provided over the conductive layer 152C. The resist mask 190R is preferably provided to cover the area from an end portion of the organic compound film 103Rf to an end portion of the conductive layer 152C (the end portion closer to the organic compound film 103Rf), as illustrated in the cross-sectional view along the line B1-B2 in FIG. 5A.

Next, as illustrated in FIG. 5B, part of the mask film 159Rf is removed using the resist mask 190R, so that the mask layer 159R is formed. The mask layer 159R remains over the conductive layers 152R and 152C. After that, the resist mask 190R is removed. Then, part of the sacrificial film 158Rf is removed using the mask layer 159R as a mask (also referred to as a hard mask), so that the sacrificial layer 158R is formed.

Each of the sacrificial film 158Rf and the mask film 159Rf can be processed by a wet etching method or a dry etching method. The sacrificial film 158Rf and the mask film 159Rf are preferably processed by isotropic etching.

The use of a wet etching method can reduce damage to the organic compound film 103Rf in processing of the sacrificial film 158Rf and the mask film 159Rf, as compared to the case of using a dry etching method. In the case of using a wet etching method, it is preferable to use a developer, an aqueous solution of tetramethylammonium hydroxide (TMAH), dilute hydrofluoric acid, oxalic acid, phosphoric acid, acetic acid, nitric acid, or a chemical solution containing a mixed solution of any of these acids, for example.

Since the organic compound film 103Rf is not exposed in the processing of the mask film 159Rf, the range of choice for a processing method for the mask film 159Rf is wider than that for the sacrificial film 158Rf. Specifically, even in the case where a gas containing oxygen is used as an etching gas in the processing of the mask film 159Rf, deterioration of the organic compound film 103Rf can be inhibited.

In the case of using a dry etching method to process the sacrificial film 158Rf, deterioration of the organic compound film 103Rf can be inhibited by not using a gas containing oxygen as the etching gas. In the case of using a dry etching method, it is preferable to use a gas containing CF₄, C₄F₈, SF₆, CHF₃, Cl₂, H₂O, BCl₃, or a Group 18 element such as He, for example, as the etching gas.

For example, in the case where an aluminum oxide film formed by an ALD method is used as the sacrificial film 158Rf, part of the sacrificial film 158Rf can be removed by a dry etching method using CHF₃ and He or a combination of CHF₃, He, and CH₄. In the case where an In—Ga—Zn oxide film formed by a sputtering method is used as the mask film 159Rf, part of the mask film 159Rf can be removed by a wet etching method using diluted phosphoric acid. Alternatively, part of the mask film 159Rf may be removed by a dry etching method using CH₄ and Ar. Alternatively, part of the mask film 159Rf can be removed by a wet etching method using diluted phosphoric acid. In the case where a tungsten film formed by a sputtering method is used as the mask film 159Rf, part of the mask film 159Rf can be removed by a dry etching method using a combination of SF₆, CF₄, and O₂ or a combination of CF₄, Cl₂, and O₂.

The resist mask 190R can be removed by a method similar to that for the resist mask 191. For example, the resist mask 190R can be removed by ashing using oxygen plasma. Alternatively, an oxygen gas and any of CF₄, C₄F₈, SF₆, CHF₃, Cl₂, H₂O, BCl₃, and a Group 18 element such as He may be used. Alternatively, the resist mask 190R may be removed by wet etching. At this time, the sacrificial film 158Rf is located on the outermost surface, and the organic compound film 103Rf is not exposed; thus, the organic compound film 103Rf can be inhibited from being damaged in the step of removing the resist mask 190R. In addition, the range of choice for the method for removing the resist mask 190R can be widened.

Next, as illustrated in FIG. 5B, the organic compound film 103Rf is processed, so that the organic compound layer 103R is formed. For example, part of the organic compound film 103Rf is removed using the mask layer 159R and the sacrificial layer 158R as a hard mask to form the organic compound layer 103R.

Accordingly, as illustrated in FIG. 5B, the stacked-layer structure of the organic compound layer 103R, the sacrificial layer 158R, and the mask layer 159R remains over the conductive layer 152R. The conductive layers 152G and 152B are exposed.

In the example illustrated in FIG. 5B, an end portion of the organic compound layer 103R is located inward from an end portion of the conductive layer 152R. This structure allows miniaturization of pixels, enabling manufacturing a high-resolution display. Although not illustrated in FIG. 5B, by the above etching treatment, a depressed portion may be formed in the insulating layer 175 in a region not overlapping with the organic compound layer 103R.

As described above, the resist mask 190R is preferably provided to cover the area from the end portion of the organic compound layer 103R to the end portion of the conductive layer 152C (the end portion closer to the organic compound layer 103R) in the cross section along the dashed-dotted line B1-B2. In that case, as illustrated in FIG. 5B, the sacrificial layer 158R and the mask layer 159R are provided to cover the area from the end portion of the organic compound layer 103R to the end portion of the conductive layer 152C (the end portion closer to the organic compound layer 103R) in the cross section along the dashed-dotted line B1-B2. Hence, the insulating layer 175 can be inhibited from being exposed in the cross section along the dashed-dotted line B1-B2, for example. This can prevent the insulating layers 175, 174, and 173 from being partly removed by etching and thus prevent the conductive layer 179 from being exposed. Accordingly, the conductive layer 179 can be inhibited from being unintentionally electrically connected to another conductive layer. For example, a short circuit between the conductive layer 179 and the common electrode 155 formed in a later step can be inhibited.

The organic compound film 103Rf is preferably processed by anisotropic etching. Anisotropic dry etching is particularly preferable. Alternatively, wet etching may be used.

In the case of using a dry etching method, deterioration of the organic compound film 103Rf can be inhibited by not using a gas containing oxygen as the etching gas.

A gas containing oxygen may be used as the etching gas. When the etching gas contains oxygen, the etching rate can be increased. Therefore, the etching can be performed under a low-power condition while an adequately high etching rate is maintained. Accordingly, damage to the organic compound film 103Rf can be reduced. Furthermore, a defect such as attachment of a reaction product generated in the etching can be inhibited.

In the case of using a dry etching method, it is preferable to use a gas containing at least one of H₂, CF₄, C₄F₈, SF₆, CHF₃, Cl₂, H₂O, BCl₃, and a Group 18 element such as He or Ar as the etching gas, for example. Alternatively, a gas containing oxygen and at least one of the above is preferably used as the etching gas. Alternatively, an oxygen gas may be used as the etching gas. Specifically, for example, a gas containing H₂ and Ar or a gas containing CF₄ and He can be used as the etching gas. For another example, a gas containing CF₄, He, and oxygen can be used as the etching gas. For another example, a gas containing H₂ and Ar and a gas containing oxygen can be used as the etching gas.

As described above, in one embodiment of the present invention, the mask layer 159R is formed in the following manner: the resist mask 190R is formed over the mask film 159Rf and part of the mask film 159Rf is removed using the resist mask 190R. After that, part of the organic compound film 103Rf is removed using the mask layer 159R as a hard mask, so that the organic compound layer 103R is formed. In other words, the organic compound layer 103R is formed by processing the organic compound film 103Rf by a lithography method. Note that part of the organic compound film 103Rf may be removed using the resist mask 190R. Then, the resist mask 190R may be removed.

Next, hydrophobization treatment for the conductive layer 152G, for example, is preferably performed. At the time of processing the organic compound film 103Rf, the properties of a surface of the conductive layer 152G change to hydrophilic properties in some cases, for example. The hydrophobization treatment for the conductive layer 152G, for example, can increase the adhesion between the conductive layer 152G and a layer to be formed in a later step (which is the organic compound layer 103G here) and inhibit film peeling. Note that the hydrophobization treatment is not necessarily performed.

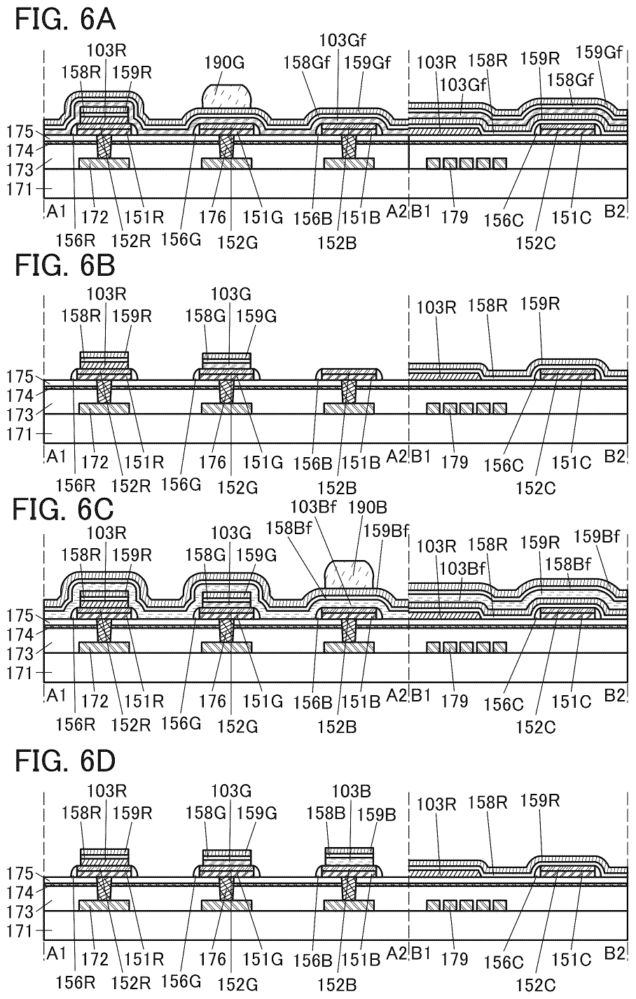

Next, as illustrated in FIG. 6A, an organic compound film 103Gf to be the organic compound layer 103G is formed over the conductive layers 152G and 152B, the insulating layers 156R, 156G, and 156B, the mask layer 159R, and the insulating layer 175.

The organic compound film 103Gf can be formed by a method similar to that for forming the organic compound film 103Rf. The organic compound film 103Gf can have a structure similar to that of the organic compound film 103Rf.

Then, as illustrated in FIG. 6A, a sacrificial film 158Gf to be a sacrificial layer 158G and a mask film 159Gf to be a mask layer 159G are sequentially formed over the organic compound film 103Gf and the mask layer 159R. After that, a resist mask 190G is formed. The materials and the formation methods of the sacrificial film 158Gf and the mask film 159Gf are similar to those of the sacrificial film 158Rf and the mask film 159Rf. The material and the formation method of the resist mask 190G are similar to those of the resist mask 190R.

The resist mask 190G is provided at a position overlapping with the conductive layer 152G.

Subsequently, as illustrated in FIG. 6B, part of the mask film 159Gf is removed using the resist mask 190G, so that the mask layer 159G is formed. The mask layer 159G remains over the conductive layer 152G. After that, the resist mask 190G is removed. Then, part of the sacrificial film 158Gf is removed using the mask layer 159G as a mask, so that the sacrificial layer 158G is formed. Next, the organic compound film 103Gf is processed, so that the organic compound layer 103G is formed. For example, part of the organic compound film 103Gf is removed using the mask layer 159G and the sacrificial layer 158G as a hard mask to form the organic compound layer 103G.

Accordingly, as illustrated in FIG. 6B, the stacked-layer structure of the organic compound layer 103G, the sacrificial layer 158G, and the mask layer 159G remains over the conductive layer 152G. The mask layer 159R and the conductive layer 152B are exposed.

Next, hydrophobization treatment for the conductive layer 152B, for example, is preferably performed. At the time of processing the organic compound film 103Gf, the properties of a surface of the conductive layer 152B change to hydrophilic properties in some cases, for example. The hydrophobization treatment for the conductive layer 152B, for example, can increase the adhesion between the conductive layer 152B and a layer to be formed in a later step (which is the organic compound layer 103B here) and inhibit film peeling. Note that the hydrophobization treatment is not necessarily performed.

Next, as illustrated in FIG. 6C, an organic compound film 103Bf to be the organic compound layer 103B is formed over the conductive layer 152B, the insulating layers 156R, 156G, and 156B, the mask layers 159R and 159G, and the insulating layer 175.

The organic compound film 103Bf can be formed by a method similar to that for forming the organic compound film 103Rf. The organic compound film 103Bf can have a structure similar to that of the organic compound film 103Rf.

Then, as illustrated in FIG. 6C, a sacrificial film 158Bf to be a sacrificial layer 158B and a mask film 159Bf to be a mask layer 159B are sequentially formed over the organic compound film 103Bf and the mask layer 159R. After that, a resist mask 190B is formed. The materials and the formation methods of the sacrificial film 158Bf and the mask film 159Bf are similar to those of the sacrificial film 158Rf and the mask film 159Rf. The material and the formation method of the resist mask 190B are similar to those of the resist mask 190R.

The resist mask 190B is provided at a position overlapping with the conductive layer 152B.

Subsequently, as illustrated in FIG. 6D, part of the mask film 159Bf is removed using the resist mask 190B, so that the mask layer 159B is formed. The mask layer 159B remains over the conductive layer 152B. After that, the resist mask 190B is removed. Then, part of the sacrificial film 158Bf is removed using the mask layer 159B as a mask, so that the sacrificial layer 158B is formed. Next, the organic compound film 103Bf is processed, so that the organic compound layer 103B is formed. For example, part of the organic compound film 103Bf is removed using the mask layer 159B and the sacrificial layer 158B as a hard mask to form the organic compound layer 103B.

Accordingly, as illustrated in FIG. 6D, the stacked-layer structure of the organic compound layer 103B, the sacrificial layer 158B, and the mask layer 159B remains over the conductive layer 152B. The mask layers 159R and 159G are exposed.

Note that side surfaces of the organic compound layers 103R, 103G, and 103B are preferably perpendicular or substantially perpendicular to their formation surfaces. For example, the angle between the formation surfaces and these side surfaces is preferably greater than or equal to 600 and less than or equal to 90°.

The distance between two adjacent layers among the organic compound layers 103R, 103G, and 103B, which are formed by a lithography method as described above, can be shortened to less than or equal to 8 μm, less than or equal to 5 μm, less than or equal to 3 μm, less than or equal to 2 μm, or less than or equal to 1 μm. Here, the distance can be specified, for example, by the distance between opposite end portions of two adjacent layers among the organic compound layers 103R, 103G, and 103B. Shortening the distance between the island-shaped organic compound layers can provide a display device having high resolution and a high aperture ratio. In addition, the distance between the first electrodes of adjacent light-emitting devices can also be shortened to be, for example, less than or equal to 10 μm, less than or equal to 8 μm, less than or equal to 5 μm, less than or equal to 3 μm, or less than or equal to 2 μm. Note that the distance between the first electrodes of adjacent light-emitting devices is preferably greater than or equal to 2 μm and less than or equal to 5 μm.

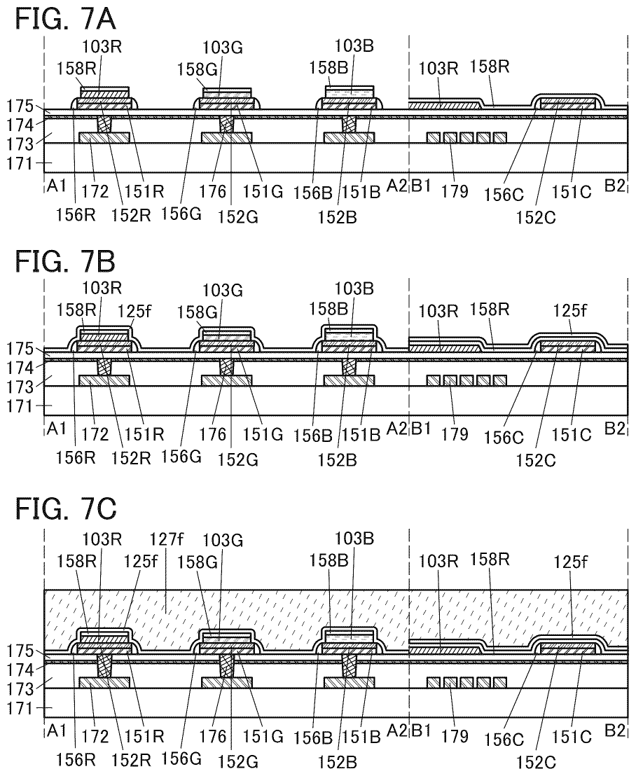

Next, as illustrated in FIG. 7A, the mask layers 159R, 159G, and 159B are preferably removed. The sacrificial layers 158R, 158G, and 158B and the mask layers 159R, 159G, and 159B remain in the display device in some cases depending on the subsequent steps. Removing the mask layers 159R, 159G, and 159B at this stage can inhibit the mask layers 159R, 159G, and 159B from being left in the display device. For example, in the case where a conductive material is used for the mask layers 159R, 159G, and 159B, removing the mask layers 159R, 159G, and 159B in advance can inhibit generation of a leakage current, formation of a capacitor, and the like due to the remaining mask layers 159R, 159G, and 159B.

This embodiment describes an example where the mask layers 159R, 159G, and 159B are removed; however, the mask layers 159R, 159G, and 159B are not necessarily removed. For example, in the case where the mask layers 159R, 159G, and 159B include the above-described material having a property of blocking ultraviolet rays, the procedure preferably proceeds to the next step without removing the mask layers 159R, 159G, and 159B, in which case the organic compound layers can be protected from ultraviolet rays.

The step of removing the mask layers can be performed by a method similar to that for the step of processing the mask films. Specifically, by using a wet etching method, damage applied to the organic compound layers 103R, 103G, and 103B at the time of removing the mask layers can be reduced as compared to the case of using a dry etching method.

The mask layers may be removed by being dissolved in a solvent such as water or an alcohol. Examples of an alcohol include ethyl alcohol, methyl alcohol, isopropyl alcohol (IPA), and glycerin.

After the mask layers are removed, drying treatment may be performed in order to remove water included in the organic compound layers 103R, 103G, and 103B and water adsorbed onto the surfaces of the organic compound layers 103R, 103G, and 103B. For example, heat treatment in an inert gas atmosphere or a reduced-pressure atmosphere can be performed. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 120° C. The heat treatment is preferably performed in a reduced-pressure atmosphere, in which case drying at a lower temperature is possible.

Next, as illustrated in FIG. 7B, the inorganic insulating film 125f to be the inorganic insulating layer 125 is formed to cover the organic compound layers 103R, 103G, and 103B and the sacrificial layers 158R, 158G, and 158B.

As described later, an insulating film 127f to be the insulating layer 127 is formed in contact with the top surface of the inorganic insulating film 125f. Therefore, the top surface of the inorganic insulating film 125f preferably has a high affinity for the material used for the insulating film (e.g., a photosensitive resin composition containing an acrylic resin). To improve the affinity, surface treatment is preferably performed so that the top surface of the inorganic insulating film 125f is made hydrophobic or its hydrophobic properties are improved. For example, it is preferable to perform the treatment using a silylation agent such as hexamethyldisilazane (HMDS). By making the top surface of the inorganic insulating film 125f hydrophobic in such a manner, the insulating film 127f can be formed with favorable adhesion. Note that the above-described hydrophobization treatment may be performed as the surface treatment.

Then, as illustrated in FIG. 7C, the insulating film 127f to be the insulating layer 127 is formed over the inorganic insulating film 125f.

The inorganic insulating film 125f and the insulating film 127f are preferably formed by a formation method by which the organic compound layers 103R, 103G, and 103B are less damaged. The inorganic insulating film 125f, which is formed in contact with the side surfaces of the organic compound layers 103R, 103G, and 103B, is particularly preferably formed by a formation method that causes less damage to the organic compound layers 103R, 103G, and 103B than the formation method of the insulating film 127f.

Each of the inorganic insulating film 125f and the insulating film 127f is formed at a temperature lower than the upper temperature limits of the organic compound layers 103R, 103G, and 103B. When the inorganic insulating film 125f is formed at a high substrate temperature, the formed inorganic insulating film 125f, even with a small thickness, can have a low impurity concentration and a high barrier property against at least one of water and oxygen.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness greater than or equal to 3 nm, greater than or equal to 5 nm, or greater than or equal to 10 nm and less than or equal to 200 nm, less than or equal to 150 nm, less than or equal to 100 nm, or less than or equal to 50 nm is preferably formed in the above-described range of the substrate temperature.

The inorganic insulating film 125f is preferably formed by an ALD method, for example. An ALD method is preferably used, in which case damage due to film formation is reduced and a film with good coverage can be formed. As the inorganic insulating film 125f, an aluminum oxide film is preferably formed by an ALD method, for example.

Alternatively, the inorganic insulating film 125f may be formed by a sputtering method, a CVD method, or a PECVD method, each of which has a higher film formation rate than an ALD method. In that case, a highly reliable display device can be manufactured with high productivity.

The insulating film 127f is preferably formed by the aforementioned wet film-formation method. The insulating film 127f is preferably formed by spin coating using a photosensitive material, for example, and specifically preferably formed using a photosensitive resin composition containing an acrylic resin.

The insulating film 127f is preferably formed using a resin composition containing a polymer, an acid-generating agent, and a solvent, for example. The polymer is formed using one or more kinds of monomers and has a structure where one or more kinds of structural units (also referred to as building blocks) are repeated regularly or irregularly. As the acid-generating agent, one or both of a compound that generates an acid by light irradiation and a compound that generates an acid by heating can be used. The resin composition may also include one or more of a photosensitizing agent, a sensitizer, a catalyst, an adhesive aid, a surface-active agent, and an antioxidant.

Heat treatment (also referred to as prebaking) is preferably performed after the insulating film 127f is formed. The heat treatment is performed at a temperature lower than the upper temperature limits of the organic compound layers 103R, 103G, and 103B. The substrate temperature in the heat treatment is preferably higher than or equal to 50° C. and lower than or equal to 200° C., further preferably higher than or equal to 60° C. and lower than or equal to 150° C., still further preferably higher than or equal to 70° C. and lower than or equal to 120° C. Accordingly, the solvent included in the insulating film 127f can be removed.

Then, part of the insulating film 127f is exposed to visible light or ultraviolet rays. Here, when a positive photosensitive resin composition containing an acrylic resin is used for the insulating film 127f, a region where the insulating layer 127 is not formed in a later step is irradiated with visible light or ultraviolet rays. The insulating layer 127 is formed in regions that are sandwiched between any two of the conductive layers 152R, 152G, and 152B and around the conductive layer 152C. Thus, the top surfaces of the conductive layers 152R, 152G, 152B, and 152C are irradiated with visible light or ultraviolet rays. Note that when a negative photosensitive material is used for the insulating film 127f, the region where the insulating layer 127 is to be formed is irradiated with visible light or ultraviolet rays.

The width of the insulating layer 127 that is to be formed later can be controlled in accordance with the exposed region of the insulating film 127f. In this embodiment, processing is performed such that the insulating layer 127 includes a portion overlapping with the top surface of the conductive layer 151.

The light used for the exposure preferably includes the i-line (wavelength: 365 nm). Furthermore, the light used for the exposure may include at least one of the g-line (wavelength: 436 nm) and the h-line (wavelength: 405 nm).

Here, when a barrier insulating layer against oxygen (e.g., an aluminum oxide film) is provided as one or both of the sacrificial layer 158 (the sacrificial layers 158R, 158G, and 158B) and the inorganic insulating film 125f, diffusion of oxygen into the organic compound layers 103R, 103G, and 103B can be inhibited. When the organic compound layer is irradiated with light (visible light or ultraviolet rays), the organic compound included in the organic compound layer is brought into an excited state and a reaction between the organic compound and oxygen in the atmosphere is promoted in some cases. Specifically, when the organic compound layer is irradiated with light (visible light or ultraviolet rays) in an atmosphere containing oxygen, oxygen might be bonded to the organic compound included in the organic compound layer. By providing the sacrificial layer 158 and the inorganic insulating film 125f over the island-shaped organic compound layer, bonding of oxygen in the atmosphere to the organic compound included in the organic compound layer can be inhibited.

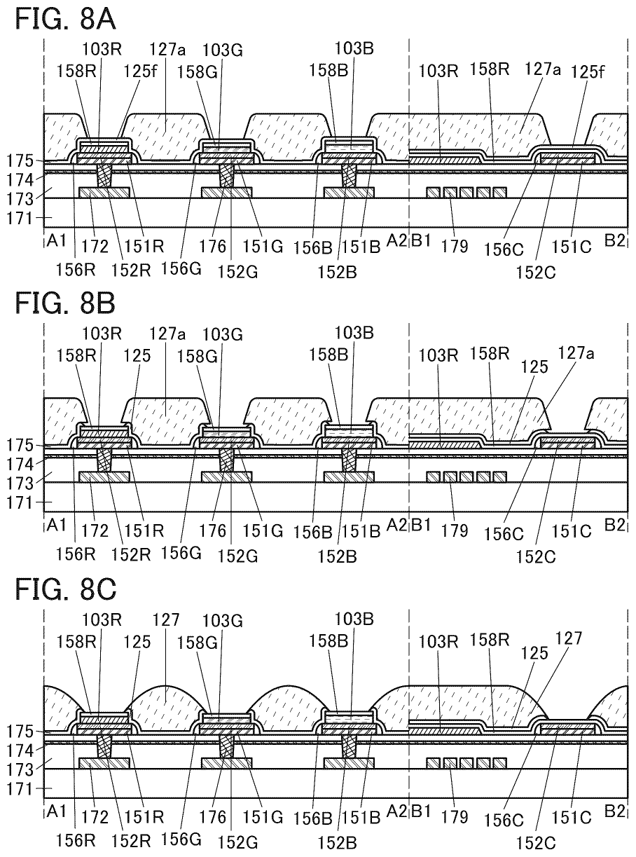

Next, as illustrated in FIG. 8A, development is performed to remove the exposed region of the insulating film 127f, so that an insulating layer 127a is formed. The insulating layer 127a is formed in regions that are sandwiched between any two of the conductive layers 152R, 152G, and 152B and a region surrounding the conductive layer 152C. Here, when an acrylic resin is used for the insulating film 127f, an alkaline solution, such as TMAH, can be used as a developer.

Then, a residue (scum) due to the development may be removed. For example, the residue can be removed by ashing using oxygen plasma.

Etching may be performed to adjust the surface level of the insulating layer 127a. The insulating layer 127a may be processed by ashing using oxygen plasma, for example. In the case where a non-photosensitive material is used for the insulating film 127f, the surface level of the insulating film 127f can be adjusted by the ashing, for example.

Next, as illustrated in FIG. 8B, etching treatment is performed using the insulating layer 127a as a mask to remove part of the inorganic insulating film 125f and reduce the thickness of part of the sacrificial layers 158R, 158G, and 158B. Thus, the inorganic insulating layer 125 is formed under the insulating layer 127a. Moreover, the surfaces of the thin portions in the sacrificial layers 158R, 158G, and 158B are exposed. Note that the etching treatment using the insulating layer 127a as a mask may be hereinafter referred to as first etching treatment.

The first etching treatment can be performed by dry etching or wet etching. Note that the inorganic insulating film 125f is preferably formed using a material similar to that of the sacrificial layers 158R, 158G, and 158B, in which case the first etching treatment can be performed collectively.

By etching using the insulating layer 127a with a tapered side surface as a mask, a side surface of the inorganic insulating layer 125 and upper end portions of side surfaces of the sacrificial layers 158R, 158G, and 158B can be made to have a tapered shape relatively easily.

In the case of performing dry etching, a chlorine-based gas is preferably used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the thin regions of the sacrificial layers 158R, 158G, and 158B can be formed with favorable in-plane uniformity.

As a dry etching apparatus, a dry etching apparatus including a high-density plasma source can be used. As the dry etching apparatus including a high-density plasma source, an inductively coupled plasma (ICP) etching apparatus can be used, for example. Alternatively, a capacitively coupled plasma (CCP) etching apparatus including parallel plate electrodes can be used. The capacitively coupled plasma etching apparatus including parallel plate electrodes may have a structure where a high-frequency voltage is applied to one of the parallel plate electrodes. Alternatively, the capacitively coupled plasma etching apparatus may have a structure where different high-frequency voltages are applied to one of the parallel-plate electrodes. Alternatively, the capacitively coupled plasma etching apparatus may have a structure where high-frequency voltages with the same frequency are applied to the parallel-plate electrodes. Alternatively, the capacitively coupled plasma etching apparatus may have a structure where high-frequency voltages with different frequencies are applied to the parallel-plate electrodes.

In the case of performing dry etching, a by-product or the like generated by the dry etching might be deposited on the top surface and a side surface of the insulating layer 127a, for example. Accordingly, a component of the etching gas, a component of the inorganic insulating film 125f, a component of the sacrificial layers 158R, 158G, and 158B, and the like might be included in the insulating layer 127 in the completed display device.

The first etching treatment is preferably performed by wet etching. The use of a wet etching method can reduce damage to the organic compound layers 103R, 103G, and 103B, as compared to the case of using a dry etching method. For example, the wet etching can be performed using an alkaline solution. For instance, TMAH, which is an alkaline solution, can be used for the wet etching of an aluminum oxide film. In this case, puddle wet etching can be performed. Note that the inorganic insulating film 125f is preferably formed using a material similar to that of the sacrificial layers 158R, 158G, and 158B, in which case the above etching treatment can be performed collectively.

The sacrificial layers 158R, 158G, and 158B are not removed completely by the first etching treatment, and the etching treatment is stopped when the thicknesses of the sacrificial layers 158R, 158G, and 158B are reduced. The sacrificial layers 158R, 158G, and 158B remain over the corresponding organic compound layers 103R, 103G, and 103B in this manner, whereby the organic compound layers 103R, 103G, and 103B can be prevented from being damaged by treatment in a later step.

Next, the insulating layer 127a is preferably irradiated with visible light or ultraviolet rays by performing light exposure on the entire substrate. The energy density for the light exposure is preferably greater than 0 mJ/cm² and less than or equal to 800 mJ/cm², further preferably greater than 0 mJ/cm² and less than or equal to 500 mJ/cm². Performing such light exposure after the development can sometimes increase the degree of transparency of the insulating layer 127a. In addition, it is sometimes possible to lower the substrate temperature required for subsequent heat treatment for changing the shape of the insulating layer 127a to a tapered shape.

Here, when a barrier insulating layer against oxygen (e.g., an aluminum oxide film) is present as each of the sacrificial layers 158R, 158G, and 158B, diffusion of oxygen into the organic compound layers 103R, 103G, and 103B can be inhibited. When the organic compound layer is irradiated with light (visible light or ultraviolet rays), the organic compound included in the organic compound layer is brought into an excited state and a reaction between the organic compound and oxygen in the atmosphere is promoted in some cases. Specifically, when the organic compound layer is irradiated with light (visible light or ultraviolet rays) in an atmosphere containing oxygen, oxygen might be bonded to the organic compound included in the organic compound layer. By providing the sacrificial layers 158R, 158G, and 158B over the island-shaped organic compound layers, bonding of oxygen in the atmosphere to the organic compounds included in the organic compound layers can be inhibited.

Then, heat treatment (also referred to as post-baking) is performed. The heat treatment can change the insulating layer 127a into the insulating layer 127 having a tapered side surface (FIG. 8C). The heat treatment is performed at a temperature lower than the upper temperature limits of the organic compound layers. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 130° C. The heating atmosphere may be an air atmosphere or an inert gas atmosphere. Moreover, the heating atmosphere may be an atmospheric-pressure atmosphere or a reduced-pressure atmosphere. The substrate temperature in the heat treatment of this step is preferably higher than that in the heat treatment (prebaking) after the formation of the insulating film 127f. In that case, adhesion between the insulating layer 127 and the inorganic insulating layer 125 can be improved, and corrosion resistance of the insulating layer 127 can be increased.

When the sacrificial layers 158R, 158G, and 158B are not completely removed by the first etching treatment and the thinned sacrificial layers 158R, 158G, and 158B are left, the organic compound layers 103R, 103G, and 103B can be prevented from being damaged and deteriorating in the heat treatment. This increases the reliability of the light-emitting devices.

Note that a side surface of the insulating layer 127 may have a concave shape depending on the material of the insulating layer 127 and the temperature, time, and atmosphere of the post-baking. For example, when the temperature of the post-baking is higher or the duration of the post-baking is longer, the insulating layer 127 is more likely to change in shape and thus the concave shape may be more likely to be formed.

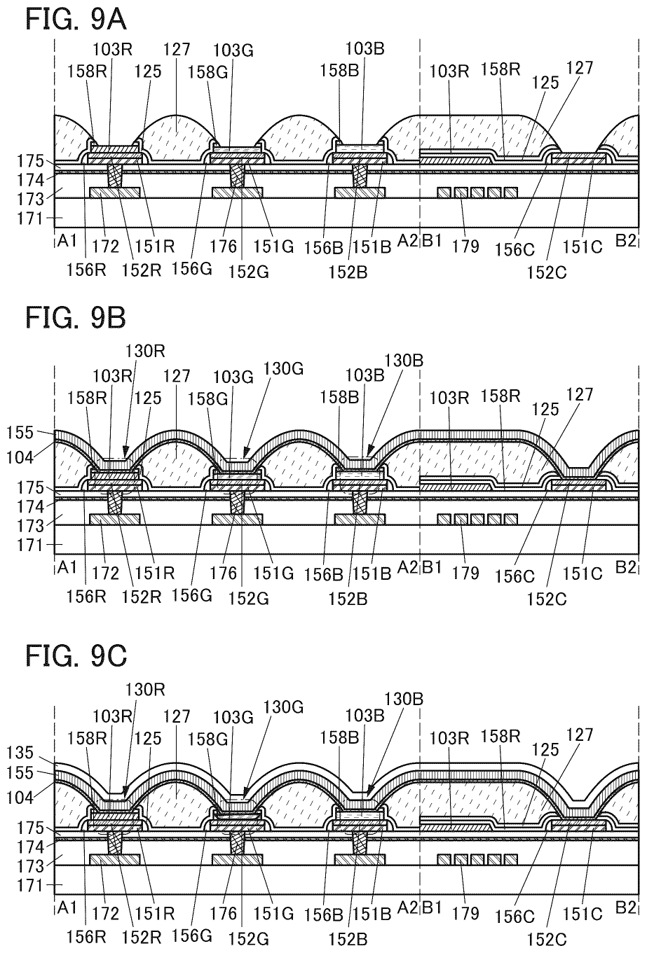

Next, as illustrated in FIG. 9A, etching treatment is performed using the insulating layer 127 as a mask to partly remove the sacrificial layers 158R, 158G, and 158B. Note that part of the inorganic insulating layer 125 is also removed in some cases. Thus, openings are formed in the sacrificial layers 158R, 158G, and 158B, and the top surfaces of the organic compound layers 103R, 103G, and 103B and the conductive layer 152C are exposed. Note that the etching treatment using the insulating layer 127 as a mask may be hereinafter referred to as second etching treatment.

An end portion of the inorganic insulating layer 125 is covered with the insulating layer 127. FIG. 9A illustrates an example where part of an end portion of the sacrificial layer 158G (specifically a tapered portion formed by the first etching treatment) is covered with the insulating layer 127 and a tapered portion formed by the second etching treatment is exposed.

If the first etching treatment is not performed and the inorganic insulating layer 125 and the mask layer are collectively etched after the post-baking, the inorganic insulating layer 125 and the mask layer under an end portion of the insulating layer 127 may disappear because of side etching and a void may be formed. The void causes unevenness on the formation surface of the common electrode 155, so that step disconnection is more likely to be caused in the common electrode 155. Even when a void is formed owing to side etching of the inorganic insulating layer 125 and the mask layer by the first etching treatment, the post-baking performed subsequently can make the insulating layer 127 fill the void. After that, the thinned mask layer is etched by the second etching treatment; thus, the amount of side etching decreases, a void is less likely to be formed, and even if a void is formed, it can be extremely small. Consequently, the formation surface of the common electrode 155 can be made flatter.

Note that the insulating layer 127 may cover the entire end portion of the sacrificial layer 158G. For example, the end portion of the insulating layer 127 may droop to cover the end portion of the sacrificial layer 158G. For another example, the end portion of the insulating layer 127 may be in contact with the top surface of at least one of the organic compound layers 103R, 103G, and 103B. As described above, when light exposure is not performed on the insulating layer 127a after the development, the shape of the insulating layer 127 may be likely to change.

The second etching treatment is performed by wet etching. The use of a wet etching method can reduce damage to the organic compound layers 103R, 103G, and 103B, as compared to the case of using a dry etching method. The wet etching can be performed using an alkaline solution such as TMAH, for example.

Meanwhile, in the case where the second etching treatment is performed by a wet etching method and gaps due to, for example, poor adhesion between the organic compound layer 103 and another layer exist at the interface between the organic compound layer 103 and the sacrificial layer 158, the interface between the organic compound layer 103 and the inorganic insulating layer 125, and the interface between the organic compound layer 103 and the insulating layer 175, the chemical solution used in the second etching treatment sometimes enters the gaps to come into contact with the pixel electrode. Here, when the chemical solution comes into contact with both the conductive layer 151 and the conductive layer 152, one of the conductive layers 151 and 152 that has a lower spontaneous potential than the other suffers from galvanic corrosion in some cases. For example, when the conductive layer 151 is formed using aluminum and the conductive layer 152 is formed using indium tin oxide, the conductive layer 152 sometimes corrodes. As a result, the yield of the display device decreases in some cases. Moreover, the reliability of the display device decreases in some cases.

As described above, when the insulating layer 156 is formed to include a region overlapping with the side surface of the conductive layer 151 and to cover the conductive layers 151 and 152, the step disconnection of the inorganic insulating layer 125 can be prevented, whereby the chemical solution can be prevented from coming into contact with a lower component such as the conductive layer 151 in the second etching treatment, for example. Thus, corrosion of the pixel electrode can be prevented.

As described above, by providing the insulating layer 127, the inorganic insulating layer 125, and the sacrificial layers 158R, 158G, and 158B, poor connection due to a disconnected portion and an increase in electrical resistance due to a locally thinned portion can be inhibited from occurring in the common electrode 155 between the light-emitting devices. Thus, the display device of one embodiment of the present invention can have improved display quality.

Heat treatment is performed after the organic compound layers 103R, 103G, and 103B are partly exposed. By the heat treatment, water included in the organic compound layers and water adsorbed onto the surfaces of the organic compound layers, for example, can be removed. The shape of the insulating layer 127 may be changed by the heat treatment. Specifically, the insulating layer 127 may be widened to cover at least one of the end portion of the inorganic insulating layer 125, the end portions of the sacrificial layers 158R, 158G, and 158B, and the top surfaces of the organic compound layers 103R, 103G, and 103B.

If the temperature of the heat treatment is too low, water included in the organic compound layers and water adsorbed onto the surfaces of the organic compound layers, for example, cannot be sufficiently removed. If the temperature of the heat treatment is too high, the organic compound layer 103 might deteriorate and the insulating layer 127 might change in shape excessively. Therefore, the temperature of the heat treatment is preferably higher than the temperature at which water is released from the organic compound layer 103 and lower than the T_{g} of the organic compound included in the organic compound layer 103, further preferably lower than T_{g} of the organic compound included in the upper surface of the organic compound layer 103. Specifically, the substrate temperature is preferably higher than or equal to 80° C. and lower than or equal to 130° C., further preferably higher than or equal to 90° C. and lower than or equal to 120° C., still further preferably higher than or equal to 100° C. and lower than or equal to 120° C., yet still further preferably higher than or equal to 100° C. and lower than or equal to 110° C. The heating atmosphere may be an air atmosphere or an inert gas atmosphere. Although the heating atmosphere may be an atmospheric-pressure atmosphere or a reduced-pressure atmosphere, a reduced-pressure atmosphere is preferably employed to prevent re-adsorption of water released from the organic compound layer 103.

By the heat treatment, water included in the organic compound layers and water adsorbed onto the surfaces of the organic compound layers, for example, can be sufficiently removed without deterioration of the organic compound layers 103R, 103G, and 103B and an excessive change in the shape of the insulating layer 127. Thus, degradation of the characteristics of the light-emitting devices can be prevented.

Next, as illustrated in FIG. 9B, the common layer 104 and the common electrode 155 are formed over the organic compound layers 103R, 103G, and 103B, the conductive layer 152C, and the insulating layer 127. The common layer 104 and the common electrode 155 can be formed by a sputtering method, a vacuum evaporation method, or the like. The common layer 104 may be formed by an evaporation method while the common electrode 155 may be formed by a sputtering method.

Then, as illustrated in FIG. 9C, the protective layer 135 is formed over the common electrode 155. The protective layer 135 can be formed by a vacuum evaporation method, a sputtering method, a CVD method, an ALD method, or the like. Note that the protective layer 135 may also function as a cap layer. For example, when a material whose ordinary refractive index (no) at a wavelength of 450 nm is greater than or equal to 1.90, ordinary refractive index (no) at a wavelength of 520 nm is greater than or equal to 1.80, or an ordinary refractive index (no) at a wavelength of 630 nm is greater than or equal to 1.75 is used for the protective layer 135, the total reflection of light from the organic compound layer 103 by the cap layer can be inhibited, leading to an improvement in light extraction efficiency.

In order to prevent air exposure of the light-emitting device before being incorporated in a display device or a light-emitting apparatus, a sealing film may be provided over the protective layer 135. The sealing film can be formed using a material that does not easily transmit an impurity such as oxygen or water. Specifically, an aluminum oxide film is preferably provided by an ALD method. Note that in order to prevent air exposure of the light-emitting device in which the sealing film has yet to be provided after the protective layer 135 is formed, the light-emitting device is preferably transferred into an ALD apparatus in a glove box containing a nitrogen atmosphere after the protective layer 135 is formed. In that case, the oxygen concentration in the glove box is preferably lower than or equal to 100 ppm, further preferably lower than or equal to 10 ppm, still further preferably lower than or equal to 1 ppm.

Then, the substrate 120 is attached onto the protective layer 135 or the sealing film using the resin layer 122, so that the display device can be manufactured. In the method for manufacturing the display device of one embodiment of the present invention, the insulating layer 156 is provided on the side surfaces of the conductive layer 151 and the conductive layer 152 as described above. This can increase the yield of the display device and inhibit generation of defects. Alternatively, a microlens array is provided over the protective layer 135 or the sealing film before bonding of the substrate 120 and then the substrate 120 is bonded, whereby a display device including the microlens array can be manufactured.

As described above, in the method for manufacturing the display device of one embodiment of the present invention, the island-shaped organic compound layers 103R, 103G, and 103B are each formed not by using a fine metal mask but by processing a film formed on the entire surface; thus, the island-shaped layers can be formed to have a uniform thickness. Consequently, a high-resolution display device or a display device with a high aperture ratio can be obtained. Furthermore, even when the resolution or the aperture ratio is high and the distance between the subpixels is extremely short, the organic compound layers 103R, 103G, and 103B can be inhibited from being in contact with each other in the adjacent subpixels. As a result, generation of a leakage current between the subpixels can be inhibited. This can prevent crosstalk, so that a display device with extremely high contrast can be obtained. Moreover, even a display device that includes tandem light-emitting devices formed by a lithography method can have favorable characteristics.

The structure described in this embodiment can be used in combination with any of the structures described in other embodiments as appropriate.

### Embodiment 4

In this embodiment, a light-emitting apparatus of one embodiment of the present invention will be described.

The light-emitting apparatus in this embodiment can be a high-resolution light-emitting apparatus. Thus, the light-emitting apparatus in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The light-emitting apparatus in this embodiment can be a high-definition light-emitting apparatus or a large-sized light-emitting apparatus. Accordingly, the light-emitting apparatus in this embodiment can be used for display portions of a digital camera, a digital video camera, a digital photo frame, a mobile phone, a portable game console, a portable information terminal, and an audio reproducing device, in addition to display portions of electronic appliances with a relatively large screen, such as a television device, desktop and notebook personal computers, a monitor of a computer and the like, digital signage, and a large game machine such as a pachinko machine.

### [Display Module]

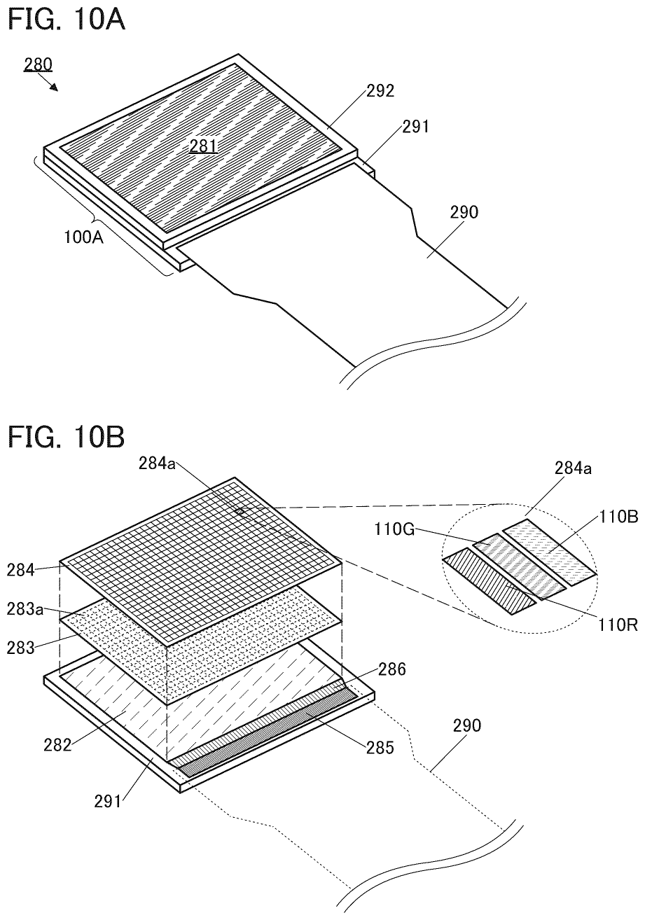

FIG. 10A is a perspective view of a display module 280. The display module 280 includes a display device 100A and an FPC 290. Note that the light-emitting apparatus included in the display module 280 is not limited to the display device 100A and may be any of display devices 100B to 100F described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 10B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion not overlapping with the pixel portion 284 over the substrate 291. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 10B. The pixels 284a can employ any of the structures described in the above embodiments. FIG. 10B illustrates an example where the pixel 284a has a structure similar to that of the pixel 178 illustrated in FIG. 3A.

The pixel circuit portion 283 includes a plurality of pixel circuits 283a arranged periodically.

One pixel circuit 283a is a circuit that controls driving of a plurality of elements included in one pixel 284a. One pixel circuit 283a can be provided with three circuits each of which controls light emission of one light-emitting device. For example, the pixel circuit 283a can include at least one selection transistor, one current control transistor (driving transistor), and a capacitor for one light-emitting device. A gate signal is input to a gate of the selection transistor, and a video signal is input to a source or a drain of the selection transistor. With such a structure, an active-matrix light-emitting apparatus is obtained.

The circuit portion 282 includes a circuit for driving the pixel circuits 283a in the pixel circuit portion 283. For example, the circuit portion 282 preferably includes one or both ofa gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be higher than or equal to 40% and lower than 100%, preferably higher than or equal to 50% and lower than or equal to 95%, further preferably higher than or equal to 60% and lower than or equal to 95%. Furthermore, the pixels 284a can be arranged extremely densely and thus the display portion 281 can have significantly high resolution. For example, the pixels 284a are preferably arranged in the display portion 281 with a resolution higher than or equal to 2000 ppi, further preferably higher than or equal to 3000 ppi, still further preferably higher than or equal to 5000 ppi, yet still further preferably higher than or equal to 6000 ppi, and lower than or equal to 20000 ppi or lower than or equal to 30000 ppi.

Such a display module 280 has extremely high resolution, and thus can be suitably used for a VR device such as an HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion. For example, the display module 280 can be favorably used in a display portion of a wearable electronic appliance, such as a wrist watch.

### [Display Device 100A]

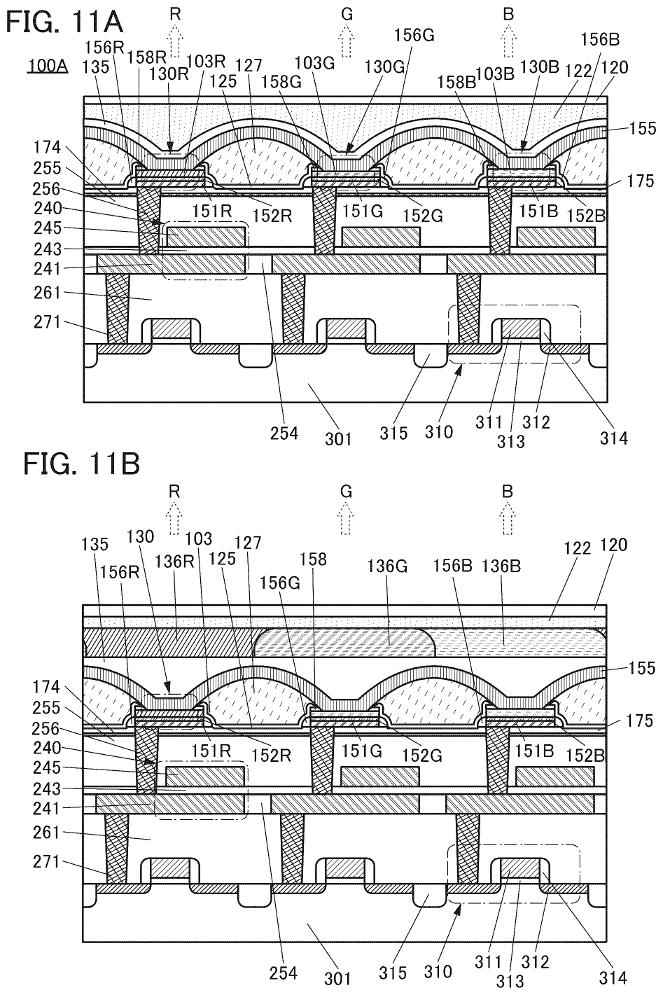

The display device 100A illustrated in FIG. 11A includes a substrate 301, the light-emitting devices 130R, 130G, and 130B, a capacitor 240, and a transistor 310.

The substrate 301 corresponds to the substrate 291 in FIGS. 10A and 10B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover a side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. FIG. 11A illustrates an example in which the light-emitting devices 130R, 130G, and 130B each have the stacked-layer structure illustrated in FIG. 1A. An insulator is provided in regions between adjacent light-emitting devices. For example, in FIG. 11A, the inorganic insulating layer 125 and the insulating layer 127 over the inorganic insulating layer 125 are provided in those regions.

The insulating layer 156R is provided to include a region overlapping with the side surface of the conductive layer 151R of the light-emitting device 130R. The insulating layer 156G is provided to include a region overlapping with the side surface of the conductive layer 151G of the light-emitting device 130G. The insulating layer 156B is provided to include a region overlapping with the side surface of the conductive layer 151B of the light-emitting device 130B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. The sacrificial layer 158R is positioned over the organic compound layer 103R of the light-emitting device 130R. The sacrificial layer 158G is positioned over the organic compound layer 103G of the light-emitting device 130G. The sacrificial layer 158B is positioned over the organic compound layer 103B of the light-emitting device 130B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. The top surface of the insulating layer 175 and the top surface of the plug 256 are level with or substantially level with each other. Any of a variety of conductive materials can be used for the plugs.

The protective layer 135 is provided over the light-emitting devices 130R, 130G, and 130B. The substrate 120 is attached onto the protective layer 135 with the resin layer 122. Embodiment 3 can be referred to for the details of the light-emitting device 130 and the components thereover up to the substrate 120. The substrate 120 corresponds to the substrate 292 in FIG. 10A.

FIG. 11B illustrates a variation example of the display device 100A illustrated in FIG. 11A. The light-emitting apparatus illustrated in FIG. 11B includes coloring layers 136R, 136G, and 136B, and each of the light-emitting devices 130 includes a region overlapping with one of the coloring layers 136R, 136G, and 136B. In the light-emitting apparatus illustrated in FIG. 11B, the light-emitting device 130 can emit white light, for example. For example, the coloring layer 136R, the coloring layer 136G, and the coloring layer 136B can transmit red light, green light, and blue light, respectively.

### [Display Device 100B]

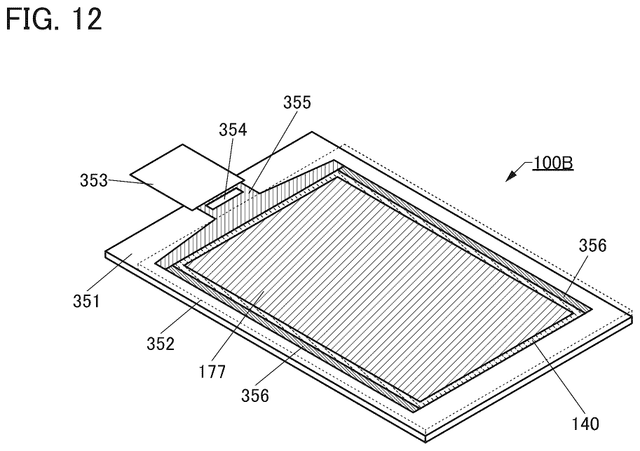

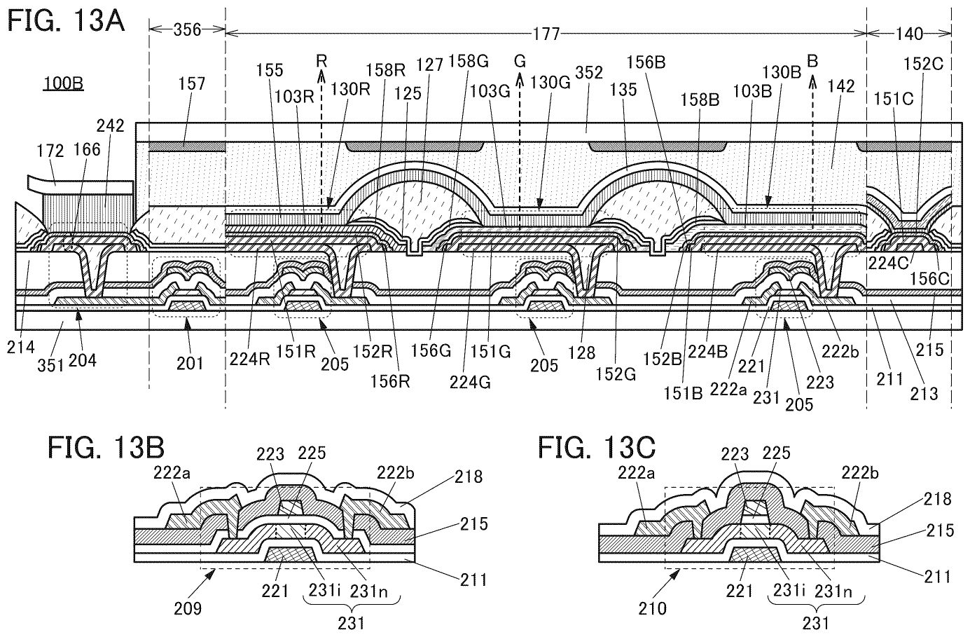

FIG. 12 is a perspective view of the display device 100B, and FIG. 13A is a cross-sectional view of the display device 100B.

In the display device 100B, a substrate 352 and a substrate 351 are bonded to each other. In FIG. 12, the substrate 352 is denoted by a dashed line.

The display device 100B includes the pixel portion 177, the connection portion 140, a circuit 356, a wiring 355, and the like. FIG. 12 illustrates an example in which an integrated circuit (IC) 354 and an FPC 353 are mounted on the display device 100B. Thus, the structure illustrated in FIG. 12 can be regarded as a display module including the display device 100B, the IC, and the FPC. Here, a light-emitting apparatus in which a substrate is equipped with a connector such as an FPC or mounted with an IC is referred to as a display module.

The connection portion 140 is provided outside the pixel portion 177. The connection portion 140 can be provided along one side or a plurality of sides of the pixel portion 177. The number of connection portions 140 may be one or more. FIG. 12 illustrates an example in which the connection portion 140 is provided to surround the four sides of the pixel portion 177. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 12 illustrates an example in which the IC 354 is provided over the substrate 351 by a chip on glass (COG) method, a chip on film (COF) method, or the like. An IC including a scan line driver circuit, a signal line driver circuit, or the like can be used as the IC 354, for example. Note that the display device 100B and the display module are not necessarily provided with an IC. Alternatively, the IC may be mounted on the FPC by a COF method, for example.

FIG. 13A illustrates an example of cross sections of part of a region including the FPC 353, part of the circuit 356, part of the pixel portion 177, part of the connection portion 140, and part of a region including an end portion of the display device 100B.

The display device 100B illustrated in FIG. 13A includes a transistor 201, a transistor 205, the light-emitting device 130R that emits red light, the light-emitting device 130G that emits green light, the light-emitting device 130B that emits blue light, and the like between the substrate 351 and the substrate 352.

The stacked-layer structure of each of the light-emitting devices 130R, 130G, and 130B is the same as that illustrated in FIG. 1A except for the structure of the pixel electrode. The above embodiments can be referred to for the details of the light-emitting devices.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B. Here, the conductive layers 224R, 151R, and 152R can be collectively referred to as the pixel electrode of the light-emitting device 130R; the conductive layers 151R and 152R excluding the conductive layer 224R can also be referred to as the pixel electrode of the light-emitting device 130R. Similarly, the conductive layers 224G, 151G, and 152G can be collectively referred to as the pixel electrode of the light-emitting device 130G; the conductive layers 151G and 152G excluding the conductive layer 224G can also be referred to as the pixel electrode of the light-emitting device 130G. The conductive layers 224B, 151B, and 152B can be collectively referred to as the pixel electrode of the light-emitting device 130B; the conductive layers 151B and 152B excluding the conductive layer 224B can also be referred to as the pixel electrode of the light-emitting device 130B.

The conductive layer 224R is connected to a conductive layer 222b included in the transistor 205 through the opening provided in an insulating layer 214. An end portion of the conductive layer 151R is positioned outward from an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G and the insulating layer 156G in the light-emitting device 130G are not described in detail because they are respectively similar to the conductive layers 224R, 151R, and 152R and the insulating layer 156R in the light-emitting device 130R; the same applies to the conductive layers 224B, 151B, and 152B and the insulating layer 156B in the light-emitting device 130B.

The conductive layers 224R, 224G, and 224B each have a depressed portion covering an opening provided in the insulating layer 214. A layer 128 is embedded in the depressed portion.

The layer 128 has a function of filling the depressed portions of the conductive layers 224R, 224G, and 224B to enable planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depressed portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 135 is provided over the light-emitting devices 130R, 130G, and 130B. The protective layer 135 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 13A, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided not to overlap with the light-emitting device. Furthermore, the space may be filled with a resin other than the frame-shaped adhesive layer 142.

FIG. 13A illustrates an example in which the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and the conductive layer 152C obtained by processing the same conductive film as the conductive layers 152R, 152G, and 152B. In the example illustrated in FIG. 13A, the insulating layer 156C is provided to include a region overlapping with the side surface of the conductive layer 151C.

The display device 100B has a top-emission structure. Light from the light-emitting device is emitted toward the substrate 352. For the substrate 352, a material having a high visible-light-transmitting property is preferably used. The pixel electrode contains a material that reflects visible light, and the counter electrode (the common electrode 155) contains a material that transmits visible light.

The transistor 201 and the transistor 205 are formed over the substrate 351. These transistors can be manufactured using the same materials in the same steps.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

A material through which impurities such as water and hydrogen do not easily diffuse is preferably used for at least one of the insulating layers covering the transistors. This is because such an insulating layer can function as a barrier layer. Such a structure can effectively inhibit diffusion of impurities to the transistors from the outside and increase the reliability of the light-emitting apparatus.

An inorganic insulating film is preferably used as each of the insulating layers 211, 213, and 215. As the inorganic insulating film, a silicon nitride film, a silicon oxynitride film, a silicon oxide film, a silicon nitride oxide film, an aluminum oxide film, or an aluminum nitride film can be used, for example. A hafnium oxide film, an yttrium oxide film, a zirconium oxide film, a gallium oxide film, a tantalum oxide film, a magnesium oxide film, a lanthanum oxide film, a cerium oxide film, a neodymium oxide film, or the like may be used. A stack including two or more of the above insulating films may also be used.

An organic insulating layer is suitable for the insulating layer 214 functioning as a planarization layer. Examples of materials that can be used for the organic insulating layer include an acrylic resin, a polyimide resin, an epoxy resin, a polyamide resin, a polyimide-amide resin, a siloxane resin, a benzocyclobutene-based resin, a phenol resin, and precursors of these resins. The insulating layer 214 may have a stacked-layer structure of an organic insulating layer and an inorganic insulating layer. The outermost layer of the insulating layer 214 preferably functions as an etching protective layer. This can inhibit formation of a depressed portion in the insulating layer 214 at the time of processing of the conductive layer 224R, 151R, or 152R or the like. Alternatively, a depressed portion may be provided in the insulating layer 214 at the time of processing of the conductive layer 224R, 151R, or 152R or the like.

Each of the transistors 201 and 205 includes a conductive layer 221 functioning as a gate, the insulating layer 211 functioning as a gate insulating layer, a conductive layer 222a and the conductive layer 222b functioning as a source and a drain, a semiconductor layer 231, the insulating layer 213 functioning as a gate insulating layer, and a conductive layer 223 functioning as a gate. Here, a plurality of layers obtained by processing the same conductive film are shown with the same hatching pattern. The insulating layer 211 is positioned between the conductive layer 221 and the semiconductor layer 231. The insulating layer 213 is positioned between the conductive layer 223 and the semiconductor layer 231.

There is no particular limitation on the structure of the transistors included in the light-emitting apparatus of this embodiment. For example, a planar transistor, a staggered transistor, or an inverted staggered transistor can be used. A top-gate transistor or a bottom-gate transistor can be used. Alternatively, gates may be provided above and below a semiconductor layer where a channel is formed.

The structure in which the semiconductor layer where a channel is formed is provided between two gates is used for the transistors 201 and 205. The two gates may be connected to each other and supplied with the same signal to operate the transistor. Alternatively, the threshold voltage of the transistor may be controlled by applying a potential for controlling the threshold voltage to one of the two gates and a potential for driving to the other of the two gates.

There is no particular limitation on the crystallinity of a semiconductor material used for the transistors, and either an amorphous semiconductor or a semiconductor having crystallinity (a microcrystalline semiconductor, a polycrystalline semiconductor, a single crystal semiconductor, or a semiconductor partly including crystal regions) can be used. A semiconductor having crystallinity is preferably used, in which case deterioration of transistor characteristics can be suppressed.

The semiconductor layer of the transistor preferably includes a metal oxide. That is, a transistor including a metal oxide in its channel formation region (hereinafter, also referred to as an OS transistor) is preferably used in the light-emitting apparatus of this embodiment.

Examples of an oxide semiconductor having crystallinity include a c-axis-aligned crystalline oxide semiconductor (CAAC-OS) and a nanocrystalline oxide semiconductor (nc-OS).

Alternatively, a transistor including silicon in its channel formation region (a Si transistor) may be used. Examples of silicon include single crystal silicon, polycrystalline silicon, and amorphous silicon. In particular, a transistor containing low-temperature polysilicon (LTPS) in its semiconductor layer (hereinafter also referred to as an LTPS transistor) can be used. The LTPS transistor has high field-effect mobility and excellent frequency characteristics.

With the use of Si transistors such as LTPS transistors, a circuit required to be driven at a high frequency (e.g., a source driver circuit) can be formed on the same substrate as the display portion. This enables simplification of an external circuit mounted on the light-emitting apparatus and a reduction in costs of parts and mounting costs.

An OS transistor has much higher field-effect mobility than a transistor containing amorphous silicon. In addition, the OS transistor has an extremely low leakage current between a source and a drain in an off state (hereinafter also referred to as an off-state current), and electric charge accumulated in a capacitor that is connected in series to the transistor can be held for a long period. Furthermore, the power consumption of the light-emitting apparatus can be reduced with the OS transistor.

To increase the luminance of the light-emitting device included in the pixel circuit, the amount of current fed through the light-emitting device needs to be increased. To increase the current amount, the source-drain voltage of a driving transistor included in the pixel circuit needs to be increased. An OS transistor has a higher breakdown voltage between a source and a drain than a Si transistor; hence, a high voltage can be applied between the source and the drain of the OS transistor. Therefore, when an OS transistor is used as the driving transistor in the pixel circuit, the amount of current flowing through the light-emitting device can be increased, so that the luminance of the light-emitting device can be increased.

Regarding saturation characteristics of a current flowing when transistors operate in a saturation region, even in the case where the source-drain voltage of an OS transistor increases gradually, a more stable current (saturation current) can be fed through the OS transistor than through a Si transistor. Thus, by using an OS transistor as the driving transistor, a stable current can be fed through light-emitting devices even when the current-voltage characteristics of the light-emitting devices vary, for example. In other words, when the OS transistor operates in the saturation region, the source-drain current hardly changes with an increase in the source-drain voltage; hence, the luminance of the light-emitting device can be stable.

As described above, by using OS transistors as the driving transistors included in the pixel circuits, it is possible to inhibit black-level degradation, increase the luminance, increase the number of gray levels, and suppress variations in light-emitting devices, for example.

The semiconductor layer preferably contains indium, M (M is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more of aluminum, gallium, yttrium, and tin.

It is particularly preferable that an oxide containing indium (In), gallium (Ga), and zinc (Zn) (also referred to as IGZO) be used for the semiconductor layer. It is preferable to use an oxide containing indium, tin, and zinc. It is preferable to use an oxide containing indium, gallium, tin, and zinc. It is preferable to use an oxide containing indium (In), aluminum (Al), and zinc (Zn) (also referred to as IAZO). It is preferable to use an oxide containing indium (In), aluminum (Al), gallium (Ga), and zinc (Zn) (also referred to as IAGZO).

When the semiconductor layer is formed using an In-M-Zn oxide, the atomic proportion of In is preferably higher than or equal to the atomic proportion of M in the In-M-Zn oxide. Examples of the atomic ratio of the metal elements in such an In-M-Zn oxide are In:M:Zn=1:1:1, 1:1:1.2, 2:1:3, 3:1:2, 4:2:3, 4:2:4.1, 5:1:3, 5:1:6, 5:1:7, 5:1:8, 6:1:6, and 5:2:5 and a composition in the neighborhood of any of the above atomic ratios. Note that the neighborhood of the atomic ratio includes±30% of an intended atomic ratio.

For example, in the case of describing an atomic ratio of In:Ga:Zn=4:2:3 or a composition in the neighborhood thereof, the case is included in which with the atomic proportion of In being 4, the atomic proportion of Ga is greater than or equal to 1 and less than or equal to 3 and the atomic proportion of Zn is greater than or equal to 2 and less than or equal to 4. In the case of describing an atomic ratio of In:Ga:Zn=5:1:6 or a composition in the neighborhood thereof, the case is included in which with the atomic proportion of In being 5, the atomic proportion of Ga is greater than 0.1 and less than or equal to 2 and the atomic proportion of Zn is greater than or equal to 5 and less than or equal to 7. In the case of describing an atomic ratio of In:Ga:Zn=1:1:1 or a composition in the neighborhood thereof, the case is included in which with the atomic proportion of In being 1, the atomic proportion of Ga is greater than 0.1 and less than or equal to 2 and the atomic proportion of Zn is greater than 0.1 and less than or equal to 2.

The transistors included in the circuit 356 and the transistors included in the pixel portion 177 may have the same structure or different structures. One structure or two or more kinds of structures may be employed for a plurality of transistors included in the circuit 356. Similarly, one structure or two or more kinds of structures may be employed for a plurality of transistors included in the pixel portion 177.

All transistors included in the pixel portion 177 may be OS transistors, or all transistors included in the pixel portion 177 may be Si transistors. Alternatively, some of the transistors included in the pixel portion 177 may be OS transistors and the others may be Si transistors.

For example, when both an LTPS transistor and an OS transistor are used in the pixel portion 177, the light-emitting apparatus can have low power consumption and high driving capability. Note that a structure in which an LTPS transistor and an OS transistor are used in combination is referred to as LTPO in some cases. For example, it is preferable that an OS transistor be used as a transistor functioning as a switch for controlling electrical continuity between wirings and an LTPS transistor be used as a transistor for controlling a current.

For example, one transistor included in the pixel portion 177 functions as a transistor for controlling a current flowing through the light-emitting device and can be referred to as a driving transistor. One of a source and a drain of the driving transistor is electrically connected to the pixel electrode of the light-emitting device. An LTPS transistor is preferably used as the driving transistor. In that case, the amount of current flowing through the light-emitting device can be increased in the pixel circuit.

Another transistor included in the pixel portion 177 functions as a switch for controlling selection or non-selection of a pixel and can be referred to as a selection transistor. A gate of the selection transistor is electrically connected to a gate line, and one of a source and a drain thereof is electrically connected to a source line (signal line). An OS transistor is preferably used as the selection transistor. In that case, the gray level of the pixel can be maintained even with an extremely low frame frequency (e.g., lower than or equal to 1 fps); thus, power consumption can be reduced by stopping the driver in displaying a still image.

As described above, the light-emitting apparatus of one embodiment of the present invention can have all of a high aperture ratio, high resolution, high display quality, and low power consumption.

Note that the light-emitting apparatus of one embodiment of the present invention has a structure including the OS transistor and the light-emitting device having a metal maskless (MML) structure. This structure can significantly reduce a leakage current that would flow through a transistor and a leakage current that would flow between adjacent light-emitting devices (sometimes referred to as a horizontal leakage current or a lateral leakage current). Displaying images on the light-emitting apparatus having this structure can bring one or more of image crispness, image sharpness, high color saturation, and a high contrast ratio to the viewer. When a leakage current that would flow through the transistor and a lateral leakage current that would flow between the light-emitting devices are extremely low, leakage of light at the time of black display (black-level degradation) or the like can be minimized.

In particular, in the case where a light-emitting device having an MML structure employs a side-by-side (SBS) structure, which is the above-described structure for separately forming or coloring light-emitting layers, a layer provided between light-emitting devices (for example, also referred to as an organic layer or a common layer which is shared by the light-emitting devices) is disconnected; accordingly, side leakage can be prevented or be made extremely low.

FIGS. 13B and 13C illustrate other structure examples of transistors.

Transistors 209 and 210 each include the conductive layer 221 functioning as a gate, the insulating layer 211 functioning as a gate insulating layer, the semiconductor layer 231 including a channel formation region 231i and a pair of low-resistance regions 231n, the conductive layer 222a connected to one of the pair of low-resistance regions 231n, the conductive layer 222b connected to the other of the pair of low-resistance regions 231n, an insulating layer 225 functioning as a gate insulating layer, the conductive layer 223 functioning as a gate, and the insulating layer 215 covering the conductive layer 223. The insulating layer 211 is positioned between the conductive layer 221 and the channel formation region 231i. The insulating layer 225 is positioned at least between the conductive layer 223 and the channel formation region 231i. Furthermore, an insulating layer 218 covering the transistor may be provided.

FIG. 13B illustrates an example of the transistor 209 in which the insulating layer 225 covers the top and side surfaces of the semiconductor layer 231. The conductive layer 222a and the conductive layer 222b are connected to the corresponding low-resistance regions 231n through openings provided in the insulating layer 225 and the insulating layer 215. One of the conductive layers 222a and 222b functions as a source, and the other functions as a drain.

In the transistor 210 illustrated in FIG. 13C, the insulating layer 225 overlaps with the channel formation region 231i of the semiconductor layer 231 and does not overlap with the low-resistance regions 231n. The structure illustrated in FIG. 13C is obtained by processing the insulating layer 225 with the conductive layer 223 as a mask, for example. In FIG. 13C, the insulating layer 215 is provided to cover the insulating layer 225 and the conductive layer 223, and the conductive layer 222a and the conductive layer 222b are connected to the corresponding low-resistance regions 231n through openings in the insulating layer 215.

A connection portion 204 is provided in a region of the substrate 351 where the substrate 352 does not overlap. In the connection portion 204, the wiring 355 is electrically connected to the FPC 353 through a conductive layer 166 and a connection layer 242. An example is described in which the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

The light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### [Display Device 100C]

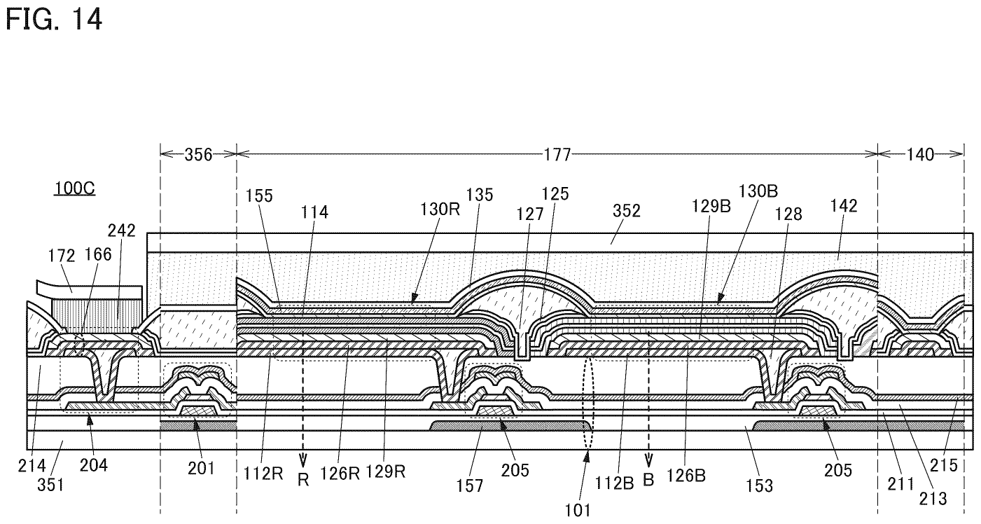

The display device 100C illustrated in FIG. 14 differs from the display device 100B illustrated in FIG. 13A mainly in having a bottom-emission structure.

Light from the light-emitting device is emitted toward the substrate 351. For the substrate 351, a material having a high visible-light-transmitting property is preferably used. By contrast, there is no limitation on the light-transmitting property of a material used for the substrate 352.

The light-blocking layer 157 is preferably formed between the substrate 351 and the transistor 201 and between the substrate 351 and the transistor 205. FIG. 14 illustrates an example in which the light-blocking layer 157 is provided over the substrate 351, an insulating layer 153 is provided over the light-blocking layer 157, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material having a high visible-light-transmitting property is used for each of the conductive layers 112R, 112B, 126R, 126B, 129R, and 129B. A material that reflects visible light is preferably used for the common electrode 155.

Although not illustrated in FIG. 14, the light-emitting device 130G is also provided.

Although FIG. 14 and the like illustrate an example in which the top surface of the layer 128 includes a flat portion, the shape of the layer 128 is not particularly limited.

### [Display Device 100D]

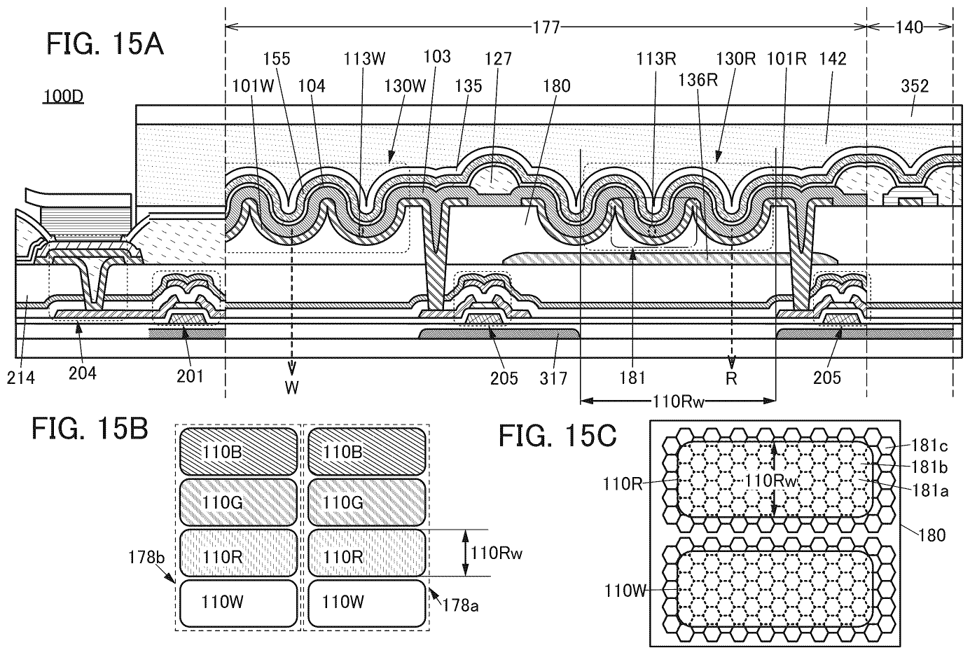

The display device 100D with a bottom-emission structure illustrated in FIG. 15A is an example of a bottom-emission display device different from the display device 100C illustrated in FIG. 14. The display device 100D is different from the display device 100C in including an organic resin layer 180. Note that in the drawings, reference numerals of some of the components that are shown in FIGS. 11A and 11B are omitted; for the details of the components, the description made with reference to FIGS. 11A and 11B is to be referred to.

FIG. 15B shows a top-view layout of the pixels 178 (a pixel 178a and a pixel 178b) each including the subpixels 110 (the subpixels 110R, 110G, and 110B, and a subpixel 110W), and FIG. 15C shows a top view of the organic resin layer 180 in a region where the subpixels 110R and 110W of the pixel 178 are formed. Note that the width between the light-blocking layer 317 and another light-blocking layer 317 corresponds to a width 110Rw in the light-emitting region of the subpixel 110R.

As illustrated in FIG. 15A, the organic resin layer 180 is provided over the insulating layer 214. As illustrated in FIG. 15C and the region surrounded by the dashed-dotted line in FIG. 15A, the organic resin layer 180 includes depressed portions 181 (depressed portions 181a and depressed portions 181b) each having a curved surface, at least in a region where the subpixels are formed. Note that the depressed portion 181 outside the light-emitting region, like a depressed portion 181c, may also be provided. When the depressed portion 181c is provided, light that has been emitted in the region overlapping with the light-blocking layer 317 or light that has progressed to the region overlapping with the light-blocking layer 317 can be refracted and extracted from the light-emitting region, increasing the emission efficiency.

A plurality of the depressed portions 181 may be formed in a matrix. The depressed portion 181a and the depressed portion 181b may be provided in contact with each other or may have a flat surface therebetween.

Although the top surface shape and the cross-sectional shape of the depressed portion are hexagonal (FIG. 15C) and semicircular (FIG. 15A), respectively, other shapes may be employed as needed. Examples of a top surface shape of the depressed portion include polygons such as a triangle, a tetragon (including a rectangle and a square), and a pentagon; these polygons with rounded corners; an ellipse; and a circle.

As the organic resin layer 180, an insulating layer including an organic material can be used. For the organic resin layer 180, an acrylic resin, a polyimide resin, an epoxy resin, an imide resin, a polyamide resin, a polyimide-amide resin, a silicone resin, a siloxane resin, a benzocyclobutene-based resin, a phenol resin, or a precursor of any of these resins can be used, for example. Alternatively, an organic material such as polyvinyl alcohol (PVA), polyvinyl butyral, polyvinylpyrrolidone, polyethylene glycol, polyglycerin, pullulan, water-soluble cellulose, or an alcohol-soluble polyamide resin may be used for the organic resin layer 180.

Further alternatively, a photosensitive resin can be used for the organic resin layer 180. A photoresist may be used as the photosensitive resin. As the photosensitive resin, a positive photosensitive material or a negative photosensitive material can be used.

The organic resin layer 180 may contain a material absorbing visible light. For example, the organic resin layer 180 itself may be made of a material absorbing visible light, or the organic resin layer 180 may contain a pigment absorbing visible light. For the organic resin layer 180, for example, a resin that can be used as a color filter transmitting red, blue, or green light and absorbing light of the other colors or a resin that contains carbon black as a pigment and functions as a black matrix can be used.

The first electrodes 101 (a first electrode 101R and a first electrode 101W) are over the organic resin layer 180, and the organic compound layer 103 is over the first electrodes 101. End portions of the first electrode 101 and the organic compound layer 103 may be covered with the insulating layer 127.

Along the depressed portion of the organic resin layer 180, the first electrode 101 formed over the organic resin layer 180 has a depressed portion in a manner similar to that of the organic resin layer 180. Furthermore, along the depressed portion of the first electrode 101, the organic compound layer 103 formed over the first electrode 101 has a depressed portion in a manner similar to that of the first electrode 101. Furthermore, along the depressed portion of the organic compound layer 103, the common layer 104 formed over the organic compound layer 103 has a depressed portion in a manner similar to that of the organic compound layer 103. Furthermore, along the depressed portion of the common layer 104, the common electrode 155 formed over the common layer 104 has a depressed portion in a manner similar to that of the common layer 104. That is, the depressed portions of the organic resin layer 180, the first electrode 101, the organic compound layer 103, the common layer 104, and the common electrode 155 overlap with each other.

The common layer 104 is provided over the organic compound layer 103 and the insulating layer 127, and the common electrode 155 is provided over the common layer 104. The protective layer 135 is provided over the common electrode 155, and the substrate 352 is bonded with the use of the adhesive layer 142.

Although not shown in FIG. 15A, the light-emitting device 130G and the light-emitting device 130B are also provided.

### [Display Device 100E]

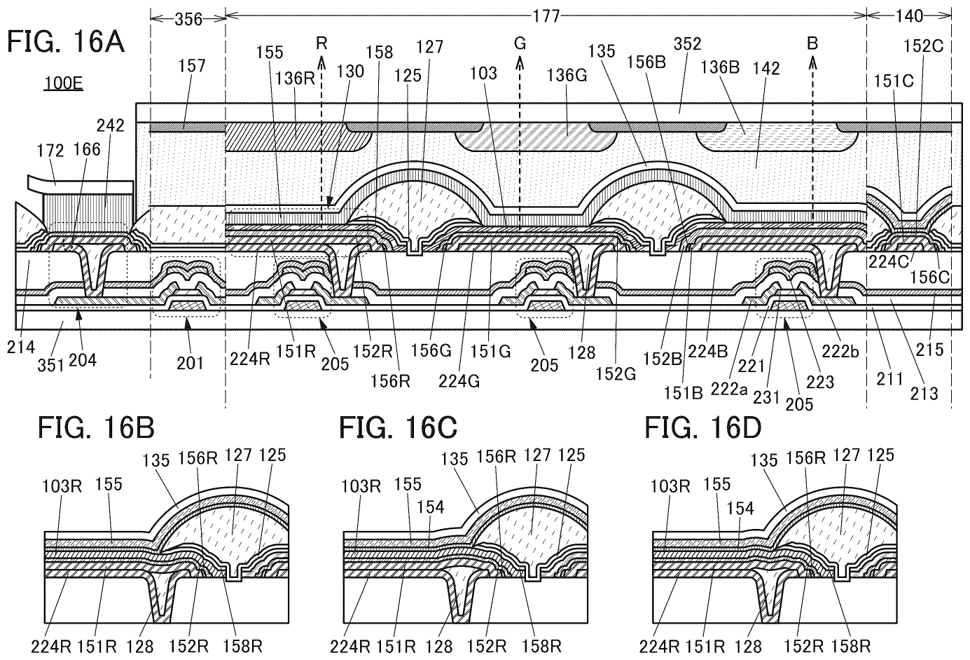

The display device 100E illustrated in FIG. 16A is a variation example of the top-emission display device 100B illustrated in FIG. 13A and differs from the display device 100B mainly in including the coloring layers 136R, 136G, and 136B.

In the display device 100E, the light-emitting device 130 includes a region overlapping with one of the coloring layers 136R, 136G, and 136B. The coloring layers 136R, 136G, and 136B can be provided on the surface of the substrate 352 on the substrate 351 side. End portions of the coloring layers 136R, 136G, and 136B can overlap with the light-blocking layer 157.

In the display device 100E, the light-emitting device 130 can emit white light, for example. For example, the coloring layer 136R, the coloring layer 136G, and the coloring layer 136B can transmit red light, green light, and blue light, respectively. Note that in the display device 100E, the coloring layers 136R, 136G, and 136B may be provided between the protective layer 135 and the adhesive layer 142.

Although FIG. 13A, FIG. 16A, and the like each illustrate an example in which the top surface of the layer 128 includes a flat portion, the shape of the layer 128 is not particularly limited. FIGS. 16B to 16D illustrate variation examples of the layer 128.

As illustrated in FIGS. 16B and 16D, the top surface of the layer 128 can have a shape in which its center and the vicinity thereof are depressed, i.e., a shape including a concave surface, in a cross-sectional view. A common layer 154 may be provided so as to be in contact with the common electrode 155.

As illustrated in FIG. 16C, the top surface of the layer 128 can have a shape in which its center and the vicinity thereof bulge, i.e., a shape including a convex surface, in a cross-sectional view.

The top surface of the layer 128 may include one or both of a convex surface and a concave surface. The number of convex surfaces and the number of concave surfaces included in the top surface of the layer 128 are not limited and can each be one or more.

The level of the top surface of the layer 128 and the level of the top surface of the conductive layer 224R may be the same or substantially the same, or may be different from each other. For example, the level of the top surface of the layer 128 may be either lower or higher than the level of the top surface of the conductive layer 224R.

FIG. 16B can be regarded as illustrating an example in which the layer 128 fits in the depressed portion of the conductive layer 224R. By contrast, as illustrated in FIG. 16D, the layer 128 may exist also outside the depressed portion of the conductive layer 224R, i.e., the top surface of the layer 128 may extend beyond the depressed portion.

### [Display Device 100F]

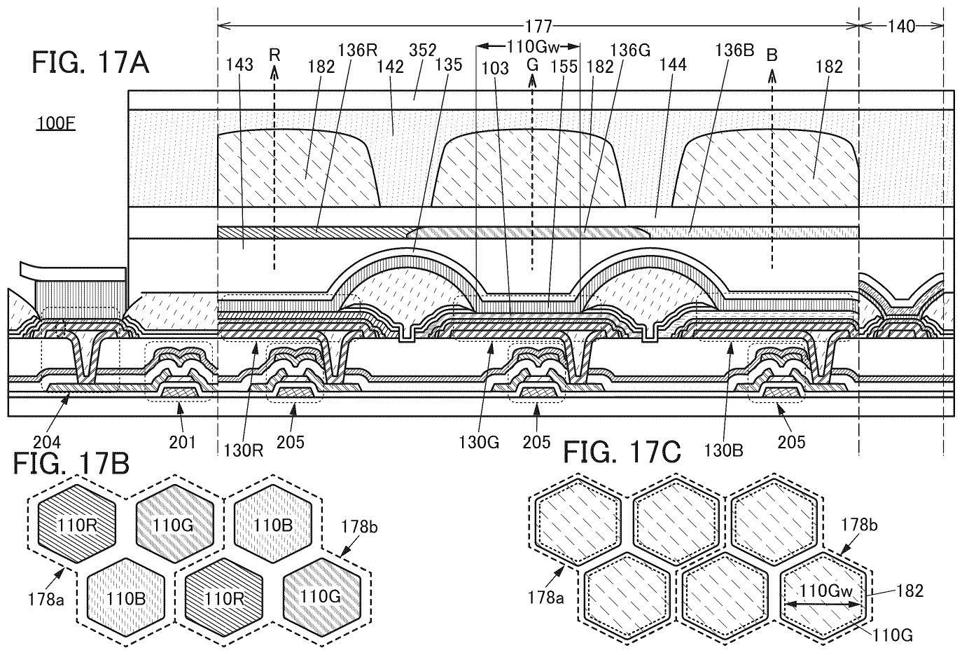

The display device 100F illustrated in FIG. 17A is a variation example of the top-emission display device 100B illustrated in FIG. 13A and includes microlenses 182 over the coloring layers 136R, 136G, and 136B. Note that in the drawings, reference numerals of some of the components that are shown in FIG. 13A are omitted; for the details of the components, the description made with reference to FIG. 13A is to be referred to.

FIG. 17B shows a top-view layout of the pixels 178 (the pixels 178a and 178b) each including the subpixels 110 (the subpixels 110R, 110G, and 110B), and FIG. 17C shows a top view of the microlenses 182 in a region where the subpixels 110R, 110G, and 110B of the pixels 178 are formed. Note that the width of the region where the common electrode 155 and the organic compound layer 103 are in contact with each other corresponds to a width 110Gw in the light-emitting region of the subpixel 110G.

In the display device 100F illustrated in FIG. 17A, a planarization film 143 is provided over the protective layer 135, and the coloring layers 136R, 136G, and 136B are provided over the planarization film 143. The planarization film 144 is provided to cover the coloring layers 136R, 136G, and 136B. The microlenses 182 are provided over the planarization film 144.

Note that as illustrated in FIG. 17C, the microlenses 182 are preferably provided on a subpixel basis in the region where the subpixels are formed.

Although the top surface shape of the microlens 182 is hexagonal in FIG. 17C, a different shape may be employed as needed. Examples of the top surface shape of the microlens 182 include polygons such as a triangle, a tetragon (including a rectangle and a square), and a pentagon; these polygons with rounded corners; an ellipse; and a circle.

The microlenses 182 can be formed using a material similar to that of the organic resin layer 180.

This embodiment can be combined as appropriate with the other embodiments or examples. In this specification, in the case where a plurality of structure examples are shown in one embodiment, the structure examples can be combined as appropriate.

### Embodiment 5

In this embodiment, electronic appliances of embodiments of the present invention will be described.

Electronic appliances of this embodiment include the light-emitting apparatus of one embodiment of the present invention in their display portions. The light-emitting apparatus of one embodiment of the present invention is highly reliable and can be easily increased in resolution and definition. Thus, the light-emitting apparatus of one embodiment of the present invention can be used for display portions of a variety of electronic appliances.

Examples of the electronic appliances include a digital camera, a digital video camera, a digital photo frame, a mobile phone, a portable game console, a portable information terminal, and an audio reproducing device, in addition to electronic appliances with a relatively large screen, such as a television device, desktop and notebook personal computers, a monitor of a computer and the like, digital signage, and a large game machine such as a pachinko machine.

In particular, the light-emitting apparatus of one embodiment of the present invention can have high resolution, and thus can be favorably used for an electronic appliance having a relatively small display portion. Examples of such an electronic appliance include watch-type and bracelet-type information terminal devices (wearable devices) and wearable devices capable of being worn on a head, such as a VR device like a head-mounted display, a glasses-type AR device, and an MR device.

The definition of the light-emitting apparatus of one embodiment of the present invention is preferably as high as HD (number of pixels: 1280×720), FHD (number of pixels: 1920×1080), WQHD (number of pixels: 2560×1440), WQXGA (number of pixels: 2560×1600), 4K (number of pixels: 3840×2160), or 8K (number of pixels: 7680×4320). In particular, definition of 4K, 8K, or higher is preferable. The pixel density (resolution) of the light-emitting apparatus of one embodiment of the present invention is preferably higher than or equal to 100 ppi, further preferably higher than or equal to 300 ppi, still further preferably higher than or equal to 500 ppi, yet still further preferably higher than or equal to 1000 ppi, yet still further preferably higher than or equal to 2000 ppi, yet still further preferably higher than or equal to 3000 ppi, yet still further preferably higher than or equal to 5000 ppi, yet still further preferably higher than or equal to 7000 ppi. With such a light-emitting apparatus having one or both of high definition and high resolution, the electronic appliance can provide higher realistic sensation, sense of depth, and the like in personal use such as portable use or home use. There is no particular limitation on the screen ratio (aspect ratio) of the light-emitting apparatus of one embodiment of the present invention. For example, the light-emitting apparatus is compatible with a variety of screen ratios such as 1:1 (a square), 4:3, 16:9, and 16:10.

The electronic appliance in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic appliance in this embodiment can have a variety of functions. For example, the electronic appliance in this embodiment can have a function of displaying a variety of information (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

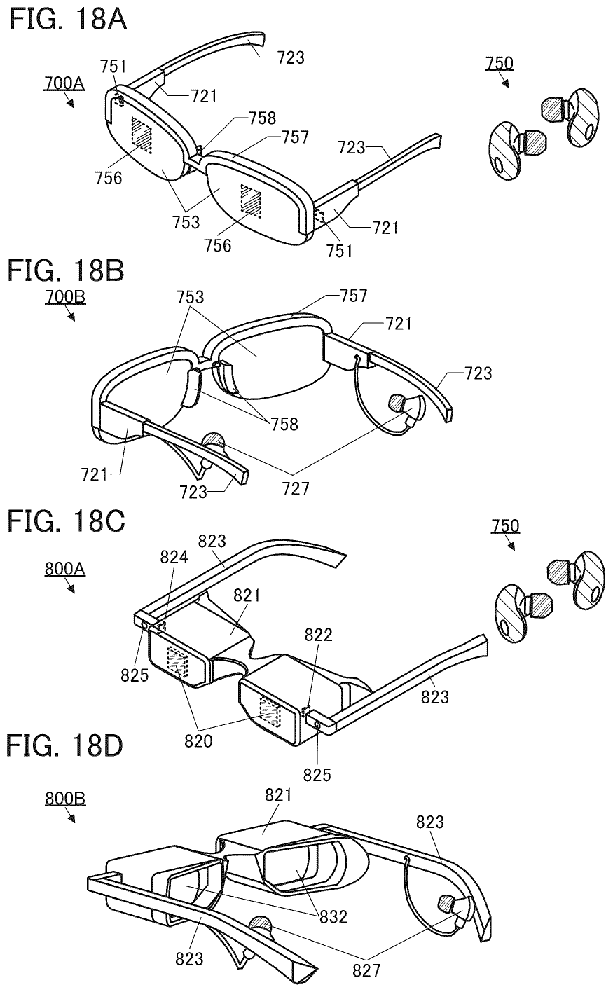

Examples of head-mounted wearable devices are described with reference to FIGS. 18A to 18D. These wearable devices have at least one of a function of displaying AR contents, a function of displaying VR contents, a function of displaying SR contents, and a function of displaying MR contents. The electronic appliance having a function of displaying contents of at least one of AR, VR, SR, MR, and the like enables the user to feel a higher level of immersion.

An electronic appliance 700A illustrated in FIG. 18A and an electronic appliance 700B illustrated in FIG. 18B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The light-emitting apparatus of one embodiment of the present invention can be used for the display panels 751. Thus, a highly reliable electronic appliance is obtained.

The electronic appliances 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic appliances 700A and 700B are electronic appliances capable of performing AR display.

In the electronic appliances 700A and 700B, a camera capable of capturing images of the front side may be provided as the image capturing portion. Furthermore, when the electronic appliances 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic appliances 700A and 700B are provided with a battery, so that they can be charged wirelessly and/or by wire.

A touch sensor module may be provided in the housing 721. The touch sensor module has a function of detecting a touch on the outer surface of the housing 721. Detecting a tap operation, a slide operation, or the like by the user with the touch sensor module enables various types of processing. For example, a moving image can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

An electronic appliance 800A illustrated in FIG. 18C and an electronic appliance 800B illustrated in FIG. 18D each include a pair of display portions 820, a housing 821, a communication portion 822, a pair of wearing portions 823, a control portion 824, a pair of image capturing portions 825, and a pair of lenses 832.

The light-emitting apparatus of one embodiment of the present invention can be used in the display portions 820. Thus, a highly reliable electronic appliance is obtained.

The display portions 820 are positioned inside the housing 821 so as to be seen through the lenses 832. When the pair of display portions 820 display different images, three-dimensional display using parallax can be performed.

The electronic appliances 800A and 800B can be regarded as electronic appliances for VR. The user who wears the electronic appliance 800A or 800B can see images displayed on the display portions 820 through the lenses 832.

The electronic appliances 800A and 800B preferably include a mechanism for adjusting the lateral positions of the lenses 832 and the display portions 820 so that the lenses 832 and the display portions 820 are positioned optimally in accordance with the positions of the user's eyes. Moreover, the electronic appliances 800A and 800B preferably include a mechanism for adjusting focus by changing the distance between the lenses 832 and the display portions 820.

The electronic appliance 800A or 800B can be mounted on the user's head with the wearing portions 823. FIG. 18C, for instance, shows an example where the wearing portion 823 has a shape like a temple (also referred to as a joint or the like) of glasses; however, one embodiment of the present invention is not limited thereto. The wearing portion 823 can have any shape with which the user can wear the electronic appliance, for example, a shape of a helmet or a band.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of fields of view, such as a telescope field of view and a wide field of view.

Although an example where the image capturing portions 825 are provided is described here, a range sensor (hereinafter also referred to as a sensing portion) capable of measuring the distance between the user and an object just needs to be provided. In other words, the image capturing portion 825 is one embodiment of the sensing portion. As the sensing portion, an image sensor or a range image sensor such as a light detection and ranging (LiDAR) sensor can be used, for example. By using images obtained by the camera and images obtained by the range image sensor, more information can be obtained and a gesture operation with higher accuracy is possible.

The electronic appliance 800A may include a vibration mechanism that functions as bone-conduction earphones. For example, at least one of the display portion 820, the housing 821, and the wearing portion 823 can include the vibration mechanism. Thus, without additionally requiring an audio device such as headphones, earphones, or a speaker, the user can enjoy video and sound only by wearing the electronic appliance 800A.

The electronic appliances 800A and 800B may each include an input terminal. To the input terminal, a cable for supplying a video signal from a video output device or the like, power for charging a battery provided in the electronic appliance, and the like can be connected.

The electronic appliance of one embodiment of the present invention may have a function of performing wireless communication with earphones 750. The earphones 750 include a communication portion (not illustrated) and have a wireless communication function. The earphones 750 can receive information (e.g., audio data) from the electronic appliance with the wireless communication function. For example, the electronic appliance 700A in FIG. 18A has a function of transmitting information to the earphones 750 with the wireless communication function. For another example, the electronic appliance 800A in FIG. 18C has a function of transmitting information to the earphones 750 with the wireless communication function.

The electronic appliance may include an earphone portion. The electronic appliance 700B in FIG. 18B includes earphone portions 727. For example, the earphone portion 727 can be connected to the control portion by wire. Part of a wiring that connects the earphone portion 727 and the control portion may be positioned inside the housing 721 or the wearing portion 723.

Similarly, the electronic appliance 800B in FIG. 18D includes earphone portions 827. For example, the earphone portion 827 can be connected to the control portion 824 by wire. Part of a wiring that connects the earphone portion 827 and the control portion 824 may be positioned inside the housing 821 or the wearing portion 823. Alternatively, the earphone portions 827 and the wearing portions 823 may include magnets. This is preferable because the earphone portions 827 can be fixed to the wearing portions 823 with magnetic force and thus can be easily housed.

The electronic appliance may include an audio output terminal to which earphones, headphones, or the like can be connected. The electronic appliance may include one or both of an audio input terminal and an audio input mechanism. As the audio input mechanism, a sound collecting device such as a microphone can be used, for example. The electronic appliance may have a function of a headset by including the audio input mechanism.

As described above, both the glasses-type device (e.g., the electronic appliances 700A and 700B) and the goggles-type device (e.g., the electronic appliances 800A and 800B) are preferable as the electronic appliance of one embodiment of the present invention.

The electronic appliance of one embodiment of the present invention can transmit information to earphones by wire or wirelessly.

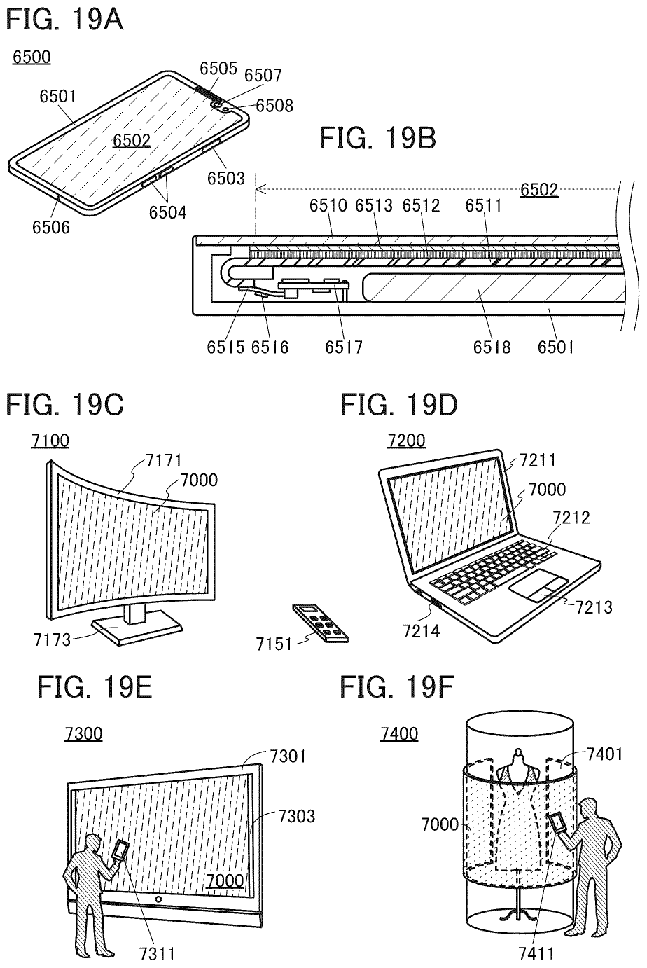

An electronic appliance 6500 illustrated in FIG. 19A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The light-emitting apparatus of one embodiment of the present invention can be used in the display portion 6502. Thus, a highly reliable electronic appliance is obtained.

FIG. 19B is a schematic cross-sectional view including an end portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The light-emitting apparatus of one embodiment of the present invention can be used in the display panel 6511. Thus, an extremely lightweight electronic appliance can be obtained. Since the display panel 6511 is extremely thin, the battery 6518 with high capacity can be mounted without an increase in the thickness of the electronic appliance. An electronic appliance with a narrow bezel can be obtained when part of the display panel 6511 is folded back so that the portion connected to the FPC 6515 is provided on the back side of a pixel portion.

FIG. 19C illustrates an example of a television device. In a television device 7100, a display portion 7000 is incorporated in a housing 7171. Here, the housing 7171 is supported by a stand 7173.

The light-emitting apparatus of one embodiment of the present invention can be used in the display portion 7000. Thus, a highly reliable electronic appliance is obtained.

Operation of the television device 7100 illustrated in FIG. 19C can be performed with an operation switch provided in the housing 7171 and a separate remote control 7151. Alternatively, the display portion 7000 may include a touch sensor, and the television device 7100 may be operated by touch on the display portion 7000 with a finger or the like. The remote control 7151 may be provided with a display portion for displaying information output from the remote control 7151. With operation keys or a touch panel of the remote control 7151, channels and volume can be controlled and video displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 19D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The light-emitting apparatus of one embodiment of the present invention can be used in the display portion 7000. Thus, a highly reliable electronic appliance is obtained.

FIGS. 19E and 19F illustrate examples of digital signage that can be used for store windows, showcases, and the like.

Digital signage 7300 illustrated in FIG. 19E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 19F shows digital signage 7400 attached to a cylindrical pillar 7401. The digital signage 7400 includes the display portion 7000 provided along a curved surface of the pillar 7401.

In FIGS. 19E and 19F, the light-emitting apparatus of one embodiment of the present invention can be used in the display portion 7000. Thus, a highly reliable electronic appliance is obtained.

A larger area of the display portion 7000 can increase the amount of information that can be provided at a time. The display portion 7000 having a larger area attracts more attention, so that the effectiveness of the advertisement can be increased, for example.

Specifically, in the case where the display device of one embodiment of the present invention is used for the digital signage 7400 shown in FIG. 19F that displays advertisements and the like, the display device being a light-transmitting panel can increase the flexibility of representation. The display device having a light-transmitting property can be manufactured, for example, by using a wiring and a support member that include a conductive film transmitting visible light and adjusting the distance between pixel electrodes.

The use of the tandem light-emitting device of one embodiment of the present invention in addition to the wiring and the support member each of which is formed of the conductive film that transmits visible light can increase the luminance per pixel. That is, favorable display can be performed even when the display device has a low aperture ratio, so that the light-transmitting property of the display portion of the display device can be increased. Thus, such a structure is suitably used in the light-transmitting display device of one embodiment of the present invention.

As illustrated in FIGS. 19E and 19F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

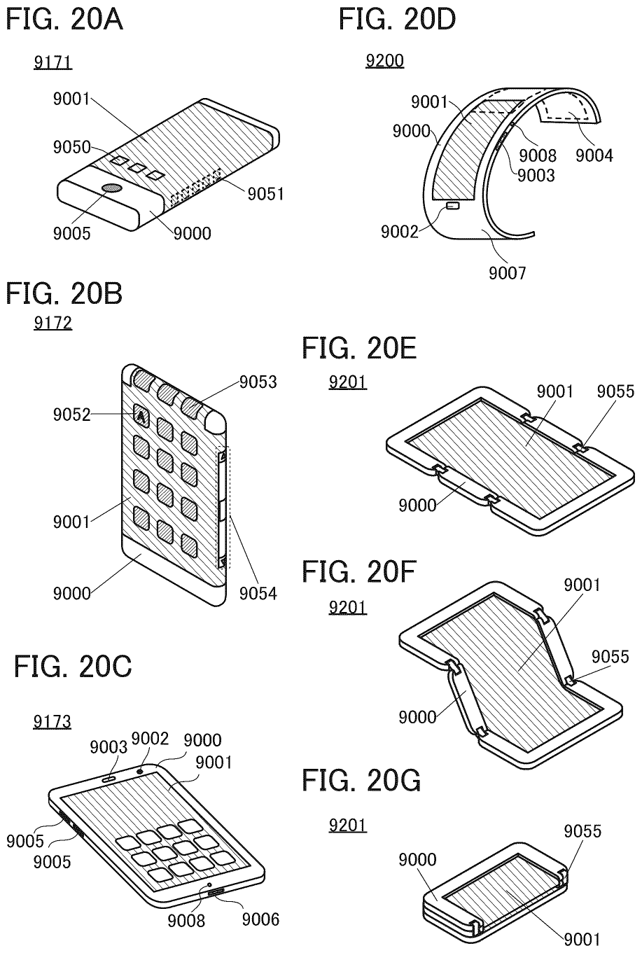

Electronic appliances illustrated in FIGS. 20A to 20G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic appliances illustrated in FIGS. 20A to 20G have a variety of functions. For example, the electronic appliances can have a function of displaying a variety of information (e.g., a still image, a moving image, and a text image) on the display portion, a touch panel function, a function of displaying a calendar, date, time, and the like, a function of controlling processing with the use of a variety of software (programs), a wireless communication function, and a function of reading out and processing a program or data stored in a recording medium. Note that the functions of the electronic appliances are not limited thereto, and the electronic appliances can have a variety of functions. The electronic appliances may include a plurality of display portions. The electronic appliances may be provided with a camera or the like and have a function of taking a still image or a moving image, a function of storing the taken image in a storage medium (an external storage medium or a storage medium incorporated in the camera), a function of displaying the taken image on the display portion, and the like.

The electronic appliances in FIGS. 20A to 20G are described in detail below.

FIG. 20A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 20A illustrates an example in which three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, the title and sender of an e-mail, an SNS message, or the like, the date, the time, remaining battery, and the radio field intensity. Alternatively, the icon 9050 or the like may be displayed at the position where the information 9051 is displayed.

FIG. 20B is a perspective view of a portable information terminal 9172. The portable information terminal 9172 has a function of displaying information on three or more surfaces of the display portion 9001. Here, an example in which information 9052, information 9053, and information 9054 are displayed on different surfaces is described. For example, the user of the portable information terminal 9172 can check the information 9053 displayed such that it can be seen from above the portable information terminal 9172, with the portable information terminal 9172 put in a breast pocket of his/her clothes. Thus, the user can see the display without taking out the portable information terminal 9172 from the pocket and decide whether to answer the call, for example.

FIG. 20C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, the camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 20D is a perspective view of a watch-type portable information terminal 9200. The portable information terminal 9200 can be used as a Smartwatch (registered trademark), for example. The portable information terminal 9200 may include the operation key 9005 as a button for operation on the left side surface of the housing 9000 and include the sensor 9007 on the bottom surface of the housing 9000. Although the curved bangle-type housing 9000 is shown as an example, the housing 9000 may include a belt or the like in combination so that the portable information terminal 9200 can be worn. The display surface of the display portion 9001 is curved, and an image can be displayed on the curved display surface. A power storage device 9004 may be curved along the housing 9000. The power storage device 9004 has flexibility and can be bent in accordance with a change in shape at the time when the user puts on or takes off the portable information terminal 9200. Note that a charge control IC connected to the power storage device 9004 may be included. Furthermore, for example, mutual communication between the portable information terminal 9200 and a headset capable of wireless communication can be performed, and thus hands-free calling is possible. The portable information terminal 9200 can perform mutual data transmission with another information terminal without a wire and perform charging operation by wireless power feeding. Note that the connection terminal 9006 may be provided in the housing 9000 and data transmission and charging operation may be performed by wire.

FIGS. 20E to 20G are perspective views of a foldable portable information terminal 9201. FIG. 20E is a perspective view showing the portable information terminal 9201 that is opened. FIG. 20G is a perspective view showing the portable information terminal 9201 that is folded. FIG. 20F is a perspective view showing the portable information terminal 9201 that is shifted from one of the states in FIGS. 20E and 20G to the other. The portable information terminal 9201 is highly portable when folded. When the portable information terminal 9201 is opened, a seamless large display region is highly browsable. The display portion 9001 of the portable information terminal 9201 is supported by three housings 9000 joined together by hinges 9055. The display portion 9001 can be folded with a radius of curvature greater than or equal to 0.1 mm and less than or equal to 150 mm, for example.

This embodiment can be combined as appropriate with the other embodiments or examples. In this specification, in the case where a plurality of structure examples are shown in one embodiment, the structure examples can be combined as appropriate.

### Example 1

### Synthesis Example 1

In this synthesis example, a synthesis method of 9,9′-di(2-naphthyl-1,3,4,5,6,7,8-d₇)-9H,9′H-3,3′-bicarbazole-1,1′,2,2′,4,4′,5,5′,6,6′,7,7′,8,8′-d₁₄ (abbreviation: BisβNCz-d28), which is the organic compound represented by Structural Formula (200) in Embodiment 1, will be specifically described.

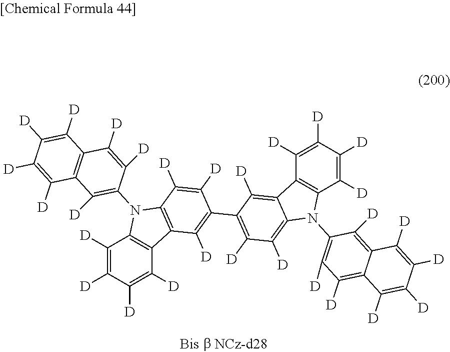 [SMILES: [2H]c1c([2H])c([2H])c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c([2H])c([2H])c([2H])c([2H])c6c5[2H])c([2H])c([2H])c43)c([2H])c([2H])c2c1[2H]]

### Step 1: Synthesis of 9-(2-naphthyl-1,3,4,5,6,7,8-d₇)-9H-carbazole-1,2,3,4,5,6,7,8-d₈

Into a 2 L three-neck flask were put 30.1 g (172 mmol) of carbazole-1,2,3,4,5,6,7,8-d₈ (produced by Angene International Limited, product number: AG00BZO3), 36.9 g (172 mmol) of 2-bromonaphthalene-1,3,4,5,6,7,8-d₇ (produced by ChemScene, catalog No. CS-0639480), 23.8 g (172 mmol) of potassium carbonate, 1.87 g (7.07 mmol) of 18-crown-6, and 850 mL of toluene. This mixture was degassed by being stirred under reduced pressure. Then, the atmosphere in the flask was replaced with nitrogen. After that, the mixture was heated at 60° C. To this reaction solution were added 9.0 mL (3.36 mmol) of tri-tert-butylphosphine (abbreviation: P(tBu)₃) (10 wt % hexane solution) and 1.55 g (1.69 mmol) of tris(dibenzylideneacetone)dipalladium(0) (abbreviation: Pd₂(dba)₃), and stirring was performed at 120° C. for two hours. The following day, the reaction solution was heated to 60° C., and then 3.45 g (16.1 mmol) of 2-bromonaphthalene-1,3,4,5,6,7,8-d₇, 2.88 g (7.01 mmol) of 2-dicyclohexylphosphino-2′,6′-dimethoxybiphenyl (abbreviation: SPhos), and 1.71 g (1.86 mmol) of Pd₂(dba)₃ were added and stirring was performed at 120° C. for six hours. To this reaction solution was added 500 mL of toluene, stirring was performed at 80° C., and suction filtration was performed through alumina, Celite (FUJIFILM Wako Pure Chemical Corporation, Catalog No: 537-02305), and Florisil (FUJIFILM Wako Pure Chemical Corporation, Catalog No: 066-05265). The obtained filtrate was concentrated to give a toluene solution containing the target substance, hexane was then added, and ultrasonic cleaning was performed, whereby a pale yellow solid was precipitated. This solid was collected by suction filtration to give 50.2 g of the target pale yellow solid in a yield of 95%. Synthesis Scheme (s1-1) of Step 1 is shown below.

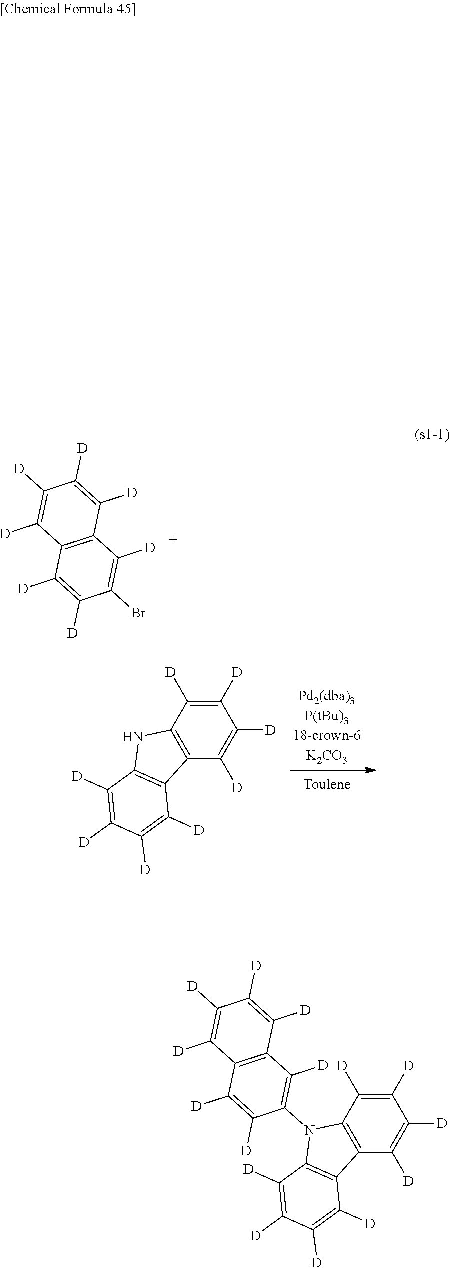 [SMILES: [2H]c1c([2H])c([2H])c2c([2H])c(Br)c([2H])c([2H])c2c1[2H] ; [2H]c1c([2H])c([2H])c2c([nH]c3c([2H])c([2H])c([2H])c([2H])c32)c1[2H] ; [2H]c1c([2H])c([2H])c2c([2H])c(-n3c4c([2H])c([2H])c([2H])c([2H])c4c4c([2H])c([2H])c([2H])c([2H])c43)c([2H])c([2H])c2c1[2H]]

The molecular weight of the pale yellow solid obtained in Step 1 was measured by LC/MS, in which case m/z 309 (a proton adduct of 9-(2-naphthyl-1,3,4,5,6,7,8-d₇)-9H-carbazole-1,2,3,4,5,6,7,8-d₈) was observed with respect to the calculated mass 308 of the target substance. The results reveal that 9-(2-naphthyl-1,3,4,5,6,7,8-d₇)-9H-carbazole-1,2,3,4,5,6,7,8-d₈ is obtained.

### Step 2: Synthesis of 6-bromo-9-(2-naphthyl-1,3,4,5,6,7,8-d₇)-9H-carbazole-1,2,3,4,5,7,8-d₇

Into a 2 L conical flask were put 50.2 g (162 mmol) of 9-(2-naphthyl-1,3,4,5,6,7,8-d₇)-9H-carbazole-1,2,3,4,5,6,7,8-d₈ synthesized in Step 1, 29.0 g (163 mmol) of N-bromosuccinimide (NBS), 740 mL of toluene, and 325 mL of ethyl acetate (AcOEt), and stirring was performed at room temperature for 16 hours. After that, stirring was performed at 80° C. for seven hours. After that, pure water was added, and solution separation was performed. The organic layer was dehydrated using magnesium sulfate and suction filtration was performed after a predetermined time elapsed. The filtrate was concentrated, hexane was added, and ultrasonic cleaning was performed, so that a white solid was precipitated. The solid was collected by suction filtration to give 66.1 g of the target white solid. The white solid was used in a reaction in Step 3. Synthesis Scheme (s1-2) of Step 2 is shown below.

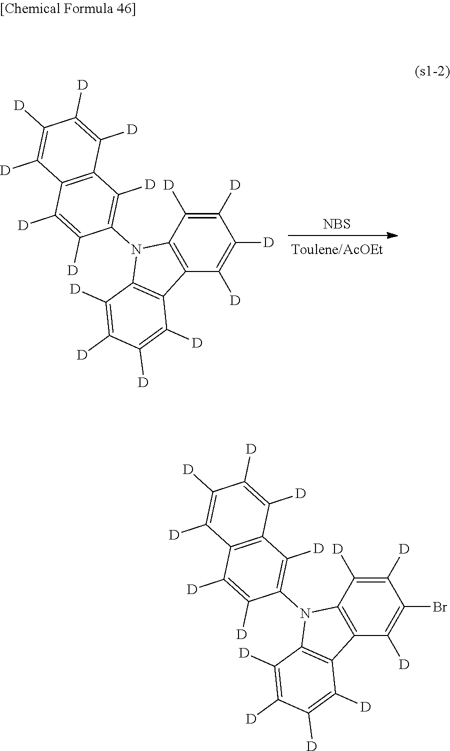

The molecular weight of the white solid obtained in Step 2 was measured by LC/MS, in which case m/z 385 was observed with respect to the calculated mass 385 of the target substance. The results reveal that 6-bromo-9-(2-naphthyl-1,3,4,5,6,7,8-d₇)-9H-carbazole-1,2,3,4,5,7,8-d₇ is obtained.

### Step 3: Synthesis of 6-(4,4,5,5-tetramethyl-1,3,2-dioxaborolane)-9-(2-naphthyl-1,3,4,5,6,7,8-d₇)-9H-carbazole-1,2,3,4,5,7,8-d₇

Into a 2 L three-neck flask were put 18.1 g (46.8 mmol) of a white solid of 6-bromo-9-(2-naphthyl-1,3,4,5,6,7,8-d₇)-9H-carbazole-1,2,3,4,5,7,8-d₇ synthesized in Step 2, 12.1 g (47.8 mmol) of bis(pinacolato)diboron, 13.7 g (140 mmol) of potassium acetate (AcOK), and 230 mL of dioxane. This mixture was degassed by being stirred under reduced pressure. Then, the atmosphere in the flask was replaced with nitrogen. After that, the mixture was heated at 60° C. To this reaction solution was added 410 mg (0.50 mmol) of [1,1′-bis(diphenylphosphino)ferrocene]palladium(II) dichloride dichloromethane adduct (abbreviation: Pd(dppf)₂Cl₂·CH₂Cl₂), and the mixture was stirred at 110° C. for six hours. After the temperature was cooled down to room temperature, toluene and pure water were added and solution separation was performed. The organic layer was dehydrated with magnesium sulfate and suction filtration was performed. A black oily substance obtained by concentration of the filtrate was purified by silica gel column chromatography (developing solvent: toluene) to give 17.3 g of the target pale yellow solid in a yield of 86%. Synthesis Scheme (s1-3) of Step 3 is shown below.

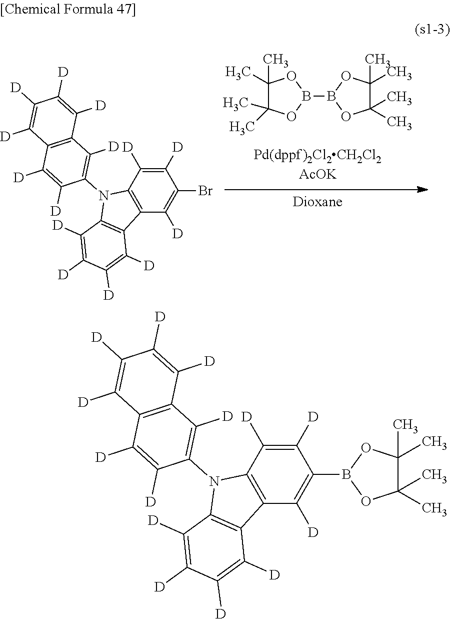

The molecular weight of the pale yellow solid obtained in Step 3 was measured by LC/MS, in which case m/z 434 (a proton adduct of 6-(4,4,5,5-tetramethyl-1,3,2-dioxaborolane)-9-(2-naphthyl-1,3,4,5,6,7,8-d₇)-9H-carbazole-1,2,3,4,5,7,8-d₇) was observed with respect to the calculated mass 433 of the target substance. The results reveal that 6-(4,4,5,5-tetramethyl-1,3,2-dioxaborolane)-9-(2-naphthyl-1,3,4,5,6,7,8-d₇)-9H-carbazole-1,2,3,4,5,7,8-d₇ is obtained.

### Step 4: Synthesis of BisβNCz-d28

Into a 500 mL three-neck flask were put 2.9 g (7.5 mmol) of the white solid of 6-bromo-9-(2-naphthyl-1,3,4,5,6,7,8-d₇)-9H-carbazole-1,2,3,4,5,7,8-d₇ synthesized in Step 2, 3.2 g (7.5 mmol) of 6-(4,4,5,5-tetramethyl-1,3,2-dioxaborolane)-9-(2-naphthyl-1,3,4,5,6,7,8-d₇)-9H-carbazole-1,2,3,4,5,7,8-d₇ synthesized in Step 3, 11 ml of 2 mol/L aqueous solution of potassium carbonate (abbreviation: K₂CO₃ aq.), 49 mg (0.16 mmol) of tris(o-tolyl)phosphine (abbreviation: P(o-tolyl)₃), 37 ml of toluene, and 8 ml of ethanol. This mixture was degassed by being stirred under reduced pressure. Then, the atmosphere in the flask was replaced with nitrogen. After that, the mixture was heated at 60° C. To this reaction solution, 22 mg (0.98 mmol) of palladium(II) acetate (abbreviation: Pd(OAc)₂) was added, and the mixture was stirred at 90° C. for four hours. After the temperature was cooled down to room temperature, chloroform and pure water were added to the mixture. Then, solution separation was performed. The organic layer was dehydrated using magnesium sulfate, and after a predetermined time elapsed, this mixture was subjected to gravity filtration. A white solid obtained by concentration of the filtrate was recrystallized with toluene, so that 2.4 g of the target white solid was obtained in a yield of 52%. The purity of the target white solid was 90% when measured by liquid chromatography-mass spectrometry (LC/MS). Recrystallization was performed once as a purification step, and it was found that the target substance can be obtained with high purity even in a simple purification step as described above, and that the synthesis method is suitable for increasing the purity. Note that LC/MS was performed using Waters Xevo G2 T of produced by Waters Corporation. Synthesis scheme (s1-4) of Step 4 is shown below.

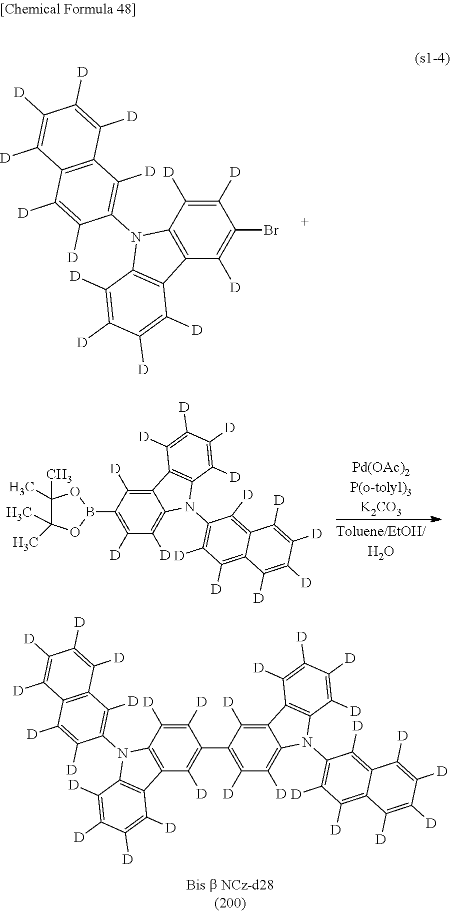

By a train sublimation method, 2.2 g of the obtained white solid was purified. In the sublimation purification, the solid was heated at 300° C. for 46 hours under a pressure of 3.25×10⁻² Pa. After the sublimation purification, 1.4 g of a white solid which was the target substance was obtained at a collection rate of 65%. The purity of the white solid subjected to the sublimation purification was 99% when measured by LC/MS. Recrystallization and purification by sublimation were each performed once as a purification step, and it was found that the target substance can be obtained with high purity even in a simple purification step as described above. Also, it was confirmed again that the synthesis method is suitable for increasing the purity. The apparatus used for the LC/MS is the same as the apparatus used after the recrystallization is performed once.

The molecular weight of the white solid obtained in Step 4 was measured by LC/MS, in which case m/z 612 was observed with respect to the calculated mass 612 of the target substance.

Next, the white solid obtained after the sublimation purification in Step 4 was subjected to structural analysis with an electron diffraction crystallography structural analysis apparatus. For the electron diffraction crystallography structural analysis, an electron diffractometer (a 200 keV electron beam source XtaLAB Synergy-ED (model number: JEM-2300) manufactured by JEOL Ltd.) was used.

A measurement method is described. First, the white solid obtained after the purification by sublimation was supported by a grid (produced by TED PELLA, INC., product number: 01840, product name: Carbon Film only on 200 mesh, Copper). The grid supporting the white solid was put on a sample holder and fixed with a clamp. Next, the sample holder where the grid supporting the white solid was fixed was set in XtaLAB Synergy-ED. Next, measurement and structural analysis were performed. The white solid supported by the grid was irradiated with an electron beam and the obtained electron diffraction pattern was analyzed, whereby the molecular structure of the white solid was obtained.

Note that for analysis of electron diffraction patterns, CRYSALIS^{Pro}forED (manufactured by Rigaku Holdings Corporation) was used. The structural analysis was performed by an intrinsic phasing method using Olex2 (Non-Patent Document 1) and SHELXT (Non-Patent Document 2). Structural refinement was performed by a least-squares method using SHELXT (Non-Patent Document 3).

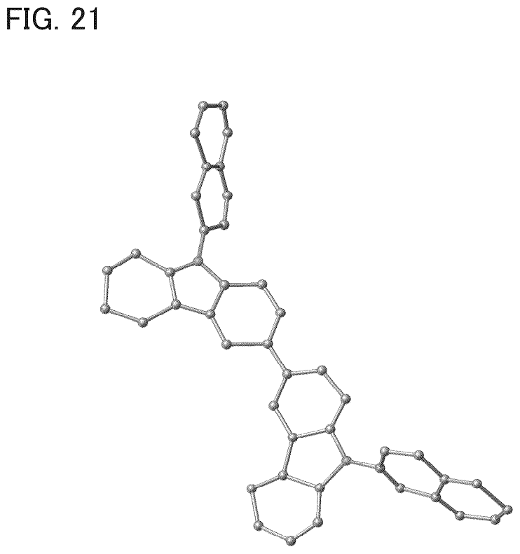

The obtained molecular structure is shown in FIG. 21. In addition, crystal structure data is shown below.

Crystal Data for C44N2 (M=556.50 g/mol): Resolution=0.80 Å, reflections measured (0.204°≤2θ≤1.798°), 5296 unique (Rᵢₙₜ=0.3354, R_{sigma}=0.4299), orthorhombic, space group Pna2₁ (no. 33), Unit cell, a=12.39(16) Å, b=8.6(2) Å, c=29.7(2) Å, V=3176(99) Å³, Z=4, T=293(2)K, Completeness=80.1, 1/sig=2.3, Redundancy=4.4, μ (electron)=0.000 mm⁻¹, D_{calc}=1.164 g/cm³, 12262, GooF=1.01 which were used in all calculations. The final R₁ was 0.2022 (I>2σ(I)) and wR₂ was 0.4947 (all data).

From the above, a coupling reaction was performed in Step 1 using carbazole-1,2,3,4,5,6,7,8-d₈ (product number: AGOOBZO3, produced by Angene International Limited) and 2-bromonaphthalene-1,2,3,4,5,6,8-d₇ (Catalog No. CS-0639480 produced by ChemScene) as source materials, and it was found that a compound finally obtained through Steps 2, 3, and 4 was BisβNCz-d28. That is, the molecular weight obtained from LC/MS of the compound obtained through the above steps was m/z 612, and by comprehensively judging from FIG. 21 obtained by the electron diffraction crystallography, the compound was identified as BisβNCz-d28.

### <Measurement of Physical Properties>

Next, the ultraviolet-visible absorption spectrum (hereinafter, referred to as “absorption spectrum”) and emission spectrum (photoluminescence (PL) spectrum, hereinafter referred to as PL spectrum) of BisβNCz-d28 in a toluene solution and in a solid thin film were measured.

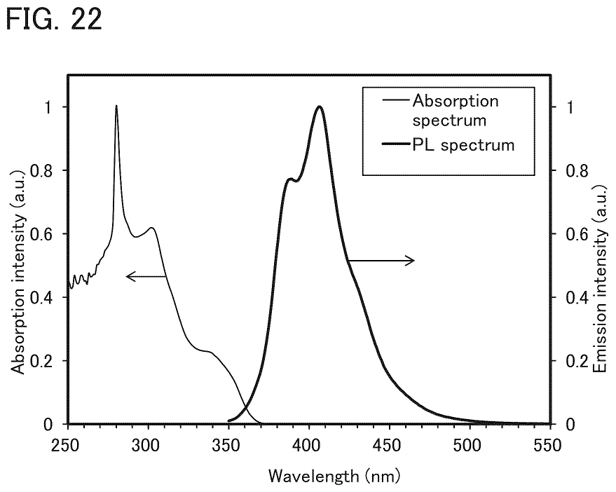

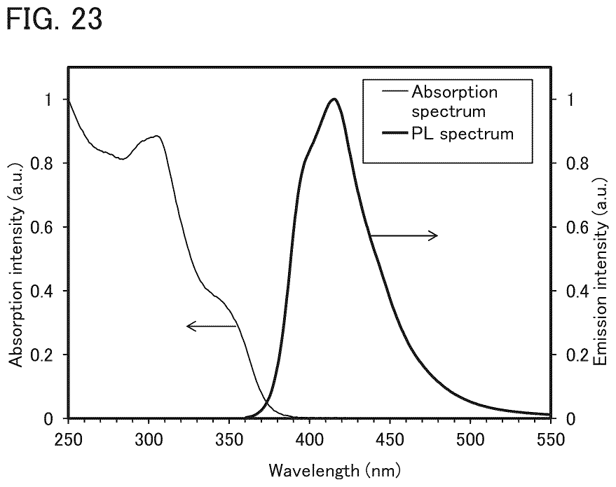

The absorption spectrum of the solution was measured with an ultraviolet-visible spectrophotometer (V-770DS, manufactured by JASCO Corporation), and the absorption spectrum of the thin film was measured with an ultraviolet-visible spectrophotometer (U-4100, manufactured by Hitachi, Ltd.). To calculate the absorption spectrum of the toluene solution of BisβNCz-d28, the absorption spectrum of toluene put in a quartz cell was measured and then subtracted from the absorption spectrum of the toluene solution of BisβNCz-d28 put in a quartz cell. The PL spectrum was measured with a fluorescence spectrophotometer (FP-8600DS, manufactured by JASCO Corporation). FIG. 22 shows the measurement results of the absorption spectrum and the PL spectrum of the toluene solution of BisβNCz-d28, and FIG. 23 shows the measurement results of the absorption spectrum and the PL spectrum of the thin film of BisβNCz-d28.

As shown in FIG. 22, the absorption spectrum of the toluene solution of BisβNCz-d28 exhibited an absorption peak at around 340 nm and a shoulder peak at around 352 nm. The results reveal that the solution of BisβNCz-d28 does not absorb light in a region with a wavelength greater than or equal to 400 nm, which indicates that the material of the present invention can be suitably used for a light-emitting device. In addition, as shown in FIG. 22, an emission peak was observed at around 428 nm (excitation wavelength: 340 nm) in the PL spectrum of the toluene solution of BisβNCz-d28.

As shown in FIG. 23, the absorption spectrum of the thin film of BisβNCz-d28 exhibited the maximum absorption peak at around 406 nm and a shoulder peak at around 430 nm. An absorption peak was also observed at around 353 nm. The results reveal that the thin film of BisβNCz-d28 also does not absorb light in a region with a wavelength greater than or equal to 400 nm, which indicates that the material of the present invention can be suitably used for a light-emitting device. In addition, as shown in FIG. 23, the PL spectrum of the thin film of BisβNCz-d28 exhibited an emission peak at around 415 nm (excitation wavelength: 342 nm).

The thermogravimetry-differential thermal analysis (TG-DTA) of BisβNCz-d28 was performed. For the measurement, a high-sensitivity differential type differential thermogravimeter (STA 2500 Regulus produced by NETZSCH Japan K.K.) was used. The measurement was performed under two conditions. The first measurement was performed at atmospheric pressure at a temperature rising rate of 10° C./min under a nitrogen stream (flow rate: 200 mL/min). The second measurement was performed at a temperature rising rate of 10° C./min at 1.70×10⁻³ Pa.

In TG-DTA of BisβNCz-d28 performed under the first measurement conditions, it was revealed that the temperature (i.e., the sublimation or decomposition temperature) at which the weight obtained by thermogravimetry measurement was reduced by 5% of the weight at the beginning of the measurement was 466° C. under atmospheric pressure. The result shows that the sublimation or decomposition temperature of BisβNCz-d28 under atmospheric pressure is 466° C. and has high heat resistance.

In TG-DTA of BisβNCz-d28 performed under the second measurement conditions, it was revealed that the temperature (i.e., the sublimation or decomposition temperature) at which the weight obtained by thermogravimetry measurement was reduced by 5% of the weight at the beginning of the measurement was 225° C. at 1.70×10⁻³ Pa. The results reveal that the sublimation or decomposition temperature of BisβNCz-d28 at 1.70×10⁻³ Pa is 225° C.

The above results reveal that the sublimation or decomposition temperature (225° C.) of BisβNCz-d28 at 1.70×10⁻³ Pa is lower than the sublimation or decomposition temperature (466° C.) at atmospheric pressure by 241° C. This indicates that BisβNCz-d28 can be deposited by evaporation at a temperature sufficiently lower than the sublimation or decomposition temperature under atmospheric pressure. Thus, it was indicated that the organic compound of one embodiment of the present invention is less likely to be decomposed in evaporation and a high-purity film can be formed by evaporation.

Differential scanning calorimetry (DSC) measurement of BisβNCz-d28 was performed with DSC8500 manufactured by PerkinElmer, Inc. The DSC measurement was performed in the following manner: the temperature was raised from −10° C. to 350° C. at a temperature rising rate of 40° C./min and held for two minutes, and then the temperature was decreased to −10° C. at a temperature decreasing rate of 100° C./min. This operation was performed twice in succession. Next, the DSC measurement was performed in the following manner: the temperature was raised from −10° C. to 350° C. at a temperature rising rate of 50° C./min and held for two minutes, and then the temperature was decreased to −10° C. at a temperature decreasing rate of 100° C./min. This operation was performed once.

The DSC measurement result of the second temperature rising process revealed that T_{g} of BisβNCz-d28 was 131° C. It was found that the use of the organic compound of one embodiment of the present invention for an organic semiconductor element such as a light-emitting device can increase the heat resistance of the organic semiconductor element.

Next, the results of analyzing the spin density distribution of the Ti level in BisβNCz-d28 by calculation are described.

### <Calculation Method>

Spin density distribution of the Ti level was analyzed by analyzing the spin density in the most stable structure where the lowest triplet excited state (Ti) level of BisβNCz-d28 is the lowest. A density functional theory (DFT) method was used as the calculation method. The total energy calculated by the DFT is represented as the sum of potential energy, electrostatic energy between electrons, electronic kinetic energy, and exchange-correlation energy including all the complicated interactions between electrons. In the DFT, an exchange-correlation interaction is approximated by a functional (a function of another function) of one electron potential represented in terms of electron density to enable high-speed calculations. Here, B3LYP which is a hybrid functional was used to specify the weight of each parameter related to exchange-correlation energy. As a basis function, 6-311G (d,p) was used. Gaussian 09 was used as a computational program.

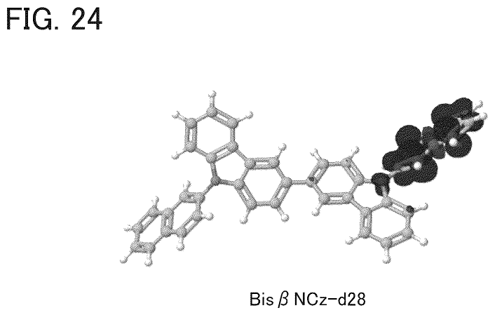

The analysis results are shown in FIG. 24. In FIG. 24, spheres in the diagrams represent atoms included in the compounds, and clouds around some of the atoms represent spin density distribution at the time when the density value in atomic units is 0.003 e/a₀³ (where e represents elementary charge (1 e=1.60218×10⁻¹⁹ C) and a₀ represents a Bohr radius (1 a₀=5.29177×10⁻¹¹ m)).

FIG. 24 shows that in BisβNCz-d28, spin density of the Ti level is distributed over one 2-naphthyl group.

The result shows that, as described in Embodiment 1, spin density is distributed over the 2-naphthyl group in the T₁ level in the organic compound of one embodiment of the present invention. Thus, in the case where BisβNCz-d28 is used as the organic compound of one embodiment of the present invention, the excitation energy is dispersed to two places. Accordingly, it was found that in the case where the light-emitting device is driven at the same current density (in the case where the excitation frequency per molecule is the same), the excitation frequency per 2-naphthyl group is decreased, so that the molecule is less likely to deteriorate.

### Example 2

In this example, a light-emitting device 1 including BisβNCz-d28 (Structural Formula (200)), whose synthesis method is described in Example 1, in its light-emitting layer and a comparative light-emitting device 2 including BisβNCz, which is a comparative organic compound, in its a light-emitting layer were fabricated. The results of measuring the device characteristics are described.

The structural formulae of the organic compounds used in the light-emitting device 1 and the comparative light-emitting device 2 are shown below.

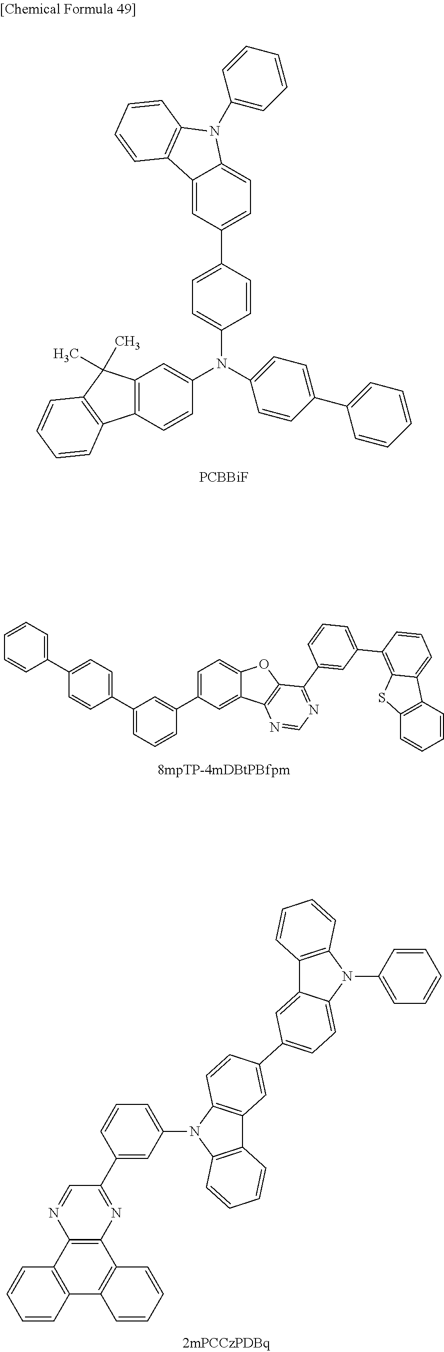

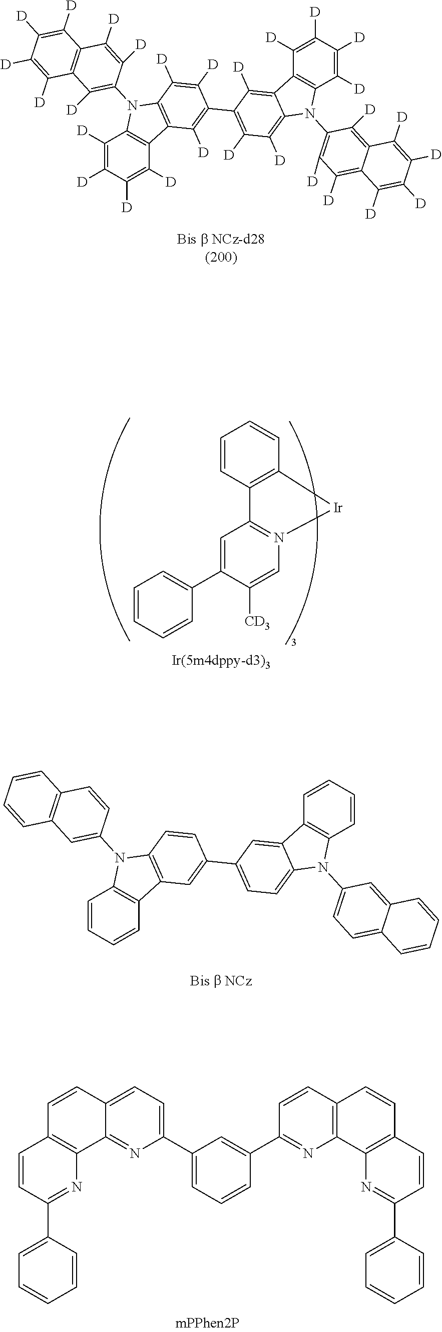

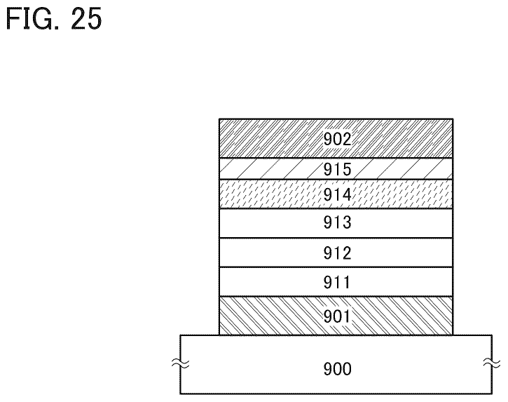

In each of the light-emitting devices, as illustrated in FIG. 25, a hole-injection layer 911, a hole-transport layer 912, a light-emitting layer 913, an electron-transport layer 914, and an electron-injection layer 915 are stacked in this order over a first electrode 901 formed over a glass substrate 900, and a second electrode 902 is stacked over the electron-injection layer 915.

### <Fabrication Method of Light-Emitting Device 1>

Indium tin oxide containing silicon oxide (ITSO) was formed by a sputtering method over the glass substrate 900 to a thickness of 70 nm, so that the first electrode 901 as a transparent electrode was formed. Note that the electrode area was set to 4 mm² (2 mm×2 mm).

Next, in pretreatment for fabricating the light-emitting device over the substrate, the surface of the substrate was washed with water, and baking was performed at 200° C. for one hour. Then, the substrate was transferred into a vacuum evaporation apparatus where the internal pressure was reduced to approximately 1×10⁻⁴ Pa, and vacuum baking was performed at 170° C. for 30 minutes in a heating chamber of the vacuum evaporation apparatus. After that, natural cooling was performed.

Then, the substrate provided with the first electrode 901 was fixed to a substrate holder provided in the vacuum evaporation apparatus such that the surface on which the first electrode 901 was formed faced downward. The hole-injection layer 911 was formed to a thickness of 10 nm over the first electrode 901 by co-evaporation of N-(biphenyl-4-yl)-N-[4-(9-phenyl-9H-carbazol-3-yl)phenyl]-9,9-dimethyl-9H-fluoren-2-amine (abbreviation: PCBBiF) and an electron acceptor material containing fluorine and having a molecular weight of 672 (OCHD-003) at the weight ratio of 1:0.03.

Next, PCBBiF was deposited by evaporation to a thickness of 50 nm over the hole-injection layer 911, whereby the hole-transport layer 912 was formed.

Subsequently, over the hole-transport layer 912, by an evaporation method using resistance heating, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm), BisβNCz-d28, and tris{2-[5-(methyl-d₃)-4-phenyl-2-pyridinyl-N]phenyl-κC}iridium(III) (abbreviation: Ir(5m4dppy-d3)₃) were deposited by co-evaporation to a thickness of 40 nm such that the weight ratio of 8mpTP-4mDBtPBfpm to BisβNCz-d28 to Ir(5m4dppy-d3)₃ was 0.5:0.5:0.1, so that the light-emitting layer 913 was formed.

Next, over the light-emitting layer 913, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 10 nm, and then 2,2′-(1,3-phenylene)bis(9-phenyl-1,10-phenanthroline) (abbreviation: mPPhen2P) was deposited by evaporation to a thickness of 20 nm, whereby the electron-transport layer 914 was formed.

Then, over the electron-transport layer 914, lithium fluoride (LiF) was deposited by evaporation to a thickness of 1 nm, whereby the electron-injection layer 915 was formed.

Subsequently, over the electron-injection layer 915, aluminum (Al) was deposited by evaporation to a thickness of 200 nm, whereby the second electrode 902 was formed.

### <Fabrication Method of Comparative Light-Emitting Device 2>

The comparative light-emitting device 2 is different from the light-emitting device 1 in that BisβNCz-d28 used for the light-emitting layer 913 was replaced with 9,9′-di(2-naphthyl)-9H,9′H-3,3′-bicarbazole (abbreviation: BisβNCz), which is a comparative organic compound obtained by replacing all deuteriums contained in BisβNCz-d28 with protium. That is, in the comparative light-emitting device 2, 8mpTP-4mDBtPBfpm, BisβNCz, and Ir(5m4dppy-d3)₃ were deposited by co-evaporation to a thickness of 40 nm such that the weight ratio of 8mpTP-4mDBtPBfpm to BisβNCz to Ir(5m4dppy-d3)₃ was 0.5:0.5:0.1, whereby the light-emitting layer 913 was formed. Components other than the light-emitting layer 913 of the comparative light-emitting device 2 were fabricated in a manner similar to those of the light-emitting device 1.

The structures of the light-emitting device 1 and the comparative light-emitting device 2 are listed in the following table.

TABLE 1

| | Thickness | | Light-emitting device 1 | Comparative light-emitting device 2 |
|---|---|---|---|---|
| Second electrode | 200 | nm | Al | |
| Electron-injection layer | 1 | nm | LiF | |
| Electron-transport layer | 20 | nm | mPPhen2P | |
| | 10 | nm | 2mPCCzPDBq | |
| Light-emitting layer | 40 | nm | 8mpTP- | 8mpTP- |
| | | | 4mDBtPBfpm:BisβNCz- | 4mDBtPBfpm:BisβNCz:Ir(5m4dppy-d3)₃ |
| | | | d28:Ir(5m4dppy-d₃)₃ | (0.5:0.5:0.1)(weight ratio) |
| | | | (0.5:0.5:0.1)(weight ratio) | |

| | | | |
|---|---|---|---|
| Hole-transport layer | 50 | nm | PCBBiF |
| Hole-injection layer | 10 | nm | PCBBiF:OCHD-003 (1:0.03) |
| First electrode | 70 | nm | ITSO |

### <Characteristics of Light-Emitting Devices>

The light-emitting devices were sealed using a glass substrate in a glove box containing a nitrogen atmosphere so as not to be exposed to the air (a sealing material was applied to surround the devices and UV treatment and heat treatment at 80° C. for one hour were performed at the time of sealing). Then, the characteristics of the light-emitting devices were measured.

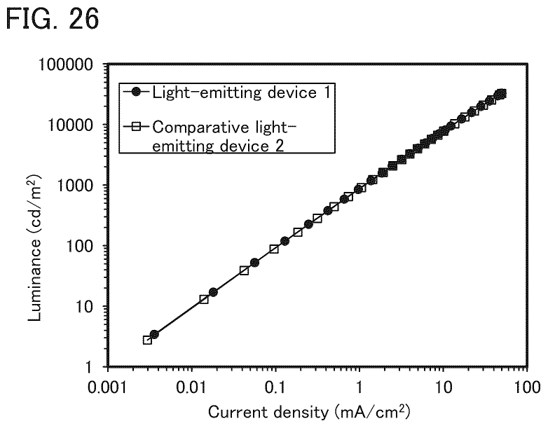

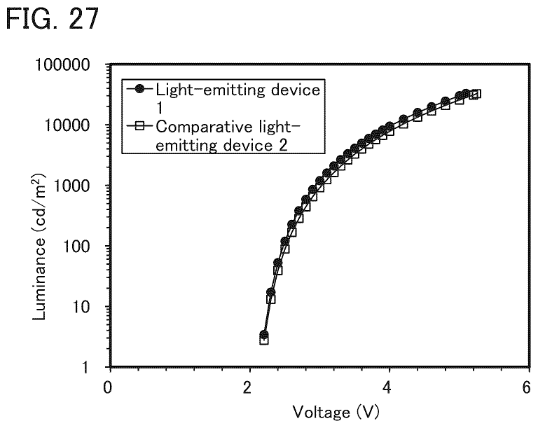

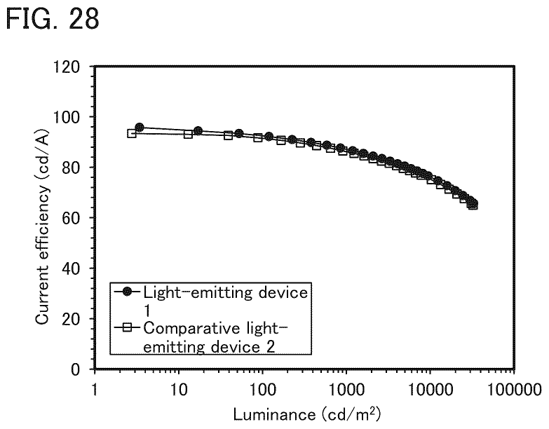

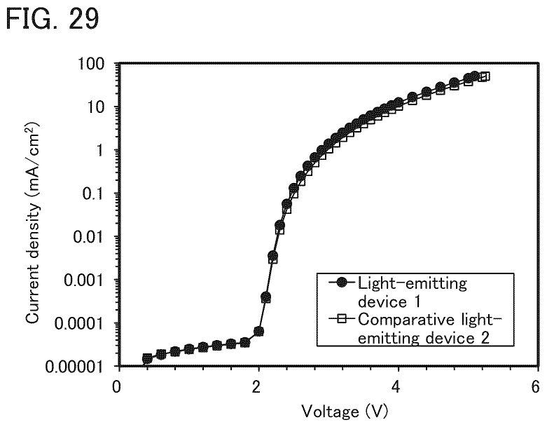

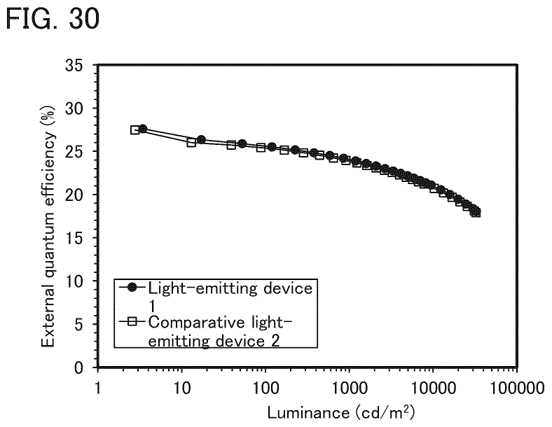

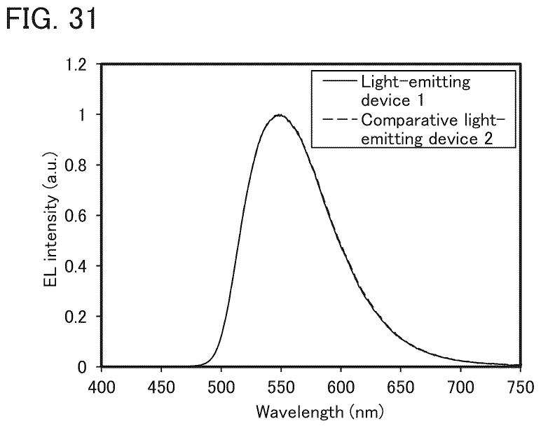

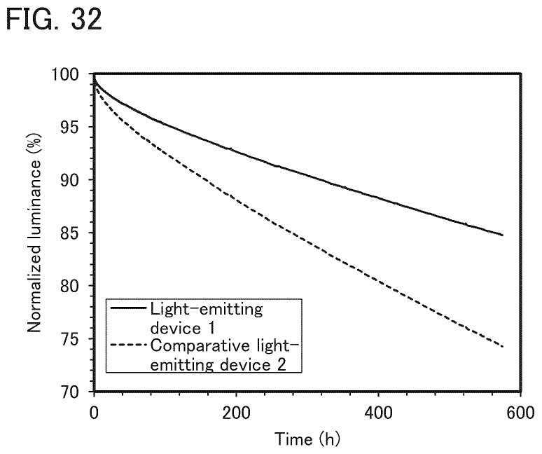

FIG. 26 shows the luminance-current density characteristics of the light-emitting devices. FIG. 27 shows the luminance-voltage characteristics of the light-emitting devices. FIG. 28 shows the current efficiency-luminance characteristics of the light-emitting devices. FIG. 29 shows the current density-voltage characteristics of the light-emitting devices. FIG. 30 shows the external quantum efficiency-luminance characteristics of the light-emitting devices. FIG. 31 shows the electroluminescence spectra of the light-emitting devices. Moreover, FIG. 32 shows a luminance change over driving time when the light-emitting devices were driven at a constant current of 2 mA (50 mA/cm²).

The following table shows the main characteristics of the light-emitting devices at a luminance of approximately 1000 cd/m². Note that the luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 2

| | | | | | | | | External |
|---|---|---|---|---|---|---|---|---|
| | | | Current | | | | Current | quantum |
| | Voltage | Current | density | Chroma- | Chroma- | Luminance | efficiency | efficiency |
| | (V) | (mA) | (mA/cm²) | ticity x | ticity y | (cd/m²) | (cd/A) | (%) |
| Light-emitting | 2.90 | 0.0389 | 0.97 | 0.402 | 0.579 | 852 | 87.6 | 24.2 |
| device 1 | | | | | | | | |
| Comparative | 3.00 | 0.0421 | 1.05 | 0.403 | 0.578 | 910 | 86.5 | 23.9 |
| light-emitting | | | | | | | | |
| device 2 | | | | | | | | |

FIGS. 26 to 32 and the above table reveal that the light-emitting device 1 and the comparative light-emitting device 2 exhibit green light emission derived from Ir(5m4dppy-d3)₃. It was also found that the initial characteristics of the light-emitting device 1 and the comparative light-emitting device 2 were the same.

As shown in FIG. 32, the comparison between the light-emitting device 1 and the comparative light-emitting device 2 reveals that the light-emitting device 1 has a smaller change in luminance over driving time and has a longer lifetime. Thus, it was found that, when BisβNCz-d28, which is the organic compound of one embodiment of the present invention, is used for the light-emitting layer, the light-emitting device can have a longer lifetime than the case where BisβNCz is used for the light-emitting layer. Since BisβNCz-d28 includes deuterium, it can be said that the lifetime of the light-emitting device including BisβNCz-d28 in its light-emitting layer is longer than the light-emitting device including BisβNCz in its light-emitting layer because BisβNCz-d28 has higher stability than BisβNCz that does not include deuterium.

The above results show that the use of the organic compound of one embodiment of the present invention enables fabrication of a light-emitting device with a long lifetime and high reliability.

### Example 3

### Synthesis Example 2

In this synthesis example, a synthesis method of 9-(1-naphthyl-2,3,4,5,6,7,8-d₇)-9′-(2-naphthyl-1,3,4,5,6,7,8-d₇)-9H,9′H-3,3′-bicarbazole-1,1′,2,2′,4,4′,5,5′,6,6′,7,7′,8,8′-d₁₄ (abbreviation: βNCCαN-d28), which is the organic compound represented by Structural Formula (287) in Embodiment 1, will be described. The structure of βNCCαN-d28 is shown below.

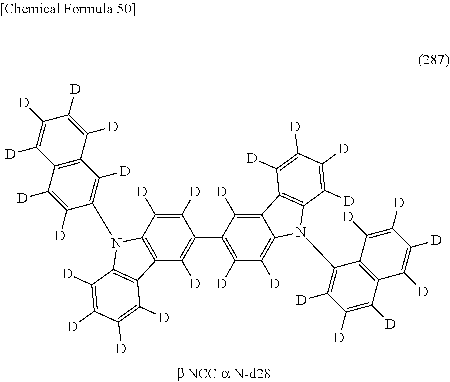 [SMILES: [2H]c1c([2H])c([2H])c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c([2H])c([2H])c([2H])c([2H])c56)c([2H])c([2H])c43)c([2H])c([2H])c2c1[2H]]

### Step 1: Synthesis of 6-bromo-9-(1-naphthyl-2,3,4,5,6,7,8-d₇)-9H-carbazole-1,2,3,4,5,7,8-d₇

Into a 300 mL conical flask were put 3.7 g (12 mmol) of 9-(1-naphthyl-2,3,4,5,6,7,8-d₇)-9H-carbazole-1,2,3,4,5,7,8-d₇, 55 mL of toluene, and 24 mL of ethyl acetate (AcOEt). After the reaction system was stirred and dissolved, 2.2 g (12 mmol) of N-bromosuccinimide (NBS) was added and stirring was performed at room temperature for 21 hours. After that, pure water was added to the reaction system, the mixed solution was transferred to a separatory funnel, and extraction was performed with toluene. The organic layer was dehydrated using magnesium sulfate, and after a predetermined time elapsed, gravity filtration was performed using a pleated filter paper. The filtrate was concentrated to give 4.9 g of a brown viscous solid containing the target substance. It was considered that the yield of the brown viscous solid was 100% and the solid was used for a reaction in Step 2. Synthesis Scheme (s2-1) of Step 1 is shown below.

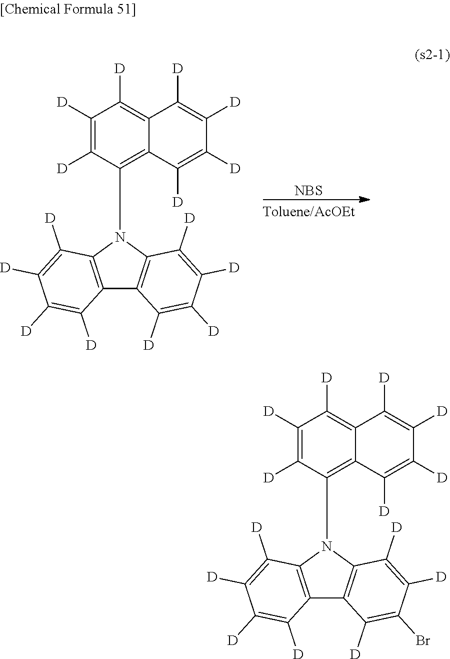

### Step 2: Synthesis of βNCCαN-d28

Into a 200 mL three-neck flask were put 1.6 g (4.0 mmol) of 6-bromo-9-(1-naphthyl-2,3,4,5,6,7,8-d₇)-9H-carbazole-1,2,3,4,5,7,8-d₇ synthesized in Step 1, 1.6 g (4.0 mmol) of 6-(4,4,5,5-tetramethyl-1,3,2-dioxaborolane)-9-(2-naphthyl-1,3,4,5,6,7,8-d₇)-9H-carbazole-1,2,3,4,5,7,8-d₇, 1.7 g (12 mmol) of potassium carbonate (abbreviation: K₂CO₃), 29 mg (95 μmol) of tris(o-tolyl)phosphine (abbreviation: P(o-tolyl)₃), 6.2 mL of water, 5 mL of ethanol, and 20 mL of toluene. This mixture was degassed by being stirred under reduced pressure. Then, the atmosphere in the flask was replaced with nitrogen. After that, the mixture was heated at 60° C. To this mixture, 23 mg (0.10 mmol) of palladium(II) acetate (abbreviation: Pd(OAc)₂) was added, and the mixture was stirred at 90° C. for three hours. Then, water was added to the reaction system, the mixed solution was transferred to a separatory funnel, and extraction was performed with toluene. The organic layer was dehydrated using magnesium sulfate, and after a predetermined time elapsed, gravity filtration was performed using a pleated filter paper. The filtrate was concentrated to give 3.1 g of a gray solid. This solid was purified by silica gel column chromatography (developing solvent: toluene) to give a white solid. This solid was purified by high performance liquid chromatography (mobile phase: chloroform) to give 0.73 g of the target white solid in a yield of 29%. Synthesis Scheme (s2-2) of βNCCαN-d28 is shown below.

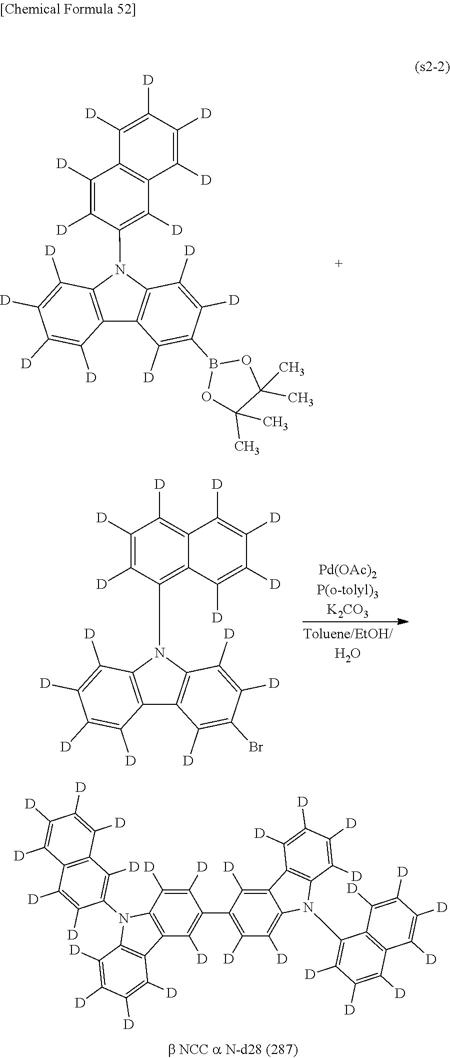

By a train sublimation method, 2.6 g of the white solid obtained by a similar method was purified. In the sublimation purification, the solid was heated at 300° C. for 27 hours under a pressure of 2.77 Pa with an argon flow rate of 10 mL/min. After the purification by sublimation, 0.92 g of the target white solid was obtained at a collection rate of 35%.

The molecular weight of the white solid obtained in Step 2 was measured by LC/MS analysis. As a result, m/z 612 was observed with respect to the calculated mass 612 of the target substance. This indicates that βNCCαN-d28 was obtained.

### <Measurement of Physical Properties>

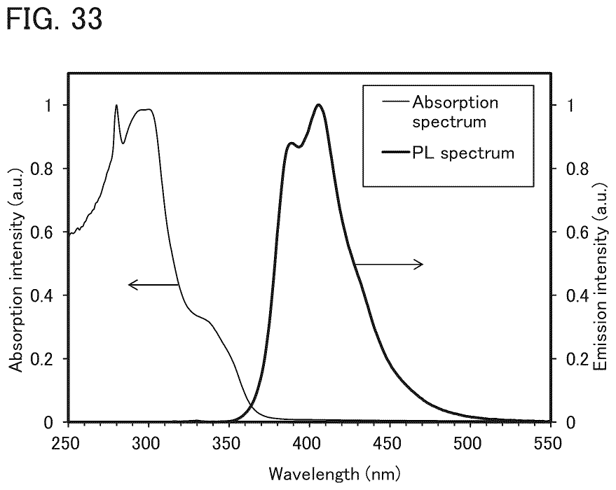

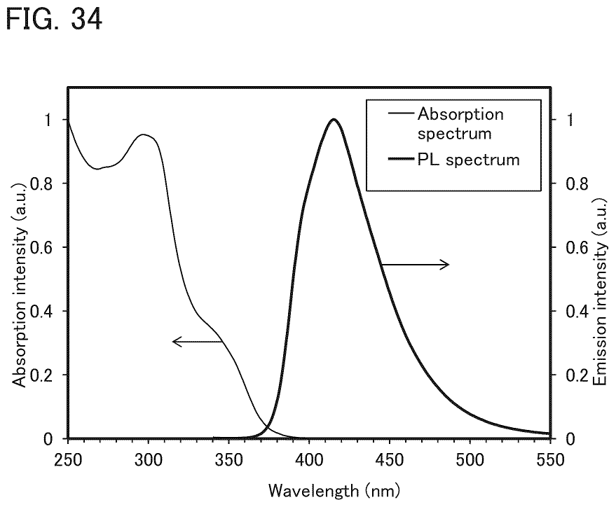

Next, as in Example 1, an absorption spectrum and a PL spectrum of each of a toluene solution of βNCCαN-d28 and a solid thin film of βNCCαN-d28 were measured. FIG. 33 shows the measurement results of the absorption spectrum and the PL spectrum of the toluene solution of βNCCαN-d28, and FIG. 34 shows the measurement results of the absorption spectrum and the PL spectrum of the solid thin film of βNCCαN-d28.

As shown in FIG. 33, the absorption spectrum of the toluene solution of βNCCαN-d28 exhibited an absorption peak at around 340 nm and a shoulder peak at around 350 nm. The results reveal that the solution of βNCCαN-d28 does not absorb light in a region with a wavelength greater than or equal to 400 nm, which indicates that the material of the present invention can be suitably used for a light-emitting device. In addition, as shown in FIG. 33, an emission peak was observed at around 406 nm (excitation wavelength: 300 nm) in the PL spectrum of the toluene solution of βNCCαN-d28.

As shown in FIG. 34, the absorption spectrum of the thin film of βNCCαN-d28 exhibited the maximum absorption peak at around 295 nm and a shoulder peak at around 335 nm. FIG. 34 reveals that βNCCαN-d28 does not absorb light in a region with a wavelength greater than or equal to 400 nm also in the thin film, which indicates that the material of the present invention can be suitably used for a light-emitting device. As shown in FIG. 34, an emission peak was observed at around 415 nm (excitation wavelength: 330 nm) in the PL spectrum of the thin film of βNCCαN-d28.

The TG-DTA of βNCCαN-d28 was performed. For the measurement, a high-sensitivity differential type differential thermogravimeter (STA 2500 Regulus produced by NETZSCH Japan K.K.) was used. The measurement was performed under an atmospheric pressure at a temperature rising rate of 10° C./min under a nitrogen stream (flow rate: 200 mL/min).

In TG-DTA of βNCCαN-d28, it was revealed that the temperature (i.e., the sublimation or decomposition temperature) at which the weight obtained by thermogravimetry measurement was reduced by 5% of the weight at the beginning of the measurement was 466° C. under atmospheric pressure. The result shows that the sublimation or decomposition temperature of βNCCαN-d28 under atmospheric pressure is 466° C. and has high heat resistance.

Differential scanning calorimetry (DSC) measurement of βNCCαN-d28 was performed with DSC8500 manufactured by PerkinElmer, Inc. The DSC measurement was performed in the following manner: the temperature was raised from −10° C. to 350° C. at a temperature rising rate of 40° C./min and held for three minutes; then the temperature was decreased to −10° C. at a temperature decreasing rate of 100° C./min and held for three minutes. This operation was performed twice in succession. Subsequently, the DSC measurement was performed in the following manner: the temperature was raised from −10° C. to 350° C. at a temperature rising rate of 50° C./min and held for three minutes; then, the temperature was decreased to −10° C. at a temperature decreasing rate of 100° C./min. This operation was performed once.

The DSC measurement result of the temperature rising process measured at the second temperature rising rate of 40° C./min revealed that T_{g} of βNCCαN-d28 was 138° C. It was found that the use of the organic compound of one embodiment of the present invention for an organic semiconductor element such as a light-emitting device can increase the heat resistance of the organic semiconductor element.

### Example 4

### Synthesis Example 3

In this synthesis example, a synthesis method of 9,9′-di(1-naphthyl-2,3,4,5,6,7,8-d₇)-9H,9′H-3,3′-bicarbazole-1,1′,2,2′,4,4′,5,5′,6,6′,7,7′,8,8′-d₁₄ (abbreviation: BisαNCz-d28), which is the organic compound represented by Structural Formula (217) in Embodiment 1, will be described. The structure of BisαNCz-d28 is shown below.

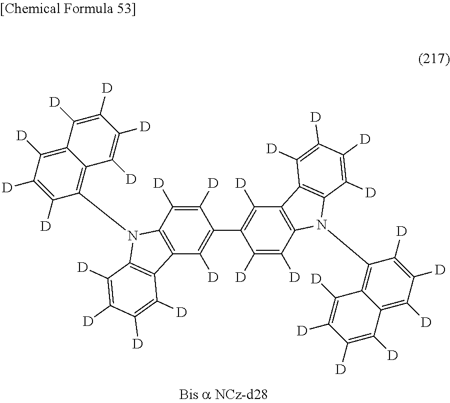 [SMILES: [2H]c1c([2H])c([2H])c2c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c([2H])c([2H])c([2H])c([2H])c56)c([2H])c([2H])c43)c([2H])c([2H])c([2H])c2c1[2H]]

### Step 1: Synthesis of 6-(4,4,5,5-tetramethyl-1,3,2-dioxaborolane)-9-(1-naphthyl-2,3,4,5,6,7,8-d₇)-9H-carbazole-1,2,3,4,5,7,8-d₇

Into a 200 mL three-neck flask were put 3.1 g (8.1 mmol) of 6-bromo-9-(1-naphthyl-2,3,4,5,6,7,8-d₇)-9H-carbazole-1,2,3,4,5,7,8-d₇, 2.3 g (8.9 mmol) of bis(pinacolato)diboron, 2.4 g (24 mmol) of potassium acetate (AcOK), and 40 mL of dioxane. This mixture was degassed by being stirred under reduced pressure. Then, the atmosphere in the flask was replaced with nitrogen. After that, the mixture was heated at 60° C. To the mixture was added 0.15 g (0.16 mmol) of [1,1′-bis(diphenylphosphino)ferrocene]palladium(II) dichloride dichloromethane adduct (abbreviation: Pd(dppf)₂Cl₂·CH₂Cl₂), and the mixture was stirred at 110° C. for seven hours. After the mixture was cooled to room temperature, toluene and pure water were added and solution separation was performed. The organic layer was dehydrated with magnesium sulfate and suction filtration was performed. A black oily substance obtained by concentration of the filtrate was purified by silica gel column chromatography (developing solvent: toluene) to give 2.4 g of a target pale yellow solid in a yield of 77%. Synthesis Scheme (s3-1) of Step 1 is shown below.

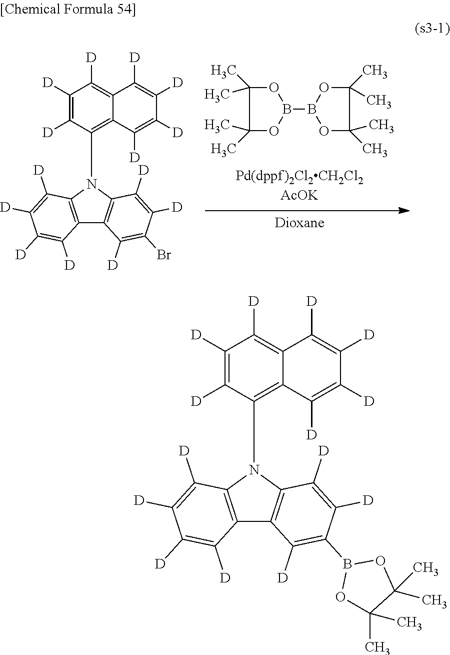

### Step 2: Synthesis of BisαNCz-d28

Into a 200 mL three-neck flask were put 2.4 g (6.2 mmol) of 6-(4,4,5,5-tetramethyl-1,3,2-dioxaborolane)-9-(1-naphthyl-2,3,4,5,6,7,8-d₇)-9H-carbazole-1,2,3,4,5,7,8-d₇, 2.3 g (6.0 mmol) of 6-bromo-9-(1-naphthyl-2,3,4,5,6,7,8-d₇)-9H-carbazole-1,2,3,4,5,7,8-d₇, 2.6 g (19 mmol) of potassium carbonate (abbreviation: K₂CO₃), 58 mg (0.19 mmol) of tris(o-tolyl)phosphine (abbreviation: P(o-tolyl)₃), 9.0 mL of water, 6 mL of ethanol, and 30 mL of toluene. This mixture was degassed by being stirred under reduced pressure. Then, the atmosphere in the flask was replaced with nitrogen. After that, the mixture was heated at 60° C. To this mixture was added 18 mg (80 μmol) of palladium(II) acetate (abbreviation: Pd(OAc)₂), and stirring was performed at 90° C. for six hours. After the mixture was air-cooled to room temperature, toluene and pure water were added and solution separation was performed. The organic layer was dehydrated with magnesium sulfate, and after a predetermined time elapsed, this mixture was subjected to gravity filtration with a pleated filter paper. The filtrate was concentrated to give 3.7 g of white solid. The solid was purified by high performance liquid chromatography (mobile phase: chloroform) to give 2.8 g of the target white solid in a yield of 74%. Synthesis Scheme (s3-2) of BisαNCz-d28 is shown below.

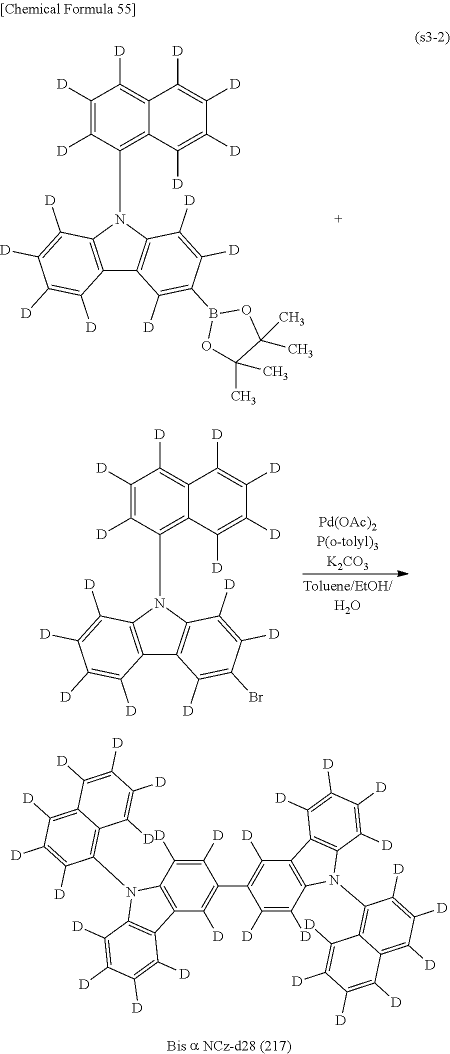

By a train sublimation method, 2.2 g of the obtained white solid was purified. In the sublimation purification, the solid was heated at 280° C. for 40 hours under a pressure of 4.30 Pa with an argon flow rate of 10 mL/min. After the purification by sublimation, 1.4 g of the target white solid was obtained at a collection rate of 64%.

The molecular weight of the white solid obtained in Step 2 was measured by LC/MS analysis. As a result, m/z 612 was observed with respect to the calculated mass 612 of the target substance. This indicates that BisαNCz-d28 was obtained.

### <Measurement of Physical Properties>

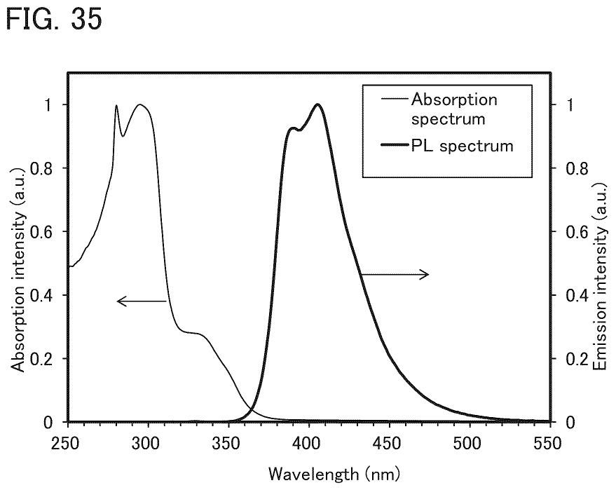

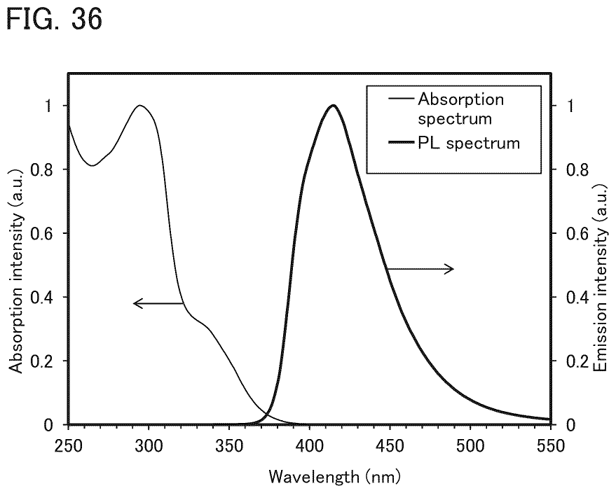

Next, as in Example 1, an absorption spectrum and a PL spectrum of each of a toluene solution of BisαNCz-d28 and a solid thin film of BisαNCz-d28 were measured. FIG. 35 shows the measurement results of the absorption spectrum and the PL spectrum of the toluene solution of BisαNCz-d28 and FIG. 36 shows the measurement results of the absorption spectrum and the PL spectrum of the solid thin film of BisαNCz-d28.

As shown in FIG. 35, the absorption spectrum of the toluene solution of BisαNCz-d28 exhibited an absorption peak at around 335 nm and a shoulder peak at around 350 nm. The results reveal that the solution of BisαNCz-d28 does not absorb light in a region with a wavelength greater than or equal to 400 nm, which indicates that the material of the present invention can be suitably used for a light-emitting device. In addition, as shown in FIG. 35, an emission peak was observed at around 405 nm (excitation wavelength: 300 nm) in the PL spectrum of the toluene solution of BisαNCz-d28.

As shown in FIG. 36, the absorption spectrum of the thin film of BisαNCz-d28 exhibited the maximum absorption peak at around 295 nm and a shoulder peak at around 335 nm. The results reveal that BisαNCz-d28 does not absorb light in a region with a wavelength greater than or equal to 400 nm also in the thin film, which indicates that the material of the present invention can be suitably used for a light-emitting device. In addition, as shown in FIG. 36, the PL spectrum of the thin film of BisαNCz-d28 exhibited an emission peak at around 415 nm (excitation wavelength: 330 nm).

When TG-DTA of BisαNCz-d28 was performed in a manner similar to that in Example 3, it was revealed that the temperature (i.e., the sublimation or decomposition temperature) at which the weight obtained by thermogravimetry measurement was reduced by 5% of the weight at the beginning of the measurement was 465° C. under atmospheric pressure. The results reveal that the sublimation or decomposition temperature of BisαNCz-d28 under atmospheric pressure is 465° C. and has high heat resistance.

The DSC measurement of BisαNCz-d28 was performed in a manner similar to that in Example 3. The DSC measurement result of the temperature rising process measured at the second temperature rising rate of 40° C./min revealed that T_{g} of BisαNCz-d28 was 150° C. When the organic compound of one embodiment of the present invention is used for an organic semiconductor element such as a light-emitting device, it was found that the heat resistance of the organic semiconductor element can be increased.

### Example 5

### Synthesis Example 4

In this synthesis example, a synthesis method of 9,9′-di(10-phenanthryl-1,2,3,4,5,6,7,8,9-d₉)-9H,9′H-3,3′-bicarbazole-1,1′,2,2′,4,4′,5,5′,6,6′,7,7,8,8′-d₄ (abbreviation: BisPnCz-d32), which is the organic compound of the present invention represented by Structural Formula (260) in Embodiment 1, will be described. The structure of BisPnCz-d32 is shown below.

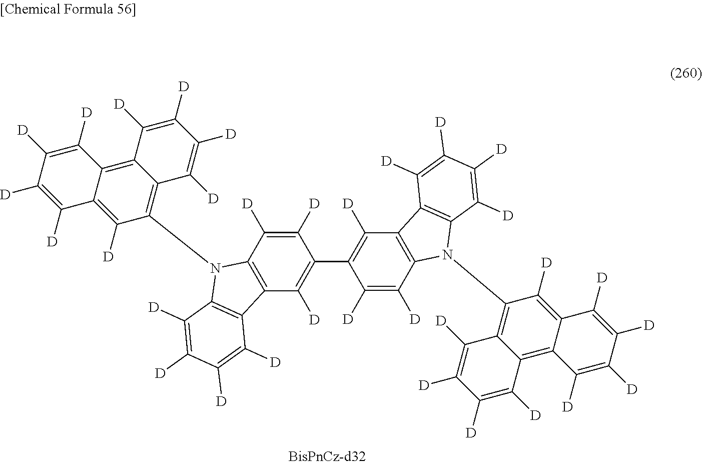 [SMILES: [2H]c1c([2H])c([2H])c2c(c1[2H])c([2H])c(-n1c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c5c([2H])c([2H])c([2H])c([2H])c5c5c([2H])c([2H])c([2H])c([2H])c45)c([2H])c([2H])c31)c1c([2H])c([2H])c([2H])c([2H])c12]

### <Synthesis of BisPnCz-d32>

Into a 200 mL three-neck flask were put 446 mg (1.3 mmol) of 3,3′-bi-9H-carbazole-1,1′,2,2′,4,4′,5,5′,6,6′,7,7′,8,8′-d₁₄, 1.2 g (4.5 mmol) of 10-bromophenanthrene-1,2,3,4,5,6,7,8,9-d₉, 1.1 g (5.0 mmol) of tripotassium phosphate (abbreviation: K₃PO₄), 85 pL (0.081 mg, 0.71 mmol) of (1R, 2R)-(−)-1,2-cyclohexanediamine, and 15 mL of dioxane. The mixture was degassed by being stirred under reduced pressure. Then, the atmosphere in the flask was replaced with nitrogen. After that, the mixture was heated at 80° C. To this reaction solution, 0.071 g (0.37 mmol) of copper(I) iodide (abbreviation: CuI) was added, and stirring was performed at 130° C. for four hours. Water was added to the reaction system, the mixed solution was transferred to a separatory funnel, and extraction was performed with dichloromethane. The organic layer was dehydrated with magnesium sulfate, and after a predetermined time elapsed, gravity filtration was performed with a pleated filter paper. The filtrate was concentrated to give a brown viscous solid containing the target substance. This brown solid was purified by silica gel column chromatography with the developing solvent of hexane and dichloromethane, whose ratio was changed from 3:1 to 1:1 (hexane: dichloromethane) to perform a gradient, whereby 60 mg of the target white solid in a yield of 6% is obtained. Synthesis Scheme (s4-1) of BisPnCz-d32 is shown below.

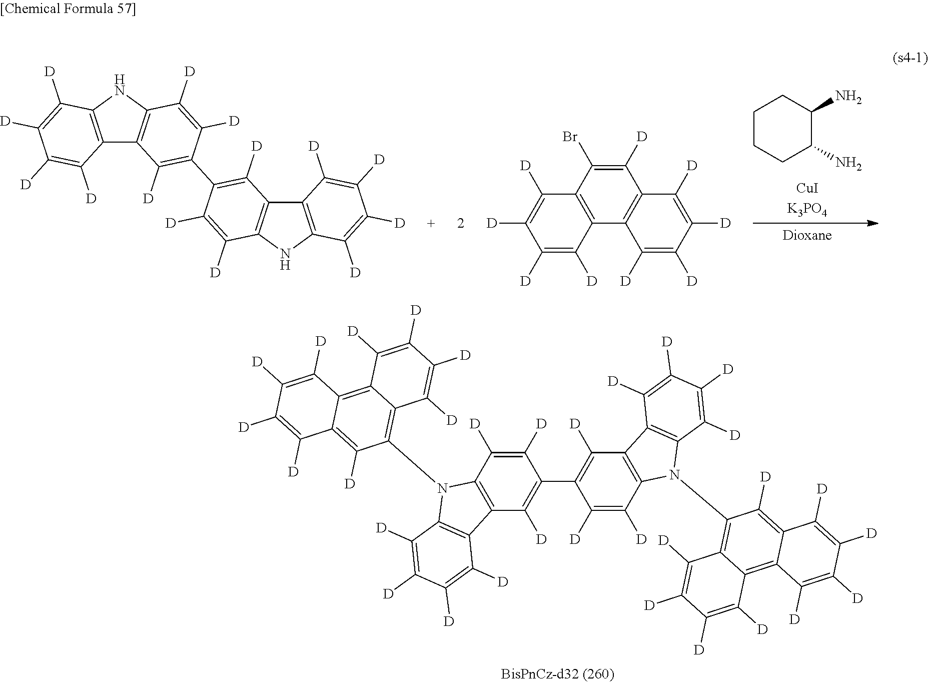

The molecular weight of the obtained white solid was measured by LC/MS analysis. As a result, m/z 716 was observed with respect to the calculated mass 716 of the target substance. This indicates that BisPnCz-d32 was obtained.

### Example 6

### Synthesis Example 5

In this synthesis example, a synthesis method of 9,9′-di(11-triphenylenyl)-9H,9′H-3,3′-bicarbazole-1,1′,2,2′,4,4′,5,5′,6,6′,7,7,8,8′-d₁₄ (abbreviation: BisTpCz-d14), which is the organic compound of the present invention represented by Structural Formula (270) in Embodiment 1, will be described. The structure of BisTpCz-d14 is shown below.

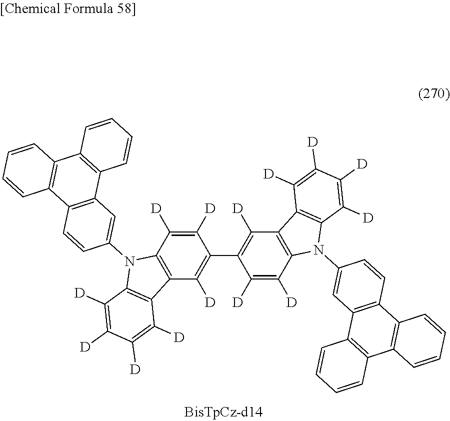 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c5ccccc5c5ccccc5c4c3)c([2H])c([2H])c1n2-c1ccc2c3ccccc3c3ccccc3c2c1]

### Step 1: Synthesis of 9-(2-triphenylenyl)-9H-carbazole-1,2,3,4,5,6,7,8-d₈

Into a 200 mL three-neck flask were put 4.8 g (16 mmol) of carbazole-1,2,3,4,5,6,7,8-d₈ (Angene International Limited, product number: AGOOBZO3), 2.8 g (16 mmol) of 2-bromotriphenylene, 1.1 g (8.0 mmol) of potassium carbonate, 0.10 g (0.39 mmol) of 18-crown-6, and 68 mL of toluene. This mixture was degassed by being stirred under reduced pressure. Then, the atmosphere in the flask was replaced with nitrogen. After that, the mixture was heated at 60° C. To this reaction solution were added 0.7 mL (0.35 mmol) of tri-tert-butylphosphine (abbreviation: P(tBu)₃) (10 wt % hexane solution) and 0.14 g (0.16 mmol) of tris(dibenzylideneacetone)dipalladium(0) (abbreviation: Pd₂(dba)₃), and stirring was performed at 130° C. for two hours. The following day, this reaction solution was heated to 90° C., 0.5 mL (0.25 mmol) of tri-tert-butylphosphine (abbreviation: P(tBu)₃) (10 wt % hexane solution) and 0.12 g (0.13 mmol) of Pd₂(dba)₃ were then added, and stirring was performed at 130° C. for six hours. Furthermore, the day after, the reaction solution was heated to 90° C., 0.14 g (0.15 mmol) of Pd₂(dba)₃ was then added, and stirring was performed at 130° C. for six hours. Toluene was added to this reaction solution, stirring was performed at 80° C., and the mixed solution was subjected to suction filtration through alumina, Celite, and Florisil. The obtained filtrate was concentrated to give a toluene solution containing the target substance, ethanol was then added thereto, and ultrasonic irradiation was performed, whereby a white solid was precipitated. The white solid was collected by suction filtration to give 3.8 g of the target white solid in a yield of 57%. Synthesis Scheme (s5-1) of Step 1 is shown below.

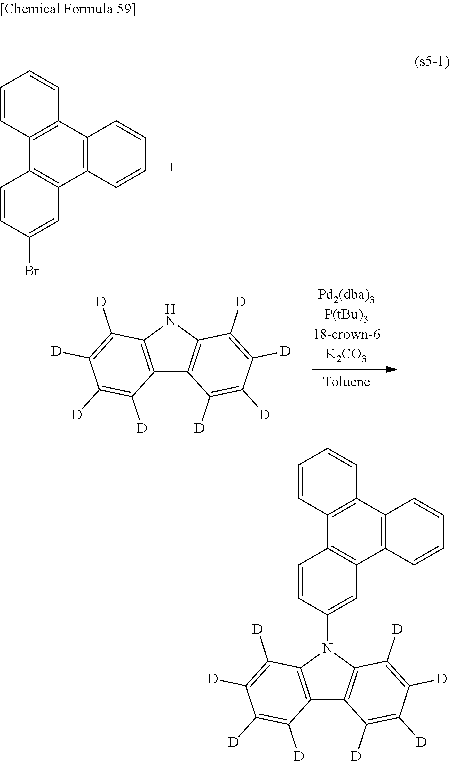 [SMILES: Brc1ccc2c3ccccc3c3ccccc3c2c1 ; [2H]c1c([2H])c([2H])c2c([nH]c3c([2H])c([2H])c([2H])c([2H])c32)c1[2H] ; [2H]c1c([2H])c([2H])c2c(c1[2H])c1c([2H])c([2H])c([2H])c([2H])c1n2-c1ccc2c3ccccc3c3ccccc3c2c1]

The molecular weight of the white solid obtained in Step 1 was measured by LC/MS, in which case m/z 402 (a proton adduct of 9-(2-triphenylenyl)-9H-carbazole-1,2,3,4,5,6,7,8-d₈) was observed with respect to the calculated mass 401 of the target substance. The results reveal that 9-(2-triphenylenyl)-9H-carbazole-1,2,3,4,5,6,7,8-d₈ is obtained.

### Step 2: Synthesis of 6-bromo-9-(2-triphenylenyl)-9H-carbazole-1,2,3,4,5,7,8-d₇)

Into a 300 mL three-neck flask equipped with a 100 mL dropping funnel were put 3.8 g (9.4 mmol) of 9-(2-triphenylenyl)-9H-carbazole-1,2,3,4,5,6,7,8-ds synthesized in Step 1 and 80 mL of dimethylformamide (DMF), and 9-(2-triphenylenyl)-9H-carbazole-1,2,3,4,5,6,7,8-d₈ was dissolved by being heated at approximately 60° C. Into the 100 mL dropping funnel was added 16 mL of a DMF solution in which 1.7 g (9.4 mmol) of N-bromosuccinimide (NBS) was dissolved. While the mixture solution in the three-neck flask was stirred at room temperature, the DMF solution of NBS was dropped into the mixture solution for approximately 30 minutes. After a predetermined time elapsed, pure water was added to the reaction system, so that a white solid was precipitated. The precipitated white solid was collected by suction filtration to give 4.3 g of the target white solid in a yield of 97%. Synthesis Scheme (s5-2) of Step 2 is shown below.

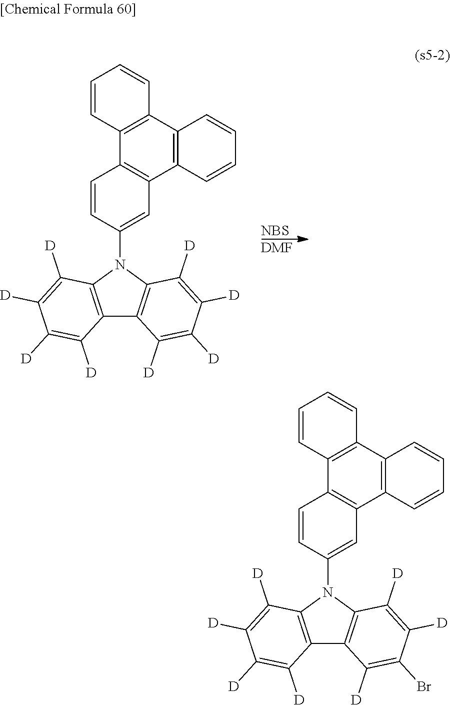

The molecular weight of the white solid obtained in Step 2 was measured by LC/MS, in which case m/z 478 was observed with respect to the calculated mass 478 of the target substance. The results reveal that 6-bromo-9-(2-triphenylenyl)-9H-carbazole-1,2,3,4,5,7,8-d₇ is obtained.

### Step 3: Synthesis of 6-(4,4,5,5-tetramethyl-1,3,2-dioxaborolane)-9-(2-triphenylenyl)-9H-carbazole-1,2,3,4,5,7,8-d₇

Into a 200 mL three-neck flask were put 2.3 g (4.8 mmol) of 6-bromo-9-(2-triphenylenyl)-9H-carbazole-1,2,3,4,5,7,8-d₇ synthesized in Step 2, 1.4 g (5.3 mmol) of bis(pinacolato)diboron, 1.6 g (16 mmol) of potassium acetate (AcOK), and 73 mL of dioxane. This mixture was degassed by being stirred under reduced pressure. Then, the atmosphere in the flask was replaced with nitrogen. After that, the mixture was heated at 60° C. To this reaction mixture was added 50 mg (61 μmol) of [1,1′-bis(diphenylphosphino)ferrocene]palladium(II) dichloride dichloromethane adduct (abbreviation: Pd(dppf)₂Cl₂—CH₂C1₂), and the mixture was stirred at 130° C. for four hours. After the mixture was cooled to room temperature, pure water was added to the reaction system, the mixture was transferred to a separatory funnel, and extraction was performed with toluene. The organic layer was dehydrated with magnesium sulfate, and after a predetermined time elapsed, gravity filtration was performed with a pleated filter paper. A brown oily substance obtained by concentration of the filtrate was purified by silica gel column chromatography (developing solvent: toluene) to give 1.5 g of the target white solid in a yield of 60%. Synthesis Scheme (s5-3) of Step 3 is shown below.

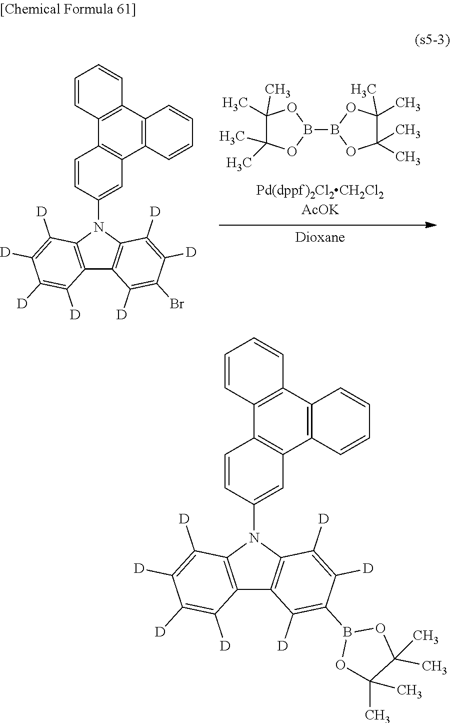

The molecular weight of the white solid obtained in Step 3 was measured by LC/MS, in which case m/z 527 (a proton adduct of the target substance) was observed with respect to the calculated mass 526 of the target substance. The results indicate that 6-(4,4,5,5-tetramethyl-1,3,2-dioxaborolane)-9-(2-triphenylenyl)-9H-carbazole-1,2,3,4,5,7,8-d₇ was obtained.

### Step 4: Synthesis of BisTpCz-d14

Into a 200 mL three-neck flask were put 1.4 g (2.9 mmol) of 6-bromo-9-(2-triphenylenyl)-9H-carbazole-1,2,3,4,5,7,8-d₇ synthesized in Step 2, 1.5 g (2.9 mmol) of 6-(4,4,5,5-tetramethyl-1,3,2-dioxaborolane)-9-(2-triphenylenyl)-9H-carbazole-1,2,3,4,5,7,8-d₇ synthesized in Step 3, 12 mL of an aqueous solution of potassium carbonate (2 mol/L) (abbreviation: K₂CO₃ aq.), 29 mg (95 μmol) of tris(o-tolyl)phosphine (abbreviation: P(o-tolyl)₃, 60 mL of toluene, and 5 mL of ethanol. This mixture was degassed by being stirred under reduced pressure. Then, the atmosphere in the flask was replaced with nitrogen. After that, the mixture was heated at 60° C. To this reaction mixture was added 10 mg (44 μmol) of palladium(II) acetate (abbreviation: Pd(OAc)₂), and stirring was performed at 90° C. for three hours. After the mixture was cooled to room temperature, pure water was added to the reaction system, the mixture was transferred to a separatory funnel, and extraction was performed with toluene. The organic layer was dehydrated with magnesium sulfate, and after a predetermined time elapsed, gravity filtration was performed with a pleated filter paper. The filtrate was concentrated to give 2.9 g of an ocher solid, which was then purified by high performance liquid chromatography (mobile phase: chloroform); thus, 0.53 g of the target white solid was obtained in a yield of 23%. Synthesis scheme (s5-4) of Step 4 is shown below.

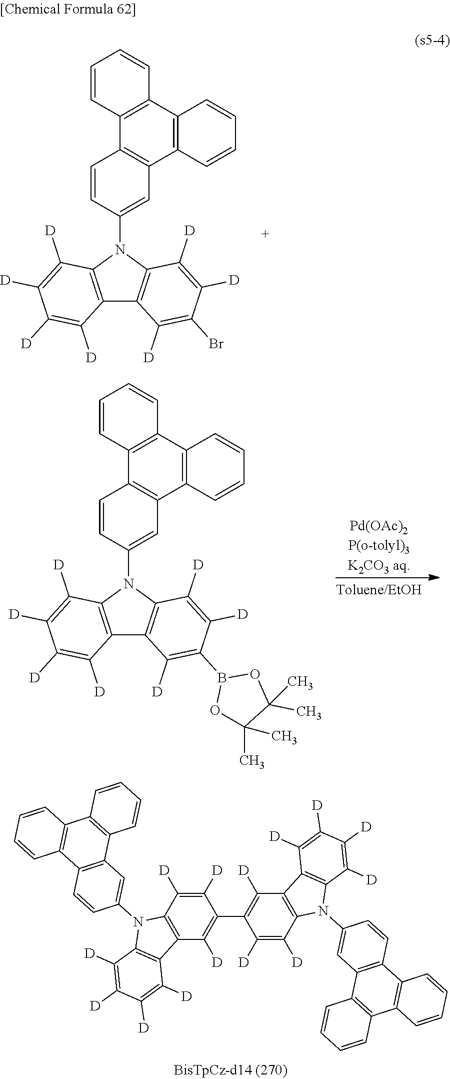

Analysis results by nuclear magnetic resonance (¹H-NMR) spectroscopy of the obtained solid are shown below.

¹H NMR (dichloromethane-d₂, 500 MHz): δ=8.96 (d, J=9.0 Hz, 4H), 8.80-8.75 (m, 6H), 8.65 (d, J=8.0, 2H), 7.97 (d, J=8.5 Hz, 2H), 7.78-7.74 (m, 6H), 7.69 (t, J=8.0 Hz, 2H)

The molecular weight of the obtained white solid was measured by LC/MS, in which case m/z 798 was observed with respect to the calculated mass 798 of the target substance. The results reveal that BisTpCz-d14 is obtained.

### Example 7

### Synthesis Example 6

In this synthesis example, a synthesis method of 9,9′-di(11-triphenylenyl-1,2,3,4,5,6,7,8,9,10,12-d₁₁)-9H,9′H-3,3′-bicarbazole-1,1′,2,2′,4,4′,5,5′,6,6′,7,7′,8,8′-d₁₄ (abbreviation: BisTpCz-d36), which is the organic compound of the present invention represented by Structural Formula (278) in Embodiment 1, will be described. The structure of BisTpCz-d36 is shown below.

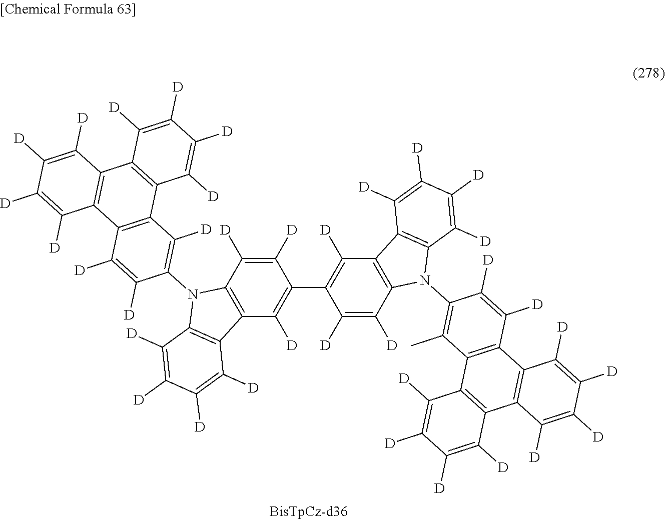 [SMILES: [2H]c1c([2H])c([2H])c2c(c1[2H])c1c([2H])c([2H])c([2H])c([2H])c1c1c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c7c([2H])c([2H])c([2H])c([2H])c7c7c([2H])c([2H])c([2H])c([2H])c7c6c5C)c([2H])c([2H])c43)c([2H])c([2H])c21]

### Synthesis of BisTpCz-d36

Into a 200 mL three-neck flask were put 0.99 g (2.9 mmol) of 3,3′-bi-9H-carbazole-1,1′,2,2′,4,4′,5,5′,6,6′,7,7′,8,8′-d₁₄, 2.0 g (6.3 mmol) of 11-bromotriphenylene-1,2,3,4,5,6,7,8,9,10,12-d₁₁, 2.4 g (12 mmol) of tripotassium phosphate (abbreviation: K₃PO₄), 0.15 mL (0.16 mg, 1.2 mmol) of (1R, 2R)-(−)-1,2-cyclohexanediamine, and 70 mL of dioxane. This mixture was degassed by being stirred under reduced pressure. Then, the atmosphere in the flask was replaced with nitrogen. After that, the mixture was heated at 80° C. Subsequently, 0.29 g (1.5 mmol) of copper(I) iodide (abbreviation: CuI) was added, and the mixture was stirred at 130° C. for eight hours. After the mixture was cooled to room temperature, pure water was added to the reaction system, the mixture was transferred to a separatory funnel, and extraction was performed with dichloromethane. The organic layer was washed with saturated brine, and the organic layer was then dehydrated with magnesium sulfate. After a predetermined time elapsed, gravity filtration was performed with a pleated filter paper. The mixture obtained by concentration of the filtrate was purified by silica gel column chromatography with the developing solvent of hexane and dichloromethane, whose ratio was changed from 3:1 to 2:1 (hexane: dichloromethane) to give 0.7 g of the target white solid in a yield of 30%. Synthesis scheme (s6-1) is shown below.

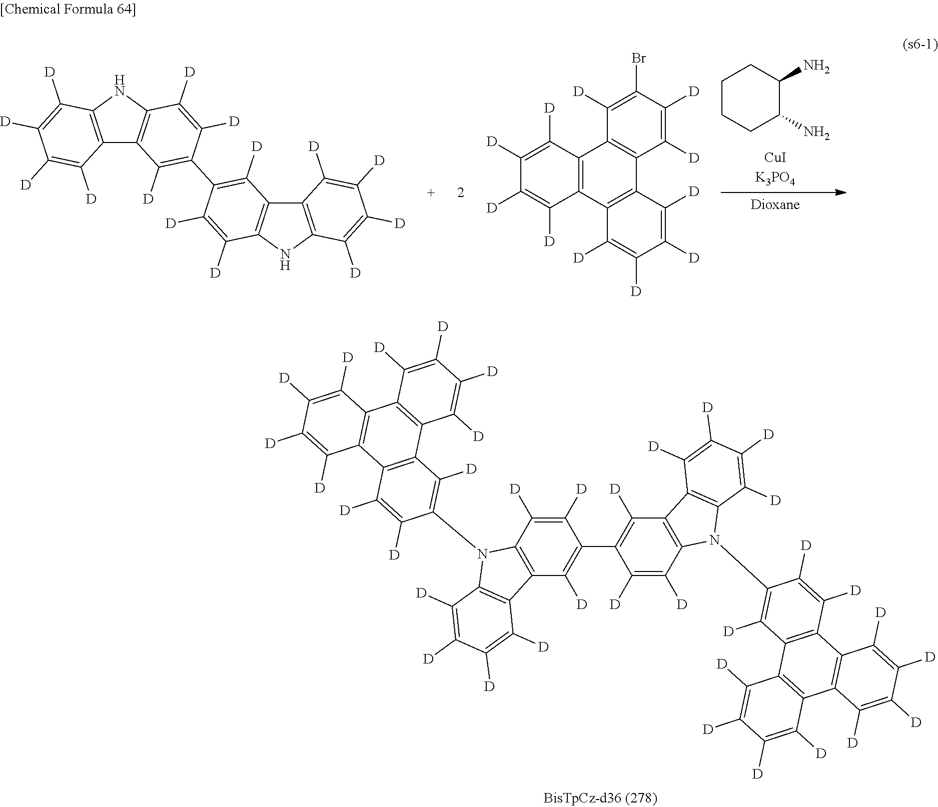

The molecular weight of the obtained white solid was measured by LC/MS analysis. As a result, m/z 821 was observed with respect to the calculated mass 821 of the target substance. This indicates that BisTpCz-d36 was obtained.

### Example 8

In this example, a light-emitting device 3 including βNCCαN-d28 (Structural Formula (287)), whose synthesis method is described in Example 3, in its light-emitting layer and a light-emitting device 4 including BisαNCz-d28 (Structural Formula (217)), whose synthesis method is described in Example 4, in its light-emitting layer were fabricated. The results of measuring the device characteristics are described.

The structural formulae of the organic compounds used in the light-emitting devices 3 and 4 are shown below.

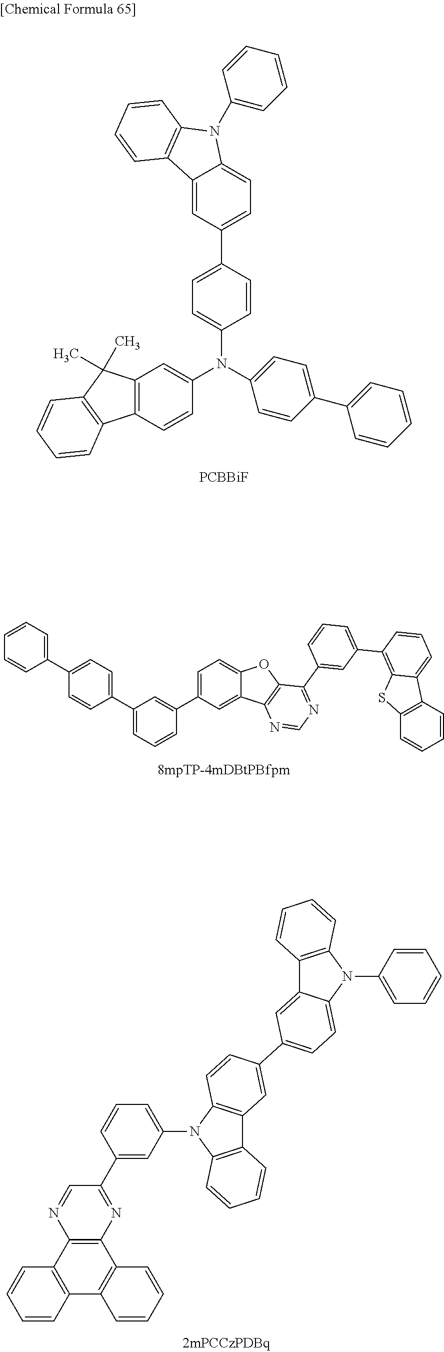

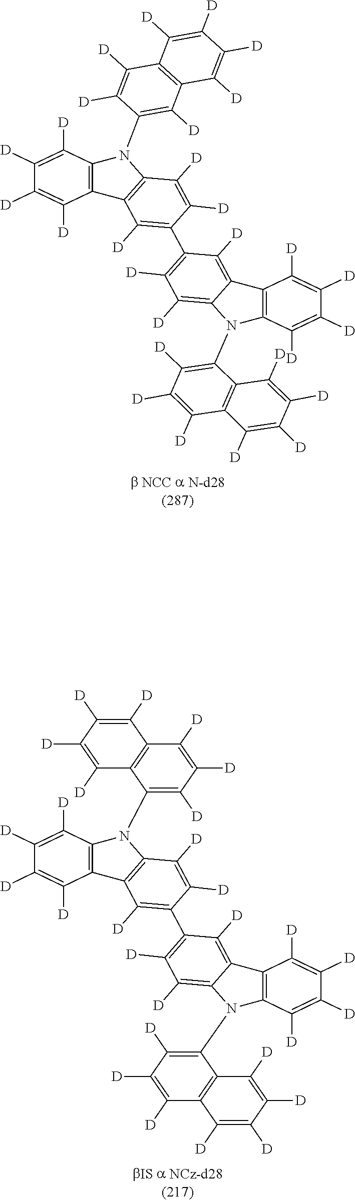 [SMILES: [2H]c1c([2H])c([2H])c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c([2H])c([2H])c([2H])c([2H])c56)c([2H])c([2H])c43)c([2H])c([2H])c2c1[2H] ; [2H]c1c([2H])c([2H])c2c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c([2H])c([2H])c([2H])c([2H])c56)c([2H])c([2H])c43)c([2H])c([2H])c([2H])c2c1[2H]]

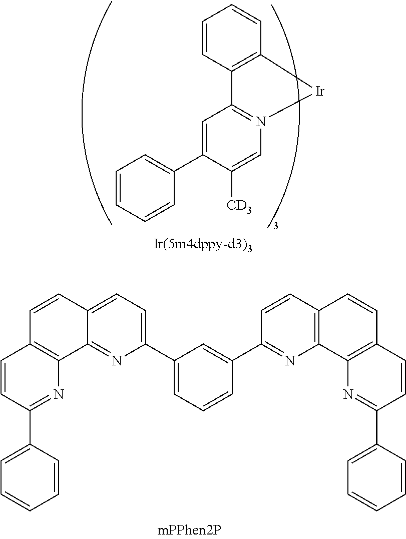

### <Method for Fabricating Light-Emitting Device 3>

The light-emitting device 3 is different from the light-emitting device 1 in that the thickness of the hole-transport layer 912 was 45 nm and that BisβNCz-d28 used for the light-emitting layer 913 was replaced with βNCCαN-d28. In the light-emitting device 3, 8mpTP-4mDBtPBfpm, βNCCαN-d28, and Ir(5m4dppy-d₃)₃ were deposited by co-evaporation to a thickness of 40 nm such that the weight ratio of 8mpTP-4mDBtPBfpm to βNCCαN-d28 to Ir(5m4dppy-d₃)₃ was 0.5:0.5:0.1, whereby the light-emitting layer 913 was formed. Components other than the hole-transport layer 912 and the light-emitting layer 913 of the light-emitting device 3 were fabricated as in the light-emitting device 1.

### <Method for Fabricating Light-Emitting Device 4>

The light-emitting device 4 is different from the light-emitting device 1 in that the thickness of the hole-transport layer 912 was 45 nm and that BisβNCz-d28 used for the light-emitting layer 913 was replaced with BisαNCz-d28. In the light-emitting device 4, 8mpTP-4mDBtPBfpm, BisαNCz-d28, and Ir(5m4dppy-d3)₃ were deposited by co-evaporation to a thickness of 40 nm such that the weight ratio of 8mpTP-4mDBtPBfpm to BisαNCz-d28 to Ir(5m4dppy-d3)₃ was 0.5:0.5:0.1, whereby the light-emitting layer 913 was formed. Components other than the hole-transport layer 912 and the light-emitting layer 913 of the light-emitting device 4 were fabricated as in the light-emitting device 1.

The device structures of the light-emitting devices 3 and 4 are listed in the following table.

TABLE 3

| | Thickness | | Light-emitting device 3 | Light-emitting device 4 |
|---|---|---|---|---|
| Second electrode | 200 | nm | Al | |
| Electron-injection layer | 1 | nm | LiF | |
| Electron-transport layer | 20 | nm | mPPhen2P | |
| | 10 | nm | 2mPCCzPDBq | |
| Light-emitting layer | 40 | nm | 8mpTP- | 8mpTP- |
| | | | 4mDBtPBfpm:βNCCαN- | 4mDBtPBfpm:BisαNCz- |
| | | | d28:Ir(5m4dppy-d3)₃ | d28:Ir(5m4dppy-d3)₃ |
| | | | (0.5:0.5:0.1)(weight ratio) | (0.5:0.5:0.1)(weight ratio) |

| | | | |
|---|---|---|---|
| Hole-transport layer | 45 | nm | PCBBiF |
| Hole-injection layer | 10 | nm | PCBBiF:OCHD-003 (1:0.03) |
| First electrode | 70 | nm | ITSO |

### <Characteristics of Light-Emitting Devices>

The light-emitting devices were sealed using a glass substrate in a glove box containing a nitrogen atmosphere so as not to be exposed to the air (a sealing material was applied to surround the devices and UV treatment and heat treatment at 80° C. for one hour were performed at the time of sealing). Then, the characteristics of the light-emitting devices were measured.

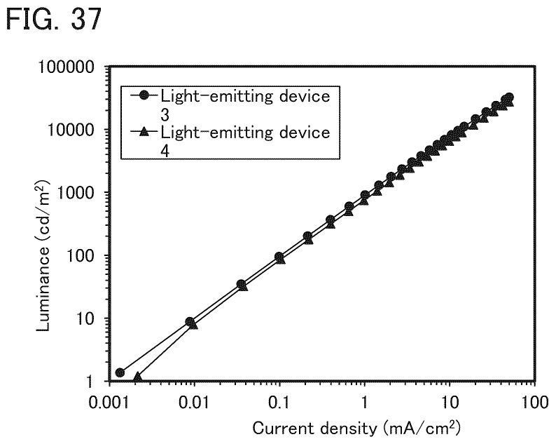

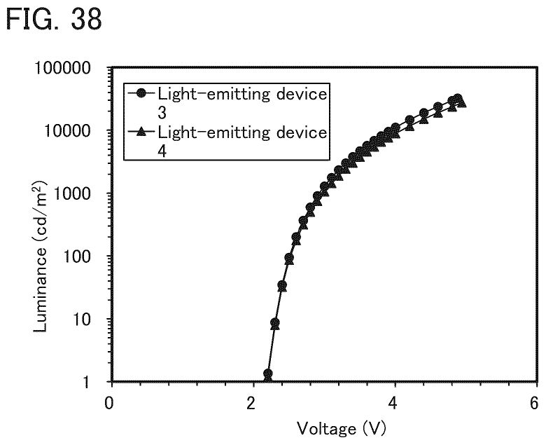

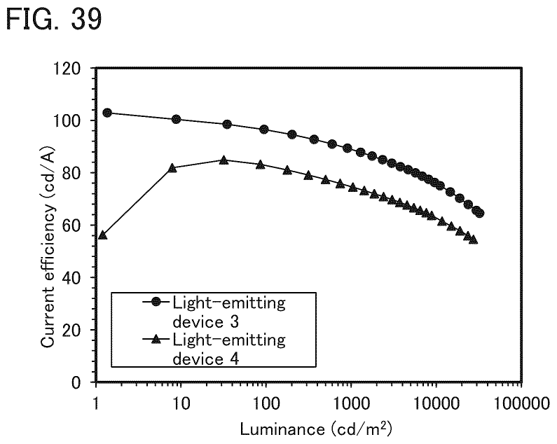

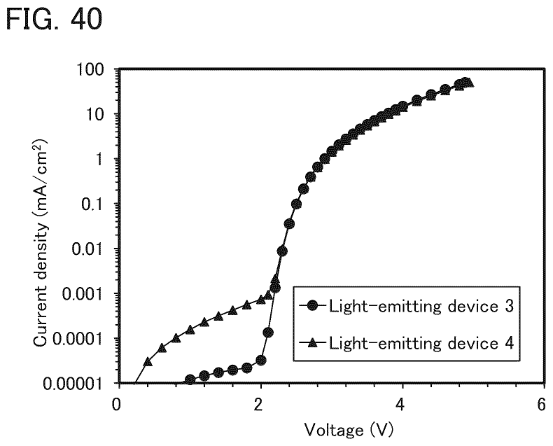

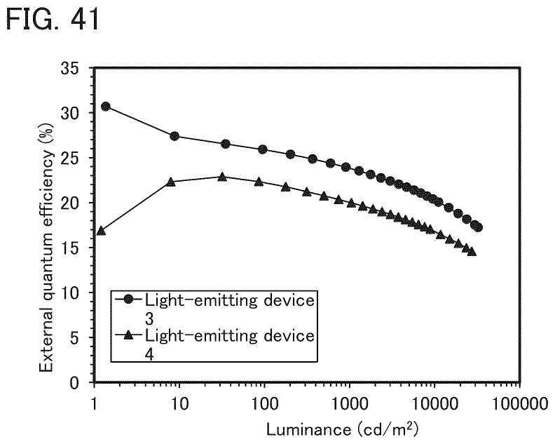

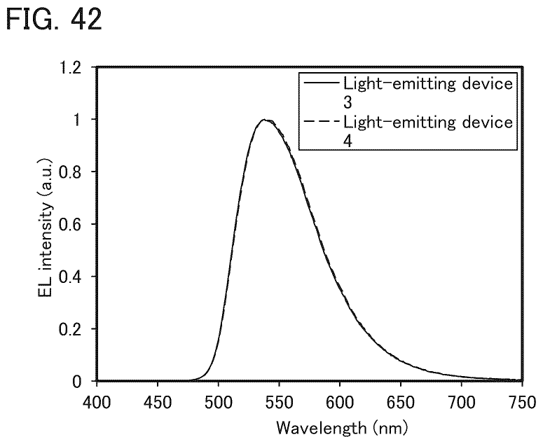

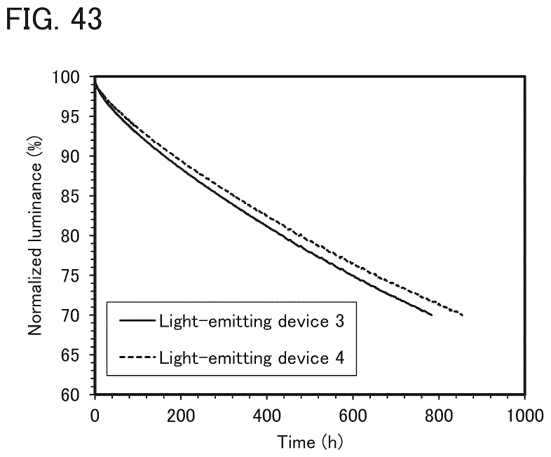

FIG. 37 shows the luminance-current density characteristics of the light-emitting devices, FIG. 38 shows the luminance-voltage characteristics thereof, FIG. 39 shows the current efficiency-luminance characteristics thereof, FIG. 40 shows the current density-voltage characteristics thereof, FIG. 41 shows the external quantum efficiency-luminance characteristics thereof, and FIG. 42 shows the electroluminescence spectra thereof. Furthermore, FIG. 43 shows a luminance change over driving time when the light-emitting devices were driven at a constant current of 2 mA (50 mA/cm²).

The following table shows the main characteristics of the light-emitting devices at a luminance of approximately 1000 cd/m². Note that the luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 4

| | | | | | | | | External |
|---|---|---|---|---|---|---|---|---|
| | | | Current | | | | Current | quantum |
| | Voltage | Current | density | Chroma- | Chroma- | Luminance | efficiency | efficiency |
| | (V) | (mA) | (mA/cm²) | ticity x | ticity y | (cd/m²) | (cd/A) | (%) |
| Light-emitting | 2.90 | 0.0404 | 1.01 | 0.371 | 0.603 | 903 | 89.3 | 23.9 |
| device 3 | | | | | | | | |
| Light-emitting | 3.00 | 0.0563 | 1.41 | 0.373 | 0.602 | 1048 | 74.5 | 20.0 |
| device 4 | | | | | | | | |

FIGS. 37 to 42 and the above table reveal that the light-emitting devices 3 and 4 exhibit green light emission derived from Ir(5m4dppy-d3)₃ and have favorable characteristics.

FIG. 43 reveals that the light-emitting devices 3 and 4 each have a small luminance change over driving time and a long lifetime.

The above results show that the use of the organic compound of one embodiment of the present invention enables fabrication of a light-emitting device with a long lifetime and high reliability.

### Reference Synthesis Example

In this synthesis example, a synthesis method of 9-(biphenyl-2,2′,3′,4,4′,5,5′,6,6′-d₉-3-yl)-9′-(naphthyl-1,3,4,5,6,7,8-d₇-2-yl)-3,3′-bi-9H-carbazole-1,1′,2,2′,4,4′,5,5′,6,6′,7,7′,8,8′-d₁₄ (abbreviation: βNCCmBP-d30), which is an organic compound usable in the light-emitting device of one embodiment of the present invention and is represented by Structural Formula (126) in Embodiment 1, will be described. The structure of βNCCmBP-d30 is shown below.

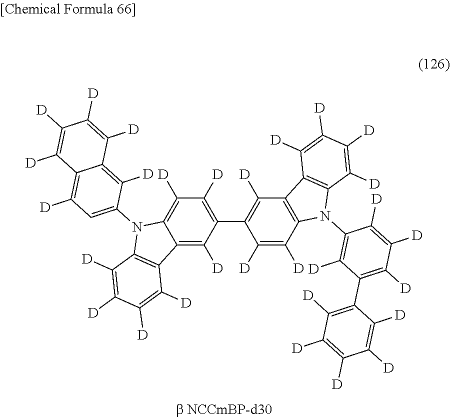 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cc([2H])c6c([2H])c([2H])c([2H])c([2H])c6c5[2H])c([2H])c([2H])c43)c2[2H])c([2H])c1[2H]]

### Step 1: Synthesis of 9-(biphenyl-2,2′,3′,4,4′,5,5′,6,6′-d₉-3-yl)-9H-carbazole-1,2,3,4,5,6,7,8-d₈

Into a 2 L three-neck flask were put 18.6 g (106 mmol) of 9H-carbazole-1,2,3,4,5,6,7,8-d₈, 25.6 g (106 mmol) of 5′-bromobiphenyl-2,2′,3,3′,4,4′,5,6,6′-d₉, 21.8 g (157 mmol) of potassium carbonate (abbreviation: K₂CO₃), 1.21 g (4.58 mmol) of 18-crown-6, and 600 mL of toluene. This mixture was degassed by being stirred under reduced pressure. Then, the atmosphere in the flask was replaced with nitrogen. After that, the mixture was heated at 60° C. To this mixture was added 1.60 g (3.13 mmol) of bis(tri-tert-butylphosphine)palladium(0) (abbreviation: Pd[P(tBu)₃]2), and stirring was performed at 120° C. for 12 hours. The mixture was air-cooled to room temperature and subjected to suction filtration through alumina, Celite, and Florisil. The obtained filtrate was concentrated to give a pale yellow solid. The solid was purified by high performance liquid chromatography (mobile phase: chloroform) to give 34.3 g of the target white solid in a yield of 96%. Synthesis Scheme (s8-1) of Step 1 is shown below.

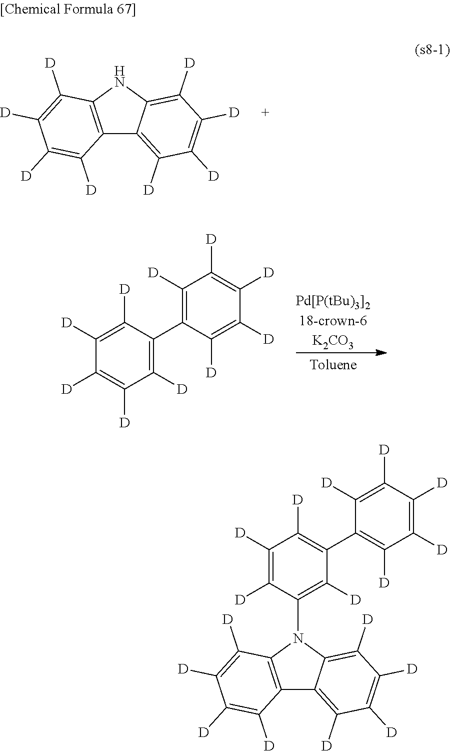

The molecular weight of the white solid obtained in Step 1 was measured by LC/MS analysis. As a result, m/z 336 was observed with respect to the calculated mass 336 of the target substance. This indicates that 9-(biphenyl-2,2′,3′,4,4′,5,5′,6,6′-d₉-3-yl)-9H-carbazole-1,2,3,4,5,6,7,8-d₈ was obtained.

### Step 2: Synthesis of 6-bromo-9-(biphenyl-2,2′,3′,4,4′,5,5′,6,6′-d₉-3-yl)-9H-carbazole-1,2,3,4,5,7,8-d₇)

Into a 2 L conical flask were put 34.3 g (102 mmol) of 9-(biphenyl-2,2′,3′,4,4′,5,5′,6,6′-d₉-3-yl)-9H-carbazole-1,2,3,4,5,6,7,8-d₈ and 800 mL of dichloromethane (CH₂Cl₂), and the mixture was stirred at room temperature until 9-(biphenyl-2,2′,3′,4,4′,5,5′,6,6′-d₉-3-yl)-9H-carbazole-1,2,3,4,5,6,7,8-d₈ was dissolved. Then, 18.1 g (102 mmol) of N-bromosuccinimide (NBS) was dissolved in 700 mL of ethyl acetate (AcOEt), and the obtained solution was transferred to a 1 L separatory funnel. While the dichloromethane solution was stirred, the ethyl acetate solution was dropped into the dichloromethane solution with use of the separatory funnel for 20 minutes; then, the obtained solution was stirred at room temperature for 65 hours. After the stirring, the obtained reaction solution was subjected to suction filtration through alumina, Celite, and Florisil. The obtained filtrate was concentrated to give 38.1 g of a pale yellow viscous solid containing the target substance. The solid was purified by high performance liquid chromatography (mobile phase: chloroform) to give 29.6 g of the target white solid in a yield of 70%. Synthesis Scheme (s8-2) of Step 2 is shown below.

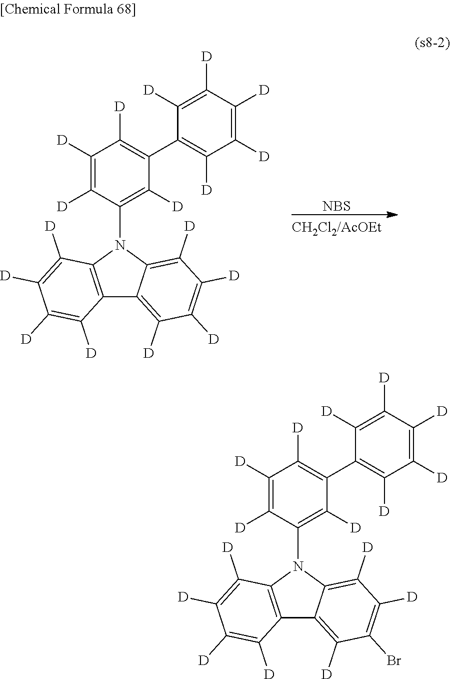 [SMILES: [2H]c1c([2H])c([2H])c(-c2c([2H])c([2H])c([2H])c(-n3c4c([2H])c([2H])c([2H])c([2H])c4c4c([2H])c([2H])c([2H])c([2H])c43)c2[2H])c([2H])c1[2H] ; "NBS" ; [2H]c1c([2H])c([2H])c(-c2c([2H])c([2H])c([2H])c(-n3c4c([2H])c([2H])c([2H])c([2H])c4c4c([2H])c(Br)c([2H])c([2H])c43)c2[2H])c([2H])c1[2H]]

The molecular weight of the white solid obtained in Step 2 was measured by LC/MS analysis. As a result, m/z 413 was observed with respect to the calculated mass 413 of the target substance. This indicates that 6-bromo-9-(biphenyl-2,2′,3′,4,4′,5,5′,6,6′-d₉-3-yl)-9H-carbazole-1,2,3,4,5,7,8-d₇ was obtained.

### Step 3: Synthesis of βNCCmBP-d30

Into a 300 mL three-neck flask were put 3.23 g (7.79 mmol) of 6-bromo-9-(biphenyl-2,2′,3′,4,4′,5,5′,6,6′-d₉-3-yl)-9H-carbazole-1,2,3,4,5,7,8-d₇, 3.72 g (8.54 mmol) of 6-(4,4,5,5-tetramethyl-1,3,2-dioxaborolane)-9-(2-naphthyl-1,3,4,5,6,7,8-d₇)-9H-carbazole-1,2,3,4,5,7,8-d₇, 3.24 g (23.4 mmol) of potassium carbonate (abbreviation: K₂CO₃), 99.2 mg (324 μmol) of tris(o-tolyl)phosphine (abbreviation: P(o-tolyl)₃, 12 mL of water, 8 mL of ethanol, and 60 mL of toluene. This mixture was degassed by being stirred under reduced pressure. Then, the atmosphere in the flask was replaced with nitrogen. After that, the mixture was heated at 80° C. To this mixture, 40.1 mg (178 μmol) of palladium(II) acetate (abbreviation: Pd(OAc)₂) was added, and the mixture was stirred at 120° C. for five hours. The reaction mixture was air-cooled to room temperature and was heated at 80° C. again; then, 35.3 mg (157 μmol) of Pd(OAc)₂ and 100 mg (326 μmol) of P(o-tolyl)₃ were added, and stirring was performed at 120° C. for three hours. The reaction solution was air-cooled to room temperature, suction filtration was performed through Celite and alumina, and then the obtained filtrate was concentrated to give an orange solid. The obtained solid was dissolved in toluene again, the obtained toluene solution was subjected to suction filtration through Celite and Florisil, and then the obtained filtrate was concentrated to give 5.13 g of a white solid. The obtained solid was purified by high performance liquid chromatography (mobile phase: chloroform) to give 3.94 g of the target white solid in a yield of 79%. Synthesis Scheme (s8-3) of βNCCmBP-d30 is shown below.

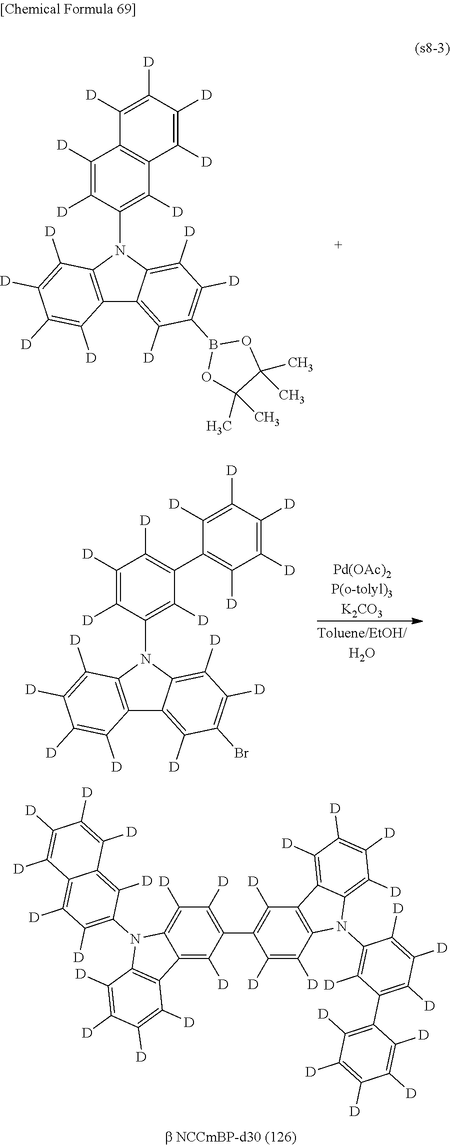

The molecular weight of the white solid obtained in Step 3 was measured by LC/MS analysis. As a result, m/z 640 was observed with respect to the calculated mass 640 of the target substance. This indicates that βNCCmBP-d30 was obtained. This application is based on Japanese Patent Application Serial No. 2024-096666 filed with Japan Patent Office on Jun. 14, 2024, the entire contents of which are hereby incorporated by reference.

What is claimed is:

1. An organic compound represented by General Formula (G4): 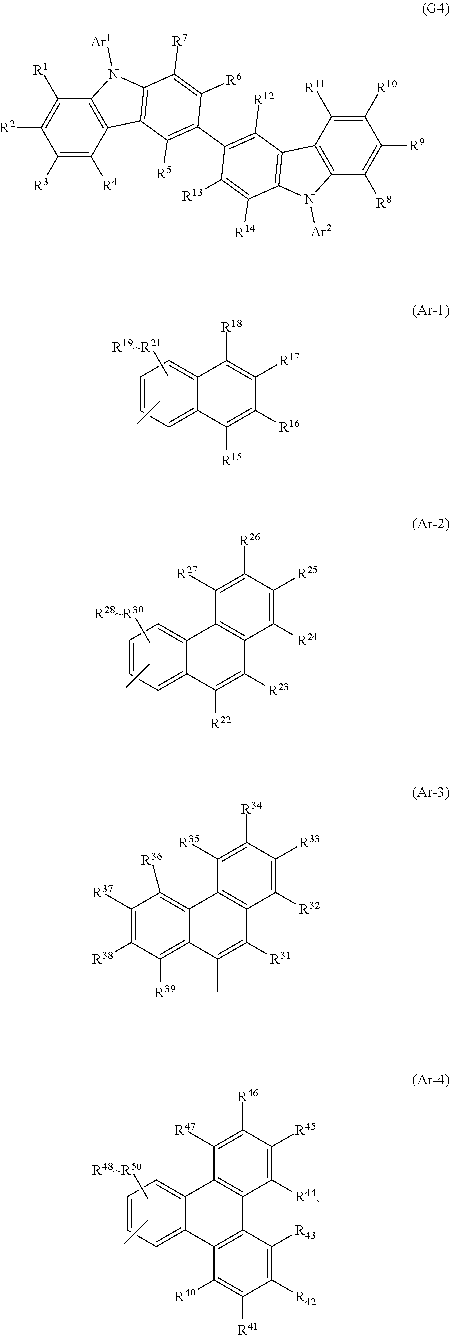
wherein, Ar¹ and Ar² are each independently a group represented by any one of General Formulae (Ar-1) to (Ar-4) and comprise the same fused ring,
wherein R¹ to R¹⁴ each independently represent hydrogen,
wherein R¹⁵ to R⁵⁰ each independently represent hydrogen or an alkyl group having 1 to 6 carbon atoms, and
wherein the organic compound represented by General Formula (G4) comprises two or more deuteriums.

2. An organic compound represented by General Formula (G5): 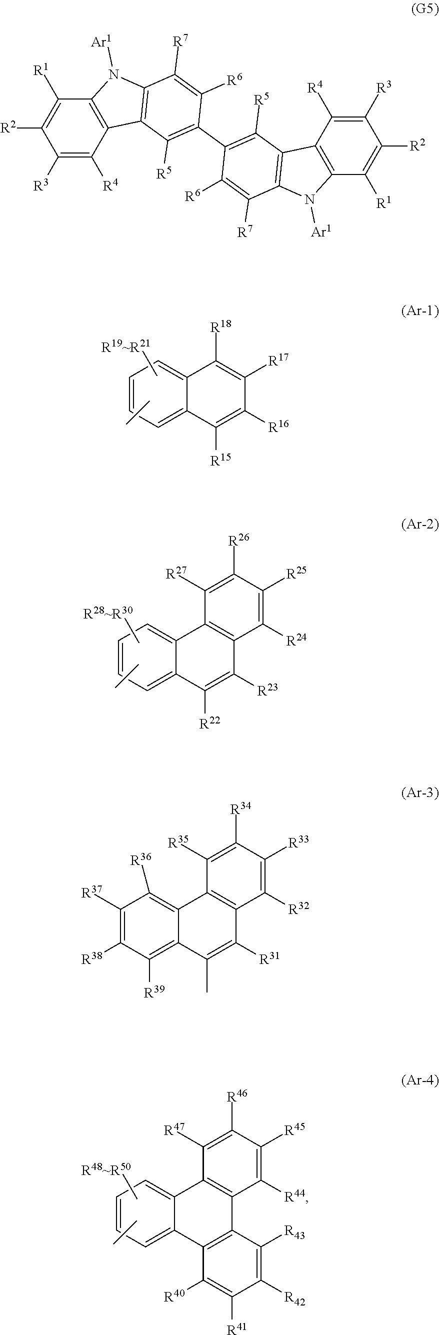
wherein Ar¹ is a group represented by any one of General Formulae (Ar-1) to (Ar-4),
wherein R¹ to R⁷ each independently represent hydrogen,
wherein R¹⁵ to R⁵⁰ each independently represent hydrogen or an alkyl group having 1 to 6 carbon atoms, and
wherein the organic compound represented by General Formula (G5) comprises two or more deuteriums.

3. The organic compound according to claim 2, wherein the organic compound is represented by General Formula (G6): 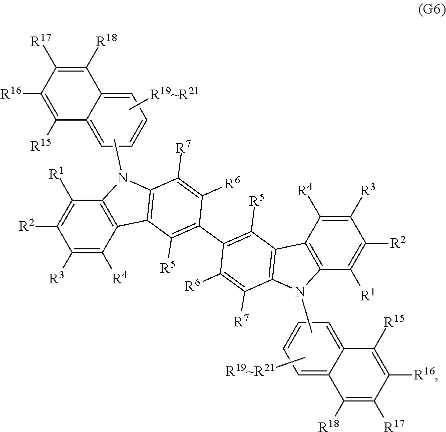
wherein the organic compound represented by General Formula (G6) comprises two or more deuteriums.

4. The organic compound according to claim 3, wherein the organic compound is represented by General Formula (G7): 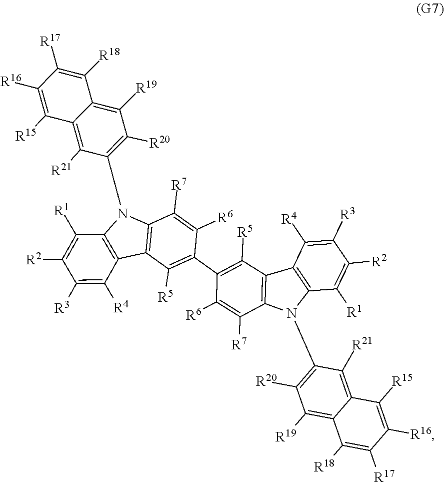
wherein the organic compound represented by General Formula (G7) comprises two or more deuteriums.

5. An organic compound represented by Structural Formula (200), (217), (260), (270), (278), or (287):

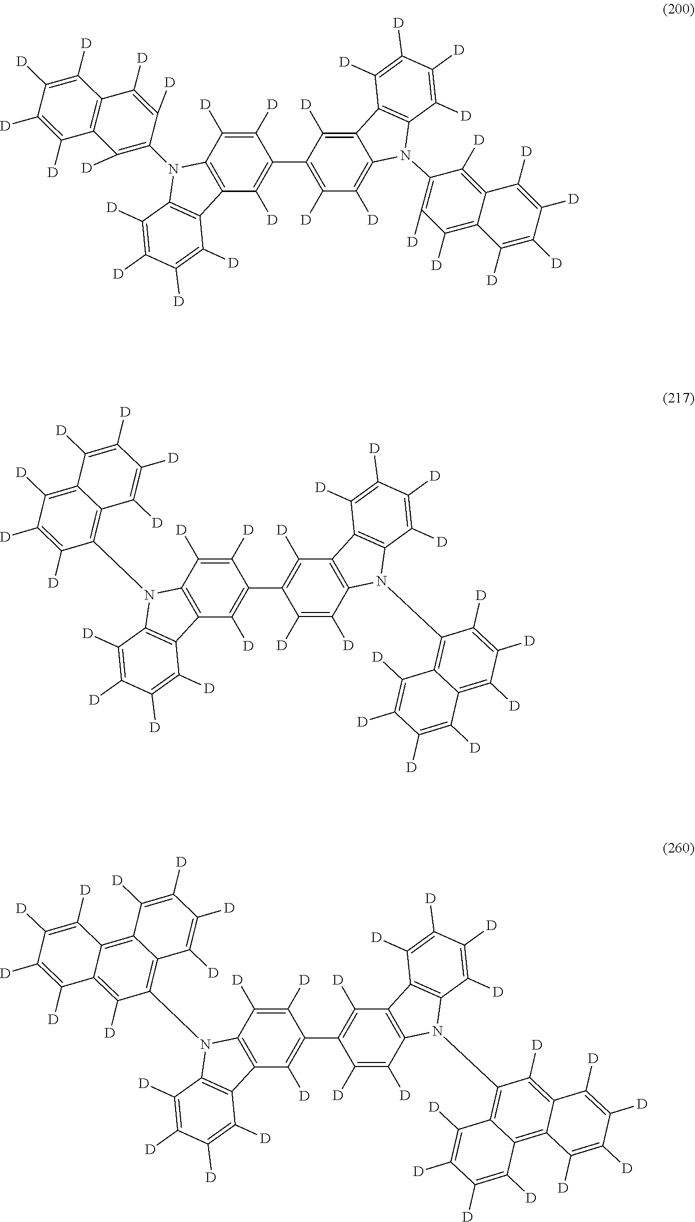

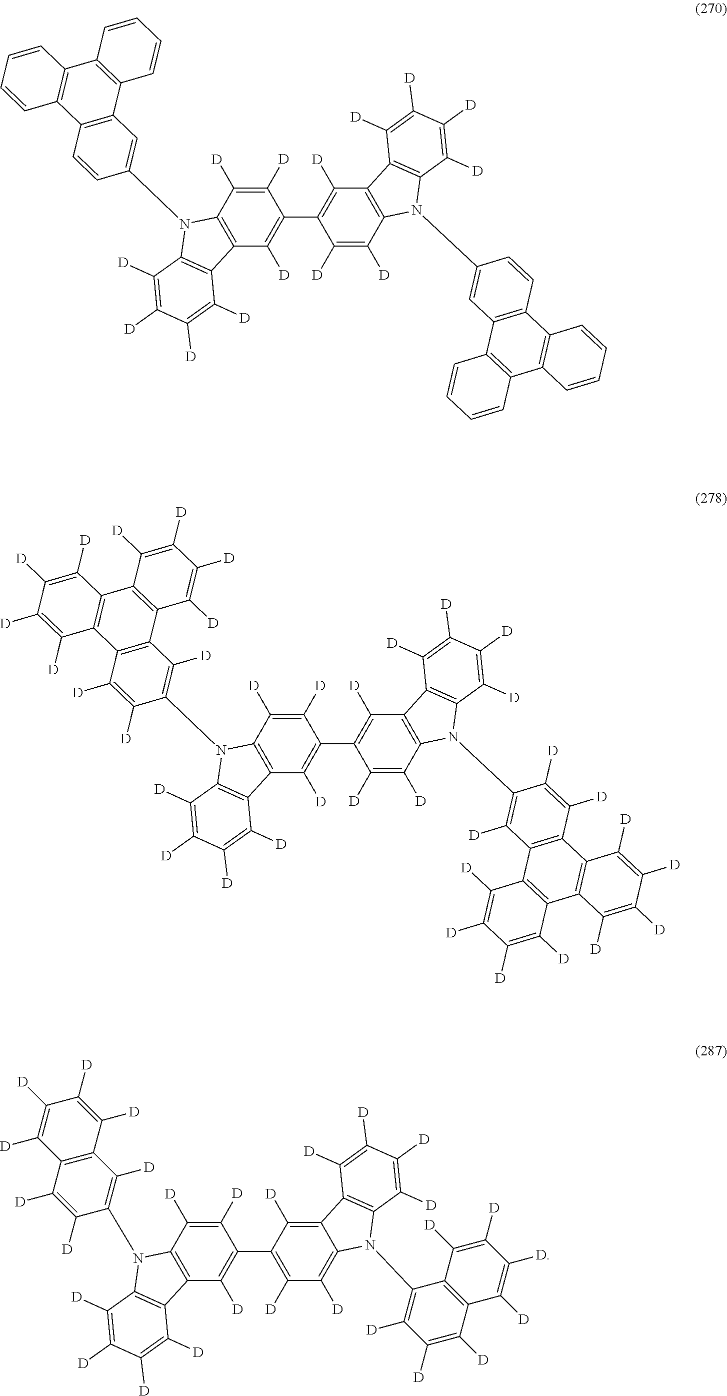

6. A light-emitting device comprising the organic compound according to claim 1.

7. A light-emitting device comprising the organic compound according to claim 2.

8. A light-emitting device comprising the organic compound according to claim 5.
